(19) United States
(12) Patent Application Publication
(10) Pub. No.: US 2021/0087236 A1
(43) Pub. Date: Mar. 25, 2021

(54) TARDIGRADE DISORDERED PROTEINS AS PROTEIN STABILIZERS

(71) Applicants: The University of North Carolina at Chapel Hill, Chapel Hill, NC (US); North Carolina State University, Raleigh, NC (US)

(72) Inventors: Thomas Christopher Clark Boothby, Chapel Hill, NC (US); Robert Patrick Goldstein, Carrboro, NC (US); Gary Joseph Pielak, Chapel Hill, NC (US); Samantha Piszkiewicz, Chapel Hill, NC (US); Alexandra Harrison Brozena, Chapel Hill, NC (US)

(21) Appl. No.: 16/325,467

(22) PCT Filed: Aug. 4, 2017
(86) PCT No.: PCT/US2017/045511

§ 371 Date: Feb. 14, 2019

Related U.S. Application Data
(60) Provisional application No. 62/375,238, filed on Aug. 15, 2016.

(51) Int. Cl. C07K 14/435; A61K 47/42; C12N 15/82; C12N 15/74; C12N 15/81; A23L 33/18; A23J 1/00

(52) U.S. Cl. CPC C07K 14/43504; A61K 47/42; C12N 15/8273; A23V 2002/00; C12N 15/81; A23L 33/18; A23J 1/008; C12N 15/74

(57) ABSTRACT

The present invention relates to methods and compositions for stabilizing proteins. The invention provides compositions comprising at least one tardigrade disordered protein (TDP) and at least one heterologous polypeptide and/or peptide of interest. Further provided are methods for stabilizing proteins and for producing organisms and cells having increased tolerance to desiccation and/or drought.

### STATEMENT OF PRIORITY

This application claims the benefit, under 35 U.S.C. § 119 (e), of U.S. Provisional Application No. 62/375,238, filed on Aug. 15, 2016 in the United' States Patent and Trademark Office, the entire contents of which is incorporated by reference herein.

### STATEMENT OF FEDERAL SUPPORT

This invention was made with Government support under NNX15AB446G awarded by the National Aeronautics and Space Administration and under MCB 1051819 awarded by the National Science Foundation. The United States Government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5470-793PR_ST25.bct, 487,526 bytes in size, generated Aug. 11, 2016 and filed via EFS-Web, is provided in lieu of a paper copy. This Sequence Listing is hereby incorporated herein by reference into the specification for its disclosures.

### FIELD OF THE INVENTION

The invention relates to methods and compositions for stabilizing proteins using tardigrade proteins.

### BACKGROUND OF THE INVENTION

Many vaccines and protein based pharmaceuticals have limited shelf lives and are structurally and functionally unstable, requiring them to be produced, transported, and stored using a system of refrigerators and freezers known as the “cold-chain.” This makes many of these lifesaving drugs difficult and expensive to manufacture and deliver.

Although numerous molecules are used as crowding agents to stabilize pharmaceuticals in liquid formulations, these additives can be flawed. For example, non-reducing sugars like manitol, sorbitol, and trehalose are effective in solution but are prone to crystallization and phase separation upon freezing. (Shire, S. J. Curr. Opin. Biotechnol. 20, 708-714 (2009)). Sucrose does not have this problem, but its hydrolysis results in unwanted glycosylation of pharmaceuticals (Shire, S. J. Curr. Opin. Biotechnol. 20, 708-714 (2009)). Surfactants are also common additives; however, surfactants, such as polysorbate 20 and 80, produce peroxides that oxidize methionine groups (Shire, S. J. Curr. Opin. Biotechnol. 20, 708-714 (2009)). Recombumin®, human serum albumin heterologously expressed in and purified from yeast, is also used as a stabilizer in drug formulation. However, formulations containing Recombumin® still require refrigeration (AlbumedFix. RECOMBUMINC FORMULATE WITH CONFIDENCE (2016)). These stabilizers and others have extended the half-lives of many pharmaceuticals, but none have eliminated the requirement of low-temperature storage for liquid formulations. Furthermore, many potential protein-based pharmaceuticals never make it to the market because they are deemed too unstable even with low-temperature storage and the addition of stabilizing additives.

Some protein-based pharmaceuticals can be stored at room temperature if they are lyophilized (freeze dried); however, most protein-based pharmaceuticals denature as a result of either the freezing or drying process. Sometimes crowding agents can protect protein-based pharmaceuticals during lyophilization, but none of these crowding agents work universally. The most effective additives for a given pharmaceutical is highly dependent on factors including the pI, β-sheet content, and melting temperature of the drug (Roughton et al. Comput. Chem. Eng. 58, 369-377 (2013)). Even with the addition of stabilizers, many protein-based pharmaceuticals are too unstable to survive lyophilization (Roughton et al. Comput. Chem. Eng. 58, 369-377 (2013)).

The present invention overcomes previous shortcomings in the art by providing new compositions and methods for stabilizing proteins and other biomedical material.

### SUMMARY OF THE INVENTION

One aspect of the invention provides a liquid composition comprising: at least one tardigrade disordered protein (TDP); and at least one heterologous polypeptide and/or peptide of interest.

A second aspect provides a solid composition comprising: at least one tardigrade disordered protein (TDP); and at least one heterologous polypeptide and/or peptide of interest.

A third aspect of the invention provides a recombinant nucleic acid construct comprising: (a) a nucleotide sequence of any one of SEQ ID NOs:106-210, or a complement thereof; (b) a nucleotide sequence of any one of SEQ ID NOs:211-315; (c) a nucleotide sequence that encodes a polypeptide comprising an amino acid sequence of any one of SEQ ID NOs: 1-105; (d) a nucleotide sequence having at least 80% sequence identity to the nucleotide sequence of any one of (a) to (c); (e) a nucleotide sequence which anneals under stringent hybridization conditions to the nucleotide sequence of any one of (a) to (d), or a complement thereof; (f) a nucleotide sequence that differs from the nucleotide sequences of any one of (a) to (e) above due to the degeneracy of the genetic code; (g) a functional fragment of a nucleotide sequence of any one of (a) to (f); and (h) any combination of the nucleotide sequences of (a)-(g). In some embodiments, the nucleotide sequence is operatively linked to a heterologous promoter.

In a fourth aspect, an isolated polypeptide is provided comprising: (a) an amino acid sequence of any one of SEQ ID NOs: 1-105; (b) an amino acid sequence encoded by a nucleotide sequence of any one of SEQ ID NOs:106-210, or a complement thereof; (c) an amino acid sequence encoded by a nucleotide sequence of any one of SEQ ID NOs:211-315; or (d) an amino acid sequence having at least about 80% sequence identity to the amino acid sequence of any one of (a) to (c).

In a fifth aspect, the present invention provides a method of stabilizing at least one heterologous polypeptide and/or peptide of interest, comprising contacting the at least one heterologous polypeptide and/or peptide of interest with at least one tardigrade disordered protein (TDP), to produce a liquid composition comprising the at least one heterologous polypeptide and/or peptide of interest and at least one TDP, thereby stabilizing the at least one heterologous polypeptide and/or peptide of interest.

In a sixth aspect, a method of stabilizing a heterologous cell, tissue or organ is provided, comprising contacting the heterologous cell, tissue or organ with a solution comprising at least one tardigrade disordered protein (TDP), thereby stabilizing the heterologous cell, tissue or organ.

In a seventh aspect, a method of producing a transgenic cell having increased tolerance to drought or desiccation is provided, comprising: introducing into a cell a heterologous nucleotide sequence encoding a tardigrade disordered protein (TDP), thereby producing a transgenic cell having increased tolerance to drought or desiccation.

In an eighth aspect, a method of increasing drought or desiccation tolerance in an organism is provided comprising introducing into the organism a heterologous nucleotide sequence encoding a tardigrade disordered protein (TDP), to produce a transgenic organism expressing the heterologous nucleotide sequence, thereby increasing the drought or desiccation tolerance of the transgenic organism.

Further provided are transgenic organisms and/or transgenic cells comprising the heterologous nucleotide sequences or recombinant nucleic acid constructs of the invention.

These and other aspects of the invention are set forth in more detail in the description of the invention below.

### BRIEF DESCRIPTION OF THE DRAWINGS

FIG. 1A-1B show that tardigrades upregulate genes encoding tardigrade-specific intrinsically disordered proteins as they dry. FIG. 1A shows published data on the survival versus relative humidity for Hypsibius dujardini (circles), Paramacrobiotus richtersi (squares), and Milnesium tardigradum (triangles). Data from Table 1 of Wright (J. Exp. Biol. 142, 267-292 (1989)) Animals desiccated at lower relative humidity experience increased rates of desiccation compared to those desiccated at higher relative humidity. FIG. 1B shows survival of H. dujardini after slow drying (95% RH), quick drying (70% RH) and slow drying followed by quick drying. T-test: ns=not significant, **p<0.001.

FIG. 2A-2B show that TDPs are essential for efficient survival of desiccation. Survival after RNAi injection targeting GFP (control), CAHS, or SAHS transcripts in hydrated (FIG. 2A) and dry (FIG. 2B) Hypsibius dujardini specimens. Dots represent individual trials. N=10 for each individual trial (30 total). T-test: ns=not significant, *p<0.01, ** p<0.001. RNA abundance fold change values given above each bar (e.g., 17X), indicate the increase in abundance in dry relative to hydrated conditions.

FIGS. 3A-3B show divergence in H dujardini's response to drying and freezing. FIG. 3A provides a heat map showing correlation between expression profiles of transcriptomes derived from dry, frozen, and hydrated H dujardini specimens. FIG. 3B shows survival under frozen conditions of H dujardini specimens injected with RNAi constructs targeting control (1st bar), CAHS (2nd through 5th bars), and SAHS (6th through 9th bars) genes. Dots represent individual trials with N=10 for each individual trial (30 total). T-test: ns=not significant. RNA abundance fold change values given above each bar (e.g. 1.2X), indicate the increase in abundance of that transcript in frozen relative to hydrated conditions.

FIG. 4A-4B shows that exogenous expression of CAHS proteins is sufficient to increase desiccation tolerance in prokaryotic and eukaryotic cells. FIG. 4A shows desiccation tolerance (% survival) of yeast expressing CAHS genes. FIG. 4B shows desiccation tolerance (number of colony forming units/10⁸ cells) of E. coli BL21 bacteria expressing CAHS or control (α-synuclein) IDPs. Dots represent individual trials. T-test: ns=not significant, *p<0.01, ** p<0.001, *** p<0.0001.

FIG. 5A-F: Drying induces TDPs to form bioglasses, which correlates with desiccation tolerance. (FIG. 5A) Overlaid differential scanning calorimetry (DSC) thermograms from preconditioned (upper curve) and nonconditioned (lower curve) Hypsibius dujardini specimens. Step-like peak in preconditioned sample indicative of a glassy material transitioning to a liquid state. (FIG. 5B) Overlaid thermograms showing glass transition of purified a TDP (CAHS107838) measured in triplicate. Additional thermograms are presented in FIG. S5. (FIG. 5C) Overlaid thermograms showing the lack of glass transition of dry purified lysozyme measured in triplicate. (FIG. 5D) Overlaid thermograms of yeast control (empty vector; upper three curves) and TDP expressing (CAHS59302) strains (lower three curves). Shaded region highlights range of CAHS glass transition. (FIG. 5E) Desiccation tolerance (% survival) of H dujardini (tardigrade) specimens after heating to various temperatures. Shaded region highlights glass transition temperature range (see FIG. 5A). Dots represent individual trials with n=10 for each individual trial (total 30). (FIG. 5F) Desiccation tolerance (% survival) of yeast expressing TDPs heated to various temperatures. Shaded region highlights glass transition temperature range (see FIG. 5D). Dots represent individual trials.

FIG. 6 shows that TDPs stabilize protein folding under hydrated conditions. ¹⁹F NMR spectra comparing SH3 suspended in 36 g/L CAHS G (broken line) to SH3 in buffer alone (solid line). Arrow indicates decrease in unfolded state which occurs when SH3 is incubated with TDPs.

FIG. 7 shows that TDPs increase and maintain protein function under desiccated conditions. 0.1 g/L of LDH was desiccated and rehydrated without additives (black) and in the presence of various concentrations of TDPs: CAHS G (first curve) and CAHS D (second curve), or other non-TDP additives: BSA (third curve) and trehalose (fourth curve). The percent activity remaining was determined by comparison to a control of the same solution that had been stored at 4° C. All experiments were run in triplicate.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to Illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like refers to variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “express,” “expresses,” “expressed” or “expression,” and the like, with respect to a nucleic acid molecule and/or a nucleotide sequence (e.g., RNA or DNA) indicates that the nucleic acid molecule and/or a nucleotide sequence is transcribed and, optionally, translated. Thus, a nucleic acid molecule and/or a nucleotide sequence may express a polypeptide of interest or a functional untranslated RNA.

As used herein, “contact,” “contacting,” “contacted,” and grammatical variations thereof, refers to placing the components of a desired reaction together under conditions suitable for carrying out the desired reaction (e.g., stabilizing the polypeptide, peptide, cell, tissue or organ). The term “contact” may comprise any method in which a polypeptide, peptide, cell, organ and/or tissue is exposed to, provided with, or in which a TDP is applied.

A “heterologous polypeptide and/or peptide of interest” as used herein, refers to a non-tardigrade polypeptide and/or peptide, or a polypeptide and/or peptide that is heterologous to the organism, to the genus or to the species from which the particular TDP is derived.

A “heterologous cell, tissue or organ” as used herein, refers to a cell, tissue or organ that is heterologous to the organism, to the genus or to the species that naturally produces the particular TDP.

As used herein, “stabilizing” a heterologous polypeptide and/or peptide (and/or the polypeptides and/or peptides in cells, tissues, and/or organs) means maintaining the structure (1°, 2°, 3° and/or 4° structure) and the function of the polypeptide and/or peptide under either aqueous conditions or dried conditions, or after being frozen and/or dried and then thawed and/or rehydrated. In some embodiments, the at least one heterologous polypeptide and/or peptide of interest (and/or the polypeptides and/or peptides in cells, tissues, and/or organs) may be stable at a temperature from about −80° C. to about 100° C. once the at least one heterologous polypeptide and/or peptide of interest (and/or cell, tissue, and/or organ) is contacted with the at least one TDP. In some embodiments, at least about 10% to about 100% (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any range or value therein) of the structure and function of the stabilized polypeptide and/or peptide (and/or cell, tissue and/or organ) is maintained. Thus, in some embodiments, about 10% to about 90%, about 10 to about 85% about 10% to about 80%, about 10% to about 75%, about 10% to about 70%, about 10% to about 60%, about 10% to about 50%, about 20% to about 90%, about 20% to about 85%, about 20% to about 80%, about 20% to about 75%, about 20% to about 70%, about 20% to about 60%, about 20% to about 50%, about 30% to about 90%, about 30 to about 85%, about 30% to about 80%, about 30% to about 75%, about 30% to about 70%, about 30% to about 60%, about 30% to about 50%, about 40% to about 90%, about 40 to about 85%, about 40% to about 80%, about 40% to about 75%, about 40% to about 70%, about 40% to about 60%, about 40% to about 50%, about 50% to about 90%, about 50 to about 85%, about 50% to about 80%, about 50% to about 75%, about 50% to about 70%, about 50% to about 60%, and the like, of the structure and function of the stabilized polypeptide and/or peptide (and/or cell, tissue and/or organ) is maintained. In representative embodiments, when dried (e.g., solid compositions), the polypeptides and/or peptides (and/or the polypeptides and/or peptides in cells, tissues, and/or organs) may be stablized over a range of temperature from about -80° C. to about 100° C. In further representative embodiments, the polypetides and/or peptides (and/or the polypeptides and/or peptides in cells, tissues, and/or organs) in solution (liquid composition) may be stabilized over a range of temperatures from about −80° C. to about 40° C.

As used herein, “stabilizing” a cell, organ or tissue means maintaining the structure and function of a cell, organ or tissue under either aqueous conditions or dried conditions, or after being frozen and/or dried and then thawed and/or rehydrated.

As used herein, a “cell, organ and/or tissue” refers to any cell, organ or tissue from an organism useful with this invention (e.g., a fungus, a bacterium, a plant, an animal). In some embodiments, an organ and/or tissue may include, but is not limited to, lung, liver, bladder, kidney, heart, brain, stomach, intestines (large and small), eye or any part thereof (e.g., lens, cornea), ear or any part thereof (e.g., earlobe, cochlea), gallbladder, esophagus, salivary gland, tongue, teeth, pancreas, ureter, urethra, ovary, uterus, vagina, fallopian tube, testes, vas deferens, penis, pituitary gland, thyroid gland, adrenal gland, lymph node, spleen, thymus, bone marrow, skin (including subcutaneous skin), connective tissue, muscle tissue, nervous tissue, epithelial tissue, mineralized tissue, meristematic tissue, petal, sepal, stamen, pistil, anther, pollen, flower, fruit, flower bud, ovule, seed, embryo, petiole, stem, root, coleoptile, stalk, shoot, branch, apical meristem, axillary bud, cotyledon, hypocotyl, and leaf, callus tissue, protoplast, hyphae, and/or hymenium.

As used herein, the terms “increase,” “increasing,” “increased,” “enhance,” “enhanced,” “enhancing,” and “enhancement” (and grammatical variations thereof) describe an elevation of at least about 25%, 50%, 75%, 100%, 150%, 200%, 300%, 400%, 500% or more as compared to a control.

An “increased tolerance to drought or desiccation” as used herein refers to the ability of an organism or part thereof that has been either contacted with at least one TDP, or transformed with at least one heterologous nucleotide sequence encoding a TDP to withstand exposure to drought, or desiccation (e.g., water loss) better than a control organism or part thereof (i.e., an organism or part thereof that has been exposed to drought or desiccation but was not contacted with the at least one TDP or transformed with at least one heterologous nucleotide sequence encoding a TDP as described herein). Increased tolerance to drought or desiccation can be measured using a variety of parameters including, but not limited to, survival, metabolic capacity, reproductive capacity, ability to germinate, developmental potential, structural integrity, functional integrity, viability, morphological integrity, decreased necrosis/apoptosis, time required to recover to predesiccation/drought levels of metabolism, cell division, reproduction, germination, development, and/or function as compared to an organism or part thereof exposed to the same stress but not having been contacted with said composition.

A “part of an organism” (e.g., part thereof) refers to a multicellular organism and includes but is not limited to a cell, an organ, and other tissues from the organism. A “part of an organism” may also include, but is not limited to, nucleic acids, proteins, lipids, carbohydrates, and the like, that are present in an organism.

An isolated cell refers to a cell that is separated from other components with which it is normally associated in its natural state. For example, an isolated cell can be a cell in culture medium and/or a cell in a pharmaceutically acceptable carrier.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount. A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism. A “homologous” nucleic acid sequence is a nucleotide sequence naturally associated with a host cell into which it is introduced.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

The terms “complementary” or “complementarity,” as used herein, refer to the natural binding of polynucleotides under permissive salt and temperature conditions by base-pairing. For example, the sequence “A-G-T” binds to the complementary sequence “T-C-A.”

Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands. The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

In some embodiments, the recombinant nucleic acid molecules, nucleotide sequences and polypeptides of the invention are “isolated.” An “isolated” nucleic acid molecule, an “isolated” nucleotide sequence or an “isolated” polypeptide is a nucleic acid molecule, nucleotide sequence or polypeptide that, by the hand of man, exists apart from its native environment and is therefore not a product of nature. An isolated nucleic acid molecule, nucleotide sequence or polypeptide may exist in a purified form that is at least partially separated from at least some of the other components of the naturally occurring organism or virus, for example, the cell or viral structural components or other polypeptides or nucleic acids commonly found associated with the polynucleotide. In representative embodiments, the isolated nucleic acid molecule, the isolated nucleotide sequence and/or the isolated polypeptide is at least about 1%, 5%, 10%, 20%, 30%, 40%, 50%, 60%, 70%, 80%, 90%, 95%, or more pure.

In other embodiments, an isolated nucleic acid molecule, nucleotide sequence or polypeptide may exist in a non-native environment such as, for example, a recombinant host cell. Thus, for example, with respect to nucleotide sequences, the term “isolated” means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the recombinant nucleic acid molecules, nucleotide sequences and their encoded polypeptides are “isolated” in that, by the hand of man, they exist apart from their native environment and therefore are not products of nature, however, in some embodiments, they can be introduced into and exist in a recombinant host cell.

In some embodiments, the nucleotide sequences and/or recombinant nucleic acid molecules of the invention can be operatively associated with a variety of promoters for expression in soybean plant cells. Thus, in representative embodiments, a recombinant nucleic acid of this invention can further comprise one or more promoters operably linked to one or more nucleotide sequences.

By “operably linked” or “operably associated” as used herein, it is meant that the indicated elements are functionally related to each other, and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

A “promoter” is a nucleotide sequence that controls or regulates the transcription of a nucleotide sequence (i.e., a coding sequence) that is operably associated with the promoter. The coding sequence may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). Promoters can include, for example, constitutive, inducible, temporally regulated, developmentally regulated, chemically regulated, tissue-preferred and/or tissue-specific promoters for use in the preparation of recombinant nucleic acid molecules, i.e., “chimeric genes” or “chimeric polynucleotides.” In particular aspects, a “promoter” useful with the invention is a promoter capable of initiating transcription of a nucleotide sequence in a cell of interest. The choice of promoter will vary depending on the temporal and spatial requirements for expression, and also depending on the host cell to be transformed.

The terms “coding region” and “coding sequence” are used interchangeably and refer to a polynucleotide region that encodes a polypeptide or functional RNA and, when placed under the control of appropriate regulatory sequences, expresses the encoded polypeptide or functional RNA. The boundaries of a coding region are generally determined by a translation start codon at its 5′ end and a translation stop codon at its 3′ end. A coding region can encode one or more polypeptides or functional RNAs. For instance, a coding region can encode a polypeptide or functional RNA that is subsequently processed into two or more polypeptides or functional RNAs. A regulatory sequence or regulatory region is a nucleotide sequence that regulates expression of a coding region to which it is operably linked. Nonlimiting examples of regulatory sequences include promoters, transcription initiation sites, translation start sites, internal ribosome entry sites, translation stop sites, and terminators. “Operably linked” refers to a juxtaposition wherein the components so described are in a relationship permitting them to function in their intended manner. A regulatory sequence is “operably linked” to a coding region when it is joined in such a way that expression of the coding region is achieved under conditions compatible with the regulatory sequence.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention.

As used herein, a “functional” polypeptide or “functional fragment” is one that substantially retains at least one biological activity normally associated with that polypeptide (e.g., target protein binding). In particular embodiments, the “functional” polypeptide or “functional fragment” substantially retains all of the activities possessed by the unmodified peptide. By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as protein binding can be measured using assays that are well known in the art and as described herein.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity (e.g., at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to said nucleotide sequence of the invention.

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

As used herein, the phrase “substantially identical,” in the context of two nucleic acid molecules, nucleotide sequences or protein sequences, refers to two or more sequences or subsequences that have at least about 80%, least about 85%, at least about 90%, at least about 95%, at least about 96%, at least about 97%, at least about 98%, or at least about 99% nucleotide or amino acid residue identity, when compared and aligned for maximum correspondence, as measured using one of the following sequence comparison algorithms or by visual inspection.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math. 2:482 (1981); Smith et al., Nucleic Acids Res. 11:2205 (1983)).

Useful methods for determining sequence identity are also disclosed in Guide to Huge Computers (Martin J. Bishop, ed., Academic Press, San Diego (1994)), and Carillo, H., and Lipton, D., Applied Math 48:1073(1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

Two nucleotide sequences can be considered to be substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the T. for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2× SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is 1× SSC at 45° C. for 15 minutes. An example of a low stringency wash for a duplex of, e.g., more than 100 nucleotides, is 4-6× SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code.

The following are examples of sets of hybridization/wash conditions that may be used to clone homologous nucleotide sequences that are substantially identical to reference nucleotide sequences of the invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2× SSC, 0.1% SDS at 50° C. In another embodiment, the reference nucleotide sequence hybridizes to the “test” nucleotide sequence in 7% sodium dodecyl sulfate (SDS), 0.5 M NaPO₄, 1 mM EDTA at 50° C. with washing in 1× SSC, 0.1% SDS at 50° C. or in 7% sodium dodecyl sulfate (SDS), 0.5 M NaPO₄, 1 mM EDTA at 50° C. with washing in 0.5× SSC, 0.1% SDS at 50° C. In still further embodiments, the reference nucleotide sequence hybridizes to the “test” nucleotide sequence in 7% sodium dodecyl sulfate (SDS), 0.5 M NaPO₄, 1 mM EDTA at 50° C. with washing in 0.1× SSC, 0.1% SDS at 50° C., or in 7% sodium dodecyl sulfate (SDS), 0.5 M NaPO₄, 1 mM EDTA at 50° C. with washing in 0.1× SSC, 0.1% SDS at 65° C.

In some embodiments, a recombinant nucleic acid molecule of the invention can be an “expression cassette” or can be comprised within ann expression cassette. As used herein, “expression cassette” means a recombinant nucleic acid molecule comprising a nucleotide sequence of interest (e.g., the nucleotide sequences of the invention; e.g., a nucleotide sequence encoding an amino acid sequence having at least about 80% identity to of any of SEQ ID NO:1-105, a nucleotide sequence having at least about 80% identity to of any of SEQ ID NOs:106-210, or the complement thereof, or a nucleotide sequence having at least about 80% identity to any of SEQ ID NOs:211-315; and/or fragments thereof), wherein said nucleotide sequence is operably associated with at least a control sequence (e.g., a promoter). Thus, some embodiments of the invention provide expression cassettes designed to express the nucleotide sequences of the invention in a cell.

An expression cassette comprising a nucleotide sequence of interest may be chimeric, meaning that at least one of its components is heterologous with respect to at least one of its other components. An expression cassette may also be one that is naturally occurring but has been obtained in a recombinant form useful for heterologous expression.

An expression cassette also can optionally include a transcriptional and/or translational termination region (i.e., termination region) that is functional in the cell in which the nucleotide sequence of interest is to be expressed. A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the host organism, or may be derived from another source (i.e., foreign or heterologous to the promoter, the nucleotide sequence of interest, the host organism, or any combination thereof). In addition, in some embodiments, a coding sequence's native transcription terminator can be used.

An expression cassette of the invention also can include a nucleotide sequence for a selectable marker, which can be used to select a transformed organism and/or cell. As used herein, “selectable marker” means a nucleotide sequence that when expressed imparts a distinct phenotype to the transformed organism or cell expressing the marker and thus allows such transformed organisms or cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening. Of course, many examples of suitable selectable markers useful in various organisms are known in the art and can be used in the expression cassettes described herein.

In addition to expression cassettes, the nucleic acid molecules and nucleotide sequences described herein can be used in connection with vectors. The term “vector” refers to a composition for transferring, delivering or introducing a nucleic acid (or nucleic acids) into a cell. A vector comprises a nucleic acid molecule comprising the nucleotide sequence(s) to be transferred, delivered or introduced. Vectors for use in transformation of animals, plants and other organisms are well known in the art. Non-limiting examples of general classes of vectors including but not limited to a viral vector, a plasmid vector, a phage vector, a phagemid vector, a cosmid vector, a fosmid vector, a bacteriophage, an artificial chromosome, or an Agrobacterium binary vector in double or single stranded linear or circular form which may or may not be self transmissible or mobilizable. A vector as defined herein can transform prokaryotic or eukaryotic host either by integration into the cellular genome or exist extrachromosomally (e.g., an autonomous replicating plasmid with an origin of replication). Additionally included are shuttle vectors by which is meant a DNA vehicle capable, naturally or by design, of replication in two different host organisms, which may be selected from prokaryotic and eukaryotic organisms. In some representative embodiments, the nucleic acid in the vector is under the control of, and operably linked to, an appropriate promoter or other regulatory elements for transcription in a host cell such as a microbial, e.g. bacterial, or an animal or a plant cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell.

Tardigrades (water bears) comprise a phylum of microscopic animals renowned for their ability to survive a vast array of environmental extremes, including essentially complete desiccation for up to a decade (Goldstein and Blaxter, 2002). Despite fascinating scientists for over 250 years, we know little about how tardigrades survive such extreme environmental stresses, and no molecular mediators of tardigrade stress tolerance have been experimentally confirmed. The disaccharide trehalose has been proposed and often assumed to play a role in mediating desiccation tolerance in tardigrades (Hengherr et al., 2008; Jonsson and Persson, 2010; Westh and Ramlov, 1991). Trehalose is essential for some organisms to survive desiccation (Erkut et al., 2011; Tapia and Koshland, 2014), however, some desiccation tolerant animals do not require or even appear to make this sugar (Lapinski and Tunnacliffe, 2003). Currently, the use and presence of trehalose in tardigrades is unclear; some studies report low levels of this sugar, while others failed to identify trehalose at all in the same species (Guidetti et al., 2011; Hengherr et al., 2008; Jonsson and Persson, 2010; Westh and Ramlov, 1991).

In addition to trehalose and other sugars, a number of protein families/classes have been implicated in mediating desiccation tolerance in other systems including, heat-shock proteins, antioxidant enzymes, and some intrinsically disordered protein (IDP) families (Hoekstra et al., 2001). This latter class of proteins is enigmatic, in that unlike typical globular proteins, they lack persistent tertiary structure. In the past two decades, myriad cellular roles for IDPs have emerged, including roles in abiotic stress tolerance (Chakrabortee et al., 2012; Garay-Arroyo et al., 2000). However, the role of IDPs in tardigrade stress tolerance remains untested.

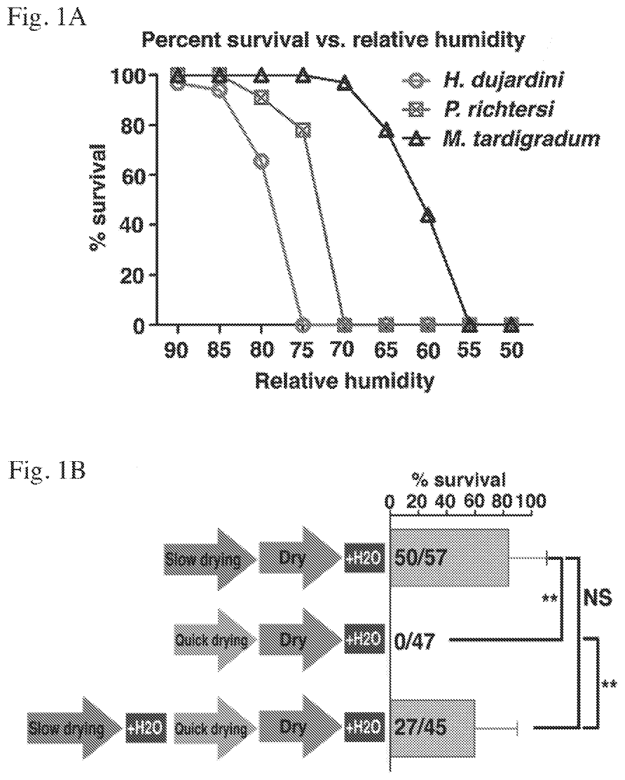

While no molecular mediators of desiccation tolerance have been identified in tardigrades, one clue as to how these animals survive desiccation comes from the observation that different tardigrade species survive drying at different rates, but all species tested die if dried too quickly (FIG. 1A). This trend suggests that tardigrades need time to produce protectants, a theory supported by the recent evidence that de novo transcription and translation are required for the tardigrade Hypsibius dujardini to robustly survive desiccation (Kondo et al., 2015).

Here it is shown that tardigrades upregulate the expression of genes encoding tardigrade-specific intrinsically disordered proteins (TDPs) in response to drying. We found TDP genes are constitutively expressed at high levels or induced during desiccation in multiple tardigrade species. Disruption of gene function for several TDPs through RNA, interference is shown to severely diminished desiccation tolerance in tardigrades. Furthermore, the expression of TDPs in both prokaryotic and eukaryotic cells is sufficient to increase desiccation tolerance in these heterologous systems. These findings identify TDPs as the first functional mediators of tardigrade desiccation tolerance and expand our understanding of the diversity and roles of IDPs and provide the basis, for example, for preservation technologies. In particular, the present inventors have discovered that heterologous polypeptides and/or peptides may be stabilized in the presence of tardigrade disordered proteins, in both aqueous (liquid) and (solid) compositions.

Accordingly, in some embodiments, a liquid composition is provided comprising, consisting essentially of, or consisting of: at least one tardigrade disordered protein (TDP); and at least one heterologous polypeptide and/or peptide of interest. In some embodiments, a solid composition is provided comprising, consisting essentially of, or consisting of: at least one tardigrade disordered protein (TDP); and at least one heterologous polypeptide and/or peptide of interest. In some embodiments, a solid composition may be produced by drying or partially drying a liquid composition of the invention. In some embodiments, a solid composition of the invention may comprise about 0% to about 5% water (e.g., about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5% water, or any range or value therein).

As used herein, “partially drying” refers to drying a composition or solution such that it comprises less water than when the drying process began. Thus, for example, “partially drying” can mean removing about about 5% to about 90% of the water that was present in the composition or solution prior to initiating the drying process. (e.g.,about 5%, 10%, 15%, 20%, 25%, 30%, 35%, 40%, 45%, 50%, 55%, 60%, 65%, 70%, 75%, 80%, 85%, or 90% (or any range or value therein). Thus, in some embodiments the amount of water removed when a composition or solution is partially dried can be from about 10% to about 90%, about 20% to about 90%, about 30% to about 90%, about 40% to about 90%, about 50% to about 90%, about 60% to about 90%, about 10% to about 80%, about 20% to about 80%, about 30% to about 80%, about 40% to about 80%, about 50% to about 80%, about 60% to about 80%, about 70% to about 80%, about 10% to about 70%, about 20% to about 70%, about 30% to about 70%, about 40% to about 70%, about 50% to about 70%, about 10% to about 50%, about 20% to about 50%, about 30% to about 50%, about 40% to about 50% (or anyu range or value therein) of the water that was present in the composition or solution prior to initiating the drying process. Of course, a partially dried composition may be dried further such that it contains less water than when the further drying began.

In other embodiments, a solid composition of the invention may comprise a hydration level of about 0 to about 10 g water per gram of dried protein (e.g., up to about 10 g water per gram of dried protein; e.g., about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 4.5, 6, 6.5, 7, 7.5, 8, 8.5, 9, 9.5, 10, and any range or value therein). In representative embodiments, a solid composition of the invention may comprise a hydration level of about 0 to about 1 g water per gram of dried protein, optionally about 0.4 g H₂O per gram of dried protein.

The amount of TDP in a liquid composition, solid composition, and/or solution of the invention can vary depending on the heterologous polypeptide and/or peptide of interest, whether it is a liquid or a solid, and/or whether the compostion is a liquid composition or solution that will be dried. Thus, in some embodiments, the TDP concentration in a liquid composition, solid composition, and/or solution of the invention may be about 1 g/L to about 100 g/L or any range or value therein (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 g/L, or any range or value therein). In some embodiments, the TDP concentration in a liquid composition or solution of the invention may be about 10 g/L to about 60 g/L. In representative embodiments, the TDP concentration in a liquid composition or a solution of the invention may be about 30 g/L to about 40 g/L, optionally about 36 g/L. In some embodiments, the TDP concentration in a solid composition of the invention may be about 1 g/L to about 20 g/L. In representative embodiments, the TDP concentration in a solid composition of the invention may be about 1 g/L to about 10 g/L, optionally about 5 g/L. The concentration of the TDP to the

In some embodiments, a liquid composition, solid composition, and/or solution may comprise about 50% to about 99.9% of TDP (total weight) (e.g., about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.99% total weight, and any range or value therein). In some embodiments, a liquid composition, solid composition, and/or solution may comprise about 90% to 99.99% of TDP (total weight) (e.g., about 90, 91, 92, 93, 94, 95, 96, 97, 98, 99, 99.1, 99.15, 99.2, 99.25, 99.3, 99.35, 99.4, 99.45, 99.5, 99.55, 99.6, 99.65, 99.7, 99.75, 99.8, 99.85, 99.9, 99.95, 99.99% total weight, and any range or value therein).

In some embodiments, the mass ratio of the at least one heterologous polypeptide and/or peptide of interest to the at least one TDP in a liquid or a solid composition may be about 1:100 to about 1:10 (e.g., about 1:100, 1:95, 1:90, 1:85, 1:80, 1:75, 1:70, 1:65, 1:60, 1:55, 1:50, 1:45, 1:40, 1:35, 1:30, 1:25, 1:20, 1:15, 1:10; and any range or value therein). In representative embodiments, the at least one heterologous polypeptide and/or peptide of interest to the at least one TDP in a liquid or a solid composition may be about 1:20 to about 1:10.

The liquid compositions, solid compositions, and/or solutions of this invention may comprise any number or combination of TDPs from various tardigrade genera or species. Thus, in some embodiments, the liquid compositions, solid compositions, and/or solutions can comprise, consist essentailly of, or consist of at least about 1, 2, 3, 4, 5, 6, 7, 8, 9, 10, 11, 12, 13, 14, 15, 16, 17, 18, 19 or 20 or more different TDPs (e.g., about 1 to about 25, about 1 to about 20, about 1 to about 15, about 1 to about 10, about I to about 5, about 2 to about 10, about 2 to about 5, about 4 to about 10, about 6 to about 10 different TDPs and the like). When a a liquid composition, solid composition, and/or solution of the invention comprises two or more TDPs, the TDPs can be from the same or from any combination of different tardigrade species or genera.

Exemplary tardigrade genera from which the at least one TDP may be obtained include Macrobiotus spp., Isohypsibius spp., Diphascon spp., Echiniscus spp., Minibiotus spp., Doryphoribius spp., Paramacrobiotus spp., Hypsibius spp., Milnesium spp., Pseudechiniscus spp., Ramazzottius spp., Batillipes spp., Bryodelphax spp., Dactylobiotus spp., Echiniscoides spp., Calcarobiotus spp., Tenuibiotus spp., Itaquascon spp., Cornechiniscus spp., and/or Halechiniscus spp. In representative embodiments, the at least one TDP may be obtained from the tardigrade genera of Hypsibius spp., Paramacrobiotus spp., Milnesium spp. and/or Ramazzottius spp. In some embodiments, the at least one TDP may be obtained from one or more of the exemplary tardigrade species provided in Table 1. In representative embodiments, the at least one TDP may be from Hypsibius dujardini, Paramacrobiotus richters, Milnesium tardigradum and/or Ramazzottius varieornatus.

The present invention further provides an isolated tardigrade polypeptide comprising consisting essentially of, or consisting of: (a) an amino acid sequence of any one of SEQ ID NOs: 1-105; (b) an amino acid sequence encoded by a nucleotide sequence of any one of SEQ ID NOs:106-210, or a complement thereof; (c) an amino acid sequence encoded by a nucleotide sequence of any one of SEQ ID NOs:211-315; (d) an amino acid sequence having at least about 80% sequence identity (e.g., 80, 81, 82, 83, 84, 85, 86, 87, 88, 89, 90 91, 92, 93, 94, 95 96, 97, 98, 99, 100% identity) to the amino acid sequence of any one of (a) to (c); or (e) a functional fragment of any one of (a) to (d).

Additionally provided herein is a recombinant nucleic acid construct comprising, consisting essentially of, or consisting of: (a) a nucleotide sequence of any one of SEQ ID NOs:106-210, or a complement thereof; (b) a nucleotide sequence of any one of SEQ ID NOs:211-315; (c) a nucleotide sequence that encodes a polypeptide comprising an amino acid sequence of any one of SEQ ID NOs: 1-105; (d) a nucleotide sequence having at least about 80% sequence identity to the nucleotide sequence of any one of (a) to (c); (e) a nucleotide sequence which anneals under stringent hybridization conditions to the nucleotide sequence of any one of (a) to (d), or a complement thereof; (f) a nucleotide sequence that differs from the nucleotide sequences of any one of (a) to (e) above due to the degeneracy of the genetic code; (g) a functional fragment of a nucleotide sequence of any one of (a) to (f); and (h) any combination of the nucleotide sequences of (a)-(g). In some embodiments, the nucleotide sequence may be operatively linked to a heterologous promoter.

Polypeptides and fragments thereof of the invention may be modified for use by the addition, at the amino- and/or carboxyl-terminal ends, of a blocking, agent. Such blocking agents can include, without limitation, additional related or unrelated peptide sequences that can be attached to the amino and/or carboxyl terminal residues of the peptide to be administered. For example, one or more non-naturally occurring amino acids, such as D-alanine, can be added to the termini. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Additionally, the peptide terminus can be modified, e.g., by acetylation of the N-terminus and/or amidation of the C-terminus. Likewise, the peptides can be covalently or noncovalently coupled to pharmaceutically acceptable “carrier” proteins prior to use.

In particular embodiments, nucleic acids of the present invention may encode any suitable epitope tag, including, but not limited to, poly-Arg tags (e.g., RRRRR (SEQ ID NO:316) and RRRRRR (SEQ ID NO:317) and poly-His tags (e.g., HHHHHH (SEQ ID NO:318)). In some embodiments, the nucleic acid may comprise a nucleotide sequence encoding a poly-Arg tag, a poly-His tag, a FLAG tag (i.e., DYKDDDDK (SEQ ID NO:319)), a Strep-tag II™ (GE Healthcare, Pittsburgh, Pa., USA) (i.e., WSHPQFEK (SEQ ID NO:320)), and/or a c-myc tag (i.e., EQKLISEEDL (SEQ ID NO:321)).

Similarly, in some embodiments, proteins of the present invention may comprise any suitable epitope tag, including, but not limited to, poly-Arg tags (e.g., RRRRR (SEQ ID NO:316) and RRRRRR (SEQ ID NO:317) and poly-His tags (e.g., HHHHHH (SEQ ID NO:318)). In some embodiments, the polypeptide may comprise a poly-Arg tag, a poly-His tag, a FLAG tag (i.e., DYKDDDDK (SEQ ID NO:319)), a Strep-tag II™ (GE Healthcare, Pittsburgh, Pa., USA) (i.e., WSHPQFEK (SEQ ID NO:320)), and/or a c-myc tag (i.e., EQKLISEEDL (SEQ ID NO:321)).

Accordingly, in some embodiments, a solid or liquid composition may comprise, consist essentially of, or consist of a TDP comprising an amino acid sequence having at least about 80% identity to any of SEQ ID NOs:1-105; an amino acid sequence encoded by a nucleotide sequence having at least about 80% identity to any one of SEQ ID NOs:106-210, or a complement thereof; or an amino acid sequence encoded by a nucleotide sequence having at least about 80% identity to any one of SEQ ID NOs:211-315; or any combination thereof. In representative embodiments, a solid or liquid composition may comprise, consistessentially, or consist of a TDP comprising an amino acid sequence having at least about 80% identity to any of SEQ ID NOs:17, 19, 32, 35, and/or 38; an amino acid sequence encoded by a nucleotide sequence having at least about 80% identity to any one of SEQ ID NOs:122, 124, 137, 140, and/or 143, or a complement thereof; or an amino acid sequence encoded by a nucleotide sequence having at least about 80% identity to any one of SEQ ID NOs:227, 229, 242, 245 and 248; or any combination thereof.

In some embodiments, the at least one heterologous polypeptide and/or peptide of interest may be a therapeutic agent or it may be part of a protein-based food. The at least one heterologous polypeptide and/or peptide of interest may be in purified form or it may be in a mixture (unpurified or partially purified). Thus, for example, the at least one heterologous polypeptide and/or peptide of interest may be obtained from, for example, an organism (bacteria, fungi, animals, plants), the cells of an organism (either isolated or cultured), from serum and/or from in vitro expression systems. The heterologous polypeptides and/or peptides so produced may then be protected (stabilized) by contacting them with at least one TDP immediately without any further isolation or purification or they may be contacted with the at least one TDP after they are purified or partially purified. Thus, a mixture may include, for example, serum, cell culture, and/or one or more constituents of an organism or cell thereof, and/or of an in vitro expression system, and the like. In addition, a protein based-food may have multiple additional components (e.g., a mixture), which additional components may or may not be proteinaceous.

A therapeutic protein may be any protein based molecule (e.g., a biologic) including, but not limited to, a vaccine, an antibody, an enzyme, hormone, and/or a globular protein.

The term “antibody” or “antibodies” as used herein refers to all types of immunoglobulins, including IgG, IgM, IgA, IgD, and IgE. The antibody can be monoclonal or polyclonal and can be of any species of origin, including (for example) mouse, rat, rabbit, horse, goat, sheep, camel, or human, or can be a chimeric antibody. See, e.g., Walker et al., Molec. Immunol. 26:403 (1989). The antibodies can be recombinant monoclonal antibodies produced according to the methods disclosed in U.S. Pat. No. 4,474,893 or U.S. Pat. No. 4,816,567. The antibodies can also be chemically constructed according to the method disclosed in U.S. Pat. No. 4,676,980. As used herein, “antibody” also refers to antibody fragments, for example, Fab, Fab′, F(ab′)₂, and Fv fragments; domain antibodies, diabodies; vaccibodies, linear antibodies; single-chain antibody molecules; and multispecific antibodies formed from antibody fragments. Also included within the scope of the present invention are antibodies, which are altered or mutated for compatibility with species other than the species in which the antibody was produced. For example, antibodies may be humanized or camelized. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin.

A “protein-based food” is any food that comprises protein including, but not limited to, meat, seafood, a food comprised of plant based proteins (tofu, tempeh), and/or fungal based proteins (tempeh, meat-substitutes) and the like. Thus, in some embodiments, a TDP may be used as a food additive to stabilize proteins in food products.

Further provided are methods of stabilizing proteins. In a particular embodiment, a method of stabilizing at least one heterologous polypeptide and/or peptide of interest is provided, comprising, contacting the at least one heterologous polypeptide and/or peptide of interest with at least one tardigrade disordered protein (TDP), to produce a liquid composition comprising the at least one heterologous polypeptide and/or peptide of interest and the at least one TDP, thereby stabilizing the at least one heterologous polypeptide and/or peptide of interest. In some embodiments, the method further comprises at least partially drying the liquid composition that comprises the at least one heterologous polypeptide of interest and the at least one tardigrade disordered protein (TDP). Drying of the liquid composition may commence any time following the contacting of the at least one heterologous polypeptide and/or peptide of interest with the at least one tardigrade disordered protein (TDP). Any method of drying a liquid composition may be used including but not limited to freeze-drying, air-drying, spray-drying, spray-freeze-drying, vacuum drying, and/or foam drying. Non-limiting examples of a heterologous polypeptide and/or peptide of interest to be stabilized may include therapeutic agents or protein-based foods as described herein.

In further embodiments, the invention provides a method of stabilizing a heterologous cell, tissue or organ, comprising, contacting the heterologous cell, tissue or organ with a solution comprising at least one tardigrade disordered protein (TDP), thereby stabilizing the heterologous cell, tissue or organ. In some embodiments, the method further comprises desiccating the heterologous cell, tissue or organ in the presence of the at least one tardigrade disordered protein (TDP). Any method of desiccating a cell, tissue or organ may be used including but not limited to freeze-drying, air-drying, spray-drying, spray-freeze-drying, vacuum drying, and/or foam drying.

Any number or combination of TDPs from any tardigrade genus or species may be used with the methods of stabilizing at least one heterologous polypeptide and/or peptide of interest, or a cell, tissue or organ. In some embodiments, the at least one TDP may be from the tardigrade genus that includes, but is not limited to, that of Macrobiotus spp., Isohypsibius spp., Diphascon spp., Echiniscus spp., Minibiotus spp., Doryphoribius spp., Paramacrobiotus spp., Hypsibius spp., Milnesium spp., Pseudechiniscus spp., Ramazzottius spp., Batillipes spp., Bryodelphax spp., Dactylobiotus spp., Echiniscoides spp., Calcarobiotus spp., Tenuibiotus spp., Itaquascon spp., Cornechiniscus spp., and/or Halechiniscus spp. In representative embodiments, the at least one TDP may be from the tardigrade genus of Hypsibius spp., Paramacrobiotus spp., Milnesium spp. and/or Ramazzottius spp. In some embodiments, the at least one TDP may be from one or more of the exemplary tardigrade species provided in Table 1. In representative embodiments, the at least one TDP may be from Hypsibius dujardini, Paramacrobiotus richters, Milnesium tardigradum and/or Ramazzottius varieornatus.

In additional embodiments, the at least one TDP may comprise, consist essentially of, or consist of an amino acid sequence having at least about 80% identity to any one of SEQ ID NOs:1-105; an amino acid sequence encoded by a nucleotide sequence having at least about 80% identity to any one of SEQ ID NOs:106-210, or a complement thereof; or an amino acid sequence encoded by a nucleotide sequence having at least about 80% identity to any one of SED ID NO: 211-315; or any combination thereof. In further embodiments, the at least one TDP may comprise, consist essentially of, or consist of an amino acid sequence having at least about 80% identity to any one of SEQ ID NOs:17, 19, 32, 35, and/or 38; an amino acid sequence encoded by a nucleotide sequence having at least about 80% identity to any one of SEQ ID NOs:122, 124, 137, 140, and/or 143, or a complement thereof; or an amino acid sequence encoded by a nucleotide sequence having at least about 80% identity to any one of SEQ ID NOs:227, 229, 242, 245 and 248; or any combination thereof.

In some embodiments, the liquid compositions, solid compositions and/or solutions of the invention can further comprise one more excipients. Exemplary excipients include, but are not limited to, trehalose, sucrose, maltose, bovine serum albumin, human serum albumin, mannitol, sorbitol, polysorbate, a buffer, a salt, an antioxidant, preservative, colorant, and/or flavorant.

In some embodiments, when a liquid composition, solid composition and/or solution of the invention comprises a salt, the concentration of the salt can be about 0.01 mM to about 100 mM or any range or value therein (e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 15, 20, 25, 30, 35, 40, 45, 50, 55, 60, 65, 70, 75, 80, 85, 90, 95, 100 mM and any range or value therein). In some embodiments, the salt concentration can be about 0.1 mM to 50 mM and any value or range therein). Any appropriate physiologically compatible salt may be used, for example, NaCl.

The pH of a liquid composition, solid composition and/or solution of the invention may be about 5 to about 9, or any range or value therein (e.g., about 5, 5.1, 5.2, 5.3, 5.5, 5.6, 5.7, 5.8, 5.9, 6, 6.1, 6.2, 6.3, 6.5, 6.6, 6.7, 6.8, 6.9, 7, 7.1, 7.2, 7.3, 7.5, 7.6, 7.7, 7.8, 7.9, 8, 8.1, 8.2, 8.3, 8.5, 8.6, 8.7, 8.8, 8.9, 9, and the like). In representative embodiments, the pH of a liquid composition, solid composition and/or solution of the invention may be, for example, about pH 6 to about pH 8, about pH 6.5 to about pH7.5, optionally about pH 7.

In some embodiments, the the liquid compositions, solid compositions and/or solutions of the invention may comprise a buffer. Any buffer may be used provided the buffer is in with the pH range of about pH 5to about pH 9, and within the salt concentration of about 0 to 100 mM.

In further embodiments, a method of producing a transgenic cell having increased tolerance to drought or desiccation is provided, comprising, consisting essentially of, or consisting of: introducing into a cell at least one heterologous nucleotide sequence encoding a tardigrade disordered protein (TDP), thereby producing a transgenic cell having increased tolerance to drought or desiccation.

Additionally provided is method of increasing drought or desiccation tolerance in an organism comprising, consisting essentially of, or consisting of: introducing into the organism at least one heterologous nucleotide sequence encoding a tardigrade disordered protein (TDP), to produce a transgenic organism expressing the heterologous nucleotide sequence, thereby increasing the drought or desiccation tolerance of the transgenic organism. In some embodiments, wherein the cell is a plant cell, the method further comprising regenerating a transgenic plant from the transgenic cell, the regenerated transgenic plant comprising the heterologous nucleotide sequence encoding a TDP in its genome.

In some embodiments, an organism useful with the invention may be, for example, a fungus, a bacterium, a plant, an animal (e.g., a mammal, an avian, a reptile, an amphibian, an insect, or a fish). A cell, tissue or organ useful with this invention may be from any organism, including but not limited to a fungus, a bacterium, a plant, an animal (e.g., a mammal, an avian, a reptile, an amphibian, an insect, or a fish). Exemplary mammals include a human, a non-human primate, a dog, a cat, a goat, a horse, a pig, a cow, a sheep, a rat, a guinea pig, a mouse, a gerbil, or a hamster. In some embodiments, the animal or mammal is not a human (e.g., a non-human animal, a non-human mammal, a non-human primate). Further, any cell type from an organism may be used with the methods of the invention including, but not limited to, a sperm cell, an egg cell, a stem cell, a red blood cell, a muscle cell, and/or a skin cell.

“Introducing,” in the context of a polynucleotide of interest (e.g., at least one heterologous nucleotide sequence encoding a tardigrade disordered protein (TDP); e.g., a nucleotide sequence encoding an amino acid sequence having at least about 80% identity to any of SEQ ID NOs:1-105, a nucleotide sequence having at least about 80% identity to any of SEQ ID NOs:106-210, or a complement thereof, or a nucleotide sequence having at least about 80% identity to any of SEQ ID NOs:211-315, and/or fragments thereof), means presenting the nucleotide sequence of interest to the cell of an organism in such a manner that the nucleotide sequence gains access to the interior of the cell. The methods of the invention do not depend on a particular method for introducing one or more nucleotide sequences into an organism, only that they gain access to the interior of at least one cell of the organism. Where more than one nucleotide sequence is to be introduced, these nucleotide sequences can be assembled as part of a single polynucleotide or nucleic acid construct, or as separate polynucleotide or nucleic acid constructs, and can be located on the same or different expression constructs or transformation vectors. Accordingly, these polynucleotides may be introduced into cells in a single transformation event, in separate transformation events, or, for example, they may be incorporated into an organism as part of a breeding protocol.

The term “transformation” as used herein refers to the introduction of a heterologous nucleic acid into a cell. Transformation of a cell may be stable or transient. Thus, in some embodiments, a cell of the invention may be stably transformed with a nucleotide sequence of the invention. In other embodiments, a cell may be transiently transformed with a nucleotide sequence of the invention.

“Transient transformation” in the context of a polynucleotide means that a polynucleotide is introduced into the cell and does not integrate into the genome of the cell.

By “stably introducing” or “stably introduced” in the context of a polynucleotide introduced into a cell is intended that the introduced polynucleotide is stably incorporated into the genome of the cell, and thus the cell is stably transformed with the polynucleotide.

“Stable transformation” or “stably transformed” as used herein means that a polynucleotide is introduced into a cell and integrates into the genome of the cell. As such, the integrated polynucleotide is capable of being inherited by the progeny thereof, more particularly, by the progeny of multiple successive generations. “Genome” as used herein also includes the nuclear, mitochondrial, and plastid genome, and therefore includes integration of the nucleic acid into, for example, the chloroplast or mitochondrial genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences, which specifically hybridize with a nucleotide sequence of a transgene introduced into an organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

A polynucleotide of the invention (e.g., a nucleotide sequence encoding an amino acid sequence having at least about 80% identity to any of SEQ ID NOs:1-105, a nucleotide sequence having at least about 80% identity to any of SEQ ID NOs:106-210, or a complement thereof, or a nucleotide sequence having at least about 80% identity to any of SEQ ID NOs:211-315, and/or fragments thereof) can be introduced into a cell by any method known to those of skill in the art. In some embodiments of the invention, transformation of a cell comprises nuclear transformation. In other embodiments, transformation of a cell comprises mitochondrial or chloroplast transformation.

Certain TDPs are secreted (Secreted Abundant Heat Soluble (SAHS)), others are produced in the cytosol (Cytosolic Abundant Heat Soluble (CAHS)) and still others are produced in the mitochondria (Mitochondrial Abundant Heat Soluble (MAHS)). It is envisioned that in some embodiments, the SAHS TDPs may be particularly useful in protecting the extracellular side of cell membranes, and therefore, these TDPs may be transformed into the cell with signal peptides directing the secretion of the TDPs to the extracellular side of cell membranes. Further, the CAHS TDPs may be particularly useful for protecting proteins in the cytosol, and therefore, in some embodiments, the CAHS TDPs may be transformed into the cell so as to be produced in the cytosol. Finally, the MAHS TDPs may be particularly useful for protecting mitochondrial proteins and therefore, in some embodiments, the MAHS TDPs may be transformed into the cell so as to be produced in the mitochondria.

Polynucleotides encoding TDPs can be delivered directly into a cell by any method known in the art, e.g., by transfection or microinjection. Those skilled in the art will appreciate that the isolated polynucleotides encoding the TDPs of the invention will typically be associated with appropriate expression control sequences, e.g., transcription/translation control signals and polyadenylation signals.

It will further be appreciated that a variety of promoter/enhancer elements can be used depending on the level and tissue-specific expression desired. The promoter can be constitutive or inducible, depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest.

The nucleotide sequences encoding TDPs can be incorporated into an expression vector. Expression vectors compatible with various host cells are well known in the art and contain suitable elements for transcription and translation of nucleic acids. Typically, an expression vector contains an “expression cassette,” which includes, in the 5′ to 3′ direction, a promoter, a coding sequence encoding a double stranded RNA operatively associated with the promoter, and, optionally, a termination sequence including a stop signal for RNA polymerase and a polyadenylation signal for polyadenylase.

Non-limiting examples of animal and mammalian promoters known in the art include, but are not limited to, the SV40 early (SV40e) promoter region, the promoter contained in the 3′ long terminal repeat (LTR) of Rous sarcoma virus (RSV), the promoters of the EIA or major late promoter (MLP) genes of adenoviruses (Ad), the cytomegalovirus (CMV) early promoter, the herpes simplex virus (HSV) thymidine kinase (TK) promoter, baculovirus IE1 promoter, elongation factor 1 alpha (EF1) promoter, phosphoglycerate kinase (PGK) promoter, ubiquitin (Ubc) promoter, an albumin promoter, the regulatory sequences of the mouse metallothionein-L promoter and transcriptional control regions, the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), mitochondrial-specific promoters, and/or pathogenesis and/or disease-related promoters. In addition, any of these expression sequences of this invention can be modified by addition of enhancer and/or regulatory sequences and the like.

Non-limiting examples of plant promoters include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdca1). PrbcS1 and Pactin are constitutive promoters and Pnr and Pdca1 are inducible promoters. Pnr is induced by nitrate and repressed by ammonium and Pdca1 is induced by salt. Other constitutive plant promoters include but are not limited to cestrum virus promoter (cmp) (U.S. Pat. No. 7,166,770), the rice actin 1 promoter, CaMV 35S promoter, CaMV 19S promoter, nos promoter, Adh promoter, sucrose synthase promoter (, and the ubiquitin promoter. Non-limiting examples of tissue-specific promoters for plants include those associated with genes encoding the seed storage proteins (such as (β-conglycinin, cruciferin, napin and phaseolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4). Non-limiting examples of promoters functional in chloroplasts include the bacteriophage T3 gene 9 5′ UTR, the S-E9 small subunit RuBP carboxylase promoter, the Kunitz trypsin inhibitor gene promoter (Kti3).and other promoters disclosed in U.S. Pat. No. 7,579,516.

The present invention further provides transgenic cells produced by the methods of the invention and comprising at least one heterologous nucleotide sequence encoding a TDP. In some embodiments, a cell having increased tolerance to drought or desiccation produced by the methods of the invention is provided. In some embodiments, the cell can be, but is not limited to, an animal cell (e.g., a mammalian cell, an avian cell, a reptile cell, an amphibian cell, an insect cell, or a fish cell, a sperm cell, an egg cell, a stem cell, a red blood cell, muscle cell, and the like), a fungal cell, a bacterial cell, or a plant cell.

In some embodiments, a transgenic organism (e.g., a transgenic animal, plant, fungus or bacterium) is provided having increased tolerance to drought or desiccation produced by the methods of the invention, wherein the transgenic organism comprises in its genome at least one heterologous nucleotide sequence encoding a TDP. In some embodiments, the invention provides a seed of a transgenic plant produced by the methods of the invention, wherein the seed comprises in its genome at least one heterologous nucleotide sequence encoding a TDP. In further embodiments, the invention provides a crop comprising a plurality of transgenic plants of the invention, planted together in an agricultural field, a golf course, a residential lawn, a road side, an athletic field, and /or a recreational field.

In some embodiments, the compositions of the invention (e.g., one or more isolated TDPs) may be provided as a coating for a seed, wherein the coating increases resistance to drought and/or desiccation in the seed and/or germinated seedling.

In some embodiments, the at least one heterologous nucleotide sequence encoding a TDP may be obtained from a tardigrade genus that includes, but is not limited to, Macrobiotus spp., Isohypsibius spp., Diphascon spp., Echiniscus spp., Minibiotus spp., Doryphoribius spp., Paramacrobiotus spp., Hypsibius spp., Milnesium spp., Pseudechiniscus spp., Ramazzottius spp., Batillipes spp., Bryodelphax spp., Dactylobiotus spp., Echiniscoides spp., Calcarobiotus spp., Tenuibiotus spp., Itaquascon spp., Cornechiniscus spp., and/or Halechiniscus spp. In representative embodiments, the at least one heterologous nucleotide sequence encoding a TDP may be from the tardigrade genus of Hypsibius spp., Paramacrobiotus spp., Milnesium spp. and/or Ramazzottius spp. In other embodiments, the at least one heterologous nucleotide sequence encoding a TDP may be from a tardigrade species that includes, but is not limited to, those listed in Table 1. In representative embodiments, the at least one heterologous nucleotide sequence encoding a TDP may be from Hypsibius dujardini, Paramacrobiotus richters, Milnesium tardigradum and/or Ramazzottius varieornatus.

In further embodiments, the at least one heterologous nucleotide sequence encoding a TDP may be a nucleotide sequence encoding an amino acid sequence having at least about 80% identity to any of SEQ ID NOs:1-105; a nucleotide sequence having at least about 80% identity to any of SEQ ID NOs:106-210, or a complement thereof; a nucleotide sequence having at least about 80% identity to any of SEQ ID NO:211-315; or any combination thereof. In representative embodiments, the at least one heterologous nucleotide sequence encoding a TDP may be a nucleotide sequence encoding an amino acid sequence having at least about 80% identity to any of SEQ ID NOs:17, 19, 32, 35, and/or 38; a nucleotide sequence having at least about 80% identity to any of SEQ ID NOs:122, 124, 137, 140, and/or 143, or a complement thereof; or a nucleotide sequence having at least about 80% identity to any of SEQ ID NOs:227, 229, 242, 245 and 248; or any combination thereof;

TABLE 1
Exemplary tardigrade species

| | | |
|---|---|---|
| Macrobiotus almadai | Macrobiotus insularis | Macrobiotus ragonesei |
| Macrobiotus altitudinalis | Macrobiotus islandicus | Macrobiotus ramoli |
| Macrobiotus alvaroi | Macrobiotus joannae | Macrobiotus rawsoni |
| Macrobiotus anderssoni | Macrobiotus kazmierskii | Macrobiotus recens |
| Macrobiotus andinus | Macrobiotus kirghizicus | Macrobiotus reinhardti |
| Macrobiotus annae | Macrobiotus kolleri | Macrobiotus rigidus |
| Macrobiotus aradasi | Macrobiotus komareki | Macrobiotus rollei |
| Macrobiotus arguei | Macrobiotus kovalevi | Macrobiotus rubens |
| Macrobiotus ariekammensis | Macrobiotus krynauwi | Macrobiotus sandrae |
| Macrobiotus armatus | Macrobiotus kurasi | Macrobiotus santoroi |
| Macrobiotus artipharyngis | Macrobiotus lazzaroi | Macrobiotus sapiens |
| Macrobiotus ascensionis | Macrobiotus lissostomus | Macrobiotus semmelweisi |
| Macrobiotus australis | Macrobiotus liviae | Macrobiotus serratus |
| Macrobiotus baltatus | Macrobiotus longipes | Macrobiotus seychellensis |
| Macrobiotus barabanovi | Macrobiotus lusitanicus | Macrobiotus shennongensis |
| Macrobiotus binieki | Macrobiotus macrocalix | Macrobiotus siamensis |
| Macrobiotus barbarae | Macrobiotus madegassus | Macrobiotus sicheli |
| Macrobiotus biserovi | Macrobiotus mandahaae | Macrobiotus simulans |
| Macrobiotus blocki | Macrobiotus marlenae | Macrobiotus sklodowskae |
| Macrobiotus brevipes | Macrobiotus martini | Macrobiotus snaresensis |
| Macrobiotus caelicola | Macrobiotus mauccii | Macrobiotus spectabilis |
| Macrobiotus carsicus | Macrobiotus meridionalis | Macrobiotus spertii |
| Macrobiotus caymanensis | Macrobiotus modestus | Macrobiotus stellaris |
| Macrobiotus contii | Macrobiotus montanus | Macrobiotus striatus |
| Macrobiotus coronatus | Macrobiotus mottai | Macrobiotus submorulatus |
| Macrobiotus creber | Macrobiotus nelsonae | Macrobiotus szeptyckii |
| Macrobiotus crenulatus | Macrobiotus neuquensis | Macrobiotus tehuelchensis |
| Macrobiotus danielisae | Macrobiotus norvegicus | Macrobiotus terminalis |
| Macrobiotus dariae | Macrobiotus nuragicus | Macrobiotus terricola |
| Macrobiotus denticulus | Macrobiotus occidentalis | Macrobiotus tetraplacoides |
| Macrobiotus diffusus | Macrobiotus ocotensis | Macrobiotus topali |
| Macrobiotus diguensis | Macrobiotus orcadensis | Macrobiotus trunovae |
| Macrobiotus dimentmani | Macrobiotus ovidii | Macrobiotus virgatus |
| Macrobiotus divergens | Macrobiotus ovostriatus | Macrobiotus vladimiri |
| Macrobiotus diversus | Macrobiotus ovovillosus | Macrobiotus wauensis |
| Macrobiotus drakensbergi | Macrobiotus pallarii | Macrobiotus wuzhishanensis |
| Macrobiotus echinogenitus | Macrobiotus papillosus | Macrobiotus yunshanensis |
| Macrobiotus erminiae | Macrobiotus patagonicus | Macrobiotus zhejiangensis |
| Macrobiotus evelinae | Macrobiotus patiens | Isohypsibius altai |
| Macrobiotus furciger | Macrobiotus perfidus | Isohypsibius annulatus |
| Macrobiotus gemmatus | Macrobiotus persimilis | Isohypsibius arbiter |
| Macrobiotus glebkai | Macrobiotus personatus | Isohypsibius archangajensis |
| Macrobiotus grandis | Macrobiotus peterseni | Isohypsibius arcuatus |
| Macrobiotus halei | Macrobiotus pilatoi | Isohypsibius asper |
| Macrobiotus hapukuensis | Macrobiotus polaris | Isohypsibius austriacus |
| Macrobiotus harmsworthi | Macrobiotus polonicus | Isohypsibius baicalensis |
| Macrobiotus hibiscus | Macrobiotus polyopus | Isohypsibius baldii |
| Macrobiotus hieronimi | Macrobiotus porteri | Isohypsibius baldiioides |
| Macrobiotus hufelandi | Macrobiotus potockii | Isohypsibius barbarae |
| Macrobiotus humilis | Macrobiotus primitivae | Isohypsibius bartosi |
| Macrobiotus hyperboreus | Macrobiotus priviterae | Isohypsibius basalovoi |
| Macrobiotus iharosi | Macrobiotus psephus | Isohypsibius belliformis |
| Macrobiotus insignis | Macrobiotus pseudocoronatus | Isohypsibius bellus |
| Macrobiotus insignis | Macrobiotus pseudofurcatus | Isohypsibius borkini |
| Macrobiotus insularis | Macrobiotus pseudoliviae | Isohypsibius brevispinosus |
| Macrobiotus islandicus | Macrobiotus pseudonuragicus | Isohypsibius brevitubulatus |
| Macrobiotus joannae | Macrobiotus punctillus | Isohypsibius brulloi |
| Macrobiotus kazmierskii | Macrobiotus radiatus | Isohypsibius bulbifer |
| Isohypsibius cameruni | Isohypsibius neoundulatus | Echiniscus barbarae |
| Isohypsibius campbellensis | Isohypsibius nipponicus | Echiniscus batramiae |
| Isohypsibius canadensis | Isohypsibius nodosus | Echiniscus becki |
| Isohypsibius ceciliae | Isohypsibius novaeguineae | Echiniscus bigranulatus |
| Isohypsibius changbaiensis | Isohypsibius palmai | Echiniscus bisculptus |
| Isohypsibius chiarae | Isohypsibius panovi | Echiniscus blumi |
| Isohypsibius costatus | Isohypsibius papillifer | Echiniscus calcaratus |
| Isohypsibius cyrilli | Isohypsibius pappi | Echiniscus calvus |
| Isohypsibius damxungensis | Isohypsibius pauper | Echiniscus canadensis |
| Isohypsibius dastychi | Isohypsibius pilatoi | Echiniscus canedoi |
| Isohypsibius deconincki | Isohypsibius pratensis | Echiniscus capillatus |
| Isohypsibius deflexus | Isohypsibius prosostomus | Echiniscus carsicus |
| Isohypsibius dudlchi | Isohypsibius pseudoundulatus | Echiniscus carusoi |
| Isohypsibius duranteae | Isohypsibius pulcher | Echiniscus cavagnaroi |
| Isohypsibius effusus | Isohypsibius pushkini | Echiniscus cervicomis |
| Isohypsibius elegans | Isohypsibius qinlingensis | Echiniscus charrua |
| Isohypsibius eplenyiensis | Isohypsibius rahmi | Echiniscus cheonyoungi |
| Isohypsibius franzi | Isohypsibius reticulatus | Echiniscus cirinoi |
| Isohypsibius fuscus | Isohypsibius roberti | Echiniscus clavispinosus |
| Isohypsibius gilvus | Isohypsibius ronsisvallei | Echiniscus clevelandi |
| Isohypsibius glaber | Isohypsibius rudescui | Echiniscus columinis |
| Isohypsibius glazovi | Isohypsibius rugosus | Echiniscus corrugicaudatus |
| Isohypsibius gracilis | Isohypsibius sabellai | Echiniscus crassispinosus |
| Isohypsibius granditintinus | Isohypsibius sattleri | Echiniscus curiosus |
| Isohypsibius granulifer | Isohypsibius schaudinni | Echiniscus dariae |
| Isohypsibius gyulai | Isohypsibius sculptus | Echiniscus darienae |
| Isohypsibius hadzii | Isohypsibius sellnicki | Echiniscus dearmatus |
| Isohypsibius heienae | Isohypsibius septentrionalis | Echiniscus dikenli |
| Isohypsibius hydrogogianus | Isohypsibius silvicola | Echiniscus diploglyptus |
| Isohypsibius hypostomoides | Isohypsibius sismicus | Echiniscus divergens |
| Isohypsibius improvisus | Isohypsibius solidus | Echiniscus dreyfusi |
| Isohypsibius indicus | Isohypsibius taibaiensis | Echiniscus duboisi |
| Isohypsibius irregibilis | Isohypsibius tetradactyloides | Echiniscus egnatiae |
| Isohypsibius jakieli | Isohypsibius theresiae | Echiniscus ehrenbergi |
| Isohypsibius jingshanensis | Isohypsibius torulosus | Echiniscus elaeinae |
| Isohypsibius jinhouensis | Isohypsibius truncorum | Echiniscus elegans |
| Isohypsibius josephi | Isohypsibius tuberculatus | Echiniscus evelinae |
| Isohypsibius kenodontis | Isohypsibius tuberculoides | Echiniscus filamentosus |
| Isohypsibius kotovae | Isohypsibius tubereticulatus | Echiniscus ganczareki |
| Isohypsibius kristenseni | Isohypsibius tucumanensis | Echiniscus glaber |
| Isohypsibius ladogensis | Isohypsibius undulatus | Echiniscus granulatus |
| Isohypsibius laevis | Isohypsibius vejdovskyi | Echiniscus heterospinosus |
| Isohypsibius latiunguis | Isohypsibius verae | Echiniscus hexacanthus |
| Isohypsibius leithaicus | Isohypsibius verrucosus | Echiniscus hoonsooi |
| Isohypsibius liae | Isohypsibius gibbus | Echiniscus homingi |
| Isohypsibius lineatus | Isohypsibius wilsoni | Echiniscus inocelatus |
| Isohypsibius longiunguis | Isohypsibius woodsae | Echiniscus insuetus |
| Isohypsibius lunulatus | Isohypsibius yunnanensis | Echiniscus jagodici |
| Isohypsibius macrodactylus | Echiniscus africanus | Echiniscus jamesi |
| Isohypsibius malawiensis | Echiniscus aliquantillus | Echiniscus japonicus |
| Isohypsibius mammillosus | Echiniscus angolensis | Echiniscus jenningsi |
| Isohypsibius marcellinoi | Echiniscus apuanus | Echiniscus kerguelensis |
| Isohypsibius marii | Echiniscus arcangelii | Echiniscus knowltoni |
| Isohypsibius mihelcici | Echiniscus arctomys | Echiniscus kofordi |
| Isohypsibius monoicus | Echiniscus arthuri | Echiniscus kosickii |
| Isohypsibius monstruosus | Echiniscus azoricus | Echiniscus lapponicus |
| Isohypsibius montanus | Echiniscus baius | Echiniscus laterosetosus |
| Isohypsibius myrops | Echiniscus baloghi | Echiniscus laterospinosus |
| Echiniscus latifasciatus | Echiniscus scabrospinosus | Minibiotus keppelensis |
| Echiniscus lichenorum | Echiniscus semifoveolatus | Minibiotus maculartus |
| Echiniscus limai | Echiniscus shaanxiensis | Minibiotus marcusi |
| Echiniscus lineatus | Echiniscus siegristi | Minibiotus milleri |
| Echiniscus longispinosus | Echiniscus simba | Minibiotus orthofasciatus |
| Echiniscus loxophthalmus | Echiniscus speciosus | Minibiotus pilatus |
| Echiniscus madonnae | Echiniscus spiculifer | Minibiotus poricinctus |
| Echiniscus maesi | Echiniscus spiniger | Minibiotus pustulatus |
| Echiniscus malpighii | Echiniscus spinulosus | Minibiotus ramazzottii |
| Echiniscus manuelae | Echiniscus storkani | Minibiotus scopulus |
| Echiniscus marcusi | Echiniscus sylvanus | Minibiotus sidereus |
| Echiniscus marginatus | Echiniscus taibaiensis | Minibiotus stuckenbergi |
| Echiniscus marginoporus | Echiniscus tamus | Minibiotus subintermedius |
| Echiniscus markezi | Echiniscus tardus | Minibiotus taiti |
| Echiniscus marleyi | Echiniscus tenuis | Minibiotus vinciguerrae |
| Echiniscus mauccii | Echiniscus tessellatus | Minibiotus weglarskae |
| Echiniscus mediantus | Echiniscus testudo | Minibiotus weinerorum |
| Echiniscus merokensis | Echiniscus trisetosus | Minibiotus wuzhishanensis |
| Echiniscus migiurtinus | Echiniscus trojanus | Minibiotus xavieri |
| Echiniscus mihelcici | Echiniscus tropicalis | Doryphoribius amazzonicus |
| Echiniscus militaris | Echiniscus tympanista | Doryphoribius berfolanii |
| Echiniscus molluscorum | Echiniscus velaminis | Doryphoribius bindae |
| Echiniscus moniliatus | Echiniscus vinculus | Doryphoribius dawkinsi |
| Echiniscus montanus | Echiniscus virginicus | Doryphoribius doryphorus |
| Echiniscus mosaicus | Echiniscus viridianus | Doryphoribius dupliglobulatus |
| Echiniscus multispinosus | Echiniscus viridis | Doryphoribius evelinae |
| Echiniscus murrayi | Echiniscus viridissimus | Doryphoribius flavus |
| Echiniscus nelsonae | Echiniscus walteri | Doryphoribius gibber |
| Echiniscus nepalensis | Echiniscus weisseri | Doryphoribius huangguoshuensis |
| Echiniscus nigripustulus | Echiniscus wendti | Doryphoribius koreanus |
| Echiniscus nobilis | Echiniscus zetotrymus | Doryphoribius korganovae |
| Echiniscus oihonnae | Minibiotus acadianus | Doryphoribius longistipes |
| Echiniscus ollantaytamboensis | Minibiotus acontistus | Doryphoribius macrodon |
| Echiniscus osellai | Minibiotus aculeatus | Doryphoribius maranguensis |
| Echiniscus pajstunensis | Minibiotus africanus | Doryphoribius mariae |
| Echiniscus palmai | Minibiotus allani | Doryphoribius mexicanus |
| Echiniscus perarmatus | Minibiotus aquatilis | Doryphoribius minimus |
| Echiniscus peruvianus | Minibiotus asteris | Doryphoribius neglectus |
| Echiniscus perviridis | Minibiotus bisoctus | Doryphoribius picoensis |
| Echiniscus phocae | Minibiotus claxtonae | Doryphoribius pilatoi |
| Echiniscus polygonalis | Minibiotus constellatus | Doryphoribius polynettae |
| Echiniscus pooensis | Minibiotus continuus | Doryphoribius qinlingense |
| Echiniscus porabrus | Minibiotus crassidens | Doryphoribius quadrituberculatus |
| Echiniscus postojnensis | Minibiotus decrescens | Doryphoribius smokiensis |
| Echiniscus pseudelegans | Minibiotus diphasconides | Doryphoribius solidunguis |
| Echiniscus pseudowendti | Minibiotus eichhomi | Doryphoribius taiwanus |
| Echiniscus punctus | Minibiotus ethelae | Doryphoribius tergumrudis |
| Echiniscus pusae | Minibiotus fallax | Doryphoribius tessellatus |
| Echiniscus quadrispinosus | Minibiotus floriparus | Doryphoribius turkmenicus |
| Echiniscus quitensis | Minibiotus furcatus | Doryphoribius vietnamensis |
| Echiniscus rackae | Minibiotus granatai | Doryphoribius zappalai |
| Echiniscus ranzii | Minibiotus gumersindoi | Doryphoribius zyxiglobus |
| Echiniscus reticulatus | Minibiotus harryiewisi | Paramacrobiotus alekseevi |
| Echiniscus reymondi | Minibiotus hispidus | Paramacrobiotus areolatus |
| Echiniscus robertsi | Minibiotus hufelandioides | Paramacrobiotus beotiae |
| Echiniscus rodnae | Minibiotus intermedius | Paramacrobiotus centesimus |
| Echiniscus rufoviridis | Minibiotus jonesorum | Paramacrobiotus chieregoi |
| Echiniscus rugospinosus | Minibiotus julietae | Paramacrobiotus corgatensis |
| Paramacrobiotus crenatus | Hypsibius thaleri | Pseudechiniscus victor |
| Paramacrobiotus csotiensis | Milnesium alabamae | Pseudechiniscus yunnanensis |
| Paramacrobiotus danielae | Milnesium almatyense | Ramazzottius affinis |
| Paramacrobiotus derkai | Milnesium antarcticum | Ramazzottius agannae |
| Paramacrobiotus garynahi | Milnesium asiaticum | Ramazzottius andreevi |
| Paramacrobiotus gerlachae | Milnesium brachyungue | Ramazzottius anomalus |
| Paramacrobiotus huziori | Milnesium dujiangensis | Ramazzottius baumanni |
| Paramacrobiotus kenlanus | Milnesium eutystomum | Ramazzottius belubellus |
| Paramacrobiotus lorenae | Milnesium granulatum | Ramazzottius bunikowskae |
| Paramacrobiotus magdalenae | Milnesium jacobi | Ramazzottius cataphractus |
| Paramacrobiotus palaui | Milnesium katarzynae | Ramazzottius caucasicus |
| Paramacrobiotus peteri | Milnesium krzysztofi | Ramazzottius edmondabouti |
| Paramacrobiotus richtersi | Milnesium longiungue | Ramazzottius homingi |
| Paramacrobiotus rioplatensis | Milnesium minutum | Ramazzottius ljudmilae |
| Paramacrobiotus savai | Milnesium reductum | Ramazzottius montivatus |
| Paramacrobiotus tonollii | Milnesium reticulatum | Ramazzottius nivalis |
| Paramacrobiotus vanescens | Milnesium sandrae | Ramazzottius novemcinctus |
| Paramacrobiotus walteri | Milnesium swolenskyi | Ramazzottius oberhaeuseri |
| Hypsibius allisoni | Milnesium tardigradum | Ramazzottius rupeus |
| Hypsibius antonovae | Milnesium tetralameliatum | Ramazzottius saltensis |
| Hypsibius arcticus | Milnesium zsalakoae | Ramazzottius semisculptus |
| Hypsibius biscuitiformis | Pseudechiniscus alberti | Ramazzottius subanomalus |
| Hypsibius calcaratus | Pseudechiniscus asper | Ramazzottius szeptycki |
| Hypsibius camelopardalis | Pseudechiniscus bartkei | Ramazzottius theroni |
| Hypsibius choucoutiensis | Pseudechiniscus beasleyi | Ramazzottius thulini |
| Hypsibius conifer | Pseudechiniscus bidenticulatus | Ramazzottius tribulosus |
| Hypsibius convergens | Pseudechiniscus bispinosus | Ramazzottius valaamis |
| Hypsibius dujardini | Pseudechiniscus brevimontanus | Ramazzottius vatieomatus |
| Hypsibius fuhrmanni | Pseudechiniscus clavatus | Batillipes acaudatus |
| Hypsibius giusepperamazzotti | Pseudechiniscus conifer | Batillipes adriaticus |
| Hypsibius heardensis | Pseudechiniscus dicrani | Batillipes africanus |
| Hypsibius hypostomus | Pseudechiniscus distinctus | Batillipes annulatus |
| Hypsibius iskandarovi | Pseudechiniscus facettalis | Batilfipes bullacaudatus |
| Hypsibius janetscheki | Pseudechiniscus goedeni | Batillipes camonensis |
| Hypsibius klebelsbergi | Pseudechiniscus gullii | Batillipes crassipes |
| Hypsibius kunmingensis | Pseudechiniscus insolitus | Batillipes dicrocercus |
| Hypsibius macrocalcaratus | Pseudechiniscus islandicus | Batifiipes ftiaufi |
| Hypsibius maculatus | Pseudechiniscus jiroveci | Batillipes gilmartini |
| Hypsibius marcelli | Pseudechiniscus juanitae | Batillipes lesteri |
| Hypsibius microps | Pseudechiniscus jubatus | Batillipes littoralis |
| Hypsibius montanus | Pseudechiniscus megacephalus | Batillipes longispinosus |
| Hypsibius morikawai | Pseudechiniscus nataliae | Batiffipes marcelli |
| Hypsibius multituberculatus | Pseudechiniscus novaezeelandiae | Batillipes mirus |
| Hypsibius novaezeelandiae | Pseudechiniscus occultus | Batillipes noerrevangi |
| Hypsibius pachyunguis | Pseudechiniscus papillosus | Batillipes orlentails |
| Hypsibius pallidus | Pseudechiniscus pilatoi | Batillipes pennaki |
| Hypsibius paffidoides | Pseudechiniscus pseudoconifer | Batillipes philippinensis |
| Hypsibius pedrottii | Pseudechiniscus pulcher | Batillipes phreaticus |
| Hypsibius pradellii | Pseudechiniscus quadrilobatus | Batiffipes roscoffensis |
| Hypsibius ragonesei | Pseudechiniscus ramazzottii | Batillipes rotundiculus |
| Hypsibius roanensis | Pseudechiniscus raneyi | Batillipes similis |
| Hypsibius runae | Pseudechiniscus santomensis | Batillipes spinicauda |
| Hypsibius scaber | Pseudechiniscus scorteccii | Batillipes tridentatus |
| Hypsibius scabropygus | Pseudechiniscus shilinensis | Batillipes tubematis |
| Hypsibius septulatus | Pseudechiniscus sinensis | Btyodelphax aaseae |
| Hypsibius seychellensis | Pseudechiniscus spinerectus | Bryodelphax alzirae |
| Hypsibius shaanxiensis | Pseudechiniscus suillus | Bryodelphax amphoterus |
| Hypsibius stiliferus | Pseudechiniscus transsylvanicus | Bryodelphax asiaticus |
| Bryodelphax atlantis | Itaquascon pawlowskii | Diphascon gerdae |
| Bryodelphax brevidentatus | Itaquascon pisoniae | Diphascon granifer |
| Bryodelphax crossotus | Itaquascon simplex | Diphascon halapiense |
| Bryodelphax dominicanus | Itaquascon umbellinae | Diphascon higginsi |
| Bryodelphax iohannis | Itaquascon unguiculum | Diphascon humicus |
| Bryodelphax lijiangensis | Cornechiniscus brachycomutus | Diphascon hydrophilum |
| Bryodelphax mateusi | Cornechiniscus ceratophorus | Diphascon harosi |
| Bryodelphax meronensis | Cornechiniscus cornutus | Diphascon iltisi |
| Bryodelphax ortholineatus | Cornechiniscus holmeni | Diphascon langhovdense |
| Bryodelphax parvulus | Cornechiniscus lobatus | Diphascon latipes |
| Bryodelphax sinensis | Cornechiniscus madagascariensis | Diphascon mirabilis |
| Bryodelphax tatrensis | Cornechiniscus schrammi | Diphascon mitrense |
| Bryodelphax weglarskae | Cornechiniscus subcomutus | Diphascon nelsonae |
| Dactylobiotus ambiguus | Cornechiniscus tibetanus | Diphascon nobllei |
| Dactylobiotus ampullaceus | Halechiniscus chafarinensis | Diphascon nodulosum |
| Dactylobiotus aqua tills | Halechiniscus greveni | Diphascon nonbullatum |
| Dactylobiotus caldarellal | Halechiniscus guiteli | Diphascon oculatum |
| Dactylobiotus detvizi | Halechiniscus jejuensis | Diphascon ongulense |
| Dactylobiotus dispar | Halechiniscus macrocephalus | Diphascon opisthoglyptum |
| Dactylobiotus grandipes | Halechiniscus paratuleari | Diphascon patanei |
| Dactylobiotus haplonyx | Halechiniscus petfectus | Diphascon pingue |
| Dactylobiotus henanensis | Halechiniscus remanei | Diphascon pinguiforme |
| Dactylobiotus kansae | Halechiniscus tuleari | Diphascon platyungue |
| Dactylobiotus lombardoi | Diphascon arduifrons | Diphascon polare |
| Dactylobiotus luci | Diphascon behanae | Diphascon puniceum |
| Dactylobiotus macronyx | Diphascon belgicae | Diphascon ramazzottii |
| Dactylobiotus octavi | Diphascon carolae | Diphascon recamieri |
| Dactylobiotus palthenogeneticus | Diphascon clavatum | Diphascon rugocaudatum |
| Dactylobiotus selenicus | Diphascon gordonense | Diphascon rugosum |
| Echiniscoides andamanensis | Diphascon greveni | Diphascon sanae |
| Echiniscoides bruni | Diphascon linzhiensis | Diphascon secchii |
| Echiniscoides higginsi | Diphascon maucci | Diphascon serratum |
| Echiniscoides hoepneti | Diphascon modestum | Diphascon sexbullatum |
| Echiniscoides horningi | Diphascon montigenum | Diphascon stappersi |
| Echiniscoides pollocki | Diphascon onorei | Diphascon tenue |
| Echiniscoides sigismundi | Diphascon prorsirostre | Diphascon trachydorsatum |
| Echiniscoides travei | Diphascon scoticum | Diphascon victoriae |
| Tenuibiotus bondavaffii | Diphascon tricuspidatum | Diphascon zaniewi |
| Tenuibiotus bozhkae | Diphascon triodon | Diphascon bicome |
| Tenuibiotus ciprianoi | Diphascon aculea turn | Diphascon coniferens |
| Tenuibiotus danilovi | Diphascon alpinum | Diphascon marcuzzii |
| Tenuibiotus higginsi | Diphascon australianum | Diphascon mariae |
| Tenuibiotus hyperonyx | Diphascon bidropion | Diphascon punctatum |
| Tenuibiotus hystricogenitus | Diphascon birklehofi | Diphascon rivulare |
| Tenuibiotus kozharai | Diphascon bisbullatum | Diphascon speciosum |
| Tenuibiotus mongollicus | Diphascon boreale | Calcarobiotus digeronimoi |
| Tenuibiotus tenuiformis | Diphascon brevipes | Calcarobiotus filmed |
| Tenuibiotus tenuis | Diphascon bullatum | Calcarobiotus gildae |
| Tenuibiotus voronkovi | Diphascon butt | Calcarobiotus hainanensis |
| Tenuibiotus willardi | Diphascon chilenense | Calcarobiotus imperialis |
| Itaquascon biserovi | Diphascon claxtonae | Calcarobiotus longinoi |
| Itaquascon cambewarrense | Diphascon dastychi | Calcarobiotus occultus |
| Itaquascon enckelli | Diphascon dolmiticum | Calcarobiotus parvicalcar |
| Itaquascon globuliferum | Diphascon elongatum | Calcarobiotus polygonatus |
| Itaquascon mongolicus | Diphascon faialense | Calcarobiotus tetrannulatus |

A further aspect of the invention relates to kits for use in the methods of the invention. The kit can comprise one or more TDPs of the invention in a form suitable for stabilizing vaccines, antibodies, a heterologous cell, tissue, organ and/or other biologics or in a form suitable for introducing into an organism. The kit can further comprise other components, such as therapeutic agents, carriers, buffers, containers, devices for administration/contacting, compositions for transformation, and the like. The kit can be designed for therapeutic use, diagnostic use, and/or research use and the additional components can be those suitable for the intended use. The kit can further comprise labels and/or instructions, e.g., for stabilizing a heterologous polypeptide, cell, tissue, or for, e.g., imparting drought or dessication resistance/tolerance to an organism. Such labeling and/or instructions can include, for example, information concerning the amount, frequency and method of administration of the one or moreTDPs.

The following examples are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### EXAMPLES

### Example 1

### Tardigrade Culture and Collection

H dujardini was cultured in glass petri-dishes filled with spring water (Deer Park) and fed unicellular Chlorococcum sp. algae as described (Gabriel et al., 2007). P. richtersi was extracted from hazel leaf litter collected at Formigine (Northern Italy; N 44° 34.253′, E 10° 50.892′, 80 m a.s.l.). Dry leaf litter was stored at −80° C. until specimen collection. To isolate P. richtersi, leaf litter was sprinkled with tap water for 15 min, and then submerged in water for 30 min. Active P. richtersi specimens were then extracted by sieves (250 μm and 37 μm mesh) under running water, and animals were isolated via direct microscopic observation. M tardigradum short reads were downloaded from NCBI (Accessions SRX426237-SRX426240).

### Example 2

### H. dujardini RNA Extraction and Library Preparation

For RNAseq experiments three biological replicates were used for each condition: wet, drying, or frozen. To isolate RNA from desiccating specimens, 400 pl of Trizol was used to wash specimens from dishes into a 1.5 mL Eppendorf tube. For frozen and wet specimens, excess liquid was removed from pelleted animals and 400 μl of Trizol was added directly to the tubes. Plastic pestles were placed in tubes and the tubes dipped into liquid nitrogen. The frozen samples were ground with pestles and allowed to thaw. Five rounds of freeze-thaw homogenization were conducted. An additional 100 μ1 of Trizol was used to wash the pestles. Chloroform (100 μl) was mixed with each sample. Tubes were capped, shaken for 20 s, and allowed to sit at room temperature for 3 min. Samples were then centrifuged at 10,000 g for 18 min at 4° C. The clear top layer was removed to a fresh tube and an equal volume of 100% ethanol was added. Samples were then processed using Qiagen's RNeasy® Mini Kit (Qiagen, Cat# 74104) according to manufacturer's instructions. RNA samples were used for library construction using the Illumina mRNA TruSeq v2 kit.

### Example 3

### P. richtersi RNA Extraction and Library Preparation

We isolated RNA from biological replicates of P. richtersi specimens (three wet replicates and two dry replicates) by methods similar to those used for H dujardini. RNA was extracted using the Epicenter MasterPure™ RNA Purification kit (Cat# MCR85102). RNA samples were used for library construction using the Illumina mRNA TruSeq® v2 kit.

### Example 4

### Transcriptome Sequencing, Assembly and Differential Expression Analysis

RNAseq libraries were multiplexed and sequenced on the Illumina HighSeq® 2000 platform. Raw transcriptome reads for M tardigradum were obtained from NCBI's SRA database (Accessions SRX426237-SRX426240). Pooled reads (H. dujardini−wet+drying+frozen; P. richtersi−wet+dry; M tardigradum—Accessions SRX426237-SRX426240) were used for de novo assembly of transcripts using the program Trinity (Haas et al., 2013). Read mapping was performed for each RNAseq library using RSEM (Li and Dewey, 2011) against the appropriate reference transcriptome. For M tardigradum, differential expression analysis was performed comparing active (SRX426237) and inactive (SRX426238) read counts. For H dujardini and P. richtersi a transcript/gene was considered ‘expressed’ if it had a sum across all sequencing libraries of mapped read counts of 100 or more. Mapped read counts were used to perform differential expression for expressed genes using the program edgeR (Robinson et al., 2010). A transcript was deemed differentially expressed (enriched) if it had both a p-value and a false discovery rate of <0.05.

### Example 5

### Protein Expression and Purification

E. coli codon optimized gBlocks encoding tardigrade CAHS proteins were synthesized (Integrated DNA Technologies) and cloned into the pET28b expression vector. BL21star (DE3) E. coli were transformed with pET28b +CAHS plasmids.

A single bacterial colony was used to inoculate 10 mL of Lennox broth (LB, 10 g/L, tryptone, 5 g/L yeast extract, 5 g/L NaCl) supplemented with 60 μg/mL of kanamycin. The culture was shaken at 37° C. overnight (New Brunswick Scientific Innova 126, 225 rpm). Three of these cultures were used to inoculate 1 L of supplemented M9 media (50 mM Na₂HPO₄, 20 mM KH₂PO₄, 9 mM NaCl, 4 g/L glucose, 1 g/L ¹⁵NH₄Cl, 0.1 mM CaCl₂ 2 mM MgSO₄, 10 mg/L thiamine, 10 mg/L biotin, and 60 μg/mL of kanamycin).

The 1 L cultures were shaken at 37° C. until the optical density at 600 nm reached 0.5. IPTG (1 mM final concentration) was then added to induce expression. After 4 h, the cells were pelleted at 1,000g at 10° C. for 30 min. The cell pellets were stored at −20° C. Pellets were resuspended in 12.5 mL of 50 mM HEPES, 50 mM NaCl (pH 8.0) supplemented with half a Roche cOmplete EDTA-free protease inhibitor tablet (Sigma-Aldrich Cat. #4693159001). Cells were then lysed by heat shock at 95° C. for 15 min. Lysates were cooled at room temperature for 30 min. Insoluble components were removed by centrifugation at 20,000g and 10° C. for 30 min.

MgCl₂ (final concentration 2 mM) was added to the heat soluble fraction before digestion with 1250 units of Benzonase (Sigma-Aldrich) at 37° C. for 1 h. Benzonase was then inactivated by heating to 95° C. After cooling to room temperature, the lysate was sterile filtered using a 0.45 μm filter and transferred to 10,000 MWCO dialysis tubing. Samples were dialyzed against 50 mM sodium phosphate (pH 7.0) overnight followed by dialysis against three changes of 17 MΩcm⁻¹ H₂O for at least 3 h each. The dialysate was again filtered before being flash frozen in CO₂(s)/ethanol and lyophilized for 48 h (Labconco FreeZone). Purity was determined by SDS-PAGE, DNA electrophoresis, and an ethidium bromide fluorescence assay.

### Example 6

### NMR

Purified CAHS proteins were dissolved at 10 g/L in 50 mM sodium phosphate (pH 7.0), 90:10 (vol/vol) H₂O:D₂O by boiling and then centrifuged at 14,000 g for 10 min to remove undissolved material. ¹⁵N-¹H HSQC spectra were acquired at 298 K on an 850 MHz Bruker Avance™ III spectrometer equipped with a TCI cryoprobe. Sweep widths were 11,000 Hz and 3,500 Hz in the ¹H and ¹⁵N dimensions, respectively. Each spectrum comprised 256 increments of 24 scans per increment. One-dimensional spectra were taken 20 mM after sample preparation using a ¹H sweep width of 13,500 Hz and comprised 128 scans. Each pair of H₂O/D₂O spectra was normalized using the methyl resonances at 0.8 ppm.

Purified ubiquitin (2 mM) was resuspended in 50 mM sodium phosphate (pH 7.0), 95:5 (vol/vol) H₂O:D₂O and centrifuged at 20,000 g for 5 mM to remove undissolved material. ¹⁵N-¹H HSQC spectra were acquired at 298 K on the 850 MHz spectrometer. Sweep widths were 14,000 Hz and 3,500 Hz in the ¹H and ¹⁵N dimensions, respectively. Each spectrum comprised 256 increments of 4 scans per increment. One-dimensional spectra were taken 20 mM after sample preparation using a ¹H sweep width of 14,000 Hz and comprised 128 scans. Each one dimensional spectrum was normalized using the methyl resonance at -0.15 ppm, and all spectra are referenced to DSS.

Purified a-synuclein (0.1 mM) was resuspended in 50 mM sodium phosphate (pH 7.0), 95:5 (vol/vol) H₂O:D₂O and centrifuged at 20,000 g for 5 min to remove undissolved material. ¹⁵N-¹H HSQC spectra were acquired at 298 K on the 850 MHz spectrometer. Sweep widths were 14,000 Hz and 3,500 Hz in the ¹H and ¹⁵N dimensions, respectively. Each spectrum comprised 256 increments of 4 scans per increment. One-dimensional spectra were taken 20 min after sample preparation using a ¹H sweep width of 14,000 Hz and comprised 128 scans. Each one dimensional spectrum was normalized using the methyl resonance at 1 ppm, and all spectra are referenced to DSS.

### Example 7

### Identification of TDP-Encoding Transcripts

Transcript sequences were used as BLASTx queries and searched against NCBI's non-redundant protein database. Reciprocal best BLAST was performed with an E-value cutoff of 1E-10.

### Example 8

### RNA Interference

Double stranded RNA (dsRNA) was made and microinjections performed with slight modification of a published protocol (Tenlen et al., 2013). dsRNAs were diluted to a concentration of 1 μg/μ1 in nuclease-free water. Specimens were not sedated with levamisole as previously described (Tenlen et al., 2013) to reduce the number of factors potentially influencing survival. Injected specimens were transferred to 30 mm plastic dishes filled with fresh spring water and left overnight. The next day, specimens were either left in spring water with fresh food added (control), desiccated, or frozen. For each RNAi treatment and stress condition three individual trials were performed, with ten tardigrades injected per trial.

### Example 9

### H. dujardini Desiccation

After injection (RNAi studies) or directly from larger cultures used for RNAseq, H dujardini specimens were transferred to 35 mm plastic petri dishes filled with fresh spring water without algal food. Specimens were starved for 24 h. Melted 2% agar (300 ul) was used to evenly coat the lid of 35 mm dishes and excess agar removed. After solidification, tardigrades were transferred to the center of coated lids. Using a mouth pipette, excess water was removed and lids were placed in humidified chambers. The relative humidity (95% for slow drying and 70% for quick drying) of each chamber was established using a mixture of glycerol and water (Forney and Brandi, 1992) and monitored using a hygrometer. Tardigrades were dried overnight, enough time for tun formation to occur, and then removed and exposed to laboratory conditions (about 35% relative humidity) for 24 h to allow for further desiccation. Rehydration was achieved by pipetting 1.5 mL of spring water into dishes. Rehydrated samples were left for 2 h before observation and quantification of survival. Coordinated movement was used to score survival.

### Example 10

### P. richtersi Desiccation

P. richtersi specimens were desiccated by placing each group of animals on a Whatman filter paper (25 mm² or 1 cm²) with mineral water (9 μl or 30 μl, respectively) and exposing them initially to 80% relative humidity (RH) and 18° C. for 24 h, then to 50% RH at 18° C. for 24 h in a climatically controlled chamber, and finally to 0-3% RH at room temperature for 12 h (Rebecchi et al., 2009). At the end of this treatment animals exhibit the typical tun shape.

### Example 11

### H. dujardini Freezing

After injection (RNAi studies) or directly from larger cultures (RNAseq), H. dujardini specimens were transferred to 35 mm plastic petri dishes filled with fresh spring water without algal food. Specimens were starved for 24 hours. Specimens were then transferred to 1.5 mL microcentrifuge tubes, and the volume of spring water adjusted to 1 mL. The tubes were centrifuged briefly to move specimens to the bottom and then placed in a styrofoam box at −80° C. for 24 h. For RNAi studies, thawing was achieved by moving tubes to ambient laboratory conditions (about 20° C.) for 2 h. Following thawing the contents of each tube were transferred to a new 35 mm dish for observation and quantification of survival. Coordinated movement was used to score for survival. For RNAseq, thawing was accelerated by warming the specimens by hand and then rapidly moving on to RNA extraction.

### Example 12

### Bacterial Heterologous Expression and Desiccation Survival Assay

Cloning and transformation of bacteria was performed as described above. For expression, 10 mL cultures were grown overnight. The following day an aliquot of overnight culture was added to fresh culture media at a ratio of 1:200. Cultures were grown to log phase (0D₆₀₀ 0.4-0.8). Expression of CAHS genes was then induced with 1 mM IPTG and the cultures grown for an additional 4 h. Optical densities were measured again and approximately 10⁸ cells were transferred to 1.5 ml microcentrifuge tubes and spun at 4,000g for 20 mM. Excess culture media was removed, and cells were washed with water and re-pelleted. Water was quickly removed with a pipette and pellets were dried overnight in a SpeedVac (Savant SpeedVac SC100). The tubes, caps open, were transferred to a sealed desiccator filled with Drierite (Sigma-Aldrich, Cat. #238937) for 1 week.

Rehydration and pellet dispersal was achieved by adding 1 ml of culture media to dry pellets and vortexing for 10 mM Cells were then transferred to kanamycin plates and grown overnight at 37° C. The following day colonies were counted and survival reported as colony forming units/10⁸ cells plated.

### Example 13

### Yeast Heterologous Expression and desiccation Survival Assay

The strain MAT a his3Δ1leu2Δ0 lys2Δ0 ura3Δ0 nth1::G418^{R} can1::P_{TDH3}-AGT1 was used. This strain is a haploid alpha strain, with the nthl trehalase gene deleted and replaced with G418 and with the AGT1 trehalose transporter under a constitutive highly expressed TDH3 promoter.

Tardigrade CAHS coding sequences were cloned into the p413-GPD plasmid. Tardigrade genes were under the same TDH3 promoter on CEN plasmids, with histidine selection.

Standard yeast propagation and transformation procedures were used. Strains were grown in selective, synthetic complete, media (2% glucose without histidine). Cultures were grown to logarithmic phase from an overnight culture by incubation, overnight at 30° C. Cultures were re-diluted to an OD₆₀₀ of about 0.05 and allowed to reach mid-log phase (OD₆₀₀ 0.4-0.6).

Desiccation tolerance assays were performed as follows. Approximately 10⁷ cells were withdrawn from liquid cultures, washed twice in water and brought to a final volume of 1 ml. Undesiccated controls were plated for colony counting. Aliquots (200 μl) were then transferred to a 96-well tissue culture plate (Becton Dickinson, 353075), centrifuged and most of the water removed without disturbing the cell pellet. Cells were desiccated in a 23° C. incubator with a constant 60% RH, with the lid raised, for 48 h. Samples were resuspended in water and plated for colony counting. Data were entered into a spreadsheet (Microsoft Excel 2008 for Mac version 12.3), and cell density (CFU/ml) for each plate was determined. For each experiment, density for the two controls was averaged. The relative viability of each of the two experimental samples was determined by dividing the CFU/ml for that sample by the average CFU/ml of the control plates. These two relative viability values were then averaged using the AVE worksheet function and their standard deviation was computed using the STDEV worksheet function. Experiments were repeated at least three times on separate days with separate isolates when appropriate.

### Example 14

### Identification of Likely Mediators of Tardigrade Desiccation Tolerance

To test whether tardigrades produce protectants that are sufficient to protect against desiccation, we assayed whether slowly dried tardigrades can survive subsequent drying at higher, typically non-survivable, rates. Specimens of the tardigrade H. dujardini that had been dried slowly could subsequently survive more rapid desiccation (FIG. 1B), suggesting that a sufficient protectant(s) was made during slow drying. This finding, in addition to the fact that H dujardini requires de novo transcription and translation to robustly survive desiccation (Kondo et al., 2015), makes H dujardini attractive for differential gene expression studies.

To identify potential mediators of desiccation tolerance, genes induced by drying, in an unbiased fashion we sequenced and performed differential gene expression analysis on transcriptomes of hydrated and slowly drying (preconditioned) H dujardini specimens in triplicate.

Our differential gene expression analysis revealed that 11 of 17 Cytosolic Abundant Heat Soluble (CARS) protein transcripts expressed by H dujardini are enriched 4- to 22-fold during desiccation relative to hydrated conditions (cutoff: p-value<0.05 and false discovery rate <0.05). H dujardini expresses 19 Secreted Abundant Heat Soluble (SAHS) protein transcripts, and while only two are enriched 2- to 5-fold during drying, several SAHS transcripts are expressed constitutively at extremely high levels. For example, one SAHS transcript was the sixth most abundant transcript detected. H. dujardini expresses two Mitochondrial Abundant Heat Soluble (MAHS) protein transcripts, neither of which is particularly abundant or differentially expressed between hydrated and dry conditions.

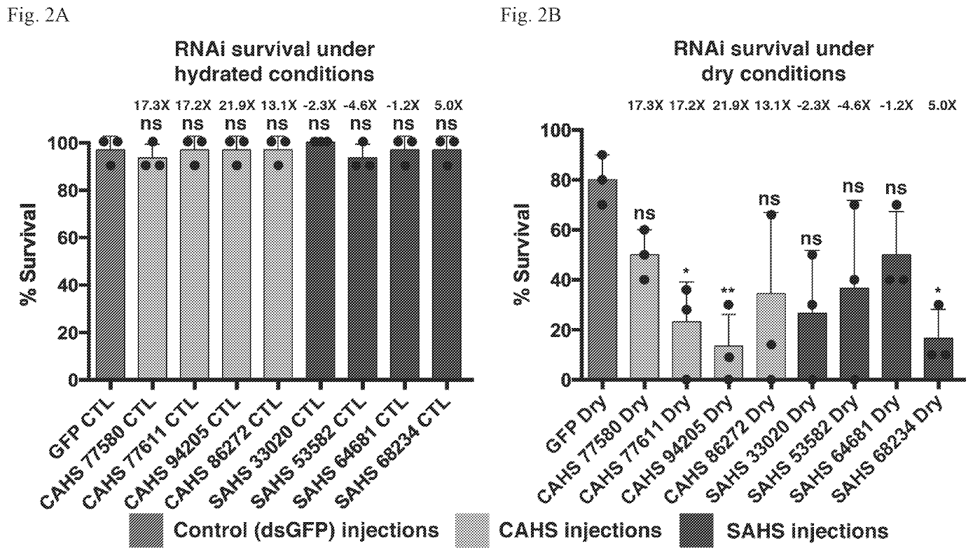

These gene families, CAHS, SANS, and MAHS, were identified in a proteomic analysis of tardigrades, and all three encode intrinsically disordered proteins (IDPs; FIG. 2; Tanaka et al., 2015; Yamaguchi et al., 2012). We refer to these tardigrade-specific intrinsically disordered proteins as TDPs to distinguish them from other IDPs, because, at the sequence level, no homologs of TDPs are found outside the phylum tardigrade (Tanaka et al., 2015; Yamaguchi et al., 2012). IDPs lack persistent secondary structure (Theillet et al., 2014; Yamaguchi et al., 2012), which we confirmed for TDPs by examining CAHS proteins using nuclear magnetic resonance spectroscopy (NMR). To do this we mapped the chemical environment of the covalent bond between each backbone amide nitrogen and its attached proton based on the Heteronuclear Single Quantum Coherence (HQSC) spectra of the protein. In this experiment, each bond gives rise to a feature called a crosspeak at the chemical shift coordinates of the two nuclei for each non-proline residue. For structured proteins like ubiquitin, the crosspeaks occur over a range of about 7.5 to about 10 ppm in the proton dimension (FIG. 2, upper panel). For a-synuclein, a known disordered protein, and for TDPs, the crosspeaks occur over a narrower window, from about 8.0 to about 8.6 ppm, which coincides with the range for amide protons in the central residue of unstructured tripeptides (Schwarzinger et al., 2000). To further test our conclusion that these proteins are disordered, we assessed backbone proton-deuterium exchange. Amide protons in tripeptides exchange with deuterons from D₂O in seconds (Bai et al., 1993), but are protected in the interior of stable globular proteins for days to weeks (Englander and Kallenbach, 1983). After acquiring the HSQC spectra, we removed two aliquots from each sample. One aliquot was diluted ten-fold with H₂O and the other was diluted ten-fold with D₂O. For the disordered proteins tested (a-synuclein and the TDPs) nearly all the amide protons were exchanged for deuterons within 20 minutes as shown by the decrease in intensity of the one-dimensional proton spectrum. In contrast, very little exchange was observed for the structured protein ubiquitin in 20 minutes. These data show that tardigrade CAHS proteins are disordered.

Several families of IDPs, such as Late Embryogenesis Abundant (LEA) proteins and hydrophilins, have known or suspected roles in stress tolerance in organisms spanning all kingdoms of life (Chakrabortee et al., 2012; Garay-Arroyo et al., 2000) and a recent study speculates that MAHS proteins may play a role in desiccation tolerance in tardigrades (Tanaka et al., 2015). These observations, coupled with the fact that TDPs are induced by drying, suggests that they play a role in tardigrade stress tolerance (Yamaguchi et al., 2012). However, until now no studies have been conducted to directly examine the effect of environmental conditions on the expression of genes encoding TDPs or their involvement in tardigrade stress tolerance.

Constitutive Expression or Enrichment of TDPs During Desiccation Is Conserved Among Eutardigrades. We hypothesized that high levels of TDP transcripts in drying H dujardini is a characteristic of desiccation tolerant tardigrades more generally. To test this hypothesis, we sequenced hydrated and dry transcriptomes from a second desiccation tolerant tardigrade species, Paramacrobiotus richtersi, which also cannot tolerate rapid drying (FIG. 1A) (Wright, 1989). These experiments recapitulated our H dujardini results with 20 of 31 CAHS transcripts, 2 of 19 SAHS transcripts, and 0 of 2 MAHS transcripts enriched in dry P. richtersi specimens.

To test if the extent to which a tardigrade species requires preconditioning mirrors the induction of TDPs upon desiccation, we assembled and analyzed the transcriptome (from publically available short reads) of a third tardigrade species, Milnesium tardigradum, which requires much less preconditioning (FIG. 1A) (Wright, 1989). M tardigradum did not significantly enrich expression any TDPs during desiccation. However, several CAHS transcripts were expressed at constitutively high levels. For example, one CAHS transcript was the third most abundant transcript identified.

Combined, these data demonstrate that the expression level of TDPs in different tardigrade species mirrors the degree to which that species requires preconditioning. In species requiring extensive preconditioning (H. dujardini and P. richtersi) many TDPs are upregulated upon desiccation, while in a tardigrade requiring relatively little preconditioning (M tardigradum) these genes do not respond to drying but are constitutively expressed at high levels.

Tardigrade-specific Intrinsically Disordered Proteins Are Required for Desiccation Tolerance. To test if TDPs are required for tardigrades to survive desiccation, we performed RNAi (Tenlen et al., 2013) to disrupt the function of specific genes. We targeted both highly induced (CAHSs and SAHSs) and constitutively active (SAHSs) TDPs and tested the ability of H. dujardini to survive under control (hydrated) and dry conditions. For all treatments, under hydrated conditions there were no significant decreases in survival (FIG. 2A). However, targeting 2 of 4 highly induced (13- to 22-fold) CAHS genes had significantly (p-value <0.01) reduced survival after desiccation compared to a control treatment, GFP RNAi (FIG. 2B). Additionally, RNAi targeting of an induced (5-fold) SAHS gene resulted in a significant (p-value <0.01) decrease in survival after desiccation compared to the GFP RNAi controls (FIG. 2B). These results demonstrate that some TDPs expressed at high levels in drying tardigrades are also essential for tardigrades to survive desiccation.

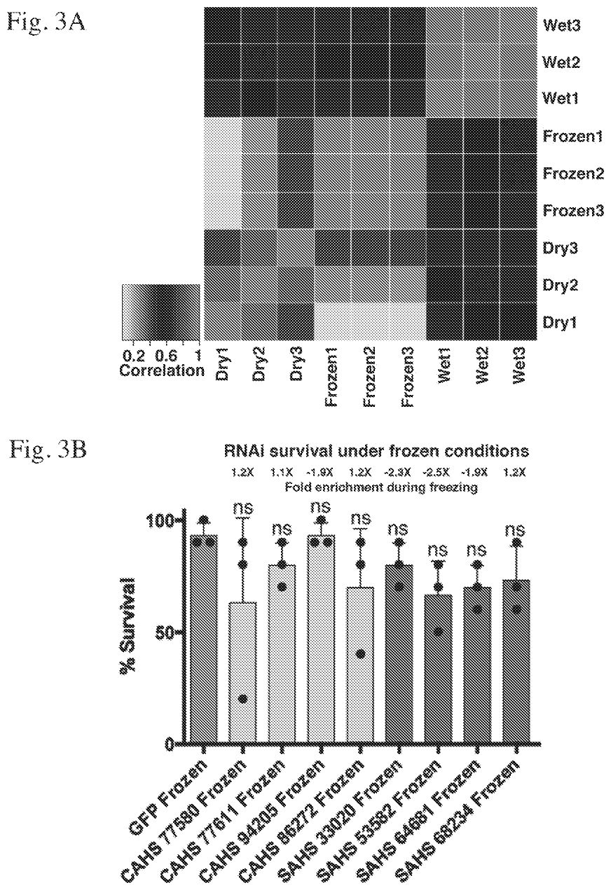

It has been suggested that tardigrades may have first evolved the ability to survive drying and acquired resistances to other stresses (cross-tolerance) as a byproduct of desiccation tolerance (Jonsson, 2003). If true, one would anticipate that different forms of stress would induce similar changes in gene expression (Sinclair et al., 2013). To test this idea, we sequenced transcriptomes of gradually frozen H. dujardini specimens and compared changes in gene expression induced by freezing to those induced by drying. Changes in expression under these stress conditions were divergent, with gene expression in either stress condition (frozen or dry) being more similar to control conditions (hydrated) than to the other stress condition (FIG. 3A). Additionally, only 2 of 17 CAHS transcripts were enriched during freezing (as opposed to 11 of 17 under drying conditions), and these genes were expressed at relatively low levels and underwent small changes in expression. No SAHS or MAHS transcripts were enriched during freezing in H. dujardini. Interestingly, none of our CAHS or SAHS RNAi treatments significantly decreased survival of frozen tardigrades relative to double stranded GFP RNAi controls (FIG. 3B). Our RNAi results, coupled with the observed divergence between frozen and drying transcriptomes, suggest that different stresses may be less mechanistically linked than previously suspected.

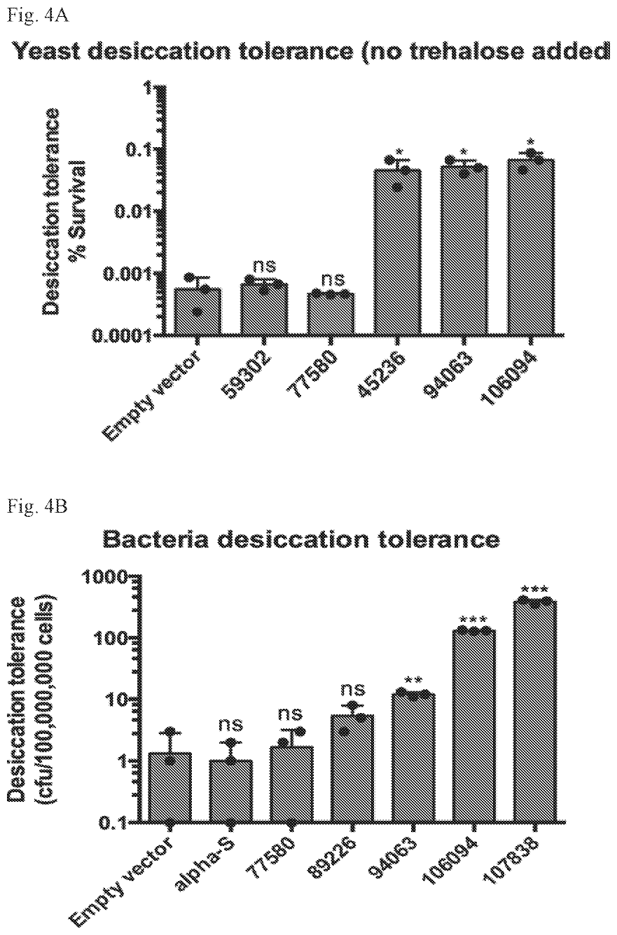

Tardigrade-specific Intrinsically Disordered Proteins Are Sufficient to Increase Desiccation Tolerance in Heterologous Systems. To test if TDPs might be good protectants, we assessed their ability to increase the desiccation tolerance of other systems by quantifying the desiccation tolerance (percent survival) of yeast and bacteria engineered to exogenously express CAHS proteins (FIG. 4A-4B). Several CAHS TDP proteins were sufficient to increase the desiccation tolerance of yeast nearly 100-fold (FIG. 4A). Similar results were obtained in bacteria, with exogenous expression of some CAHS proteins resulting in over two orders of magnitude increases in desiccation tolerance (FIG. 4B). Importantly, a-synuclein, a protein that exists as a disordered monomer in cells (Fauvet et al., 2012; Theillet et al., 2016) and has no known connection to stress tolerance (Drescher et al., 2012; Theillet et al., 2014), did not increase survival under drying conditions (FIG. 4B), demonstrating that something beyond intrinsic disorder of TDPs is essential for their protective capabilities.

In summary, we have demonstrated that tardigrades express TDPs in response to drying and/or constitutively express TDPs at high levels. The level of TDP enrichment during drying mirrors different tardigrade species' requirement for preconditioning (slow drying) to survive desiccation. We find that several TDPs contributed functionally to H. dujardini 's ability to survive desiccation. Additionally, this study shows that changes in tardigrades' gene expression induced by different stress conditions are more divergent than suspected. Our study demonstrates that exogenous expression of TDP proteins in both prokaryotic and eukaryotic cells is sufficient to increase desiccation tolerance in these systems. TDPs represent the first functional mediators of tardigrade stress tolerance to be identified.

### Example 15

### Stabilization of Protein by TDPs

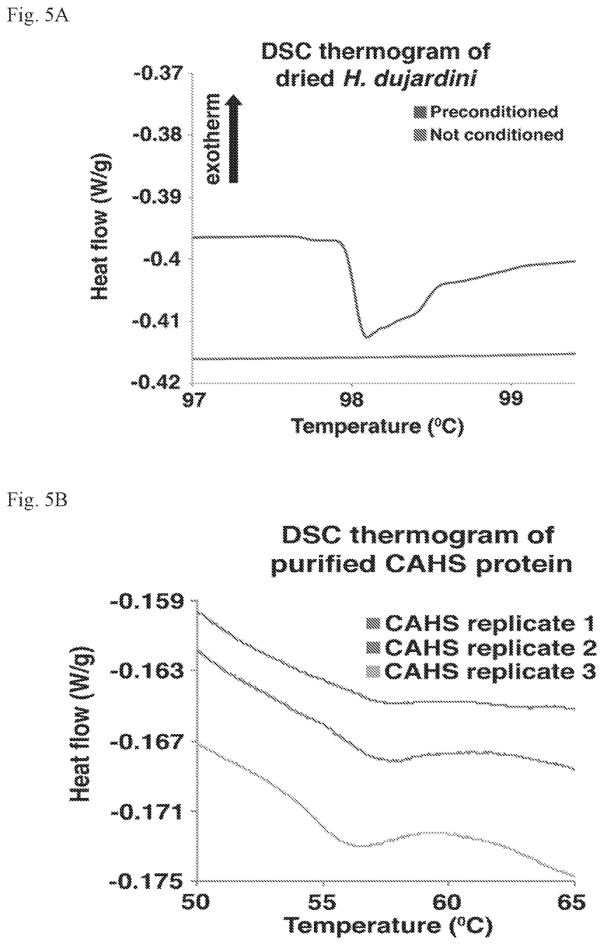

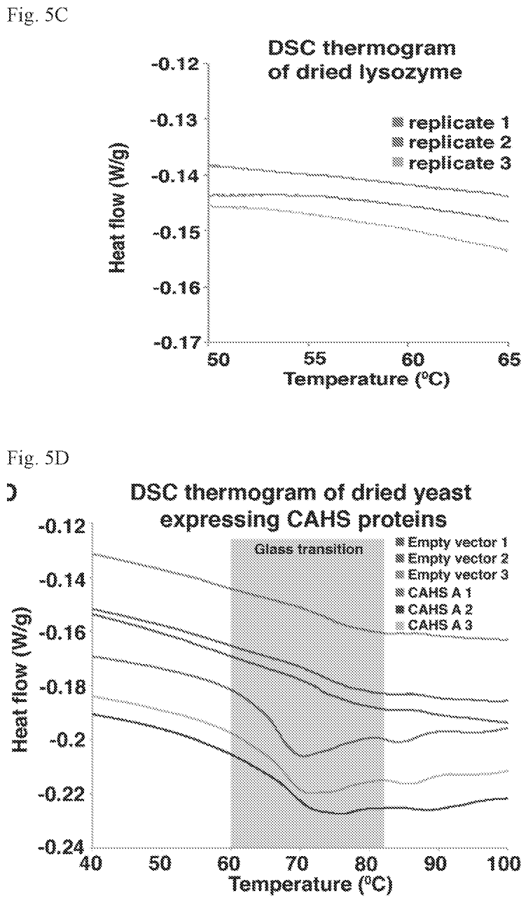

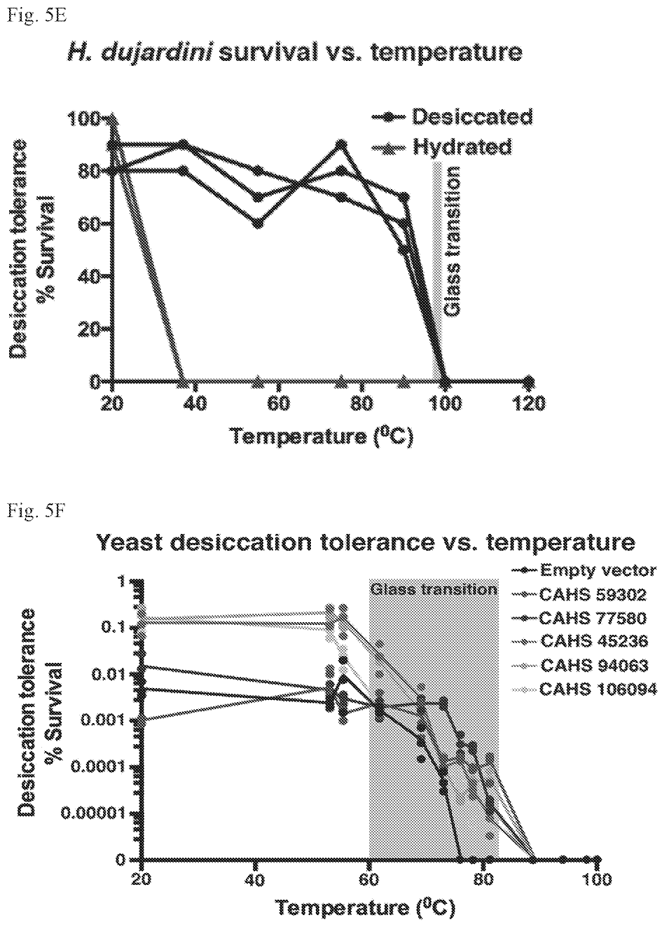

We wondered how CAHS proteins might mechanistically function in desiccation tolerance. The vitrification hypothesis posits that organisms produce amorphous solids, called bioglasses, during desiccation to help prevent proteins from denaturing and aggregating, and to maintain the integrity of membranes under dry conditions (Sun, Wet al. Comp. Biochem. Physiol. A Physiol. 117, 327-333 (1997); Crowe, et al. Annu. Rev. Physiol. 60, 73-103 (1998)). Some tardigrade species are known to vitrify upon desiccation and this vitrified state appears essential for their survival of high temperatures under desiccated conditions, however the molecule(s) responsible for producing this vitrified state in tardigrades are unknown (Hengherr et al. Physiol. Biochem. Zool. 82, 749-755 (2009)). To test if H. dujardini produce glassy material as they dry we used differential scanning calorimetry (DSC), a well-established method of glass characterization^{16,17}, to assay for the presence of glassy material in H. dujardini specimens that had been dried slowly (allowing for production of TDPs) or quickly (not allowing time for production of TDPs) (FIG. 5A). DSC thermograms showed the presence of a glassy material in specimens that had been dried slowly, but glassy material was not detected in specimens dried quickly (FIG. 5A). These results suggest that material capable of vitrifying upon desiccation is made as H. dujardini dry out, and that tardigrades must dry slowly to allow production of this vitrifying material.

Since TDP genes are induced and abundantly expressed during desiccation, we tested the ability of proteins encoded by these genes to form bioglasses. We found that TDPs formed bioglasses in vitro or in vivo when exogenously expressed in yeast (FIGS. 5B and 5D). Together these data demonstrate that TDPs form bioglasses, which may serve a protective role during desiccation.

The ability of multiple species of tardigrades to survive high temperatures while desiccated has been correlatively linked to the presence of glassy material (Hengherr et al. Physiol. Biochem. Zool. 82, 749-755 (2009)). To test if the glassy state H dujardini and of TDPs specifically might play a role in desiccation tolerance we tested the ability of dried H. dujardini specimens and yeast expressing TDP genes to survive desiccation after being heated below, at, and above the experimentally measured glass transition temperature. Though correlative, this approach has been used before to assess the role of vitrification in the desiccation tolerance of organism (Sakurai et al. Proc. Natl. Acad. Sci. 105, 5093-5098 (2008); Hengherr et al. Physiol. Biochem. Zool. 82, 749-755 (2009)). Glassy material remains in its glassy state below the transition temperature, whereas at or above the temperature, the material transitions into a rubbery or molten solid, with a higher degree of molecular motion. Preconditioned H. dujardini specimens have a sharp transition, starting just below 98° C. and ending around 101° C. (FIG. 5A). Slowly dried tardigrades heated to various temperatures survived heating until ˜100° C., after which no tardigrades survived (FIG. 5E). Dried yeast expressing different CAHS proteins have novel glass transitions that range between ˜55° C. and ˜82° C. (FIG. 5D).

We speculate that the higher glass transition temperature in tardigrades relative to yeast expressing TDPs is likely due to interactions of TDPs with other endogenous tardigrade molecules, which may strengthen or work synergistically with bioglasses (Wolkers et al. Biochim. Biophys. Acta 1544, 196-206 (2001)). Similar to slow dried H. dujardini specimens, dried yeast expressing TDPs did not show major decreases in desiccation tolerance when heated below the glass transition temperature (FIG. 5F). However, at temperatures within their glass transition range, survival decreased and no survival was observed after heating to 81° C. In concordance with the hypothesis that the glassy state of TDPs is important for their protective capabilities, the maximal heat tolerance of dried yeast was increased from about 76° C. in wild type yeast to above 81° C. in yeast engineered to express TDPs (FIG. 5F). These data suggest that the glassy state of dried CAHS proteins maybe essential for their function in desiccation and thermotolerance.

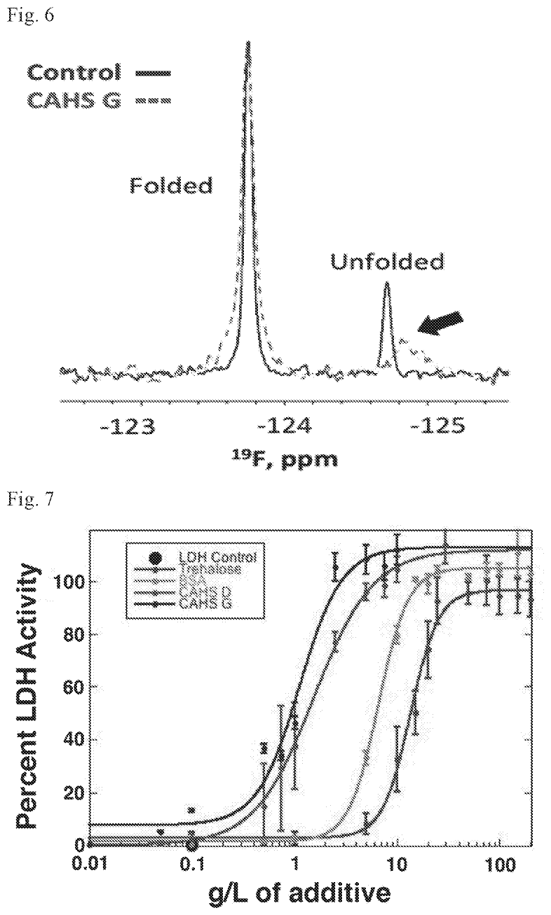

When living organisms desiccate there are a number of things that can go wrong within their cells, which have evolved to function in a hydrated state. One of the detrimental effects of desiccation is the denaturing or unfolding of proteins. To test if TDPs can help stabilize proteins in their folded state we used F¹⁹ NMR to test the effect TDPs have on the dynamics of SH3 (N-terminal SH3 (SRC Homology 3) domain of the Drosophila drk (downstream of receptor kinase) protein folding. SH3 is an unstable protein that in normal aqueous solutions is unfolded about 50% of the time. Using F¹⁹ NMR we measured the relative amounts of SH3 protein in a folded and unfolded state (FIG. 6). As we previously reported (Senske, et al. Angew. Chem. Int. Ed. 55, 3586-3589 (2016); Smith et al. Proc. Natl. Acad. Sci. 113, 1725-1730 (2016)), we found that by itself SH3 is unstable with a substantial population of protein being in an unfolded state (FIG. 6). However, mixing SH3 with TDPs results in the stabilization of the SH3 protein, with essentially all the SH3 protein now being in a folded state (FIG. 6). These experiments demonstrate at TDPs can stabilize the structural integrity of other, more sensitive proteins, maintaining them in their folded conformation.

The proper folding of most proteins is essential for their function. If they unfold or denature they cannot perform their cellular functions. Since tardigrades require TDPs to survive desiccation, and yeast and bacterial desiccation tolerance is increased by TDPs, we were curious if TDPs preserve the functional integrity of proteins under desiccated conditions. To test this we assessed the activity of lactate dehydrogenase (LDH) before and after being desiccated. We found that LDH alone, when desiccated and then rehydrated, loses most of its functional ability, working at only about 2% of its original activity (FIG. 7). In stark contrast, LDH desiccated in the presence of TDPs, at concentrations >10 g/L and then rehydrated, functions at 100% its original activity (FIG. 7). Furthermore, TDPs achieve a higher level of protection and protect LDH at lower concentrations than other additives (trehalose and BSA; FIG. 7). These data demonstrate the TDPs can efficiently stabilize and preserve the function of proteins in a desiccated state.

The foregoing is illustrative of the present invention, and is not to be construed as limiting thereof. The invention is defined by the following claims, with equivalents of the claims to be included therein.

### REFERENCES

Bai, Y., Milne, J. S., Mayne, L., and Englander, S. W. (1993). Primary structure effects on peptide group hydrogen exchange. Proteins Struct. Funct. Genet. 17, 75-86.
Bemm, F., WeiB, C. L., Schultz, J., and Forster, F. (2016). Genome of a tardigrade: Horizontal gene transfer or bacterial contamination? Proc. Natl. Acad. Sci. 113, E3054-E3056.
Boothby, T. C., Tenlen, J. R., Smith, F. W., Wang, J. R., Patanella, K. A., Osborne Nishimura, E., Tintori, S. C., Li, Q., Jones, C. D., Yandell, M., et al. (2015). Evidence for extensive horizontal gene transfer from the draft genome of a tardigrade. Proc. Natl. Acad. Sci. 112, 15976-15981.
Chakrabortee, S., Tripathi, R., Watson, M., Kaminski Schierle, G. S., Kurniawan, D. P., Kaminski, C. F., Wise, M. J., and Tunnacliffe, A. (2012). Intrinsically disordered proteins as molecular shields. Mol BioSyst 8, 210-219,
Drescher, M., Huber, M., and Subramaniam, V. (2012). Hunting the Chameleon: Structural Conformations of the Intrinsically Disordered Protein Alpha-Synuclein. ChemBioChem 13, 761-768.
Englander, S. W., and Kallenbach, N. R. (1983). Hydrogen exchange and structural dynamics of proteins and nucleic acids. Q Rev Biophys 16, 521-655.
Erkut, C., Penkov, S., Khesbak, H., Vorkel, D., Verbavatz, J.-M., Fahmy, K., and Kurzchalia, T. V. (2011). Trehalose Renders the Dauer Larva of Caenorhabditis elegans Resistant to Extreme Desiccation. Curr. Biol. 21, 1331-1336.

Fauvet, B., Mbefo, M. K., Fares, M.-B., Desobry, C., Michael, S., Ardah, M. T., Tsika, E., Coune, P., Prudent, M., Lion, N., et al. (2012). -Synuclein in Central Nervous System and from Erythrocytes, Mammalian Cells, and Escherichia coli Exists Predominantly as Disordered Monomer. J. Biol. Chem. 287, 15345-15364.

Forney, C. F., and Brandl, D. G. (1992). Control of Humidity in Small Controlled environment Chambers using Glycerol-Water Solutions. HortTechnology 2, 52-54.

Gabriel, W. N., McNuff, R., Patel, S. K., Gregory, T. R., Jeck, W. R., Jones, C. D., and Goldstein, B. (2007). The tardigrade Hypsibius dujardini, a new model for studying the evolution of development. Dev. Biol. 312, 545-559.
Garay-Arroyo, A., Colmenero-Flores, J. M., Garciarrubio, A., and Covarrubias, A. A. (2000). Highly hydrophilic proteins in prokaryotes and eukaryotes are common during conditions of water deficit. J. Biol. Chem. 275, 5668-5674.
Goldstein, B., and Blaxter, M. (2002). Tardigrades. Curr. Biol. 12, R475.
Guidetti, R., Altiero, T., and Rebecchi, L. (2011). On dormancy strategies in tardigrades. J. Insect Physiol. 57, 567-576.
Haas, B. J., Papanicolaou, A., Yassour, M., Grabherr, M., Blood, P. D., Bowden, J., Couger, M. B., Eccles, D., Li, B., Lieber, M., et al. (2013). De novo transcript sequence reconstruction from RNA-seq using the Trinity platform for reference generation and analysis. Nat. Protoc. 8, 1494-1512.

Hengherr, S., Heyer, A. G., Köhler, H.-R., and Schill, R. O. (2008). Trehalose and anhydrobiosis in tardigrades—evidence for divergence in responses to dehydration: Trehalose and anhydrobiosis in tardigrades. FEBS J. 275, 281-288.

Hengherr, S., Worland, M. R., Reuner, A., Brümmer, F., and Schill, R. O. (2009). High-Temperature Tolerance in Anhydrobiotic Tardigrades Is Limited by Glass Transition. Physiol. Biochem. Zool. 82, 749-755.

Hoekstra, F. A., Golovina, E. A., and Buitink, J. (2001). Mechanisms of plant desiccation tolerance. Trends Plant Sci. 6, 431-438.

Jönsson, K. I. (2003). Causes and Consequences of Excess Resistance in Cryptobiotic Metazoans. Physiol. Biochem. Zool. 76, 429-435.

Jönsson, K. I., and Persson, O. (2010). Trehalose in three species of desiccation tolerant tardigrades. Open Zool. J. 3, 1-5.

Kondo, K., Kubo, T., and Kunieda, T. (2015). Suggested Involvement of PP1/PP2A Activity and De Novo Gene Expression in Anhydrobiotic Survival in a Tardigrade, Hypsibius dujardini, by Chemical Genetic Approach. PLOS ONE 10, e0144803.

Koutsovoulos, G., Kumar, S., Laetsch, D. R., Stevens, L., Daub, J., Conlon, C., Maroon, H., Thomas, F., Aboobaker, A. A., and Blaxter, M. (2016). No evidence for extensive horizontal gene transfer in the genome of the tardigrade Hypsibius dujardini. Proc. Natl. Acad. Sci. 201600338.

Lapinski, J., and Tunnacliffe, A. (2003). Anhydrobiosis without trehalose in bdelloid rotifers. FEBS Lett. 553, 387-390.

Levin, M., Anavy, L., Cole, A. G., Winter, E., Mostov, N., Khair, S., Senderovich, N., Kovalev, E., Silver, D. H., Feder, M., et al. (2016). The mid-developmental transition and the evolution of animal body plans. Nature 531, 637-641.

Li, B., and Dewey, C. N. (2011). RSEM: accurate transcript quantification from RNA-Seq data with or without a reference genome. BMC Bioinformatics 12, 323.

Mali, B., Grohme, M. A., Förster, F., Dandekar, T., Schnölzer, M., Reuter, D., We\lnicz, W., Schill, R. O., and Frohme, M. (2010). Transcriptome survey of the anhydrobiotic tardigrade Milnesium tardigradum in comparison with Hypsibius dujardini and Richtersius coronifer. BMC Genomics 11, 168.

Parra, G., Bradnam, K., and Korf, I. (2007). CEGMA: a pipeline to accurately annotate core genes in eukaryotic genomes. Bioinformatics 23, 1061-1067.

Rebecchi, L., Altiero, T., Guidetti, R., Cesari, M., Bertolani, R., Negroni, M., and Rizzo, A. M. (2009). Tardigrade resistance to space effects: first results of experiments on the LIFE-TARSE mission on FOTON-M3 (September 2007). Astrobiology 9, 581-591.

Robinson, M. D., McCarthy, D. J., and Smyth, G. K. (2010). edgeR: a Bioconductor package for differential expression analysis of digital gene expression data. Bioinformatics 26, 139-140.

Schwarzinger, S., Kroon, G. J., Foss, T. R., Wright, P. E., and Dyson, H. J. (2000). Random coil chemical shifts in acidic 8 M urea: implementation of random coil shift data in NMR View. J. Biomol. NMR 18, 43-48.

Sinclair, B. J., Ferguson, L. V., Salehipour-shirazi, G., and MacMillan, H. A. (2013). Cross-tolerance and Cross-talk in the Cold: Relating Low Temperatures to Desiccation and Immune Stress in Insects. Integr. Comp. Biol. 53, 545-556.

Tanaka, S., Tanaka, J., Miwa, Y., Horikawa, D. D., Katayama, T., Arakawa, K., Toyoda, A., Kubo, T., and Kunieda, T. (2015). Novel Mitochondria-Targeted Heat-Soluble Proteins Identified in the Anhydrobiotic Tardigrade Improve Osmotic Tolerance of Human Cells. PLOS ONE 10, e0118272.

Tapia, H., and Koshland, D. E. (2014). Trehalose Is a Versatile and Long-Lived Chaperone for Desiccation Tolerance. Curr. Biol. 24, 2758-2766.

Tenlen, J. R., McCaskill, S., and Goldstein, B. (2013). RNA interference can be used to disrupt gene function in tardigrades. Dev. Genes Evol. 223, 171-181.

Theillet, F.-X., Binolfi, A., Frembgen-Kesner, T., Hingorani, K., Sarkar, M., Kyne, C., Li, C., Crowley, P. B., Gierasch, L., Pielak, G. J., et al. (2014). Physicochemical Properties of Cells and Their Effects on Intrinsically Disordered Proteins (IDPs). Chem. Rev. 114, 6661-6714.

Theillet, F.-X., Binolfi, A., Bekei, B., Martorana, A., Rose, H. M., Stuiver, M., Verzini, S., Lorenz, D., van Rossum, M., Goldfarb, D., et al. (2016). Structural disorder of monomeric α-synuclein persists in mammalian cells. Nature 530, 45-50.

Wang, C., Grohme, M. A., Mali, B., Schill, R. O., and Frohme, M. (2014). Towards Decrypting Cryptobiosis—Analyzing Anhydrobiosis in the Tardigrade Milnesium tardigradum Using Transcriptome Sequencing. PLoS ONE 9, e92663.

Westh, P., and Ramloøv, H. (1991). Trehalose accumulation in the tardigrade Adorybiotus coronifer during anhydrobiosis. J. Exp. Zool. 258, 303-311.

Wright, J. C. (1989). Desiccation tolerance and water-retentive mechanisms in tardigrades. J. Exp. Biol. 142, 267-292.

Yamaguchi, A., Tanaka, S., Yamaguchi, S., Kuwahara, H., Takamura, C., Imajoh-Ohmi, S , Horikawa, D. D., Toyoda, A., Katayama, T., Arakawa, K., et al. (2012). Two Novel Heat-Soluble Protein Families Abundantly Expressed in an Anhydrobiotic Tardigrade. PLoS ONE 7, e44209.

1. A liquid composition comprising:
at least one tardigrade disordered protein (TDP); and
at least one heterologous polypeptide and/or peptide of interest.

2. The liquid composition of claim 1, further comprising one or more excipients.

3. A solid composition produced by drying the liquid composition of claim 1.

4-5. (canceled)

6. The liquid composition of claim 1, wherein the concentration of the at least one TDP is about 1 g/L to about 100 g/L, optionally wherein the mass ratio of the at least one heterologous polypeptide and/or peptide of interest to the at least one TDP is about 1:100 to about 1:10, optionally about 1:10 to about 1:20.

7. (canceled)

8. The liquid composition of claim 1, wherein the at least one TDP is selected from the group of amino acid sequences having at least about 80% identity to any one of SEQ ID NOs:1-105; amino acid sequences encoded by a nucleotide sequence having at least about 80% identity to any one of SEQ ID NOs:106-210, and a complement thereof; and amino acid sequences encoded by a nucleotide sequence having at least about 80% identity to any one of SEQ ID NO:211-315; and any combination thereof.

9. The liquid composition of claim 1, wherein the at least one TDP is selected from the group of amino acid sequences having at least about 80% identity to any one of SEQ ID NOs:17, 19, 32, 35, and 38; amino acid sequences encoded by a nucleotide sequence having at least about 80% identity to any one of SEQ ID NOs:122, 124, 137, 140, and 143, and a complement thereof amino acid sequences encoded by a nucleotide sequence having at least about 80% identity to any one of SEQ ID NOs:227, 229, 242, 245 and 248; and any combination thereof.

10. The composition of claim 1, wherein the at least one heterologous polypeptide and/or peptide of interest is a therapeutic agent, optionally wherein the therapeutic agent is a protein based vaccine, an antibody, an enzyme, hormone, and/or a globular protein.

11-18. (canceled)

19. A method of stabilizing at least one heterologous polypeptide and/or peptide of interest, comprising,
contacting the at least one heterologous polypeptide and/or peptide of interest with at least one tardigrade disordered protein (TDP) to produce a liquid composition comprising the at least one heterologous polypeptide and/or peptide of interest and the at least one TDP, thereby stabilizing the at least one heterologous polypeptide and/or peptide of interest, optionally wherein the at least one heterologous polypeptide and/or peptide of interest is a therapeutic agent or is part of a protein-based food.

20. The method of claim 19, further comprising at least partially drying the liquid composition comprising the at least one heterologous polypeptide of interest and the at least one tardigrade disordered protein (TDP).

21-23. (canceled)

24. A method of stabilizing a heterologous cell, tissue or organ, comprising,
contacting the heterologous cell, tissue or organ with a solution comprising at least one tardigrade disordered protein (TDP), thereby stabilizing the heterologous cell, tissue or organ.

25. The method of claim 24, wherein the concentration of the solution comprising the at least one TDP is about 1 g/L to about 100 g/L.

26. The method of claim 24, further comprising desiccating the heterologous cell, tissue or organ that is contacted with the TDP.

27-28. (canceled)

29. The method of claim 19, wherein the at least one TDP is selected from the group consisting of amino acid sequences having at least about 80% identity to any one of SEQ ID NOs:1-105; amino acid sequences encoded by a nucleotide sequence having at least about 80% identity to any one of SEQ ID NOs:106-210, and a complement thereof; amino acid sequences encoded by a nucleotide sequence having at least about 80% identity to any one of SEQ ID NO:211-315; and any combination thereof.

30. The method of claim 19, wherein the at least one TDP is selected from the group consisting of amino acid sequences having at least about 80% identity to any one of SEQ ID NOs:17, 19, 32, 35, and 38; amino acid sequences encoded by a nucleotide sequence having at least about 80% identity to any one of SEQ ID NOs:122, 124, 137, 140, and 143, a complement thereof; amino acid sequences encoded by a nucleotide sequence having at least about 80% identity to any one of SEQ ID NOs:227, 229, 242, 245 and 248; and any combination thereof.

31-34. (canceled)

35. A method of increasing drought or desiccation tolerance in an organism comprising:
introducing into the organism at least one heterologous nucleotide sequence encoding a tardigrade disordered protein (TDP), to produce a transgenic organism expressing the heterologous nucleotide sequence, thereby increasing the drought or desiccation tolerance of the transgenic organism.

36. (canceled)

37. The method of claim 35, wherein the at least one heterologous nucleotide sequence encoding a TDP is selected from the group consisting of nucleotide sequences having at least about 80% identity to any one of SEQ ID NOs:106-210, and a complement thereof; having at least about 80% identity to any one of SEQ ID NO:211-315; encoding an amino acid sequence having at least about 80% identity to any one of SEQ ID NOs:1-105; and any combination thereof.

38. The method of claim 37, wherein the at least one heterologous nucleotide sequence encoding a TDP is selected from the group consisting of nucleotide sequences having at least about 80% identity to any one of SEQ ID NOs:122, 124, 137, 140, and 143, and a complement thereof; having at least about 80% identity to any one of SEQ ID NOs:227, 229, 242, 245 and 248; encoding an amino acid sequence having at least about 80% identity to any one of SEQ ID NOs:17, 19, 32, 35, and 38; and any combination thereof.

39. A transgenic plant, fungus or bacterium having increased tolerance to drought or desiccation produced by the method of claim 35.

40. (canceled)

41. The transgenic plant, fungus or bacterium of claim 39, wherein the at least one heterologous nucleotide sequence encoding a TDP is selected from the group consisting of nucleotide sequences having at least about 80% identity to any one of SEQ ID NOs:106-210, and a complement thereof; having at least about 80% identity to any one of SEQ ID NO:211-315; encoding an amino acid sequence having at least about 80% identity to any one of SEQ ID NOs:1-105; and any combination thereof.

42. The transgenic plant, fungus or bacterium of claim 39, wherein the at least one heterologous nucleotide sequence encoding a TDP is selected from the group consisting of nucleotide sequences having at least about 80% identity to any one of SEQ ID NOs:122, 124, 137, 140, and 143, and a complement thereof; having at least about 80% identity to any one of SEQ ID NOs:227, 229, 242, 245 and 248; encoding an amino acid sequence having at least about 80% identity to any one of SEQ ID NOs:17, 19, 32, 35, and 38; and any combination thereof.

43. (canceled)


SEQUENCE LISTING (321 sequences)

SEQ ID NO: 1 - protein, 266 aa, Paramacrobiotus richtersi
MSGRIEQHMEAEECQGGAYCPPNCRYHSRGMKQEHEEKHVYRESVTPGHAERREERRDEQYQRPSESYPESNRQVEKEAVNTARVHTTVSAPIVAPPAPVISVAPVAEELASGYTGSAARYTASSEVTILPNPKLTEEARRDEIARQKEADDIARRHEQDLAKRSEQYRKQAEAEAEKIRKELEKQHDKDIAFRKSLIDSAVTRQQREIDLEAKMAKKELLREAEQAKESLEKTRAATTVEVDFTTAVGHTHSAGVTASETIRTNQ

SEQ ID NO: 2 - protein, 267 aa, Paramacrobiotus richtersi
MSGRIEQHMEAEECQGGAYCPPNCRYHSRGMKQEHEEKQVYRESITPGHAERRVEERRDEQYQRPSESYPESNRQVEKEVVNTARVHTTVSAPIVAPPAPVITVAPVAEELASGFTGSAARYTASSEVTILPNPKLTEEARRDEIARQKEADDIARRHEQDLAKRSEQYRKQAEAEAEKIRKELEKQHDKDIAFRKSLIDSAVTRQQREIDLEAKMAKKELLREAEQAKESLEKTRAATTVEVDFTTAVGHTHSAGVTASETIRTNQ

SEQ ID NO: 3 - protein, 278 aa, Paramacrobiotus richtersi
MERKVVEKIEVHTTGNVPPVIPAACSNVTCTTVCDPKCTERHEHHHHTGVACNTVCSPACGERHEHHQHHQHHEHSGSCTETSEKSTHYTHTEVKAPVLNPSAPFVVTSASGLAQEIVSEGFSASAARISGESVGTIVHESAASSKQAAVDLEKYEREKAAIAKQHEKELEKKTESYRKQAEAEAEKIRKELEKQHARDVEFRKDVLETTIERQKKEVELEAKMAKKELEHEKKLAMDALEHSKMSTNIEVKFDSAAGHTTTEGVVVSESVNVAHPRM

SEQ ID NO: 4 - protein, 87 aa, Paramacrobiotus richtersi
RKELEKQHQRDIDFKEELVGEAIAAQKKQVDLEANLAKRQLEREGQAARDALEKSKMTTNVEVNFDTAIGHAVSGSSTVESAVHKTH

SEQ ID NO: 5 - protein, 87 aa, Paramacrobiotus richtersi
RKELEKQHQRDIDFKEELVGEAIAAQKKQVDLEANLAKRQLEREGQAARDALEKSKMTTNVEVNFDTAIGHAVSGSTTVESEVHKTH

SEQ ID NO: 6 - protein, 546 aa, Paramacrobiotus richtersi
MPLFGSSKKDKHKDDIVVTNQDIDVERDRDSVVSRDRDSVVSTHLDRDLTTVPGDKHSHEFKYERVEEIHVDADGNAELRDVRVDRGGEDPGMNFKDKRPPALVPGAPVGYVPEIHELDSVATQRQGIQNYFADSSSVSHTQRKSREPSLLEEREQTIIREGSMASGASINNNAGIPPTVPLERFSQRSVSQSGDDTSSIASSVSSVSSVSSASTATAGSRVSQNTTGTRDRVNIARQEAPALQRDVDYIQQGIENLQNLPIMNPARDTYVTERRTDASYVQNIPPVVEMGRAPIYRQEQDIIIPGRHREVIERTEVIQSAAPRQGSVEVIQIPIHRMEAAQMEHIRSGVTYTNDKELIIPGAMLAPPIPSVTHDLLAQGSGGTHAEIYADTNIDLLANTQLKQSPEEYARYRASVEALAREHEMDTAQRAAMYRNQVEADAELIRRTLERQHIRDIEFRKEMVETAVDRQQHEIQLEAEYAMRALEKEREAASRALEQAKAQTHIDVRVDTAIGTTISKGDVQTAAGREIRENVGRVESYPATRF

SEQ ID NO: 7 - protein, 538 aa, Paramacrobiotus richtersi
MPLFGSSKKDKHKDDIVVTNQDIDVERDRDSVVSTHLDRDLTTVPGDKHSHEFKYERVEEIHVDADGNAELRDVRVDRGGEDPGMNFKDKRPPALVPGAPVGYVPEIHELDSVATQRQGIQNYFADSSSVSHTQRKSREPSLLEEREQTIIREGSMASGASINNNAGIPPTVPLERFSQRSVSQSGDDTSSIASSVSSVSSVSSASTATAGSRVSQNTTGTRDRVNIARQEAPALQRDVDYIQQGIENLQNLPIMNPARDTYVTERRTDASYVQNIPPVVEMGRAPIYRQEQDIIIPGRHREVIERTEVIQSAAPRQGSVEVIQIPIHRMEAAQMEHIRSGVTYTNDKELVIPGAMLAPPIPSVTHDLLAQGSGGTHAEIYADTNIDLLANTQLKQSPEEYARYRASVEALAREHEMDTAQRAAMYRNQVEADAELIRRTLERQHIRDIEFRKEMVETAVDRQQHEIQLEAEYAMRALEKEREAASRALEQAKAQTHIDVRVDTAIGTTISKGDVQTAAGREIRENVGRVESYPATRF

SEQ ID NO: 8 - protein, 298 aa, Paramacrobiotus richtersi
MSHHHEEKFERVEERKVDPARGVQEVRVGMDTGHGDPALNFQPTDATLVKGRTVVGGVDAAGMTTGATQYSGASTVQSGTNTFEAEKNTSYTHTEVRAPLVTPAAPFISTGVTGLAQDVVGEGFTASAARITAGSASAVVTETAEMRDKSMKEQERYLREKEAIARSHEKDLEKKTEAYRKEAEAEAEKIRKELEKQHARDVEFRKDMVDETIERQKREVELEAKYAKKELEHERQMAQNALDQSKMATNIEVSMDTAAGRTVSGGTTVSESFETHHEEHGKEKKSLGEKIKDTFLGR

SEQ ID NO: 9 - protein, 298 aa, Paramacrobiotus richtersi
MSHHHEEKFERVEERKVDPARGVQEVRVGMDTGHGDPALNFQPTDATLVKGRTVVGGVDAAGMTTGATQYSGASTVQSGTNTYEAEKNTSYTHTEVRAPLVTPAAPFISTGVTGLAQDVVGEGFTASAARITAGSASAVVTETAEMRDKSMKEQERYLREKEAIARSHEKDLEKKTEAYRKEAEAEAEKIRKELEKQHARDVEFRKDMVDETIERQKREVELEAKYAKKELEHERQMAQNALDQSKMATNIEVSMDTAAGRTVSGGTTVSESFETHHEEHGKEKKSLGEKIKDTFLGR

SEQ ID NO: 10 - protein, 222 aa, Paramacrobiotus richtersi
MPHTHEHKEVKEVRTSDGGHLVESIKNVSSTTHVDTDTLDTATTHTTIHAPLIHPTGTVSVHAVSGLAQELLGEGITASVERVTAGTRDEVIYETPEQLERKRDRDEKYYQAKEKIREKHEKEIGKLTEDYREKTERETAKIRKEMEKQHERDVEFRSKLVEDAIKRQKEELELEAKYAKKELERQRELALDALENSRMHTDISVNMDTTVGHTVSSGRIDS

SEQ ID NO: 11 - protein, 278 aa, Paramacrobiotus richtersi; identical to SEQ ID NO: 3

SEQ ID NO: 12 - protein, 328 aa, Paramacrobiotus richtersi
MPLFGSNKDKDSKSSYKEEHHESHTERRMEDNCPPPMLSKDMPTNLSGKVVVERHETVSTIPDVKPVVEMSRTPMYRQEADIHIQAGHREVVERTDVIKSKAQAQKEVEIVSIPIQKMAAAQMEHVRTGVTFTQDKEMIIPGPMVAPPIPSVTHDLLVQGSGGTSAEIHASTNVDLLANAQLAGQSPEEYARYRAGVEQLAHQHEVETTQKAEAYRHQVEADAELIRRTLERQHVRDIEFRKDMVSTAVDRQQQEIKMEAEYAMKALEQERIAAERALDQAKMETHIDVKVDTAIGTTISKGEVRTAAGREIRESVGPVTVHHGATRI

SEQ ID NO: 13 - protein, 327 aa, Paramacrobiotus richtersi
MPLFGSNKDKDSKSSYKEEHHESHTERRMEDNCPPPMLSKDMPTNLSEKVIVERHETVSTIPDVKPVVEMSRTPMYRQEADIHIQAGHREVVERTDVIKSKAQAQKEVEIVSIPIQKMAAAQMEHVRTGVTFTQDKEMIIPGAMVAPPIPSVTHDLLVQGSGGTSAEIHASTNVDLLANAQLAGQSPEEYARYRAGVEQLAHQHEVETTQKAEAYRHQVEADAELIRRTLERQHVRDIEFRKDMVSTAVDRQQQEIKMEAEYAMKALEQERIAAERALDQAKMETHIDVKVDSAIGTTVSKGDVLTAAGKEIRENVGPVTRDHPARH

SEQ ID NO: 14 - protein, 263 aa, Paramacrobiotus richtersi
MEHTEVHKTTESATGRSHTIQTETTVKDQTYVPLREQADHSPTSSHRSFQERQTVHTHTDARKPSLGTIHPVSISSASGLAQEIVAEGYHASAASVHSTTAATTIAESPQTYELKLKDLEHYRREQEAIARKYEKEVEKLTEKYRRKTEAEADKIRKELEKQHARDVEFREKLVQEAIARQKEEIVLEAKYATKELDRQRMLALEALERSRHQSNIQVNLETVAGHTVSESQNVTSHYESHDSINDHKSIGAKIKEAIMGKPE

SEQ ID NO: 15 - protein, 227 aa, Paramacrobiotus richtersi
MEAMNMNIPRDAMFVPPPESEQNGYHEKSEVQQTSYMQSQVKVPHYNFPTPYFTTSFSAQELLGEGFQASISRISAVTEDMQSMEIPEFVEEARRDYAAKTRENEMLGQQYEKELERKSEAYRKHQEVEADKIRKELEKQHMRDIEFRKEIAELAIENQKRMIDLECRYAKKDMDRERTKVRMMLEQQKFHSDIQVNLDSSAAGTESGGHVVSQSEKFTERNREMKR

SEQ ID NO: 16 - protein, 263 aa, Paramacrobiotus richtersi
MEHTEVHKTTESATGRSHTIQTETTVKDQTYVPLREQADHSPTSSHRSFQERQTVHTHTDARKPSLGTIHPVSISSASGLAQEIVAEGYHASAASVHSTTAATTIAESPQTYELKLRDLEHYRREQEAIARKYEKEVEKLTEKYRRKTEAEADKIRKELEKQHARDVEFREKLVQEAIARQKEEIVLEAKYATKELDRQRMLALEALERSRHQSNIQVNLETVAGHTVSESQNVTSHYESHDSINDHKSIGAKIKEAIMGKPE

SEQ ID NO: 17 - protein, 227 aa, Paramacrobiotus richtersi; identical to SEQ ID NO: 15

SEQ ID NO: 18 - protein, 174 aa, Paramacrobiotus richtersi
IVAHAAGIAEEVVGKGFTASAARITGTSQQVDVTPSPQLQQEVRRDEERYMREKDAIAAQHEKELERKTEAYRKTAEAEAERIRKELEKQHQRDVEFRKDLVDSAINRQKQEVELEAKLAKKELEREAAMAKEALERSKLSTNIEVNFDSAVGHTQSAGTTVSESESISRTVKK

SEQ ID NO: 19 - protein, 229 aa, Paramacrobiotus richtersi
MSAEAMNMNMNQDAVFIPPPEGEQYERKEKQEIQQTSYLQSQVKVPLVNLPAPFFSTSFSAQEILGEGFQASISRISAVSEELSSIEIPELAEEARRDFAAKTREQEMLSANYQKEVERKTEAYRKQQEVEADKIRKELEKQHLRDVEFRKDIVEMAIENQKKMIDVESRYAKKDMDRERVKVRMMLEQQKFHSDIQVNLDSSAAGTETGGQVVSESQKFTERNRQIKQ

SEQ ID NO: 20 - protein, 216 aa, Paramacrobiotus richtersi
MEARQHEQQFQKQEVEQTSYMQTQVKVPVMKLSAPSIITVPLAQELVGEGFQASISRISGVSQEIQQIDSAQLDEEVRRDYESKQREAELLQQQFDKEVEKKTEAYRKQQEIEAEIIRKMLEKQHIRDVEFRKELVEHAIENQKRQIDIESRYAKKELERERTKARMLLERQKFHSDIQVNLDSTAATTHAGEQVVSESEKFTQNSKMSCGQQRAG

SEQ ID NO: 21 - protein, 229 aa, Paramacrobiotus richtersi; identical to SEQ ID NO: 19

SEQ ID NO: 22 - protein, 260 aa, Paramacrobiotus richtersi
MAHELNPHETRTDFSDAEGGSYEKQIHSELRAPSATPSGHSQSDKRETTVTYTYTDVRTPQMNPPAPVLIIPSAAGLAQEIVGEGFTASAARVTGASPQVTVTETLTSQEKYLREQENYRREQEALVRKYERSIEKMNEEYRKKTEQEADKIRKEMEKQHERDIEFRKELMDKAIERQKEEIALEAKYARKELERQREMAMEALDKTKKQADVQVNLDTLAGHTVSESQSQLTPDADIPADHREPHKSLSSKLRETFTGK

SEQ ID NO: 23 - protein, 222 aa, Paramacrobiotus richtersi
MERKVEVCQEKHTHSEKCRTETHGHQETVHTGYTHTEVRAPLVVPPPPIVAHAAGIAEEVVGKGFTASAARITGTSQQVDVTPSPQLQQEVRRDEERYMREKDAIAAQHEKELERKTEAYRKTAEAEAERIRKELEKQHQRDVEFRKDLVDSTINRQKQEVELEAKLAKKELEREAAMAKEALDKSKLATSIEVNFDSAVGHTQSAGTTVSESESVTRTVKK

SEQ ID NO: 24 - protein, 260 aa, Paramacrobiotus richtersi; identical to SEQ ID NO: 22

SEQ ID NO: 25 - protein, 259 aa, Paramacrobiotus richtersi
MPLFGSSKKDKHHHGAEFPITDRDNDIENRNLEHFDREVDQFPDRSPGREGSLIQERHISRSPVHKTVTERRSEVSYVQSVPAAVEISRQPMYHQEANIIIPGERREVVEKTEVIRSATPRRESVEVISIPIHKVSGAQMEHVRSGVTYTNDKELIIPGAMIAPMIPSCTQDLLARGSGGTHAEIHADTNINLLANAHLDSSPEEYNRYRASVEELAHQHEIDTAQKAALYRTQVEADAELIRRTLERQHIRDIEFRKE

SEQ ID NO: 26 - protein, 259 aa, Paramacrobiotus richtersi; identical to SEQ ID NO: 25

SEQ ID NO: 27 - protein, 86 aa, Paramacrobiotus richtersi
RETAKIRKEMEKQHERDVEFRSKLVEDAIKRQKEELELEAKYAKKELERQRELALDALENSRMHTDISVNMDTTVGHTVSSGRIDS

SEQ ID NO: 28 - protein, 149 aa, Paramacrobiotus richtersi
MSHAHHAHNYECYTHTADGKAAATATATSLASGEKEVHTTSYTHVDAKLPLLQDLPSPLTTTGIAGLGQTLVGEGFTASVVRASGESEQVTVAPSERLTEEARRDQERYQRDKDAINERQKHSVENKAENYRKEAEQQAERIRKELEKQ

SEQ ID NO: 29 - protein, 92 aa, Paramacrobiotus richtersi
RKELEKQHERDVEFRKGLIDSAIERQKREVELEAKMAKRELDREAQLAKEALERSKLATNVEVNFDSAVGHTASVGTTVSESESITRDVRKN

SEQ ID NO: 30 - protein, 149 aa, Paramacrobiotus richtersi; identical to SEQ ID NO: 28

SEQ ID NO: 31 - protein, 77 aa, Paramacrobiotus richtersi
MEKQHERDVEFRSKLVEDAIKRQKEELELEAKYAKKELERQRELALDALENSRMHTDISVNMDTTVGHTVSSGRIDS

SEQ ID NO: 32 - protein, 224 aa, Hypsibius dujardini
MSNYQQESSYQYSDRSNNGQQQEQQEKKEVEHSSYTHTDVKVNMPNLIAPFISSSAGLAQELVGEGFQASVSRITGASGELTVIDTEAETEEARRDMEAKAREQELLSRQFEKELERKTEAYRKQQEVETEKIRKELEKQHLRDVEFRKELMEQTIENQKRQIDLEARYAKKELERERNKVKRVLERSKFHTDIQVNMEAAAGSTHSGSSSVAVSESEKFQTNN

SEQ ID NO: 33 - protein, 224 aa, Hypsibius dujardini
MSNYQQESSYQYSDRSNNGQQQEQQEKKEVEHSSYTHTDVKVNMPNLIAPFISSSAGLAQELVGEGFQASVSRITGASGELTVIDTEAETEEARRDLEAKAREQELLSRQFEKELERKTEAYRKQQEVETEKIRKELEKQHLRDVEFRKELMEQTIENQKRQIDLEARYAKKELERERNKVKRVLERSKFHTDIQVNMEAAAGSTHSGSSSVAVSESEKFQTNN

SEQ ID NO: 34 - protein, 227 aa, Hypsibius dujardini
MSGRNVESHMERNEKVVVNNSGHADVKKQQQQVEHTEFTHTEVKAPLIHPAPPIISTGAAGLAEEIVGQGFTASAARISGGTAEVHLQPSAAMTEEARRDQERYRQEQESIAKQQEREMEKKTEAYRKTAEAEAEKIRKELEKQHARDVEFRKDLIESTIDRQKREVDLEAKMAKRELDREGQLAKEALERSRLATNVEVNFDSAAGHTVSGGTTVSTSDKMEIKRN

SEQ ID NO: 35 - protein, 227 aa, Hypsibius dujardini; identical to SEQ ID NO: 34

SEQ ID NO: 36 - protein, 237 aa, Hypsibius dujardini
MSQQYEKKVERTEVVYGGDRRVEGSASASAEKTTNYTHTEIRAPMVNPLPPIISTGAAGLAQEIVGEGFTASATRISGAAATTQVLESQASREQAFKDQEKYSREQAAIARAHDKDLEKKTEEYRKTAEAEAEKIRKELEKQHARDVEFRKDLVESAIDRQKREVDLEAKYAKKELEHERELAMNALEQSKMATNVQVQMDTAAGTTVSGGTTVSEHTEVHDGKEKKSLGEKIKSLF

SEQ ID NO: 37 - protein, 358 aa, Hypsibius dujardini
MSELPGFSMHRLCRQWNFPLDKSHCAANTFHAGISRSLSRTPQFLNFRFLQIFLPLYFRSLLHRKESPLIIMSHTHEQKFERVEERTIDEKKGTEEVRVGIDTGYGDPALNFQPTDATLVRTPCVGGDVMSSNRSSACSSGVAGASQFASHSMRDSSSGNVVKEAEKTTSYTHTEAHAPLITPSQPFIVTGAAGLAQEIVGEGFTASASRISGGAVNTKVIETAEMRQKELREQEQFAREQAAIIQHHDKDLARKTEKYQKEAEAEAEKIRKELEKQHARDVEFRKDLVETAIDRQKQEIDLEAKKAKADLERERQMAKEALDNSKMQTNIEVQMNSAAGMTTSGGTSVSESHVSKNF

SEQ ID NO: 38 - protein, 414 aa, Hypsibius dujardini
MATKESKYERVEKVNVDADGATLVKNIGEDRGKEDPGMNFQDKRPANLVPGAPAGVIPNRIESLPTDRAGQRLREHLSESERLRVSRSSTSSKSSSFVEPSLKYRGEIGPIGKNGEFVASSNRQNSSSNVSSSDNSERASPASRNSNPGMNNGMTTQRTTVITESSVQGLGAQRTVPIQPHQQREDHEVITHESHARAPETTVVTIPTTRFESAQLESRRDGRTYTEDKELTIPAPVVAPQIHAHQQVNMSGGTSATIHATTDLHLASEAQINDMGPEEYERYRAKVEALARIHEDETSRKAAAYRNAVEADAELIRQTLERQHMRDIEFRKDLVESSVDRQQQEIRLEAEYAMRALEQERVNARAALDQAMASTNIDVNIDSAIGTTHSQGRVTTTSESRTSQARGPATAAVI

SEQ ID NO: 39 - protein, 414 aa, Hypsibius dujardini
MATKESKYERVEKVNVDADGATLVKNIGEDRGKEDPGMNFQDKRPANLVPGAPAGVIPNRIESLPTDRAGQRLREHLSESERLRVSRSSTSSKSSSFVEPSLKYRGEIGPIGKNGEFVASSNRQNSSSNVSSSDNSERASPASRNSNPGMNNGMTTQRTTVITESSVQGLGAQRTVPIQPHQQREDHEVITHESHARAPETTVVTIPTTRFESAQLESRRDGRTYTEDKELTIPAPVVAPQIHAHQQVSMSGGTSATIHATTDLHLASEAQINDMGPEEYERYRAKVEALARIHEDETSRKAAAYRNAVEADAELIRQTLERQHMRDIEFRKDLVESSVDRQQQEIRLEAEYAMRALEQERVNARAALDQAMASTNIDVNIDSAIGTTHSQGRVTTTSESRTSQARGPATAAVI

SEQ ID NO: 40 - protein, 358 aa, Hypsibius dujardini; identical to SEQ ID NO: 37

SEQ ID NO: 41 - protein, 227 aa, Hypsibius dujardini
MQQNNENFERVVERSEVRQECQQPCREEESRQEEHNSSYLHTEVRAPVPNIPPPMMSGSAGLGQALVGEGFQASAARISGGSQEMNIQPSEKLLQEAAMDKERYAREQEAIQNRLQSETERKTEAYRKTAEAEAERIRKELEKQHERDIEFRKDLVQGTIDSQKKQVELGAIMAKRELDREAKLARDALEQSKMATNVEVNFDSAAGHTVSGGQTVSQSTKVTREKK

SEQ ID NO: 42 - protein, 298 aa, Hypsibius dujardini
MSSIEHPNVYVSERQDVFRSAGEVPPPLPARPVGGEFIRETITTGPAGTIHSTLTTSTLSQPGTLSPQGTLSPQGILSQPGTLNQQGTLSQQGTLNHSHVVTTTGDSTSYTHTEIKAPLHVTSPILISSAEGLAQEIVGEGFTASAARVAGTATQEYVHETVETSRQAALDRQRRDREMEAVARRSEEEVAKKTEAYRKTAEAEAEKIRRELEKQHARDVEFRKDLVESAIDRQKREVDLEAKYAKTELEHERKLALEALERSKLESNIEVNFDSAAGRTVTESHVVSQHTDISHPRM

SEQ ID NO: 43 - protein, 90 aa, Hypsibius dujardini
MVDESVKRQKKELELEVKYAKKELDHERLLAKEALEQSKMHTDVLVNLDTSAGHTVSGGSHVTEEEYSEHHTEHKKTIAEKLKETFTGHH

SEQ ID NO: 44 - protein, 286 aa, Hypsibius dujardini
MSSIEHPNVYVSERQDVFRSAGEVPPPLPARPVGGEFIRETITTGPAGTTHSTLNTSTLSQQGTLSQPGTLSQPGTLSPQGTLNHSHVVTTTGDSTSYTHTEIKAPLHVTSPILISSAEGLAQEIVGEGFTASAARVAGTATQEYVHETVETSRQAALDRQRRDREMEAVARRSEEEVAKKTEAYRKTAEAEAEKIRRELEKQHARDVEFRKDLVESAIDRQKREVDLEAKYAKTELEHERKLALEALERSKLESNIEVNFDSAAGRTVTESHVVSQHTDISHPRM

SEQ ID NO: 45 - protein, 90 aa, Hypsibius dujardini; identical to SEQ ID NO: 43

SEQ ID NO: 46 - protein, 227 aa, Hypsibius dujardini; identical to SEQ ID NO: 41

SEQ ID NO: 47 - protein, 186 aa, Hypsibius dujardini
MTHYKEDEELLEHLREDSGFQAFKTKAVDDVVAGNGNTHSELHETVKEKASVSSASSSSSSSPPSTGRSSVERHVTYTHTEAKSGPLIHTTHPVVLSSASGMLAHEIMEEQSGFMASATHVSGSEHGVAAAHESPELREQRLKDEAKYREKQDEIARKHDKHLEKVTEEYRKKTEAEAEKIRKELE

SEQ ID NO: 48 - protein, 186 aa, Hypsibius dujardini; identical to SEQ ID NO: 47

SEQ ID NO: 49 - protein, 237 aa, Ramazzottius varieornatus
MPYEKHVEQTVVEKTEQPGHSSTHHAPAQRTVAREQEEVVHKEFTHTDIRVPHIDAPPPIIAASAVGLAEEIVSHGFQASAARISGASTEVDMRPSPKLAEEARRDAERYQKEHEMINRQAEATLQKKAEEYRHQTEAEAEKIRRELEKQHERDIQFRKDLIDQTIEKQKREVDLEAKMAKRELDREAQLAKEALERSRMATNVEVTLDTAAGHTVSGGTTVSSVDKVETVRERKHH

SEQ ID NO: 50 - protein, 216 aa, Ramazzottius varieornatus
MSRDQGSTEYDANQRQEQHQEQHNTSYTHTDVRTNIPNIPAPFISTGVSGLGQQLVGEGFTASAARISGQSSETHVQMTPEMEAEARKDRERYERELQAINERHQRDIEGKTEAYRKQAEQEAERLRKELEKQHQRDIEFRKSLVQGTIENQKRQVELEAQLAKRELDREARLATQALDQSKMATDVQVNFDSAVGHTVSGATTVSQSEKVTQSKH

SEQ ID NO: 51 - protein, 303 aa, Ramazzottius varieornatus
MSSRQNQQSSSQHSSSSQQGGQGGQGVQGSSSYSRTEVHTSSGGPTIGGAQRTVPVPPGSHSEVHEEREVIKHGTKTESETHVVTVPVTTFGSTNMESVRTGFTVTQDKNLTVAAPNIAAPIHSNLDLNLGGGARAEITAGTTVDLSKIQRKDLGPEEYARYKAKVEQLARQDEQDAGMRAAQYREEVERDAELIRQILERQHIRDLEFRKEMVENQVNRQEREIQLEAEYAMRALELERNAAKEALESAKAQTNVNVKVESAIGTTVSKGAIQTSADKSSTTKTGPTTVTQIKHTEQHTERR

SEQ ID NO: 52 - protein, 378 aa, Milnesium tardigradum
MSTHRDRDSANNEYIAETVSSVTTSTAADLTTGRTLYATPVTSTARHHDTTTSSHTSQRMATDYTTGAGTVYTEKTVMREPVNVVHTQIDRVTAVPITETQVHAETQHYLHTQMRTPVVESHPPQLPAHTDVAGSILNDSAFSSTAHISTNAMHAQAVPVDAAERARQEEHFRREADRIALQHQRAIDEKSEAYRRDTEAQAERIRQELEKQHLRDVQFRQELVDDAITRQKREVQLEAQAVMADLELERRRAHEALERSKMSTDINVNIDTLAGSTTAGGTTVIEKTEVQKGVAYHTTPVGVTTHVEYADRPTTTHRTETTTTAHSTHTTEGHHRGSDASYIGGRDDDRMSIGTQGSDADEHKKRGLLGKIKDSLTK

SEQ ID NO: 53 - protein, 249 aa, Milnesium tardigradum
MSHQQTREVTKEIHVESSGQSGASSHASGHVVAGHETSAVEHTKYLHTETKVPMATPAPPIIHASSGLQHMEGMTASAARITAGSAETTNVQVSEEVRRRDQAQFEREAAAIAARHEKDVQAKTEAYRKETEEQAEKIRRELEKQHQKDVEFRKDMVDDTINRQKREVELESAMAKRQLEREAEAAKAALDKSKLSTDIHVELNTAAGNTVAGGTTTSVSQSERHESASVHESKSLGDKVKDALGFGSK

SEQ ID NO: 54 - protein, 259 aa, Milnesium tardigradum
MNPTSEHISETTTTVKTTDTGVGLQNVSASHHASGIHHDSSAASSTESTKFVHTETKVPMATPAPPIISAATGIADSIVSEGMTASAARISAGANESIVPVVDTQKAAADYDKYQREAAAIAAAHERDVAKKTEAYRKETEEQAEKIRKELEKQHAKDIEFRKDLVEDAITRQKREIELEAKMAKKELEREAEAALAALDKSKLSTDIAVSINTAAGSTVAGGTVTTVTEKTESTHSHEHEHEHRSLGEKIKDTLLGRK

SEQ ID NO: 55 - protein, 89 aa, Milnesium tardigradum
KELEKQHAKDIEFRKEILEDTIARQKREVELEAKMAKRELDREAAAAREALDRSKLATDISVSIDTAAGHTVATETMKSTEHTFSHQRM

SEQ ID NO: 56 - protein, 147 aa, Milnesium tardigradum
MSRIAEEHERKVKQRTEAYRKETEMQAEKLRVEMQKQHIQEQQYRRELSEATIAREKQEALLAYRAKLTELERTQQALKSAADQARLSSEIEVTISTSAGETITGISIDSKSEASMLEMNGTQTHAKSQEEVRSLGDKLKTIVLGRP

SEQ ID NO: 57 - protein, 132 aa, Milnesium tardigradum
GHYGRGDYRKGLAAKQVDCQKQQVELEVHHYLIITDYDSTARNLSGKLIFVEQAKMAKKELERELTAAKEALDATKSATNIHVNIETLAGVTMAGATTHSQITEVLDESEMDNDRKLTLGQKIKEKLSKGKL

SEQ ID NO: 58 - protein, 45 aa, Milnesium tardigradum
MPHTRPQPQRRPGDLHSAHRNQNTNHRTCSTSNGYLNWSGFRNYW

SEQ ID NO: 59 - protein, 167 aa, Milnesium tardigradum
DDRSRREFRGIPKSTRCAKALFCGNRGRKVDGRTEKEGDKYHHVICFPQADYHQDIPFKIGEAGSHQIKNTTVNYTYTLKTKDDKPVLHADFKADAAGGRPAMEFSSNFHFSDTGFVHTYKKGNVTAMRTLKRFRSVLYLCVDLLCSVRIFFCICIHIFCHFFCFRI

SEQ ID NO: 60 - protein, 167 aa, Milnesium tardigradum; identical to SEQ ID NO: 59

SEQ ID NO: 61 - protein, 169 aa, Ramazzottius varieornatus
MSRAAVAIALLGCVVAAYGAPAEGHDDAKAEWTGKSWMGKWESTDRIENFDAFISALGLPLEQYGGNHKTFHKIWKEGDHYHHQISVPDKNYKNDVNFKLNEEGTTQHNNTEIKYKYTEDGGNLKAEVHVPSRNKVIHDEYKVNGDELEKTYKVGDVTAKRWYKKSSSS

SEQ ID NO: 62 - protein, 174 aa, Ramazzottius varieornatus
MHRFVLALVVFAGAAIVWAADDAAHEEGVEWTGKPWMGKWESDPSKDENVEEFKKKLQLPMSHSEMNKNSKVWIHHYKKGDEYHHKIIINDAHYKNDIVFKLGQESAGSYNGSSFSVKYEDKDGALVGSVHYTGTKEQSLDKTINNVFKLEGDHLVKTSTIEGVTMKRHYNKRQ

SEQ ID NO: 63 - protein, 178 aa, Paramacrobiotus richtersi
MTFKVFILIALVAAVKARPAEGEHKDQQDIAADADHPWIGKWESIDGRQENFQNFINALGFAHYTHEHKVWHKLWKEGDHYHHRIKVPEKGYKLDVEFKLGEEGTGSYNNTQFKYKYTEENKDLHVEINLVTHNKVIKDDYHVEGEELVKTYKVGDVTAKRWYKRAQKKPKAEAAASA

SEQ ID NO: 64 - protein, 178 aa, Paramacrobiotus richtersi; identical to SEQ ID NO: 63

SEQ ID NO: 65 - protein, 168 aa, Paramacrobiotus richtersi
MVLLAALLFGVVTCVACHGQADPKTIPADPDHPWIGKWESISDRHENFDNFVQHLGMAHYKSENKVYHKFWKEEDHFHHGIIVPDKNFKQFLEFKLGEQGTLTWNGTDFKYKYTEQNKDLHVEVNVPSKNKVIHDVYHVEGEEMVKTYKVDTIEAKRWFKKAPAESIL

SEQ ID NO: 66 - protein, 168 aa, Paramacrobiotus richtersi; identical to SEQ ID NO: 65

SEQ ID NO: 67 - protein, 174 aa, Paramacrobiotus richtersi
MSYLAFVLLGLAVFAAADHHDESGSDPKEIPADPAHPWIGKWESIEGRSENFANFVKKLDAPINYSDDMKVYHKLWKEGDHFHHGIAIPDKQFKKFFQFKLGEEGSFTFNNTEFKYTYTEKDKDLHAEVKCPSKNKVVHDVYHVEGEELVKSYQVDDVKAKKWFKKAASKPANA

SEQ ID NO: 68 - protein, 174 aa, Paramacrobiotus richtersi; identical to SEQ ID NO: 67

SEQ ID NO: 69 - protein, 173 aa, Paramacrobiotus richtersi
MKWLIVVVLGISAALAEDHPTPNNIPLDSAHQWIGKWKSTGRHEHFDDFMKALGLPNHDVADPETTHVLWKEGDKFHHKISAPSVNYKKHICFTLGEEGNSSYNGTAFTYKYTELPDKDLVLVATLPSYNKSVHATFHATGNELMKTFKVDQVVAKRWYARVDQTAAPKPAAK

SEQ ID NO: 70 - protein, 173 aa, Paramacrobiotus richtersi; identical to SEQ ID NO: 69

SEQ ID NO: 71 - protein, 154 aa, Paramacrobiotus richtersi
MQVSSVLFVLGCVIVTIEGGGLHQFLGKWESTEKRENTQAFAEALNQVDQVDINSKIFNEFSLDQASADGYHHKFSVPDKNYVQDVTFKLGVEGQKTFNGTTYKYKYTLDGDTLKSHFELPDRQVDQEFSLVNNELVKTYKVNNVVAKVWFKKV

SEQ ID NO: 72 - protein, 150 aa, Paramacrobiotus richtersi
MNVCIASLCLGCLIVAVEGAGLGIFMGKWESTNKRESTQAFAEAVEHVDIDSKIVNEFSVKNGGEEYHHKFSVPDKNYIQDLPFKLNEERQTTFNGTTYKYKYTLEGDTLKSHFELPDRQVDQEFNLVSNELVKTYKVNNESAKVWFKKV

SEQ ID NO: 73 - protein, 180 aa, Paramacrobiotus richtersi
MLLLLVDKHSFRVPNKKSICCNRITIVFSAMNVCIASLCLGCLIVAVEGAGLGIFMGKWESTNKRESTQAFAEAVEHVDIDSKIVNEFSVKNGGEEYHHKFSVPDKNYIQDLPFKLNEERQTTFNGTTYKYKYTLEGDTLKSHFELPDRQVDQEFNLVSNELVKTYKVNNESAKVWFKKV

SEQ ID NO: 74 - protein, 197 aa, Paramacrobiotus richtersi
MKKFEALFRNPASKFSAHIKFSDNMRYIGLLLLGLAACSRLEPEQGSHMSLKDIHPDPEHPWIGSWESIEGRFQSVDTDRNEIGIAPYMNDANTKVYRQFWREGDHFYHVVAAPERGFRTEFRFRLGEESVVILNGTEYKFIYSEKGKDLHAIVKIPSTSTVFTDVYHVQNEDMLKTFTRGAVQAKRWFKKIKSQPS

SEQ ID NO: 75 - protein, 151 aa, Paramacrobiotus richtersi
MQVSSVLFVLGCVIVTIEGGGLHQFLGKWESTEKRENTQAFAEALNQVDINSKIFNEFSVDEANINGYHHKFSVPEKKYVQDVTFKLGEEGQKTFNGTTYKYKYTLDGDTLKSHFELPDRQVDQEFSLVNNELVKTYKVNNVVAKVWFKKV

SEQ ID NO: 76 - protein, 170 aa, Paramacrobiotus richtersi
MRYIGLLLLGLAACSRLEPEQGSHMSLKDIHPDPEHPWIGSWESIEGRFQSVNTDRNKIGIAPYMNDANTKVYRQFWREGDHFYHVVAAPERGFRTEFRFRLGEESVVILNGTEYKFIYSEKGKDLHAIVKIPSTSTVFTDVYHVQNEDMLKTFTRGAVQAKRWFKKIRS

SEQ ID NO: 77 - protein, 167 aa, Paramacrobiotus richtersi
MFASLVIFGLVAACANAAPADQANQSSHSDHSHAWLGKWESTPEGEENMQQLVDQIKDAIPHYTTKKITHEYIEQGDEFVHKVQIEGGQNYEVKFKLNQEHSWHLGEEPEIKYKYTEEGPNKLKVHMNIPSKNKELKECYNVEGDKINKEYESGSVKAKRVYKKVQK

SEQ ID NO: 78 - protein, 167 aa, Paramacrobiotus richtersi; identical to SEQ ID NO: 77

SEQ ID NO: 79 - protein, 180 aa, Paramacrobiotus richtersi
MAQMLFIIGFLCCAGVYGSQSVGRVHMKDHYGNRGDSFENVAHQWLGKWESVEGTEENFDQLLDAIREAFPYYSQATIIHDFSKKSDDEFIHKIKIGSDEDHYQLTFKLDQEGTLRKPGAPEMKYTYEEVSGNKLVVQQSVPSKNIMLEESYKVQGDQILKEYATGGVRAKRTFQRMNHL

SEQ ID NO: 80 - protein, 180 aa, Paramacrobiotus richtersi
MAQMLFIIGFLCCAGVYGSQSVGRVHMKDHYGNRGDSFENVAHQWLGKWESVEGTEENFDQLLDAIREAFPYYSTATIIHDFSKKSDDEFIHKIKIGSDEDHYQLTFKLDQEGTLRKPGAPEMKYTYEEVSGNKLVVQQSVPSKNIMLEESYKVQGDQILKEYATGGVRAKRTFQRMNHL

SEQ ID NO: 81 - protein, 189 aa, Paramacrobiotus richtersi
MTGVPRPSSAYFVIAFYCFSCVTAESTETTPPRGSGNGTSIAVEAAKPVLIPFGKFEATDQVENFASYLSSLRVEFKGFSAGNLKGKVQHEFSRAPDNKYSHAFWIAGTPYKQKLSFELGKEHQQTYNGTGFKYRYYQEPSQLGLHAVFHVPADNPLPIEHLYTTSPDGFVLTYKIGDVTAKRAYKRIP

SEQ ID NO: 82 - protein, 172 aa, Hypsibius dujardini
MSRTIVALILLGLAALAAADHHEGHGAEKEWAGKAWLGKWVSTDRSENWDAFVEALGLPLAAYGGNHKTVHKLWKEGDHYHHQIIIADKSYKQDIQFKLGEEGRTAHNGTEVTFKYTEVGDNLQNEVKIPSKNKTISDSYVVKGDELEKTYKINDVVAKRWYKKHAHEPSTA

SEQ ID NO: 83 - protein, 172 aa, Hypsibius dujardini; identical to SEQ ID NO: 82

SEQ ID NO: 84 - protein, 163 aa, Hypsibius dujardini
MARLFVAVALFGVVAFAAAEKEWTGKTWLGSWASTDRAENWEAFVDALGLPSDQYPREVQRTIHTIYKQGDKYHHEVSIPSKNFKKAIEYTLGTETDVQHGPHTIKLKYTEDGEKLVADVQIPSKNKQIHDIYEVQGDTLTKTYKVGDVVAKRWFTREANPTA

SEQ ID NO: 85 - protein, 163 aa, Hypsibius dujardini; identical to SEQ ID NO: 84

SEQ ID NO: 86 - protein, 165 aa, Hypsibius dujardini
MSRVLVALALFGVVALAAASGDAQKEWTGKSWLGKWQSLPTDKSENWEAFVNALAIPEQYTRDLQKTVHTFYKQGDHYHHIFAIPDKNFEKNIEFNLGAESSAKHGEHEVKIKYAEDGDKLVADVRIAAKNKHIHDVYEVQGEELVKTYKVGDVVAKRWFKKIAQ

SEQ ID NO: 87 - protein, 165 aa, Hypsibius dujardini; identical to SEQ ID NO: 86

SEQ ID NO: 88 - protein, 168 aa, Hypsibius dujardini
MARFLVALALFGVVAMTAASGDAPKEWSGKPWLGKFVAEVSDKSENWEAFVDALGLPDQYPRAQLKTIHSFYKQGEHYHHILSLPDKNINKDIEFTLGQEVEIKHGEHSLKIKYFEDGNKLVADVSIPAKGKSIHDVYDVQGDQLIKSYKVGDVVAKKWFKKVANPAA

SEQ ID NO: 89 - protein, 174 aa, Hypsibius dujardini
MARFLVALALFGVVAMTAATGDAPKEWSGKPWLGKFVAEVTDKSENWEAFVDALGLPEQFGRAPVKTIQKIYKQGDHYHHIFALPDKNFEKDIEFTLGQEVEIKQGEHIAKTKYSEDGEKLVADVSIPTKGKTIRSEYEVQGDQLIKTYKTGDIVAKKWFKKVANPTEAPAQAA

SEQ ID NO: 90 - protein, 168 aa, Hypsibius dujardini; identical to SEQ ID NO: 88

SEQ ID NO: 91 - protein, 172 aa, Hypsibius dujardini
MAHLTILLALSVTGFFVSTVADHKAEKHGGKFDGKSWLGKWESTNHTENLETFVSQLGYPSAEHVTDQKVFQKFWQDGEHFHHKITVPTKNYTLQHKFTLGQPGKATFNNVEFKYLYAELGNDLHVEITVPSKNKTVSDTYHVFQNGTELEKTYKTGDTVAKRWYKKVISCH

SEQ ID NO: 92 - protein, 172 aa, Hypsibius dujardini; identical to SEQ ID NO: 91

SEQ ID NO: 93 - protein, 156 aa, Hypsibius dujardini
MARLSLIVLMGVVAVASASQPWLGSWTTTDKAPENWDQVVAALGLPAAYGGNPKSTLSITREGETYTSKLEVPSNNFSSTWTFKIGEEGTKVEPKFENTEVKYTFTEEGEKLLVTVKIPARGKEVTEVYEVTGDELVKTYKIDGIVAKRYLKRQAV

SEQ ID NO: 94 - protein, 156 aa, Hypsibius dujardini; identical to SEQ ID NO: 93

SEQ ID NO: 95 - protein, 157 aa, Hypsibius dujardini
MSRILLVLALFVMVSVTSAAQPWLGVWTNSEKAPENWDQFVAALGLPLEQFSGNPKATITITRDDGDNYKVLLDVPAINFTSTWNLRLGEEMVMDEFGSGMRYNFTEDGDKLQAHVKISAIGKQYNENYEVVGQELIITYKMDGIVAKRFLKRDQSS

SEQ ID NO: 96 - protein, 161 aa, Hypsibius dujardini
MAAIDPTPATVLSVQQENCRPWLGMWVSAGKKENWPAVMEALGLPEMYSEKNTFVLKLWCDGEDFHYDAGILEAKFKHSVTFKLGTPTELNHGNKIVITYTEEDGKLIADGVIAAKNLILHNVFAAQGDVLIKTYRVGNVVAKSWYRRLSSTADSNILSFL

SEQ ID NO: 97 - protein, 154 aa, Hypsibius dujardini
MEFAASIFVLCFGLSAVTAAGLPFVGHYVSTGQRFNTAAFAAATGFDDPPVENRLHNEFLDQGNGEYLYKFRVENAAYKQELPFKLGETRKSTYNGTEFSYKFTVDGELLKFESKILPDGREVTHTYYPNADGFVKQFQLKDVIAKVWFKKDSA

SEQ ID NO: 98 - protein, 154 aa, Hypsibius dujardini; identical to SEQ ID NO: 97

SEQ ID NO: 99 - protein, 207 aa, Hypsibius dujardini
MISLFLLFAVGGLAVDGALPPGEVAAVLLPPSMVNIIPVPLGEFVPTGQKENYANYVHSLEFEFRGLAAQGILGDKGKDVRHKFSRSADGKENSYVHKFGNDGGGKYNHTVPFVLDEEKLVHTNATSLKYKYWFEPGQGLHADYNIPPENPLQIQHLYAVTDEGFTLIYKLGNVIAKNYYKRAPSSDAAPEVTSKTTVAPITTKKKA

SEQ ID NO: 100 - protein, 207 aa, Hypsibius dujardini; identical to SEQ ID NO: 99

SEQ ID NO: 101 - protein, 236 aa, Ramazzottius varieornatus
MSRYLLRDVQAVLRGVRKVAESSLKLETEKVSLRLGDFRSQPSLRSVPASLTSRSQAFSLQEIAARAGVVLRGVQQQFRNVTGVNAAPVVAFDNGSVLYSERIHSQSSQKQAPTTVPTGSVSNSPQPEGKANEAAERAKQFMNPPVAPMDPVDKNEFVAMPEMGRSNGNGENKQAADFMKNQGDTDMDSQYAPDSSKNTKSVPTKEIVAEDGSMSIEDIKKATQVTPGVAVKNEGV

SEQ ID NO: 102 - protein, 247 aa, Hypsibius dujardini
MAKYLLHDMQAMLRGIKQVAQISLKIQAAEINERVCQSSLRPRWSNLASSSASSPASSSSPRSSFNVQEIASRAGAVLRGLEEQVKIVAGIQAPAPILAFDNGFTLYSDKIGSAQNRATRDHPTTADIDDENGHGKPEGEAGKAAKRAEKFMNPPVAPLDESDVSVLANNSLEGDDSHNLKNFNNGSLDAAEAEGKEETSHLKQDRFSKDSKKTFIDSGGDNLFRPENLKKISKVPPGVPVKADSFS

SEQ ID NO: 103 - protein, 247 aa, Hypsibius dujardini
MAKYLLHDMQAMLRGIKQVAQISLKIQAAEINERVCQSSLRPRWSNLASSSASSPASSSSPRSSFNVQEIASRAGAVLRGLEEQVKIVAGIQAPAPILAFDNGFTLYSDKIGSAQNRATRDHPTTADIDDENGHGKPEGEAGKAAKRAEKFMNPPVAPLDESDVSILANNSLEGDDSHNLKNFSNGSLDAAEAEGKEETSHLKQDRFSKDSKKTFIDSGGDNLFRPENLKKISKVPPGVPVKADSFS

SEQ ID NO: 104 - protein, 243 aa, Paramacrobiotus richtersi
MARFMIKDLQAVFRGFQQVAQSSVEHQLTETALRWHTLSLRPLVQGCVNRMQESQRSTVPLREFPARVGAVVQGIQEQMKILAGFPSPALVTPEGFVFYTDKVNKDTHKEYPAVADEVHSAKLQGLKPESGEACEAAKRAKEFMNPPVSPLDPEDKNEVVRTPEMSGSTAAEDQNADESGKAAKRLGKFMNEEIAPESKPFKPFAKDSAKTTVSFTDATGENFRIQDLKKVQVFPGTPVAFES

SEQ ID NO: 105 - protein, 272 aa, Milnesium tardigradum
MSRYLLNDMEGIIRGLRSVATNAAALHRTDLSTRLQNCTFAAQSSNAVPSLLRQMQKVRPINTTDFVSRTRTVLRGLRDQSQSLFGSSQSIRHTSGTASPTTNVVTKTEKEMKEAQSKERNSKDTHSKDSYNKDAHITPSSDSTASNAASNKRDAKKSDDTNGGARMVDEGAFNNEKPIKQSSAKDHRDQSPHNRELADVERKSEVEMPEDSEDQKLTEAEQAAKRIEEFLNGPKSPEDPASKDKIVVTPEMTKHEEPIPESKAVQEIWIEF

SEQ ID NO: 106 - DNA, 4017 bp, Paramacrobiotus richtersi
aggaaataacctcaccgaacgcgaactcaacagcgcagtatataaccacgcttcgaccaaaccaaacacaactcaaacccagtttaaagcactaacaattcaagaaatctctctaaatcttcaaaaatgtccggacgtatcgagcaacacatggaagcggaggaatgccagggcggtgcctactgcccgcccaactgccgctaccacagtcgcgggatgaagcaggaacatgaggagaagcacgtgtaccgagagagtgtcacacctggtcatgcagaacggcgggaggaacgaagggatgagcagtatcagcggccgtctgagtcgtatcccgagtccaaccgacaggtcgagaaggaggcggttaacactgcccgtgttcacaccacggtgtcggcgccgattgtggcgccccctgccccggttatcagtgttgcgcccgttgcggaggagctggcttcgggttacacaggcagcgccgctcgctataccgccagcagcgaggtcaccatcctccccaaccccaaactgaccgaagaggcccgtcgcgatgagattgcccgccagaaagaggccgacgacattgcccgtcgacatgaacaggacctggccaaacgcagtgaacagtaccgtaagcaggccgaagccgaagcggagaagatccgcaaagaactggaaaagcaacacgacaaggatatcgccttccgcaagagtctcattgactcggccgtcacacggcaacaacgcgagattgacctcgaagccaagatggccaagaaggagctgctgcgcgaggcggaacaggctaaagagtcgctggagaagacgcgggcagccacgaccgtggaggtcgattttacgactgctgtgggccacacacattccgctggagtaaccgcatccgagacaatccgcaccaatcagtaactccgcccaccgcaccatggacctcaaggctttccctatattccttcattttatacgcaaatctagccaaattacgctaaaactattctttatgctcttctcggatgtaactgcacaactggttcgtttactggcaatctggcttgtatataacttaaaccacttgttttcgaataaaataaacgcaaaataacagacagtttcatcagacgcaagaaaaaaaggcttatacaaaaaaaacaagggaccacacaacaaaatgctacaaaatcatccataatcgacaaagaatacttattacaaaaaaccactacatcaatacaccaaaacatggcagtagtcaacaaaagacggacactcaacaaaaaccacctgaatcccattaaatgcacatttctcccgtattcttgagctcatccacccttcttcagcttctccagcaccagtccatcgcaattcttgacagcacgatacaccttaaccacctgcatggcctcctcctgcgtgacacatcgcaatcgtcttagcacactcaaatgtgcacacaacggcgcccaggcctccgcgacctccagtggctccacctgacgttccacaaaccgacacaagaggtcccggagcccgacggaaaacacgggcttatacgggcgggtaaacgtgttccacaggcacatgagctgcttctcggcaggggagcagtcgatgaaatcgttaatcttatcgtgctcgtactggtcctgccagtggttatcctcatcatcgctgtcgtgctccaccatgtgtttgtggtcggggcggatgggttggacggtgttgctgtgcaggtggtggcggatgggggtgacggggaggccgtagaccgtcaggaggttcttgagactgaaggcgggcagggtttcagccacccggtgctccggtcggataaaccgcttccccagccactgcatcaccgactgatgctcccggtaatgcaccaatccctcgccccgatgccgctgcgccatctcccagcgatccatcatgcccaccaccggttcccgacggaactcgagctccggggcgaaccacacatacagtcgcttcaactccggatcatactcacagcccacggagggatgcaggccttcgaggtggcggataaggtggatcagcgaatcgcaccgcatggtgcataagaggcaggcatgaccggactggcgtttactctccaggatggccatgtcgtacacttggtagctctcgccgggcgggtacaggaggacgtagacgtagtccacctcgacatactgcagcggcggatgtttccgcatggccaccttcatggaatactgctccttgacctccaaattcagcccctccttgatatccttaatattgaacgcacagcaattcggacgcttcaaatgccccgcctcaaagctgaccaccagataatcattcaaattccacccgtccaaccggaaccgattacaccccaacagcaccttgttactcagccccaccacgccctcccgcttgggggcgtgctgcaggaccgggggcagcatggcctgggagagtttctcgcgcgggttgcgacggaggatcccaatctgcagccggatcaactcgtccccctgggggttgcggtagatgggggcggggcgccgggtggtgcgggggtcgttctccttcttgctgtcggcgttgcgggtgacgatgagggattggatctccagttccacggggaagtcgtggttgccgggttgacgacgggacggcacttccaccccattccccaccggtcccggtgtcttcttccgctcccacagctgctggacatagcactcgacattccgcttcttcatggtccgatcccgcttccaactgggtaccagtggtttaaaccggcgatgtaccacactatagcacaaattccgccgcaaaaacacccgggcatcaatcaccacccccccatgcgcccacaccgcattgacctgggtctgtagcaacggaaaccgcgataccaaccccaggaggctaaacttgggaagatccggcggaatcaggacacctgggtcgcccggggtgctgcgggcggaatactcggggcacagggggtgtttcttgaagatcaggtccagttcgggtttcttggggatggcggtgggggtgcgcagggcgatggtcccgcttttacggcgtttcaactttttcggatgtggaccgttgctggtggtcttggggtggccgttgagcagtgggagggacggggagtccgtggaggtcgagggggacggggtgtccgccttgtccgttaaggaagcctgctccagtggcaagaccaatttattcccatcctctttggctttccgcttcctcttgggcacggggaccccgttcccctcggcgccccccttgcgcttcttcccattctccaacacggcgaacttcttgcgcttcctctttttcttccctccctccccctgggcggtggcccgcgtgccccaggggtcctgggcgggcgctgcggccgcggcaccccctcctcgggagcgggcacggggcggtttgctccgcatgcgtaactgggtctccggggaatacttggaacgggaactcaacgagcgggtccgacgtttccgcgaccgcccctggggacgctggggcgactggaggatttggtctaaacgggtctgctgggatttggaacgacgcgattcctcgccgtcctgggaggcgtccgtggattgggaaaggttcaggtcctccacggcgtatttcatcgtactgggaagaacacgccggaacgagcaaatcaggtctgaaagactggagggttgctgtagatgcaacggattggctaattcatgaatgcatgctggaaaagtgattttccccgtgttctgtacatagggaggcgaaatatcgctagtctgtacaataaggatccggaaggtatcccgctgggattgcaacggggatgatggaagagtgttcgtcggatccgagaaaaccttgcagattctccgcgcatcgtggttgatcgaagcaattctgcaattgggccagtttatcgacctagcacttttccagtcagagacagagcag

SEQ ID NO: 107 - DNA, 3993 bp, Paramacrobiotus richtersi
cttcgatcaaccacgatgcgcggagaatctgcaaggttttctcggatccgacgaacactcttccatcatccccgttgcaatcccagcgggataccttccggatccttattgtacagactagcgatatttcgcctccctatgtacagaacacggggaaaatcacttttccagcatgctgtaatgaattagccaatccgttgcatctacagcaaccctccagtctttcagacctgatttgctcgttccggcgtgttcttcccagtacgatgaaatacgccgtggaggacctgaacctttcccaatccacggacgcctcccaggacggcgaggaatcgcgtcgttccaaatcccagcagacccgtttagaccaaatcctccagtcgccccagcgtccccaggggcggtcgcggaaacgtcggacccgctcgttgagttcccgttccaagtattccccggagacccagttacgcatgcggagcaaaccgccccgtgcccgctcccgaggagggggtgccgcgaccgcagcgcccgcccaggacccctggggcacgcgggccaccgcccagggggagggagggaagaagaagaggaaacgcaagaagtttgctgtgttggagaatgggaagaagcgcaaggggggcgccgaggggaacggggtccccgtgcccaagaggaagcggaaaaccaaagaggatgtgaataaattggtcttgccactggagcaggcttccttaacggacaaggcggacaccccgtccccctcgacctccaccgactgcccgtccctcccactgctcaacggccaccccaagaccaccagcaacggtccacatccgaaaaagttgaaacgccgtaaaagcgggaccatcgccctgcgcactcccacggccatccccaagaaacccgaactggacctgatcttcaagaaacaccccctgtgccccgagtattccgcccgcagcaccccgggcgacccaggtgtcctgattccgccggatcttcccaagtttagcctcctggggttggtatcgcggtttccgttgctacagacccaggtcaatgcggtgtgggcgcatgggcgggtggtgattgatgcccgggtgtttttgcggcggaatttgtgctatagtgtggtacatcgccggtttaaaccactggtacccagttggaagcgggatcggaccatgaagaagcggaatgtcgagtgctatgtccagcagctgtgggagcggaagaagacaccgggaccggtggggaatggggtggaagtgccgtcccgtcgtcaacccggcaaccacgacttccccgtggaactggagatccaatccctcatcgtcacccgcaacgccgacagcaagaaggagaacgacccccgcaccacccggcgccccgcccccatctaccgcaacccccagggggacgagttgatccggctgcagattgggatcctccgtcgcaacccgcgcgagaaactctcccaggccatgctgcccccggtcctgcagcacgcccccaagcgggagggcgtggtggggctgagtaacaaggtgctgttggggtgtaatcggttccggttggacgggtggaatttgaatgattatctggtggtgagctttgaggcggggcatttgaagcgtccgaattgctgtgcgttcaatattaaggatatcaaggaggggctgaatttggaggtcaaggagcagtattccatgaaggtggccatgcggaaacatccgccgctgcagtatgtcgaggtggactacgtgtacgtcctcctgtacccgcccggcgagagctaccaagtgtacgacatggccatcctggagagtaaacgccagtccggtcatgcctgcctcttatgcaccatgcggtgcgattcgctgatccacctcattcgtcacctcgagggcctgcatccctccgtgggctgtgagtatgatccggagttgaagcggttgtatgtgtggttcgcgccggaacacgaattccgtcgggaaccggtggtgggcatgatggatcgctgggagatggcgcagcggcatcgcggcgagggactggtgcattatcgggagcatcagtcggtgatgcagtggctggggaagcggtttatccgaccggagcaccgggtggctgaaaccctgcccgccttcagtctcaagaacctcctgacggtctacggcctccccgtcacccccatccgccaccacctgcacagcaacaccgtccaacccatccgccccgaccacaaacacatggtggagcacgacagcgatgatgaggataaccactggcaggaccagtacgagcacgataagattaacgatttcatcgactgctcccctgccgagaagcagctcatgtgcctgtggaacacgtttacccgcccgtataagcccgtgttttccgtcgggctccgggacctcttgtgtcggtttgtggaacgtcaggtggagccactggaggtcgcggaggcctgggcgccgttgtgtgcacatttgagtgtgctaagacgattgcgatgtgtcacgcaggaggaggccatgcaggtggtgaaggtgtatcgtgctgtcaagaattgcgatggactggtgttggagaagctgaagaagggtggatgagctcaagaatacgggagaaatgtgcatttaatgggattcaggtggtttttgttgagtgtccgtcttttgttgactactgccatgttttggtgtattgatgtagtggttttttgtaataagtattctttgtcgattatggatgattttgtagcattttgttgtgtggtcccttgttttttttgtataagcctttttttcttgcgtctgatgaaactgtctgttattttgcgtttattttattcgaaaacaagtggtttaagttatatacaagccagattgccagtaaacgaaccagttgtgcagttacatccgagaagagcataaagaatagttttagcgtaatttggctagatttgcgtataaaatgaaggaatatagggaaagccttgaggtccatggtgcggtgggcggagttactgattggtgcggattgtctcggatgcggttactccagcggaatgtgtgtggcccacagcagtcgtaaaatcgacctccacggtcgtggctgcccgcgtcttctccagcgactctttagcctgttccgcctcgcgcagcagctccttcttggccatcttggcttcgaggtcaatctcgcgttgttgccgtgtgacggccgagtcaatgagactcttgcggaaggcgatatccttgtcgtgctgcttttccagttctttacgaatcttctccgcctcggcttcggcttgtttacggtactgttcactgcgcttggccaggtcctgttcatgtcgacgggcaatgtcgtcggcctctttctggcgggcaatctcatcgcgacgggcctcttcggtcagtttggggttggggaggatggtgacctcgctgctggcggtatagcgagcggcgctgcctgtgaaacccgaagccagctcctccgcaacgggcgcaacagtgataaccggggcagggggcgccacaatcggcgccgacaccgtggtgtgaacacgggctgtgttgaccacctccttctcgacctgtcgattggactcgggatacgactcagacggccgctgatactgctcatccctgcgttcctccacccggcgttctgcatgaccaggcgtaatactttcccggtacacctgcttctcttcgtgttcctgcttcattccccgactgtggtagcgacagttgggtgggcagtaggcgccgccctggcattcctccgcttccatgtgttgctcgatacgtccggacatttttaaggtttcagagaattttacagattcagagaactttagagatttagagaactgctggtgctttaactgagttaagagttgtgttttgttttggtagtggaagcgtggttatatactgcactggtgagtttgtgttcagtgaggtggttatttcc

SEQ ID NO: 108 - DNA, 3022 bp, Paramacrobiotus richtersi
cgcaggcaccacatacaacaacacagtcatgataacagtttagaactttagtggttagacttttgtttgttttgcataatgaaaaaggaatctcagattctgaatcctaaatgaaatgcatgttcaccaatacaaattttaccaagtcgtgaacttatcccactatattttttgaattgttccggggggaccggctggggccttggacttggttttttcattctgattttcaagatactttcacttgacgaaactgtaagcggagaacccgggctgcccacatttcgaatgtgtacgtgtgtttttgtaccttcattgttattatcttactaaatttcacaagttgaaagttggagaattaaactttaaagcgatctgccgtatcatgatgaatccactgggaaggacgataataatttgtgcattctttgttggtatcgtgactgttgtcgcatccttggagcaaaaatgccttagtggtagattccaaaaaccaaaacctggtgcggaaccgaagatagcaaattcgacgtgtgcatcttataacacgaattcgtgctgcaacgaggaagtggctaccaatataagtcggatgtccgaagatcccagtttcaaatggttgagtttgcgatggaatcattgcaaaaatctgtctacggaatgtcagaaatttttcgttcaagatatatgcttttatgaatgttctcctaatctgggaccctggctgattgaggataaaaggacgcatcgaagccagagatttaaagatgtccctctgtgtcggaaacagtgcgacgactggtatgaagcgtgcaaggatgaccaaacgtgcgtaagcgattggtcaaccatgttccggtttgataaaggaatgaacatgtgtccggatgatcaaccttgccggaaaatgagccaggttttcaacaatagtgcgaaagcattctgtgaacgaattttcggcggctcatttcagtacgtgtacgatagccaaccgtgctactcgttcaactcaacagaagttgatttgaacaacaggattgaagtatggaaaagtcagcagtccggtgcatctataccggtaatcgtgtggaactcgccccttttctttctgattgcacagcggcttgggatgcaagtgatcatgatgagaatctgagatgttaccgtgcctttctggttgcctgtgacagaatagtattttcctttgtgccttgacttgatacgacttcagatagggtatacctggtggccagaatgtttattatcttttgcttgttaatgttctttttgttttatacacagaatatcatgtgtaaattcacgatgaatctgctgttatgactgaatctttcttttcctttcttagagatgtattgacatgctacactgaacttgcaccagcaattcaggaagatgaccaacctggcataagccagtagagttggaacactgatgtcttttatgtggtaatataaaaggataattaataaaggattattaattatgaatagctgatgttatgctattgataaataagacgatgttgttgccttattatgacttactacgtaaacacaaacacacttctccgctttcacactttatcatcaaaaacacaaggaagcttccactattctctctagtcaagactgaatccccattccatcatgaatctacgagcatcgttgattctctcatctagcgcacattcaaccacaggtacaccagctgtacctgtactgaccaatcacataacataaacacatacgtcgtacttccgtacagcacgttactaggttgctttttattgtagagctgataatatattttaattagagaagagcataacaatcgtccgtggtggaattactcgttattcgcaacacatcaggaattagatcttatcgagctttcgctacacggaatgtagaaaacatcaatcaaaaaaatataacgagatatttcgggacattggccgaattcgaataagtatgcgctcacatccgtgggtgtgcaacgttcacgctttcagagacaaccacaccctccgtcgttgtgtgtccagcggccgaatcgaacttgacttcgatattcgtcgacattttggaatgctccagtgcgtccattgccagtttcttttcgtgctcgagttccttcttggccattttggcctccagttcaacttccttcttttggcgctcaatggtggtctccaggacatctttgcggaattccacatcccgtgcgtgttgcttctctagttccttgcggatcttttccgcttcggcctccgcttgcttcctgtagctctcggtcttcttttccagctccttctcatgctgcttggcaatggcggctttttctcgctcatatttctccagatcgactgcggcttgtttcgacgaagcggccgactcatggactatagtgcccacagactcaccacttattctggcggccgatgcgctgaagccttcggatacgatctcttgcgctagtccggaggcggacgtcaccacaaacggagccgatgggttgagaacgggtgccttcacttcggtatgggtgtagtgcgtcgatttctccgacgtctcggtgcagctgccggaatgttcgtgatgctggtgatgctggtgatgttcatgacgctcaccacatgccggactgcagacggtgttgcaggcgacgccagtatgatggtggtgttcatggcgttccgtgcacttgggatcgcagaccgtggtacaggttacgttggaacaggcggcagggatcacaggtggcacgttgccggttgtgtggacttcgattttctctacgactttcctctccattgtatagtatagacttgaaagattcgaacaaacagaagattcaacagttgagttgctagatcagttagtgcttgagtagcttattatctctgaaatgtgagtgaggaactgctgggctcataagctgattatatacccggttatgactgctacggcttggttggatgctgccgtgtttcaggtctcgg

SEQ ID NO: 109 - DNA, 1971 bp, Paramacrobiotus richtersi
cacactttcttatcacattactcacactacatgttggtatttttcaagatcactgttatatgcttataattctgcacatggcctttaaaaaatgcatatatgaaggtctaaatttggtcatacatgatccaaattcatcaggctttgtttactggtaaatgccctgtctaaatctcctgtatggcgccggttgtccgccatcttgaccgcagtgagctaaattgggaagacgtgtttatttggtctggcgtatatcgggatcatcaattgccattatatatacacatctcctcttggtaacctaattcggggtacgcatgctgctgacattgggcagtgttcattacttgccattaccagccagcgacatttcgtcgtccattgtcgtgcatgcgcgacgtgattgaacctgtgtacaaccggaagtgtcgtgttgtatctagacgaatgcgcccggccgctcgcctggctggcgcttgaccccgacggccgctcgaggtgcctgatcatctgcatgtgtacagtacagcgtcattgagctaaaaagggcaaaatttacatggtcggagtctgatgttgacgatccgctgtcgaagaatctggattgctgattgatatctggatcctcagtagatcagttaacgtggtgtcaagagctgataatacatcgctagtacctccgggttttctgcgctaagcccatgatgatgtccagatctgtcgtaatactatggacaacggtgcttgtgggctggttaggccgagccgacttaatgcactgcacacccatcgttgaagaggttactactgctgctaaccatgttgccgggaccaacgggaacggccacgaggaccactggcactcgctaacggacacccagcaggccttcctcctgcacgcatattttactagcgccatgcccgtggacattaagagctgcgagcgcgtcgaggagtgcccgctaacgacgcgctgcgaccagacctggaagatctgcgtcccaaaagagtcgccccactttcccgctacagatggcatgtgcggtaaacaccacgactgcaagcccatgtaccattgcaccaacagcaagtgcttgctgaccggcccgctggcctgtaaaagcatcgacgagtgcctcaccatccccgggatgcactttgagtgcgtcgaagtccccgaacagctgccgggaagacgatgctggcgcaaatgccagacgagtcgggattgcatggaatgcgacgaaccgcagctcttagcggacctgaccgtggccagcacctgccgcgtcccgctgcccttccgcaactcggtcgtctgcaaggacaactactgcaagaagaaacttccgggcgagaagtgatcatgatttgcccgcttcatactgcttatttattgagcgtcggctcagctggatttatgttcaatagtaaatagtatgagcgtttttattcgtaatgataagcacttgttaattcttttagtgtcgtcttccttaatctgcgtagtaataatctgcgggcattaatattgatcgataaatgtacagaatgtatcgtctgcggcttgttgtagtctcagttcataaaaccgtactaccaacgaaatgataaacgcgactgcgtgcaacatactaaacagctgttaaatacataataattagtttaataagcagtcggcaaattgatcggcaaacaagttagtcttcagtgggtcttgtggaccgcggactcgacggtgctacttccggagacggcgtgcccgatggccgtatcaaagttgacctccacattggtggtcatcttgctcttctcgagagcatcccttgccgcctgtccctcccgctccagctggcgcttggccaggttggcctcgagatccacctgcttcttctgcgccgcaatggcctcgccgaccagctcctccttgaagtcgatgtcgcgctggtgctgcttctccagctccttgcg

SEQ ID NO: 110 - DNA, 1952 bp, Paramacrobiotus richtersi
gcaaggagctggagaagcagcaccagcgcgacatcgacttcaaggaggagctggtcggcgaggccattgcggcgcagaagaagcaggtggatctcgaggccaacctggccaagcgccagctggagcgggagggacaggcggcaagggatgccctcgagaagagcaagatgaccaccaatgtggaggtcaactttgatacggccatcgggcacgccgtctccggaagtaccaccgtcgagtctgaggtccacaagacccactgaggactagcttgcttgccgatccattttctgactgcttattaaactaattatttatatacagctgtttaacaagttgcacgcagtcgcatttatcttttcattgtgagtacggtcttatgaactgagactgcaataagccgcagatgagaaattctgtacatttatcgatcaatattaatgcccgcagattattactacgcagattaaggaagacgacactaaaagaattaacaagtgcttatcattacgaataaaaacgctcatactatttactattgaacataaatccagctgagccgacgctcaataaataagcagtatgaagcgggcaaatcatgatcacttctcgcccggaagtttcttcttgcagtagttgtccttgcagacgaccgagttgcggaagggcagcgggacgcggcaggtgctggccacggtcaggtccgctaagagctgcggttcgtcgcattccatgcaatcccgactcgtctggcatttgcgccagcatcgtcttcccggcagctgttcggggacttcgacgcactcaaagtgcatcccggggatggtgaggcactcgtcgatgcttttacaggccagcgggccggtcagcaggcacttactgttggtgcagtggtacatgggcttgcagtcgtggtgtttaccgcacatgccatctgtagcgggaaagtggggcgactccttgggcacgcagatcttccaggtctggtcgcagcgcgtcattagcgggcactcctcgacgcgctcgcagctcttaatgtccacgggcatggcgctggtaaagtaggcgtgcaggaggaacgcctgctgggtgtcggttagcgagtgccagtggtcctcgtgtccgttcccgttggtcccggcaacatggttagcagcagtagtagcctcttcggcgatgggtatgcagtccagtaagtcggctcggcctaaccaacccacaagcactgttgtccatagtatcacgacagatctggacatcatcatggctccagccggtgaaaagccggaggtatattgtcaattttcaaccctacgttaactgatcaactgaggatccaagatatcaatcagcactccagattcttcgacggcatcaagaatcccgctatgtgtcagcgacgaatttgtccttttagctcaatgacgctgtactatacacatgcagattatcatgcacctcgagcggccgtcggggtcaagcgccagccaggcgagcggccgggcgcattcgtctagatacaacacgacacttccggttgtacacaggttcaatcacgtcgcgcatgcacgacaatggacgacgaaatgtcgctggctggtaatggcaagtaatgaacactgcccaatgtcagcagcatgcgtaccccgaattaggttaccaagaggagatgtgtatatataatggcaattgatgatcccgatatacgccagaccaaataaacacgtcttcccaatttagctcactgcggtcaagatggcggacaaccggcgccatacaggagatttagacagggcatttaccagtaaacaaagcctgatgaatttggatcatgtatgaccaaatttagaccttcatatatgcattttttaaaggccatgtgcagaattataagcatataacagtgatcttgcaaaataccaacatgtagtgtgagtaatgtgataagaaag

SEQ ID NO: 111 - DNA, 1934 bp, Paramacrobiotus richtersi
tttttttttttttttcaatgtcatatgctatttattatttcgatacagacatacagtatcagattttgtcgttcatataatgaacacttcagctggcgttatgcaacttgacataatcgtggaggataaactaaaaaaagggattacggtagccctgtaaaataagtagcaaaatcgagcatcatggacttaacaatgttctgataaaaagctgtacacctttaacgttccaggtacaacactatgtttagaagcgtgtagctggatagctttccacccggcccacattctcccggatttcccgtccagctgctgtttgtacatcacccttactgatggtcgttccaatcgcggtatccactctaacgtcgatatgagtctgcgccttggcctgttccagagcgcgtgacgccgcttcgcgttccttctccagcgcgcgcatggcatactcggcttccaactggatctcgtgctgctgccgatccaccgctgtctcgaccatttccttgcggaactcaatatcccgaatgtgctgacgctccagagtgcgacgaatcagttcggcatccgcttccacttggttacggtacatggccgcccgctgggcggtgtccatctcatgttcacgggccaacgcttcgacactggcccgatagcgggcatattcttcaggcgactgcttcagctgtgtgttggccagtaaatcgatattcgtatcagcatagatttccgcatgggtcccgccgcttccctgggccaaaaggtcatgcgtcacactgggaatgggtggcgccagcatcgcaccgggaatgatcaattccttgtcgttggtgtacgtcacacccgaccggatgtgctccatctgcgcagcctccatacggtgaatggggatctggatgacctcgaccgacccctggcgaggagcggccgactggatgacctccgtccgttcgatcacttcacggtgtcgtcctggaatgataatatcctgttcttgacggtaaatgggagcgcgacccatctccactacgggagggatattctggacgtatgaggcgtccgtacgacgctccgtgacgtatgtatcgcgagccgggttcattatgggcaaattttgtagattttcaattccctgttggatgtagtccacatcgcgttgcaaagccggcgcttcctgtcgggcaatgttgacacgatcccgtgtgccggttgtgttctgggaaacacgggacccagccgttgcagtggacgccgaggacacggatgatacagacgacacagacgaggcaatggatgacgtgtcatcccctgattgtgataccgagcgctgtgaaaaacgctccagtgggaccgttggcggaataccggcgttattgttgatagatgccccactcgccatacttccttcccgaataatagtttgttccctttcttccagcaacgacggttcacgactctttcgttgagtatgactgaccgagctactatcggcaaagtagttctggatgccctggcgctgtgtagcgacgctgtccagttcgtggatctccgggacgtatcccacgggagcaccgggaaccagggccggtgggcgtttatccttgaagttcatgccgggatcctctccaccgcggtcaacgcggacatcacgcagttccgcgttaccatcggcgtccacgtggatctcttccacgcgttcatacttgaattcgtgggaatgcttgtcaccgggtactgtagtaaggtcacggtccagatgggtcgagacgaccgagtcgcggtcacgcgagacgaccgagtcccggtcacgctcgacatcgatgtcctggttggtgacgacgatatcgtccttatgcttatccttcttagaagacccgaatagtggcattttgaaatttaattaaagtagaaagagaaactttgaaaaatcgcga

SEQ ID NO: 112 - DNA, 1911 bp, Paramacrobiotus richtersi
gcaaaacaattaaaatttctacttttcgcgatttttttaagtttctacgttaattacaatttcaaaatgccactattcgggtcttctaagaaggataagcataaggacgatatcgtcgtcaccaaccaggacatcgatgtcgagcgtgaccgggactcggtcgtctcgacccatctggaccgtgaccttactacagtacccggtgacaagcattcccacgaattcaagtatgaacgcgtggaagagatccacgtggacgccgatggtaacgcggaactgcgtgatgttcgcgtcgaccgcggcggagaggatcccggcatgaacttcaaggataaacgcccaccggccctggttcccggtgctcccgtgggatacgtcccggagatccacgaactggacagcgtcgctacacagcgccagggcatccagaactactttgccgatagtagctcggtcagtcatactcaacgaaagagtcgtgaaccgtcgttgctggaagaaagggaacaaactattattcgggaaggaagtatggcgagtggggcatctatcaacaataacgccggtattccgccaacggtcccactggagcgtttttcacagcgctcggtatcacaatcaggggatgacacgtcatccattgcctcgtctgtgtcgtctgtatcatccgtgtcctcggcgtccactgcaacggctgggtcccgtgtttctcagaacacaaccggcacacgggatcgtgtcaacattgcccgacaggaggcgccggctctacaacgtgatgtggattatatccaacaaggaattgagaatctacaaaatttgcccataatgaacccggctcgcgatacatacgtcacggagcgtcgtacggacgcctcatacgtccagaatatccctcccgtagtggagatgggtcgcgctcccatttaccgtcaagaacaggatattatcattccaggacgacaccgtgaagtgatcgaacggacggaggtcatccagtcggccgctcctcgccaggggtcggtcgaggtcatccagatccccattcaccggatggaggccgcgcagatggagcacatccgttcgggtgtaacgtacaccaacgataaggaattggtcattcccggcgcgatgctggcgccacccattcccagtgtgacgcatgaccttttggcccagggaagcggcgggacccatgcggaaatctatgctgatacgaatatcgatttactggccaacacacagctgaagcagtcgcctgaagaatatgcccgctatcgggccagtgtggaagccttggcccgtgaacatgagatggacaccgcccagcgggcggccatgtaccgtaaccaagtggaagcggatgccgaactgattcgtcgcactctggagcgtcagcacattcgggatattgagttccgtaaggaaatggtcgagaccgcggtggatcggcagcagcacgagatccagttggaagccgagtatgccatgcgcgcgctggagaaggaacgcgaagcggcgtcacgcgctctggaacaggccaaggcgcagactcatatcgacgttagagtggataccgcgattggaacgaccatcagtaagggtgatgtacaaacagcagctggacgggaaatccgggagaatgtgggccgggtggaaagctatccagctacacgcttctaaacatagtgttgtacctggaacgttaaaggtgtacagctttttatcagaacattgttaagtccatgatgctcgattttgctacttattttacagggctaccgtaatccctttttttagtttatcctccacgattatgtcaagttgcataacgccagctgaagtgttcattatatgaacgaacaaaatctgatactgtatgtatgtatcgatataataaacagcatatga

SEQ ID NO: 113 - DNA, 1758 bp, Paramacrobiotus richtersi
agcagaatcgtaaaccatttgcagattgatggcgaaattttctttgctaattactcctaaagttttgtaattatttcttgcctcaagttatggatttaaagcagtacggagttttcttataaataacatcttaacaaccggccacaataatatcatggacttcaaattaaaatgttgaaaatatctcggtgtagaaaaattcgcagatcgtacttcggtacgccacgctgctgttgatgactgataggaccacattccattgcggtaaactccacctaccgcatgcataatttgaatagctggctgtccggtatgaatggccagcccagcccacaatggggaaatacccgggctacacgaatcatcgcatttccccggatggtgaaaactccacattcatcagtgcccatataaattcggcttcaggcagacgtggagaaatcattcagaaagtttttgagcacacgacgatttacaaagattttcgaagcaatcctctgattattttctcgtttcaatacgtttaccagtgataaattacgataatgtcgcatcatcacgaagagaaatttgagcgtgtggaagagcgcaaagtggatccagcccgtggcgtgcaggaggtccgcgtcggtatggacaccggtcatggcgatccagccctcaacttccagcccaccgatgccactttggtcaagggcaggacggtcgttggcggagtggatgctgcgggcatgacgaccggagcgacccagtattccggtgcctctacggtgcaatcgggaaccaatacctttgaagccgagaagaatacttcctacactcataccgaagtgcgcgctccacttgtgacccccgccgctccgttcatttccacgggagtgactggcctggctcaggatgtcgttggtgaaggtttcacggcgtctgctgcccgcatcactgccggcagcgcgtccgccgttgtgaccgagaccgctgagatgcgtgacaagtcgatgaaggagcaggaacgttatttgcgcgagaaggaggccatcgctcgatctcatgagaaggatctggagaagaagactgaggcctaccgaaaggaagcggaggctgaagccgagaagatccgcaaggagctggagaagcagcacgcacgcgacgtggaattccgcaaggacatggtggatgagaccattgagcgccagaaacgcgaggtcgagctggaagccaagtacgccaagaaggagctggaacacgaacgccagatggctcagaatgccctggaccagagcaagatggccaccaacatcgaagtgtccatggacaccgctgccgggcgcaccgtgagcggcggtaccaccgtgtcagagtcctttgagacccaccacgaagagcacggtaaggagaagaaatccctgggcgagaagatcaaggacacctttttgggccgttaagacgtcatcgcgaacatgacgtactctgtcgttgtatccacacttatcggcacattcttccctcgtttccttattaaccgtaatctctttgatggggttttttgtatttttgcgcgcgcttgtctatgccaaaacgtatgtaattaattggctgctttgacgtggttctttttgattcagcggtttgtatcaaactggttttagccgaccggatgctcttgtgaaaaagctgtgtgtttgtgttgctgaactggcgtttggatactgtttattttttttgtaatttgtggcagtgaaaaattaaactgatcatgct

SEQ ID NO: 114 - DNA, 1607 bp, Paramacrobiotus richtersi
ttttttttttttttttttaaagcatgatccgtttaatttttcactgccacaaattacaaaaaaaataaacagtatccaaacgccagttcagcaacacaaacacacagctttttcacaagagcatccggtcggctaaaaccagtttgatacaaaccgctgaatcaaaaagaaccacgtcaaagcagccaattaattacatacgttttggcatagacaagcgcgcgcaaaaatacaaaaaccccatcaaagagattacggttaataaggaaacgagggaagaatgtgccgataagtgtggatacaacgacagagtacgtcatggtcgcgacgacgccttaacggcccaaaaaggtatccttgatcttctcgcccagggatttcttctccttgccgtgctcttcgtggtgggtctcgaaggactctgacacggtggtaccgccgctcacggtgcgcccggcagcggtgtccatggacacttcgatgttggtggccattttgctctggtccagggcattctgagccatctggcgttcgtgttccagctccttcttggcgtacttggcttccagctcgacctcgcgtttctggcgctcaatggtctcatccaccatgtccttgcggaattccacgtcgcgtgcgtgctgcttctccagctccttgcggatcttctcagcttcagcctccgcttcctttcggtaggcctcagtcttcttctccagatccttctcatgagatcgagcgatggcctccttctcgcgcaaataacgttcctgctccttcatcgacttgtcacgcatctcagcggtctcggtcacaacggcggacgcgctgccggcagtgatgcgggcagcagacgccgtgaaaccttcaccaacgacatcctgagccaggccagtcactcccgtggaaatgaacggagcggcgggggtcacaagtggagcgcgcacttcggtatgagtgtaggaagtattcttctcggcttcataggtattggttcccgactgcaccgtggaggcaccggaatactgggttgctccggtcgtcatccccgcagcatccactccgccaacgaccgtcctgcccttgaccaaagtggcatcggtgggctggaagttgagggctggatcgccatgaccggtgtccataccgacgcggacctcctgcacgccacgggctggatccactttgcgctcttccacacgctcaaatttctcttcgtgatgatgcgacattatcgtaatttatcactggtaaacgtattgaaacgagaaaataatcagaggattgcttcgaaaatctttgtaaatcgtcgtgtgctcaaaaactttctgaatgatttctccacgtctgcctgaagccgaatttatatgggcactgatgaatgtggagttttcaccatccggggaaatgcgatgattcgtgtagcccgggtatttccccattgtgggctgggctggccattcataccggacagccagctattcaaattatgcatgcggtgggtggagtttaccgcaatggaatgtggtcctatcagtcatcaacagcagcgtggcgtaccgaagtacgatctgcgaatttttctacaccgagatattttcaacatt

SEQ ID NO: 115 - DNA, 1566 bp, Paramacrobiotus richtersi
caaccattatgaaacatgtaatacagctaagaaaataggcaccttcgcacttcggacaacgttgacaaggctaggattattgtacgcttagatccgttatgcattgttgatgtttcaacaagacttccggtaaggataattaatttgattactgcgtaaggtccagtaacatacacaaaagaacagccactgacaatgagcacaagatgtaggtagaaacgaggaactgatatggttttatggaacatctctgttctgataaccgccaagcccacaaaaaaataaagtaccatgccgtgcatttaaaaaatcccttgtacgtttcagagaaggatattgccacggtttcattatattaaaatttcttcacgagaagacatttttttggtaacagatgctaagatgaaaggcgattaaccatgcgttcgatgtactgtaagtctttaagaaattttctcatttctcaccgtggaaaaggcgagcagtttatatttggcatgcattgttttttggccacggcatttacgaactcagtaatctgatcatgaacaacgggcgaaataaaaagtaatgcaaatttaaggggcgtatgcgtttgtaaaaatggcattcaaagtcttaactgaaggtgtgcacgtgaaaatatgacatcgtctgatttgatttgctaaacttgaatgctacagtagtttcatcctcctaactatcgattcggccgctgctaacagtatgcccgactgtggtgtccatgttaacggaaatgtccgtgtgcatgcggctgttttcgagtgcgtcgagtgccaattccctttgccgctccagttctttcttggcgtattttgcttccagttccagttcttctttttgcctcttgatcgcatcttccaccagcttgctacgaaactccacatcgcgctcatgctgcttctccatctctttgcgaatcttagccgtttcccgttcggttttctcgcggtaatcttccgtcagctttccaatttccttctcatgcttttcgcgaatcttttcctttgcctggtagtatttctcatcccggtcccgtttccgttccagttgttcgggagtctcgtaaatgacttcatcccgagttccggccgtcacccgctccacggatgcggttatcccttcacccagcagctcttgggccagtccactgaccgcatgaaccgagaccgtgcccgtcggatggatcaacggcgcgtgaatggtcgtatgtgtggtcgctgtgtccagtgtatccgtgtcgacgtgcgttgttgaggatacatttttaatggactcgaccaaatggccaccgtcggaagtgcgtacttccttaacttctttatgctcatgagtatgaggcattttcgaagattgaatcttaagtagatagaaagaaaaattggtaaatatttattgtcactcaagagggtacatcaaccaaatgtgtagttaccgactgtttgcatacaagatttatatcaccgggcgaggtgtaatagaaatatggatattttcatgcgaaagtgctgtcgagaggttggctggggaactaccattttc

SEQ ID NO: 116 - DNA, 1452 bp, Paramacrobiotus richtersi
aacaactccacacaaggaatctccccgagacctgaaacacggcagcatccaaccaagccgtagcagtcataaccgggtatataatcagcttatgagcccagcagttcctcactcacatttcagagataataagctactcaagcactaactgatctagcaactcaactgttgaatcttctgtttgttcgaatctttcaagtctatactatacaatggagaggaaagtcgtagagaaaatcgaagtccacacaaccggcaacgtgccacctgtgatccctgccgcctgttccaacgtaacctgtaccacggtctgcgatcccaagtgcacggaacgccatgaacaccaccatcatactggcgtcgcctgcaacaccgtctgcagtccggcatgtggtgagcgtcatgaacatcaccagcatcaccagcatcacgaacattccggcagctgcaccgagacgtcggagaaatcgacgcactacacccataccgaagtgaaggcacccgttctcaacccatcggctccgtttgtggtgacgtccgcctccggactagcgcaagagatcgtatccgaaggcttcagcgcatcggccgccagaataagtggtgagtctgtgggcactatagtccatgagtcggccgcttcgtcgaaacaagccgcagtcgatctggagaaatatgagcgagaaaaagccgccattgccaagcagcatgagaaggagctggaaaagaagaccgagagctacaggaagcaagcggaggccgaagcggaaaagatccgcaaggaactagagaagcaacacgcacgggatgtggaattccgcaaagatgtcctggagaccaccattgagcgccaaaagaaggaagttgaactggaggccaaaatggccaagaaggaactcgagcacgaaaagaaactggcaatggacgcactggagcattccaaaatgtcgacgaatatcgaagtcaagttcgattcggccgctggacacacaacgacggagggtgtggttgtctctgaaagcgtgaacgttgcacacccacggatgtgagcgcatacttattcgaattcggccaatgtcccgaaatatctcgttatatttttttgattgatgttttctacattccgtgtagcgaaagctcgataagatctaattcctgatgtgttgcgaataacgagtaattccaccacggacgattgttatgctcttctctaattaaaatatattatcagctctacaataaaaagcaacctagtaacgtgctgtacggaagtacgacgtatgtgtttatgttatgtgattggtcagtacaggtacagctggtgtacctgtggttgaatgtgcgctagatgagagaatcaacgatgctcgtagattcatgatggaatggggattcagtcttgactagagagaatagtggaagcttccttgtgtttttgatgataaagtgtga

SEQ ID NO: 117 - DNA, 1416 bp, Paramacrobiotus richtersi
caaagcaaatttcctactttccaagcgacaaattttgaattttctcaacttttttcgagttttcaattgtacaatcagctattcaacatgcctctcttcggatctaacaaggacaaggacagtaaatcatcctacaaggaggagcatcacgagagccacacggagcgccgcatggaggacaactgcccaccgcccatgctgagcaaggacatgccgaccaatctcagcgggaaagtcgtcgtcgaacgccatgaaaccgtgtcgacgatccccgatgtcaaacccgtggtggagatgagtcgcacccccatgtaccgtcaggaggccgatatccatattcaggccggccaccgcgaggtggtggagcgcaccgatgtcatcaagtccaaggcgcaggcccagaaggaggtggagattgtcagcatcccgattcagaagatggccgcggcgcagatggagcatgtgcgtacgggtgtgacctttactcaggataaggagatgatcatccccggaccgatggttgctccgcccattcccagcgtgacccacgacctgctggtccagggctcgggcggtaccagcgccgagatccacgccagcaccaacgtggacctgttggccaacgcccaactcgccggacaatccccggaagaatacgcccgctaccgcgccggcgtcgaacaactggcccaccagcacgaagtggaaacgacccagaaagccgaggcctaccgccaccaggtcgaagccgacgccgagctgatccgccgcaccctggaacgccaacacgtccgcgacattgaattccgcaaggatatggtctccaccgccgtcgaccgccaacagcaggagatcaagatggaggccgagtatgccatgaaggcgctggagcaggagcgtatcgcggcggaacgggcgttggatcaggccaagatggagacgcatatcgatgtcaaggtggacacggccatcgggacgacgatcagtaagggggaggtgcggacggcggccgggcgggagattcgggaaagtgttggaccggtgacggttcaccatggggcgacgaggatctgaagggatggagctatgcagagtttttatagtgttgatatttcggcttgatttttttatatggctctctgaatctttattagtggctgataaagagtggatttgtaatgtatagccatgtgcattatagctgttcattatatgaaccatattacggcatacggtacgtgatccagcatgtactatggaataaattacggtaatggcagacatctgtctgatggttgttttatgatgcactcatgcgtaatattttgatgtttatgctgcttatgatacacgatggttatgatacacatttacacatgatgcatatagaatattatggttgctgccagtttgccg

SEQ ID NO: 118 - DNA, 1400 bp, Paramacrobiotus richtersi
tttttttttttttttcaaagtcatttttattacaagaacatcatttattggcatttttgcatacagcataagacaaaacaatactgccattcgcaacggaagacagtatactgtaccacggaacagcacatttcaaaatgttcatataatgaacaagctgagcataaatggaaaaaaacccgtcgaaaatagaaatcaatgcaacacctgcacatctaccagacctaaaaacacattttcatgataccaagataaaacgagatacccaaacatgaaagattgcacagaagcctgtataagatcccccgaacacctgtacacaaagctgcttaatgacgcgccggatgatcccgggtaaccggtccgacattctcccgaatctccttcccggcggccgtcaacacatcccccttactaaccgtggtcccgatggcggaatccaccttgacatcgatatgcgtctccatcttggcctgatccaacgcccgttccgccgcgatccgctcctgctccagcgccttcatggcatactcggcctccattttgatctcctgttgttggcggtcgacggcggtggagaccatatccttgcggaattcaatgtcgcggacgtgttggcgttccagggtgcggcggatcagctcggcgtcggcttcgacctggtggcggtaggcctcggctttctgggtcgtttccacttcgtgctggtgggccagttgttcgacgccggcgcggtagcgggcgtattcttccggggattgtccggctagttgggcgttggctagcaggtccacgttggtgctggcgtggatctcggcgctggtaccgcccgagccctggaccagcaggtcgtgggtcacgctgggaatgggcggagcaaccatggctccgggaatgatcatctccttatcctgggtaaaggtcacacccgtgcgcacatgctccatctgcgcggcggccatcttctgaatcgggatgctgacaatctccacctccttctgggcctgcgccttggacttgataacatcggtgcgctccaccacctcgcggtggccggcctgaatatggatatcggcctcctgacggtacatgggggtgcgactcatctccacgacgggcttaacatccgggatcgtcgagacggtctcgtggcgttcgacgatgactttctcgctgagattggtcggcatgtccttgctcagcatgggcggtgggcagttgtcctccatgcggcgctccgtgtggctctcgtgatgctcctccttgtaggatgatttactgtccttgtccttgttagatccgaagagaggcatgttgaatagctgattgtacaattgaaaactcgaaaaaagttgagaaaattcaaaatttgtcgcttggaaagtaggaaatttgctttg

SEQ ID NO: 119 - DNA, 1380 bp, Paramacrobiotus richtersi
ccgcgatcgattgatattttgccaagcatcatccttttcattatcatctcggctgtttttgattatgtatacagaaagcagtttatatttccatggcgaatactaaagcagcatagccgactgagactcagggttctttatattttacggaagccattaacacttactgatccaatttcgaagcccataaaatggagcacacggaagtgcataaaacgaccgagagcgccacgggccgctcgcacactatacagacggaaacgacggtcaaagaccagacatatgtcccgttacgcgagcaagccgaccattcgcccacatcctcgcacagatcgttccaggagagacaaacggtgcatacccacacggatgcgcggaaaccgagtctgggaacaatacatcctgtcagcatatcttccgcctcgggactggcccaagaaatcgtcgccgaaggatatcacgcatcggccgctagtgtccatagtacgacggccgctacgacaattgccgaatccccacaaacctacgaactgaaactgaaggacctggaacactatcgccgcgaacaggaagccattgcccgtaagtacgaaaaggaagtggagaaactgacggaaaagtatcgtcgaaagacggaagcggaggcggataagattcggaaagaactggagaagcagcatgcccgggatgtggagtttcgcgagaagctggtgcaggaggccattgcgcggcagaaagaggagattgtcctggaggccaagtatgcgacgaaggaactggacagacaacgaatgctggcattggaggcgctggagcggagtcgtcaccagtcgaatattcaggtaaatctggaaacggtggctggacacacggtcagtgagagccagaacgtcacgtcccattacgagtcacacgacagcattaacgaccacaagtcgatcggcgcgaaaatcaaagaagcgattatgggaaaacctgagtgagcagtacgtcatgaatatgatcacagccaaggaatactgtagtgcgggaaagtattaagcaggagcttactggacgagatggtattccagtatagttatccataagcgctaaatactgccctacgtgcggtatagtgacttcatgtccccatgcaactcataatgcatacacttaattatattgttctctgttgtcctgatgagaactcacatgacgtatcttgtgaaataatcaaggcatcgacgtacggtaacaattgcataattttgtgctttgcgtaaatgttttttacgtgaactttttcggatacttgcacgacgcttgctcgacagcgcttcttacccatcccttaacagaactagtaaactgtctaccttgttagttacagttgcaaca

SEQ ID NO: 120 - DNA, 1372 bp, Paramacrobiotus richtersi
cgcgtcgtttcattattattttatacatcggaaagtcccataccgatcgcgtattcgttgctgtcataaattgcaatggtaagaatccaagtcagctggacgtttttatttagaacgtttaacgcatcttttcattcttttcgtttcacagattgtcaaaaattagaaaaaaatcagccaggtttgctttataaacatgctgcccaaagataaggtacgttacggtacatagtataaaactgttgcaagcggagacgcggtgttattggtcgatgatgtgatgcgatctgggcgcacttatcgcttcatctcgcggtttcgttcggtaaacttctcggactgtgacaccacatgaccaccagattccgtgccggcagccgacgaatccagattgacctggatatcgctgtggaacttttgttgttcgagcatcattcgaaccttggtgcgttcacggtccatatcctttttggcatagcggcattcgaggtcgatcatgcgcttctggttctcgatggccagctcggcgatctccttgcggaactcaatgtcgcgcatgtgctgcttctccagttctttacggatcttgtcggcctccacttcctgatgctttcggtacgcctccgatttccgctccaactccttctcgtactgctgccccagcatctcattctccctggtcttagcggcgtaatcgcgacgggcttcttcgacgaactcggggatctccattgattgcatatcttccgtcacggcgctgatgcgagaaatcgatgcctgaaatccctcgccaaggagctcttgtgccgagaaagatgtggtgaaatagggcgtggggaaattataatggggcaccttaacctgcgactgcatgtagctggtctgctggacctcgctcttctcatggtacccattttgctcgctctcgggcggcggcacaaacatggcatcccggggaatgttcatattcatcgcttccattgttacgaaatcaaaactttatgatataaacaaattaaaacgttccagagtttgaaaagtactagtttcactgatgatagaaaagcagtagcaaccagctgctttatacaataattattaccggacagcgggaattaccggtgaacttgtacaggaaagacatgctgaaggtcggtttgcatgcggcatcacaaactgtcaagtgcaggcctgtgcctgtgagaatgatgaaaacgaacgagtgtaaagttcacacgggtaatatttcgtcgaccatgcatggaccttggtcgagcattgccttgagcagcggcagattctctcttgccgacagtatacggtggcgcagctcggtatatcaagagtaatacaaggtaacattctatggaat

SEQ ID NO: 121 - DNA, 1349 bp, Paramacrobiotus richtersi
tctcaactgtaactaacaaggtagacagtttactagttctgttaagggatgggtaagaagcgctgtcgagcaagcgtcgtgcaagtatccgaaaaagttcacgtaaaaaacatttacgcaaagcacaaaattatgcaattgttaccgtacgtcgatgccttgattatttcattatggccacaagatacgtcatgtgagttctcatcaggacaacagagaacaatataattaagtgtatgcattatgagttgcatggggacatgaagtcactataccgcacgtagggcagtatttagcgcttatggataactatactggaataccatctcgtccagtaagctcctgcttaatactttcccgcactacagtattccttggctgtgatcatattcatgacgtactgctcactcaggttttcccataatcgcttctttgattttcgcgccgatcgacttgtggtcgttaatgctgtcgtgtgactcgtaatgggacgtgacgttctggctctcactgaccgtgtgtccagccaccgtttccagatttacctgaatattcgactggtgacgactccgctccagcgcctccaatgccagcattcgttgtctgtccagttccttcgtcgcatacttggcctccaggacaatctcctctttctgccgcgcaatggcctcctgcaccagcttctcgcgaaactccacatcccgggcatgctgcttctccagttctttccgaatcttatccgcctccgcttccgtctttcgacgatacttttccgtcagtttctccacttccttttcgtacttacgggcaatggcttcctgttcgcggcggtagtgctccaggtctcgcagtttcagttcgtaggtttgtggggattcggcaattgtcgtagcggccgtcgtactatggacactagcggccgatgcgtgatatccttcggcgacgatttcttgggccagtcccgaggcggaagatatgctgacaggatgtattgttcccagactcggtttccgcgcatccgtgtgggtatgcaccgtttgtctctcctggaacgatctgtgcgaggatgtgggcgaatggtcggcttgctcgcgtaacgggacatatgtctggtctttgaccgtcgtttccgtctgtatagtgtgcgagcggcccgtggcgctctcggttgttttatgcacttccgtgtgctccattttatgggcttcgaaattggatcagtaagtgttaatggcttccgtaaaatataaagaaccctgagtctcagtcggctatgctgctttagtattcgccatggaaatataaactgctttctgtatacataatcaaaaacagccgagatgataatg

SEQ ID NO: 122 - DNA, 1263 bp, Paramacrobiotus richtersi
taccgagctgcgccaccgtgtactgtcggcaagagagaatctgccgctgctcaaggcaatgctcgaccaaggtccatgcatggtcgacgaaatattacccgtgtgaactttacactcgttcgttttcatcattctcacaggcacaggcctgcacttgacagtttgtgatgccgcatgcaaaccgaccttcagcatgtctttcctgtacaagttcaccggtaattcccgctgtccggtaataattattgtataaagcagctggttgctactgcttttctatcatcagtgaaactagtacttttcaaactctggaacgttttaatttgtttatatcataaagttttgatttcgtaacaatggaagcgatgaatatgaacattccccgggatgccatgtttgtgccgccgcccgagagcgagcaaaatgggtaccatgagaagagcgaggtccagcagaccagctacatgcagtcgcaggttaaggtgccccattataatttccccacgccctatttcaccacatctttctcggcacaagagctccttggcgagggatttcaggcatcgatttctcgcatcagcgccgtgacggaagatatgcaatcaatggagatccccgagttcgtcgaagaagcccgtcgcgattacgccgctaagaccagggagaatgagatgctggggcagcagtacgagaaggagttggagcggaaatcggaggcgtaccgaaagcatcaggaagtggaggccgacaagatccgtaaagaactggagaagcagcacatgcgcgacattgagttccgcaaggagatcgccgagctggccatcgagaaccagaagcgcatgatcgacctcgaatgccgctatgccaaaaaggatatggaccgtgaacgcaccaaggttcgaatgatgctcgaacaacaaaagttccacagcgatatccaggtcaatctggattcgtcggctgccggcacggaatctggtggtcatgtggtgtcacagtccgagaagtttaccgaacgaaaccgcgagatgaagcgataagtgcgcccagatcgcatcacatcatcgaccaataacaccgcgtctccgcttgcaacagttttatactatgtaccgtaacgtacgttgtctttgggcagcatgtttattaagcaaacctggctgatttttttctaatttttgacaatctgtgaaaccaaaagaatgaaatgaacgggttaaacgttttgaataaaaacgtccagctgaaaaaaaaaaaaaaaaa

SEQ ID NO: 123 - DNA, 1213 bp, Paramacrobiotus richtersi
cggaagtccgtgcgcctttggtaagccactgattacgtaatacgtatgcaaaaccaaatatgtatcgagtctagctggtgaacataacatttatacagcatgaatataatatcgtaaatcgtcaaaaggaaatatctgtaaaagtgattaacgtagtacagacgtgcaaaattgcacgtctgtattgcaaaattcttctcacggtaatctgatgcgccatacgtacgtacattattcgaaatctattttacgtcatagtagcgctcagacattatgtggtggacacgcaacctggacttcattctttgtcaacgtatctgcactaacatgcgctgaatttttaggtagtaccaccgccaccgattgttgctcatgctgccggaatcgccgaagaagttgtgggaaaaggattcactgcatccgccgcccggatcacgggaaccagtcagcaagtggacgttacgcccagtcctcagctgcaacaagaagtccgccgtgatgaagaacgttatatgcgcgaaaaagatgccatcgctgcgcagcatgagaaggaattagaaaggaaaacagaagcctaccgaaaaacggccgaagcggaggctgaaagaattcgcaaggaactagaaaagcaacatcaacgtgatgttgaattccgaaaagatcttgtggacagcgctataaataggcagaaacaagaagttgaactcgaggcaaaactggccaaaaaggagctggagcgtgaagctgctatggcaaaagaagcgctggaaaggtcaaaactatccaccaatatcgaggtcaacttcgacagcgctgtgggtcacacgcagtcggcaggcaccactgtgtccgaatcggaatcgatttccagaacggttaagaagtgaaaggaccatggtggcacattgtcatctgacgggaaggacgcctctgacatttttttagtatggtgaagggacaagatacaattggattctgattacaatcaccgttgtctcaacttgtctggtatatatcagactgttacattttagaactgcaaatgtgcatgttgatgtactgtacttaattgactattaacgcctttggttatcactcggtactaggtctagattactgcgcattagtctggaatagcatgtaaaaaagacgtcatgaaaaaagatcaaaactatatggttaaatttcggcaagccagttaagtgtaccacag

SEQ ID NO: 124 - DNA, 1134 bp, Paramacrobiotus richtersi
tgctcttccgatctttttttttttcttaagagtaagttatagtgagtggaatgcgccaacacagagagataagtggttaaaatgatgttgacttataagtcccacagcgtgaaaaaatcagacatatttatcacgtattatctttatcgcatgtccgttataataagcgttattacttccgttattagagaaaagcgacatcagtagaatagtataaaacatggaaaaagcacggtaggtttatggatagccgtaaaaaatacgccatcaggtgcagtaagtactatagagacatgtccagtgagaaacttgtaacttgggacgatcgtgaagatgcggttgcggcgtaattattgctttatctggcggtttcgttcggtgaacttttgagattccgacaccacctgacctccagtttccgtgccagcagccgaagaatccagattgacctggatgtcgctgtggaacttttgctgctcgagcatcattcgaaccttgacgcgttcgcggtccatgtccttcttggcgtagcggctctccacgtcgatcattttcttctggttctcgatggccatctcgacaatgtccttgcggaactcaacgtcacgcagatgctgcttctccagttctttgcggatcttgtcggcctcgacttcctgctgcttgcggtaggcctcggtcttgcgctccacttccttctgataattggctgacagcatctcctgctcacgggttttggcagcgaagtcgcgacgggcctcttcggccagttcgggaatctcgatggacgacagctcttccgagacggcgctgatgcgcgaaatcgaagcctggaaaccttcgccgagaatttcttgggcagaaaaggaagtgctgaagaacggagcggggaggttgacaaggggcaccttgacctgggactgcaggtagctggtctgctggatctcctgcttctcctttcgctcgtactgctcaccctcgggcgggggaataaacacggcgtcctggttcatgttcatgttcatcgcttcagcagacattattaatcaggcgcttaagttaagacgaataatcgaataaacgagagtttaaaaagttttgaaagtattgtaaagcttgttaagaactgatcgc

SEQ ID NO: 125 - DNA, 1122 bp, Paramacrobiotus richtersi
caagggtcagggctagtggggactggaaatttccccggttagtaaccactgttcggattccaccggttgactgtggtatttaaaccgcagtctgtcgtttaaattttcatcaaatacttcagttctttaaattacgattaaaccgtgaaattcgctaactattcctttgactttattgtgtgcgagagtgatacttacatttttaccatggaagccagacagcacgagcaacagttccagaagcaggaggtggagcagaccagctacatgcagacccaagtgaaagtgcccgttatgaaactctcggcgccctccatcatcacggtgcccctggcgcaggaactcgttggtgaaggatttcaggcttcgatttcgcgcatcagcggtgtctcccaggagatccagcagatcgactccgcacaattagacgaggaggtccgtcgggactatgaatctaaacagcgcgaagcggaactgctgcagcagcaattcgacaaggaggtggaaaagaagaccgaggcctaccgcaaacaacaggagattgaagccgagataatccgtaagatgttggaaaagcaacacatccgcgacgtggagttccgcaaggagctcgtcgagcatgccatcgagaaccagaaacgccagatcgacattgagagccgctatgccaagaaggagctggaacgggagcgcaccaaggccagaatgctgttggagcgacagaaattccacagcgacattcaggtcaatctggattccaccgccgcgaccacccatgcaggagagcaagtggtgtccgagtcggagaagttcacccagaactccaagatgtcgtgcggccaacagcgtgccggataatccaacgttttctccgccttgttttgtactatgacgcgttctcttactttttagattattggcccaattgtggtgtgaacctatgtcgttgagttatgttggctgtatcagcgattttcgtaatttttgatgttaatgttttatagaatttagctgggtagttcacacgtactgttgtgtacacagtatatgttgcgtgattttggttaataaactttaattagaagtatgtggtatttactgcagcttcggtatgaaaaaaaa

SEQ ID NO: 126 - DNA, 1097 bp, Paramacrobiotus richtersi
gtacgagcgaaaggagaagcagggatcagttcttaacaagctttacaatactttcaaaactttttaaactctcgtttattcgattattcgtcttaacttaagcgcctgattaataatgtctgctgaagcgatgaacatgaacatgaaccaggacgccgtgtttattcccccgcccgagggtgagcagtacgagcgaaaggagaagcaggagatccagcagaccagctacctgcagtcccaggtcaaggtgccccttgtcaacctccccgctccgttcttcagcacttccttttctgcccaagaaattctcggcgaaggtttccaggcttccatttcgcgcatcagcgccgtctcggaagagctgtcgtccatcgagattcccgaactggccgaagaggcccgtcgcgacttcgctgccaaaacccgtgagcaggagatgctgtcagccaattatcagaaggaagtggagcgcaagaccgaggcctaccgcaagcagcaggaagtcgaggccgacaagatccgcaaagaactggagaagcagcatctgcgtgacgttgagttccgcaaggacattgtcgagatggccatcgagaaccagaagaaaatgatcgacgtggagagccgctacgccaagaaggacatggaccgcgaacgcgtcaaggttcgaatgatgctcgagcagcaaaagttccacagcgacatccaggtcaatctggattcttcggctgctggcacggaaactggaggtcaggtggtgtcggaatctcaaaagttcaccgaacgaaaccgccagataaagcaataattacgccgcaaccgcatcttcacgatcgtcccaagttacaagtttctcactggacatgtctctatagtacttactgcacctgatggcgtattttttacggctatccataaaccaaccgttctgttgccatgttttatacaactcgattaatgtcactttattacacggaagtatcaacgcttattataacggatatgataaaattaatgcgagataaatgtttgactttttcaccatttggagcttggcagtcaacatcattaaattagccacttatcgaaaaaaaaaaaaa

SEQ ID NO: 127 - DNA, 1095 bp, Paramacrobiotus richtersi
gtaaagaagcattgattaattacagaagagctaggattgctttgagagaatacaattagcattaaaaatctaacaacgtaaatacaatggcgcacgaactcaaccctcacgaaacccggaccgatttctcggatgcggagggaggcagctacgaaaaacaaattcattcggagttgcgagccccatcagcaacaccttccggtcattcccaaagtgataagagagaaactactgtgacttacacctacacagatgtccgaactccacagatgaatccacctgcacctgttttgatcattccatccgctgcgggactagctcaagaaatcgtcggcgagggattcaccgcatcggccgccagagtaacgggcgccagtccccaggtgaccgttactgaaacactcacctcacaagaaaaatatttacgtgagcaggagaactaccgtcgagagcaggaagccctggtccgcaagtacgaacggtctatcgagaagatgaacgaagagtaccgcaagaaaaccgaacaggaagcggacaagatccgcaaagaaatggagaagcaacacgagcgggacattgaattccgtaaggagctgatggacaaggccatcgaacggcagaaagaggagattgccctggaagccaagtatgcgcggaaggaactggaacgacaacgggagatggccatggaagcgttggataagacgaagaagcaggcggatgtgcaggttaatctggacacgttggccgggcatacggttagcgaaagccagagccagctgacgccggatgcggatatcccagccgatcacagagaaccgcataagtcgttaagctcgaaactgcgcgaaactttcacagggaaatagcgcattatgttttcttggatgttggacgttgtaaactgtaattccatatgtagcacttactttattcttacggttaattagccaaacatacaagttcaaggtgatatataactctcgcacgtgccatgcatttttttactcttcgtttcacaaagttcacattaaatttcaccaaatgcgatcgaaaaataaacagcgttaaggcagagatcggaagagcgtcgtg

SEQ ID NO: 128 - DNA, 1094 bp, Paramacrobiotus richtersi
cttattcaaaagtactagtcatcagagagctgtagttcatccacgactgcgtcggacagagttgccaaaattaaactgtgtttcgaaactttacggtgaaacaatttttatatgcagttccaaataaaagcgaaatacgtctaatcaatagaccagctggatgtatgtaagatccaagattcagaaccgttagcaattgacgtacaacatccaaatttgaaatataaagcgcagcaatctgatttgtaaccagaagctagcagatttaataaagtcaaatattttattatatttgatattccagaaaaaactgagaagccatccgtcccatcaggtaatgtaaaaccgtgctcccttcacttctttaccgttctggtaaccgattccgattcggacacggttgtgcctgctgattgcgtgtggccaacagcactgtcgaagttgacctcgatactggtggcaagttttgatttatccagcgcttcctttgccatggcagcttcgcgctccaactcttttttggccaattttgcctcgagttcgacttcttgtttctgtctgtttatggtgctgtccacaagatctttccggaactcaacgtcacgctgatgctgcttttctagttccttgcgaattctttcagcctccgcttcggccgtttttcggtaggcttctgttttcctttctaattccttctcatgctgcgcagcgatggcatctttttcgcgcatataacgttcttcatcacggcggacttcttgttgcagctgaggactgggcgtaacgtccacttgctgactggttcccgtgatccgggcggcggatgcagtaaatccttttcccacaacttcttcagcgattccggcagcatgcgcaacaatcggtggcggaggaaccaccaaaggcgcacggacttccgtgtgtgtatatccagtatgcacagtttcttgatgtccatgcgtttccgtgcggcatttttcggaatgggtatgcttctcctggcacacttcgacttttcgctccatactgcagaagagtgttacgttattgtgaattcaaaaaataactgaaagagacgtttaaaaaactgtgtc

SEQ ID NO: 129 - DNA, 1087 bp, Paramacrobiotus richtersi
tttttttttttgccttaacgctgtttatttttcgatcgcatttggtgaaatttaatgtgaactttgtgaaacgaagagtaaaaaaatgcatggcacgtgcgagagttatatatcaccttgaacttgtatgtttggctaattaaccgtaagaataaagtaagtgctacatatggaattacagtttacaacgtccaacatccaagaaaacataatgcgctatttccctgtgaaagtttcgcgcagtttcgagcttaacgacttatgcggttctctgtgatcggctgggatatccgcatccggcgtcagctggctctggctctcgctaaccgtatgcccggccagcgtgtccagattaacctgcacatccgcctgcttcttcgtcttatccaacgcttccatggccatctcccgttgtcgttccagttccttccgcgcatacttggcttccagggcaatctcctctttctgccgttcgatggccttgtccatcagctccttacggaattcaatgtcccgctcgtgttgcttctccatttctttgcggatcttgtccgcttcctgttcggttttcttgcggtactcttcgttcatcttctcgatggagcgttcgtacttgcggaccagggcttcctgctctcgacggtagttctcctgctcacgtagatatttttcttgtgaggtgagtgtttcagtaacggtcacctggggactggcgcccgttactctggcggccgatgcggtgaatccctcgccgacgatttcttgagctagtcccgcagcggatgggatgatcaaaacaggtgcaggtggattcatctgtggagttcggacatctgtgtaggtgtaagtcacagtagtttctctcttatcactttgggaatgaccggaaggtgttgctgatggggctcgcaactccgaatgaatttgtttttcgtagctgcctccctccgcatccgagaaatcggtccgggtttcgtgagggttgagttcgtgcgccattgtatttacgttgttaggtttttaatgctaattgtattctctcaaagcaatcctagctcttctgtaattaatcaatgcttctttaccc

SEQ ID NO: 130 - DNA, 1019 bp, Paramacrobiotus richtersi
ctccttgcggaactcaatgtcgcggatgtgttggcgttccagggtgcgacggatcagttccgcgtcggcttcgacttgggtgcggtagagggcggctttctgggccgtgtcgatttcgtgctggtgggcgagttcttccacgctggctcggtagcggttgtactcctcgggggatgagtcgaggtgggcgttggctaggaggttgatgttggtgtcggcgtggatttcggcgtgggtaccaccgcttcctcgagccagtaaatcctgtgtgcagctgggaatcattggcgcaatcatcgcaccaggaataatcaactccttatcattcgtatacgtcacacccgagcgtacatgttccatctgcgccccgctgaccttatgaattggtatactgatcacttcgacggattctcgtcggggcgtggctgaccggatcacctccgtcttctccaccacctcccggcgttccccagggatgatgatattcgcctcctgatgatacatgggctggcggctaatctccaccgcggctgggacactctgaacataggacacttccgaacgacgctccgtcaccgttttgtgaacgggactacgggaaatgtggcgctcttggataagcgacccttcccgccctggagagcggtcggggaattggtccacctcccggtcaaagtgttccaggttgcggttttcaatatcgttgtcccggtcggtgatggggaattcagcgccgtgatggtgcttgtccttcttggatgatccgaataaaggcatgtttgtagttaaagaatcaaggattattcagaaaaaaatagtcagttcactttcagtttgttgcaatgggagattaggttttacatcggccggtatatatgcttttccgatggtgaacgtacctggtaattgttcaacgggcaaatccctatggtaatagaaaaatccattgaacacagcacatacggcgaaatgaatggagtttatgtaactcaactttgcacatacagcggaaaagtacg

SEQ ID NO: 131 - DNA, 854 bp, Paramacrobiotus richtersi
ctaatctcccattgcaacaaactgaaagtgaactgactatttttttctgaataatccttgattctttaactacaaacatgcctttattcggatcatccaagaaggacaagcaccatcacggcgctgaattccccatcaccgaccgggacaacgatattgaaaaccgcaacctggaacactttgaccgggaggtggaccaattccccgaccgctctccagggcgggaagggtcgcttatccaagagcgccacatttcccgtagtcccgttcacaaaacggtgacggagcgtcgttcggaagtgtcctatgttcagagtgtcccagccgcggtggagattagccgccagcccatgtatcatcaggaggcgaatatcatcatccctggggaacgccgggaggtggtggagaagacggaggtgatccggtcagccacgccccgacgagaatccgtcgaagtgatcagtataccaattcataaggtcagcggggcgcagatggaacatgtacgctcgggtgtgacgtatacgaatgataaggagttgattattcctggtgcgatgattgcgccaatgattcccagctgcacacaggatttactggctcgaggaagcggtggtacccacgccgaaatccacgccgacaccaacatcaacctcctagccaacgcccacctcgactcatcccccgaggagtacaaccgctaccgagccagcgtggaagaactcgcccaccagcacgaaatcgacacggcccagaaagccgccctctaccgcacccaagtcgaagccgacgcggaactgatccgtcgcaccctggaacgccaacacatccgcgacattgagttccgcaaggag

SEQ ID NO: 132 - DNA, 717 bp, Paramacrobiotus richtersi
cttttacatcattctgcggaaaatggtagttccccagccaacctctcgacagcactttcgcatgaaaatatccatatttctattacacctcgcccggtgatataaatcttgtatgcaaacagtcggtaactacacatttggttgatgtaccctcttgagtgacaataaatatttaccaatttttctttctatctacttaagattcaatcttcgaaaatgcctcatactcatgagcataaagaagttaaggaagtacgcacttccgacggtggccatttggtcgaatccattaaaaatgtatcctcaacaacgcacgttgatacggacacactggacacagcgactacgcatacgaccattcacgcgccgttgatccatccgacgggcacggtctcggttcatgcggtcagtggactggcccaagagctgctgggtgaagggataaccgcatccgtggagcgggtgacggccggaactcgggatgaagtcatttacgagactcccgaacagctggaacgaaaacgggaccgggatgagaaatactaccaggcaaaggaaaagattcgcgaaaagcatgagaaggaaattggaaagctgacggaagattaccgcgagaaaaccgaacgggaaacggctaagattcgcaaggagatggagaagcagcatgagcgagatgtggagtttcgtagcaaactggtggaagatgcgatcaagagg

SEQ ID NO: 133 - DNA, 644 bp, Paramacrobiotus richtersi
accacgagctacacccgatgttacgactatttctgtcgcctggaaactgctggagagctggacgaaatcgacaaactccactgagaaaccgtcttcacctgtaactgtttcattgcaaacttttgattcacgtcagacaacgaacttcgtgttaatttactgacagtgagattaatcccttctgttgaattaagaaaatgtcgcacgcccaccacgcccacaactacgaatgctacacgcacacggcggacggcaaggcggcggccacggccaccgccacgtcgctggcgtcgggcgagaaggaggtgcacaccaccagctacacccacgtggacgccaaactgcccctgctgcaggacctcccgtcgcccctcaccaccacgggcatcgccggccttggccagaccctggtgggcgagggcttcacggcctcggtcgtccgtgcgtccggcgagtcggagcaggtgaccgtggcgcccagtgagcgcctgaccgaggaggcccgtcgcgaccaggagcgctaccagcgcgacaaggacgccatcaacgagcgccagaagcacagcgtggagaacaaggccgagaactaccgcaaggaggccgagcagcaggccgagcgcatccgcaaggagctggagaagcag

SEQ ID NO: 134 - DNA, 627 bp, Paramacrobiotus richtersi
gcaaggagctggagaagcagcacgaacgcgatgtcgagttccgcaagggattgattgatagtgcgattgaacgacaaaagcgggaggtcgagctggaggccaagatggccaaacgcgaactggaccgggaggcccagctggccaaggaggcgctcgagcggtccaagttggctacgaatgtcgaggtcaacttcgacagtgccgtcggtcacactgcttcggtgggaacaacggtctccgaatcggaatcgatcacgcgggatgtacgcaagaactgagccatattcaacatacagctctgtacattatctcgtcattcttttggagcggcttctcaccttcgcagcctcgtgccaaaaacctgcttttctcatatggtttgctggcttattgtgcttaataaataaaagtaattactgacaataactgtttccagctaattagtacggagactatctaattagtacttcgtgactgaagcatcgtgatgttgaactgcacgacaactttgcattcactgaataaagcgcgcttaataacatgaaaatatgatgaaatacgacgttgtcagttttcttgttctgtagaatttgctcataaaaaaacaaagaaatgcacagcacgaaa

SEQ ID NO: 135 - DNA, 611 bp, Paramacrobiotus richtersi
tgcttctccagctccttgcggatgcgctcggcctgctgctcggcctccttgcggtagttctcggccttgttctccacgctgtgcttctggcgctcgttgatggcgtccttgtcgcgctggtagcgctcctggtcgcgacgggcctcctcggtcaggcgctcactgggcgccacggtcacctgctccgactcgccggacgcacggacgaccgaggccgtgaagccctcgcccaccagggtctggccaaggccggcgatgcccgtggtggtgaggggcgacgggaggtcctgcagcaggggcagtttggcgtccacgtgggtgtagctggtggtgtgcacctccttctcgcccgacgccagcgacgtggcggtggccgtggccgccgccttgccgtccgccgtgtgcgtgtagcactcgtagttgtgggcgtggtgagcgtgcgacattttggaaactcgaaaagcaggcttccacaacaagttttatgtcaattaacacgaagttcgttgttgtttgacgtgaatcaaaagtttgcagtgaaacaggtacagctaaagacggtttctcagcggagtttgtcgatcttccaccagctctccagcagtttccag

SEQ ID NO: 136 - DNA, 822 bp, Paramacrobiotus richtersi
acgggaaacggctaagattcgcaaagagatggagaagcagcatgagcgcgatgtggagtttcgtagcaagctggtggaagatgcgatcaagaggcaaaaagaagaactggaactggaagcaaaatacgccaagaaagaactggagcggcaaagggaattggcactcgacgcactcgaaaacagccgcatgcacacggacatttccgttaacatggacaccacagtcgggcatactgttagcagcggccgaatcgatagttaggaggatgaaactactgtagcattcaagtttagcaaatcaaatcagacgatgtcatattttcacgtgcacaccttcagttaagactttgaatgccatttttacaaacgcatacgccccttaaatttgcattactttttatttcgcccgttgttcatgatcagattactgagttcgtaaatgccgtggccaaaaacaatgcatgccaaatataaactgctcgccttttccacggtgagaaatgagaaaatttcttaaagacttacagtacatcgaacgcatggttaatcgcctttcatccgagcatctgttaatgaaaagaagctcttctcgtgaaaaacttttaatgtaataaaaccatggcaatattcttctctgtatagatacaagaaatttatgaactgcgcggcttggtactttgttttctcgtgagcttgccgttaatcagagaatgtttcataaaactatatcaatttcttgttcctacatagcttatactcagcggctgtttctgtgtgtgtgatactgtaacgtgcgcagtaattaaattaattatccgtacc

SEQ ID NO: 137 - DNA, 1398 bp, Hypsibius dujardini
gtaaatacaataggaatgaataccagtggtggtcgttaatctttaaggaacatggccaattcgtacggcaaatcacgaggccaaattgttttctttgcgactttagaaaagatactatgaacagtttcccgtcagtaagggtctacaatacacacacatacagtgcaagaccgaattcgcagtcacttttacgccctgcacatgcaacgcctcatttcaacagttatagacaattagaattagtaaaaaacaggaaactctcagggagacttcaaatgaaaattattatcatcgtttctttacgccaccgttagtagcctagcagctggcactagtaagcaaaatatgggagtaaatattagtattaatatgatgaacgttaactcaacacaagcgaaataaaattgcgagttcgggcaacgggaaggactcagttgttggtctggaacttttccgactccgacacggcaacgctggatgatccggaatgagttgaacccgcagcggcttccatgttgacctggatatcggtgtggaatttggagcgttccagcacacgcttgaccttgttccgttcccgctcaagctccttcttggcatagcgtgcctccaggtcgatctgacgcttctgattctcgatggtctgctccatcaactccttgcggaactcgacatcccgcagatgttgcttctcaagttccttccggatcttctcggtctcaacttcttgctgcttgcgatacgcttcggtctttcgctccagctccttctcaaactgtcgcgacaggagctcctgctcgcgggccttggcttccatgtcccgacgtgcctcctcggtttcggcctcggtgtcgatgacggtgagctccccggatgcgccggtgatgcgagagacggacgcctggaaaccttccccgaccagttcttgagccaaacccgcggaagagctgatgaaaggtgcgattaagttgggcatgttcactttgacgtcggtgtgggtatagctggagtgttcgacctccttcttctcctgttgctcttgctgttgaccattgttgctccggtcgctgtactgatagctggattcttgctggtagttagacattgttggtagtcggaagctagacttttttgggacagtttagtcaaaaagaattataagtactgatgtatgtagtagggtggaatctcgaaatacgaaaactgtgtgatgaaacttgaggtggtttctttcatagcttttatatggtccgaccgccagccatcactttaagctgagtgaccactggggttgtgaaatttccccgttggggaaactccccagtcggcattcgttttgtgtgtgtgaaagctcgggtaatttcgggtggcagttgtccggacgaagttcgagatgga

SEQ ID NO: 138 - DNA, 1405 bp, Hypsibius dujardini
gtccggacaactgccacccgaaattacccgagctttcacacacacaaaacgaatgccgactggggagtttccccaacggggaaatttcacaaccccagtggtcactcagcttaaagtgatggctggcggtcggaccatataaaagctacgaaagaaaccacctcaagtttcatcacacagttttcgtatttcgagattccaccctactacatacatcagtacttataattctttttgactaaactgtcccaaaaaagtctagcttccgactaccaacaatgtctaactaccagcaagaatccagctatcagtacagcgaccggagcaacaatggtcaacagcaagagcaacaggagaagaaggaggtcgaacactccagctatacccacaccgacgtcaaagtgaacatgcccaacttaatcgcacctttcatcagctcttccgcgggtttggctcaagaactggtcggggaaggtttccaggcgtccgtctctcgcatcaccggcgcatccggggagctcaccgtcatcgacaccgaggccgaaaccgaggaggcacgtcgggacctggaagccaaggcccgcgagcaggagctcctgtcgcgacagtttgagaaggagctagagcgaaagaccgaagcgtatcgcaagcagcaagaagtcgagaccgagaagatccggaaggagcttgagaagcaacatctgcgggatgtcgagttccgcaaggagttgatggagcagaccatcgagaatcagaagcgtcagatcgacctggaggcacgctatgccaagaaggagcttgagcgggaacggaacaaggtcaagcgtgtgctggaacgctccaaattccacaccgatatccaggtcaacatggaagccgctgcgggttcaactcattccggatcatccagcgttgccgtgtcggagtcggaaaagttccagaccaacaactgagtccttcccgttgcccgaactcgcaattttatttcgcttgtgttgagttaacgttcatcatattaatactaatatttactcccatattttgcttactagtgccagctgctaggctactaacggtggcgtaaagaaacgatgataataattttcatttgaagtctccctgagagtttcctgttttttactaattctaattgtctataactgttgaaatgaggcgttgcatgtgcagggcgtaaaagtgactgcgaattcggtcttgcactgtatgtgtgtgtattgtagacccttactgacgggaaactgttcatagtatcttttctaaagtcgcaaagaaaacaatttggcctcgtgatttgccgtacgaattggccatgttccttaaagattaacgaccaccactggtattcattcctattgtatttacccatacgcgtgtaatgtctttc

SEQ ID NO: 139 - DNA, 1369 bp, Hypsibius dujardini
gttcttctgtgcaacaccaacggaaacgccctcggccagaatgtttttccattcaacctgcgagcagaattatcgcttaattgcttaactatccgcgaggttctgcgctgttgtattgaggaacggaaaaaccccaacgaaccaaaggggtgccgaaaaaaaacagcgaaaaagacggaaatttttttttggtgtttgaacaaaaatgttttttggtatataaaccggctcctattctgcggagcatttcatcaaacattttgcgaaacaatttccaataaattcctgttctcaaaaatctttccttgaagtacctttcgttgctaaatcagtgaaaacaatcctcaacaagatgtctggacgtaacgtagaaagccacatggagcggaatgagaaggttgtggtcaacaattccggtcatgctgacgtgaagaagcaacagcaacaagtggagcacaccgaattcacccacaccgaggttaaagccccgttgatccatcccgcacctccgatcatctcaactggagctgccggactcgccgaggagattgtgggacaagggttcaccgcgagtgccgcgcgcatcagtggaggtaccgctgaagtacatctccagccttcggccgcaatgacggaagaggcccgtcgcgatcaagagcgctaccgccaggaacaggagtcgatcgccaagcagcaggaacgtgagatggaaaagaagactgaggcataccgcaagaccgccgaggcggaagctgagaagatccgaaaggagctggagaagcaacacgcgcgtgatgtcgagttccgtaaggatctcatcgagagcacgattgaccggcaaaaacgcgaggtcgatctggaagcgaaaatggccaagcgggaattggatcgtgaagggcagttggccaaggaggcgttggaacgatcacgtttggccaccaacgttgaggtcaatttcgacagtgcagctggtcataccgtgtccggtgggacgaccatctccagctctgacaagatggaaatcaagcgcaactagactgcaatacggctgtggtattccagcggattcgacttttttcctattaccattttaattcagttatagcgcttatgaccgcatcacccttcctttgctttcgcatctgtaaagtatgtattacaacgacgggattttaagatattttactcgcggaattttccgctctttcagtatacactcgcgtactgtactaccaatgcgtattaactaaaagccggtctagcttccgtactttctatttctttgtatcgtagttatcaaatccgaagagtaatgtgaggaatatttttggtttttagacaaaaaaatacaatgttgcaatcaagacaa

SEQ ID NO: 140 - DNA, 1269 bp, Hypsibius dujardini
tgcagttctaaatttgtgttgttttttattctttcaaattggctgacaattataattatttctccgtacgtataccggacacagctgtttgaaaaaactcgcttagctgtagtgttttattttttagcgcgtattagcccaacagtccaaaaatttgcgggaaaaagaaactaaatttagattatagtgtgatttggcaaaacttcggttcattttacgtatacagaatgtgcaagtgcgcaatatactgaggtgaaagcaaacggaaagccgaccgcggcgaggtagaaaaacgtgtaatcatgcagtacagtacgaattctcataaactctaaacggagtcggagtgatctagtttcgcttgatttccatcttgtccgaagtggatacggtcgtcccaccggacacggtatgaccagctgcactgtcgaaattgacctcaacgttggtggccaaacgtgatcgttccaacgcctccttggccaactgcccttcacgatccaattcccgcttggccattttcgcttccagatcgacctcgcgtttttgccggtcaatcgtgctctcgatgagatccttacggaactcgacatcacgcgcgtgttgcttctccagctcctttcggatcttctcagcttccgcctcggcggtcttgcggtatgcctcagtcttcttttccatctcacgttcctgctgcttggcgatcgactcctgttcctggcggtagcgctcttgatcgcgacgggcctcttccgtcattgcggccgaaggctggagatgtacttcagcggtacctccactgatgcgcgcggcactcgcggtgaacccttgtcccacaatctcctcggcgagtccggcggctccagttgagatgatcggaggtgcgggatggatcaacggggctttaacctcggtgtgggtgaattcggtgtgctccacttgttgctgttgcttcttcacgtcagcatgaccggaattgttgaccacaaccttctcattccgctccatgtggctttctacgttacgtccagacatcttgttgaggattgttttcactgatttagcaacgaaaggtacttcaaggaaagatttttgagaacaggaatttattggaaattgtttcgcaaaatgtttgatgaaatgctccgcagaatgggagccggtttatataccaaaaaacatttttgttcaaacaccaaaaaaaaatttccgtctttttcgctgttttttttcggcacccctttggttcgttggggtttttccgttcct

SEQ ID NO: 141 - DNA, 2671 bp, Hypsibius dujardini
aattacaccaagagaaaacgcgcatttgtcgttgagcgaacggaaaaaaagaattcctctctccattttgatttaatttcttctcccgattgagtgcattaatcgttccgcgccccacacccattttagttttgtaatttttgtatttttcctgccgttctacaatcatggtgatgtcgatggaggagtcgatcagtcagcagcttctcgggcagtcgccttcgaaagtggagaagatttccgtggcgtcggcgcagtccgacggaggcatgatcaagggcctgagcgcggacctcgtcagcctgtcgcacctggacctcaaggatgccggtattaccagcctgaaaggcctgccgaagctgccgaagctgaggagcctcaatttagaagggaatttaattaaagacggcgaggacctggaatgggtcgccgaaaattgcccggcgttggagaacttagtactgtccgcgaaccccatcaccgatctgaccaaattggaggccttgaacaagctggccaagctcacatcgctccagcttactggttcggagatcgcgaaggtagacggttacgaggctaaggtctttgcgctactcccatcgctgaccacggtggatgggaagaagaaagacgtcgaggaggtcgaagatgacgacgacgacgacgacgaagccgaggaagaggacgaagagtctgatgacgacgcggagagtgatggcgaggagggcaatccggggctgcaggcgttgcttggtggtgaggacttaagcgacgacgatgaagatttcggtggagaggaaggcggctcagacgatgacgaagaggatgacgacgatgatgacgaagacgaggagacaggcgacgatgccaccccgaagaaggccgacgtccagccggaaggcggcaagggaatcaagcgcaaagcggaagatgatgatgccgaataaacgtttcttgtcttccctttgccgcatctcattggcttcctttatttggcagatctttttgagcgtgtttgtctttgcttgcttgcgacagtagttgtaattcgattgtctctttttggtttcatatttttttcgtgcgggtcggttagttccttgaggcgacagacagtcttgggcctcttttttccttttattgccctcgtgtatcttttccatcagctgctattaccgtatgctcgcgatctcaagggacagctaagccccgcctccctcggtatggccctgcactcgattgaacgttcaatctgattcactcttctctgattggctcatttcgcacagctttctccttgtgctcgattgattgattgtacagttgtgcttttccgtatatttgtccctacagtggtaatttggtgaacggcactgaataggcacagcccgggttggttaaaatttacgggtagggcagaaaagtgtccctcgagatcacgagcatacggtaccaaaagtagagtgctgcaagcgtgaggattattccaagaagtcatccggtcgttgtatcttttttagttctcaacgtctacaccgatgacaaggaaggacaggacaaacgttttttgtttcatcatattctcttcggagaatctagaaaaattagaagcagttctactgctaaaaaaacttactagcacagtacgtgaatacgagaaaagatcagaaagtctcacagaccaaaggaggaatttatagggaaaccaaaggatagcttgaagctgctttagcagccacctttttcataatagacaccaaataagctacgccgaaaactggatccgttggtctggccgctggttaaaaagggtgagcgatcagttctcaaaaaagggactttatcttctcgccgaggctttttttctccttcccatcatggacttcagtgtgttcggagactgtcgttcctccgctgaccgtggtaccagcagcggtgtccatttggacttgcacattggtggccatcttcgactgctctagcgcgttcatggccagttcacgctcgtgttccagctccttcttggcgtacttggcttccaaatccacctcccgtttctggcggtcaatggcggattctacgagatccttgcggaattccacatcgcgggcgtgttgcttctccagctccttgcggatcttttcagcttcagcctcggcagtcttgcgatattcctcagtcttcttctcaaggtccttgtcgtgcgcccgggcgatggcggcctgctcgcgtgagtacttctcctggtccttgaaggcctgttcgcgggatgcctgagattcgaggacttgggtggtggcggcagctccggatatacgtgtggcagaggctgtaaagccctccccaacaatctcctgtgccaggccagcggcacccgttgaaatgatcggcggcagaggattcaccatcggagcgcggatctcagtgtgggtgtagttggtggtcttctcggcggatgcggacgcggatccctcgacacgacgatctcctccgtagacgacttccgtccgctcaaccttcttctcatattgctgcgacatggctgtggtgtgagtttgcggttctgtatacttccttcgggaacaaaataatcaatatcaggacacgaactttgcaaagaaattttcagtgcgagaaagttagaatgatgccgagagactgtccagcgcgg

SEQ ID NO: 142 - DNA, 1465 bp, Hypsibius dujardini
ttttttttttttttgaattatgaagagtctttttgccactggaccaaacgacgatttgtcaagtcatatcatgttccgacaaattcgcataaaataaaatactcttaagctaagctccagggaaataatttcaatccacagcgtgatactacgggatatgtgacaaaaaaatcaggatacaaggccagaaaaaaaaacaggaagagattccacaagacaaatgctacaggaacgtttgaaaaactttcgcaaagcaagagaaaaaagcgcatccatccagctggatgacagtgcaatgaatgaatgggaatagaaatctagaaattcttcgacacatgagactctgacacggatgtgccgccgctggtggtcattccagcggcagagttcatctgaacctcgatgttggtctgcatcttgctgttatcgagcgcttccttggccatctgtcgttctcgctccaggtcggccttggccttcttcgcttccaggtcgatttcctgcttctggcgatcgatcgcagtctcaactaagtcctttcggaattccacatctcgcgcatgctgcttctccagctccttacgaatcttctccgcttccgcctcggcctccttctggtacttttcagtcttcctagccagatccttatcatgatgctggatgatagcggcctgttcacgggcaaattgttcttgctcgcggagttccttctggcgcatctcagcggtctcgatgactttggtgttgacggcgccaccgctgatgcgggaagcggacgcggtgaaaccctctccgacaatctcctgagccagtccggcggctcccgtcacgatgaacggctgacttggagtaatcagcggcgcatgagcctcggtgtgggtgtagctggtggtcttctccgcctccttcactacgttcccggaagagctgtcacgcatagagtgtgaggcgaactgcgaggccccggcgacaccgctggagcacgcggacgatctgttggaggacatgacgtctccgcccacgcaaggagtccgcaccagagtcgcatcggtcggctggaagttgagcgcgggatcgccgtatccggtgtcgatgccgacgcgaacctcctccgtgcccttcttctcgtcgatagtgcgctcctcgactcgctcgaatttttgctcgtgtgtgtgcgacatgatgatgaggggagattctttcctgtgaagtaaacttcgaaaatacaatgggagaaagatttggagaaagcgaaaatttaaaaactgcggagtgcgtgaaagagaacgggaaatcccagcatggaatgtatttgcagcgcagtgggatttgtccagcggaaaattccactggcggcaaaggcggtgcatagaaaagcccggtaactcggacatagacactgtctacttatatattcaaagactcccctgccgtactaataggaccagcacccattgtcgttaca

SEQ ID NO: 143 - DNA, 1685 bp, Hypsibius dujardini
aactgaccggtttaatagcggcagcgcagttgatacagtggaacgagcaattcacaagcgtgagcacaattaaactggaagacgttcattcatcttagactgtaaagggatacggtctggtaaaattatgcgtactgagaaaaaaaaataaacaataatttgcagacaatggggaatgagagagaacgctcagataactgcggcagtggcgggtccacgtgcctgacttgttcggctttcagaggtcgtcgtaacccttccctgggaatgggtcgtgccgatagcggagtcaatgttcacgtcgatgttcgtcgacgccatggcttggtcaagagcagcgcgagcattgacacgttcctgttccagggctcgcatggcgtactcggcctccaggcggatctcctgctgttgacgatccaccgagctttcaactagatccttgcggaattcaatgtcacgcatgtgttggcgctccagagtttggcggatgagttccgcatcggcctcaacggcattacggtaggccgccgctttgcgcgacgtttcgtcctcatgtatgcgagccaaggcttccaccttggcacggtagcgctcatactcctctggtcccatgtcattgatctgtgcctcgctagccaaatgtaaatcggtcgtggcgtggatcgtcgccgaagttcctccggacatgttaacctgctggtgggcgtggatctgcggcgcgaccaccggcgccggaatggtcagctccttgtcctcggtgtaggttctgccgtcgcggcgcgactccagctgcgcggactcgaagcgcgtcgtgggaatggtcaccacggtcgtttccggagctcgagcatgcgattcgtgggtgatcacctcgtggtcttcgcgctgctgatgcggctggatcgggacggtgcgttgcgcgccgaggccctgaaccgagctctccgtgatcaccgtggtgcgttgggtcgtcatgccgttgttcatcccggggttggagttgcgggacgccggagacgcacgctcgctgttgtcggaggacgagacgttgctgctggagttctgacggttcgacgaggcgacgaactcgccgttttttccgatcggtccgatctcaccgcggtacttgaggctgggctccacgaagctggaagacttgctgctggtgctgctgcgggagacgcgaaggcgttcgctctcgctgagatgctcacggagacgttgcccggcacgatccgtcggcaatgactcgatacggttgggaatgactccggccggcgctccgggcaccaaattggccgggcgtttgtcctggaaattcatcccgggatcctccttgccgcggtcctcgccgatattcttgaccagtgtggcgccatccgcgtccacgttgaccttctccacgcgttcatactttgattccttggtggccattgtgtggtggaagggattgcgattatatttaaactgcacgatcaggaagaagagagtttgtcgttgagtagttgcaaggcgatttttgtaagacgcaaaaatgtgattgtgtttcgtcgacgcgcagtttcggactcttatatatggacaagcgtttcaccagtggaaaacaattctttccacgctcgcaattttggcccccctagacttcgatcgatgttctctcacacgacagttggtccaacgaccaa

SEQ ID NO: 144 - DNA, 1944 bp, Hypsibius dujardini
gacagacagacagacagacagaccaaggtgaatgtagagacctgaactttgtttatcgaggaacgatcggtctggaaaatgatacatcgggtgtgaagtccggcccattccgtgcggcctctaatatttcttgcttggcagaagaatcactaataacagagtagtcgtaaactttccattgggtaggaaagtagactttccccattgaacaattggtcgttggaccaactgtcgtgtgagagaacatcgatcgaagtctaggggggccaaaattgcgagcgtggaaagaattgttttccactggtgaaacgcttgtccatatataagagtccgaaactgcgcgtcgacgaaacacaatcacatttttgcgtcttacaaaaatcgccttgcaactactcaacgacaaactctcttcttcctgatcgtgcagtttaaatataatcgcaatcccttccaccacacaatggccaccaaggaatcaaagtatgaacgcgtggagaaggtcaacgtggacgcggatggcgccacactggtcaagaatatcggcgaggaccgcggcaaggaggatcccgggatgaatttccaggacaaacgcccggccaatttggtgcccggagcgccggccggagtcattcccaaccgtatcgagtcattgccgacggatcgtgccgggcaacgtctccgtgagcatctcagcgagagcgaacgccttcgcgtctcccgcagcagcaccagcagcaagtcttccagcttcgtggagcccagcctcaagtaccgcggtgagatcggaccgatcggaaaaaacggcgagttcgtcgcctcgtcgaaccgtcagaactccagcagcaacgtctcgtcctccgacaacagcgagcgtgcgtctccggcgtcccgcaactccaaccccgggatgaacaacggcatgacgacccaacgcaccacggtgatcacggagagctcggttcagggcctcggcgcgcaacgcaccgtcccgatccagccgcatcagcagcgcgaagaccacgaggtgatcacccacgaatcgcatgctcgagctccggaaacgaccgtggtgaccattcccacgacgcgcttcgagtccgcgcagctggagtcgcgccgcgacggcagaacctacaccgaggacaaggagctgaccattccggcgccggtggtcgcgccgcagatccacgcccaccagcaggttagcatgtccggaggaacttcggcgacgatccacgccacgaccgatttacatttggctagcgaggcacagatcaatgacatgggaccagaggagtatgagcgctaccgtgccaaggtggaagccttggctcgcatacatgaggacgaaacgtcgcgcaaagcggcggcctaccgtaatgccgtcgaggccgatgcggaactcatccgccaaactctggagcgccaacacatgcgtgacattgaattccgcaaggatctagttgaaagctcggtggatcgtcaacagcaggagatccgcctggaggccgagtacgccatgcgagccctggaacaggaacgtgtcaatgctcgcgctgctcttgaccaagccatggcgtcgacgaacatcgacgtgaacattgactccgctatcggcacgacccattcccagggaagggttacgacgacctctgaaagccgaacaagtcaggcacgtggacccgccactgccgcagttatctgagcgttctctctcattccccattgtctgcaaattattgtttatttttttttctcagtacgcataattttaccagaccgtatccctttacagtctaagatgaatgaacgtcttccagtttaattgtgctcacgcttgtgaattgctcgttccactatatcaactgtactgccgctattaaaccggtcagtttaaaaaaaaaaaaaaaaaaaaaggagaaaaaaaaaaaaaaaaaaaaa

SEQ ID NO: 145 - DNA, 1472 bp, Hypsibius dujardini
cgacaatgggtgctggtcctattagtacggcagggtagtctttgaatatataagtagacagtgtctatgtccgagttaccgggcttttctatgcaccgcctttgccgccagtggaattttccgctggacaaatcccactgcgctgcaaatacattccatgctgggatttcccgttctctttcacgcactccgcagtttttaaattttcgctttctccaaatctttctcccattgtattttcgaagtttacttcacaggaaagaatctcccctcatcatcatgtcgcacacacacgagcaaaaattcgagcgagtcgaggagcgcactatcgacgagaagaagggcacggaggaggttcgcgtcggcatcgacaccggatacggcgatcccgcgctcaacttccagccgaccgatgcgactctggtgcggactccttgcgtgggcggagacgtcatgtcctccaacagatcgtccgcgtgctccagcggtgtcgccggggcctcgcagttcgcctcacactctatgcgtgacagctcttccgggaacgtagtgaaggaggcggagaagaccaccagctacacccacaccgaggctcatgcgccgctgattactccaagtcagccgttcatcgtgacgggagccgccggactggctcaggagattgtcggagagggtttcaccgcgtccgcttcccgcatcagcggtggcgccgtcaacaccaaagtcatcgagaccgctgagatgcgccagaaggaactccgcgagcaagaacaatttgcccgtgaacaggccgctatcatccagcatcatgataaggatctggctaggaagactgaaaagtaccagaaggaggccgaggcggaagcggagaagattcgtaaggagctggagaagcagcatgcgcgagatgtggaattccgaaaggacttagttgagactgcgatcgatcgccagaagcaggaaatcgacctggaagcgaagaaggccaaggccgacctggagcgagaacgacagatggccaaggaagcgctcgataacagcaagatgcagaccaacatcgaggttcagatgaactctgccgctggaatgaccaccagcggcggcacatccgtgtcagagtctcatgtgtcgaagaatttctagatttctattcccattcattcattgcactgtcatccagctggatggatgcgcttttttctcttgctttgcgaaagtttttcaaacgttcctgtagcatttgtcttgtggaatctcttcctgtttttttttctggccttgtatcctgatttttttgtcacatatcccgtagtatcacgctgtggattgaaattatttccctggagcttagcttaagagtattttattttatgcgaatttgtcggaacatgatttgacttgacaaatcgtcgtttggtccagtggcaaaaagactcttcataattcaaaaaaaaaaacaggaagagattcca

SEQ ID NO: 146 - DNA, 1227 bp, Hypsibius dujardini
tgtcaatctgcgttacagctacgtacgtagctgtagagttattttgttccccagccgagacccggacaacggtatattcccacactgtaatttcccgggcaatatccatctcgacgttgccggacattcgtagtagctcgtataaaagcctcactatgctaccaacgaaaagccaactcactttttcacagtacaacttctctccttttcaagttcgaccacatcgattccacagtcagcttaagcagcaacaatgcagcaaaacaacgaaaatttcgaacgagtcgttgagcgttccgaggtgcgccaagaatgccagcagccgtgccgggaggaagagtcccgtcaggaggagcacaactccagctacctccacactgaagtccgcgctcccgtgcccaatatcccgcctccgatgatgtccgggtccgctggtctcggacaagccctggtcggagaaggattccaggccagcgctgcccgcatttccggtggctcccaggagatgaacatccagccaagtgaaaagctgttgcaggaagccgccatggacaaggagcgatatgctcgcgagcaagaagcaattcagaaccgtctgcagtccgaaacggaacgcaagaccgaggcctaccgaaagaccgctgaggcggaggctgagcgaatccgcaaggagcttgagaagcagcatgagcgagacatcgagttccggaaggatctggttcagggaaccatcgacagccagaagaaacaagtcgagctcggagcgatcatggctaaacgcgaattggaccgtgaagcgaaacttgctcgggatgcacttgaacagagcaagatggccacaaatgttgaagtgaactttgacagtgcagccggtcacactgtgtcgggaggccagacggtgtcgcagtccaccaaagtcaccagggaaaagaaataaactatcaaccatcagaacggtcactttacagcaacctcagaattccgataatcatccatcggattagtggaagagtgttatgttacaagcacgtttttatagaggatatttgttccgttcaatcttttctagtaccaggttgtacagtaattgatttcatcactcaaacgaatttctgtaaaacacttcagtgaaacgaataggcacgaaataccagtgtcgaatcttcgaccacatcacttgaagttgatttgatgtcgactggctaaaccttttttcgccaagaaaaa

SEQ ID NO: 147 - DNA, 1308 bp, Hypsibius dujardini
ttttaaacaagaaacctttatgctgcagttagagtaaactgccacgatagcttgtatatttttaacgttttttatcttatattatatagtatactcacgtccacatgccggaggaaaaatcaataacaatgcgagccggaaattgcttttctatgcagagtacttagaatgaagattccatatcacaacttagtatgcactagctacaggatgcatatttagtactctcgttttaaattaaatattttatttcacgaatatacttttacattctgggatggctgatgtcggtgtgctgcgaaacgacatggctttccgtcactgtccgaccggcagcgctgtcaaaattcacttcgatgttgctttcaagtttggatcgttccagggcctccagtgccagttttcgctcgtgctccagctccgttttggcgtatttcgcttccaagtcaacttcccgcttttggcggtcaatggcagactccaccagatctttgcggaactccacatctcgcgcatgttgtttctccagttcccggcggattttctcagcctcagcctcggcggtcttccgataggcctcagtctttttcgccacctcttcctcactcctccgcgccaccgcttccatctcccggtctcggcgctgtcgatccaaggcagcctgccgtgaggtctccaccgtctcgtggacatactcctgcgtcgccgtgccagccactcgtgcagcggacgcggtgaacccttcaccgacaatctcctgcgccaaaccctccgccgaggagatgaggatgggcgaggtcacgtggagcggggctttgatttcggtgtgggtgtagctggtgctgtcgcccgtggtcgtgacgacatggctgtggttgagggttccctgttggcttagggttccctgttggtttagggttcccggttggcttaggattccctgcgggcttagggttccttgtgggcttagggttcccggttggcttagggtgctggtggtgagggtgctgtggatcgtgccggcgggtccggtagtgattgtttcacggataaactctccgccgaccggtcgggcgggcaagggtggagggacctctccggcggaacggaagacgtcctggcgttcggaaacatagacattggggtgctcaatgctcgacattttgatgtgatgggttctcggttgtggggaagtattacacagaaaggtcggtaaagttccgggaatgtggtcgggttctcagctaaaaactttggagactttaatgtcgtgaggtagatttgtgaggatattgtcgggtgctcaat

SEQ ID NO: 148 - DNA, 677 bp, Hypsibius dujardini
cttgtgtctgccaccaacagtcgatcgatagcaacattatcgcacgaccacgtttagtacactcatgttatttggtgcggtgcagattcgttcaagtgcgaataacttctaaaccaactcaaaacaacatgataagccaaagacacagcctcttcataaatcaacgaaacaaaaaaacaaggaaagtatcttctgcacgcaaaggataattcaacaaaaaaacagtacgcgttatgtatgtaacggagcggatgagaccgatgtgaacaatgttccttttcctacatgcgtatgtcaactgaacgaatcctatttaagagaggcagacaaccgagtaaggagccgacttttaatgatgaccagtaaaagtttccttgagcttttccgcgatggtcttcttatgctcggtatgatgctcgctgtattcctcctcggtgacgtgactgccgccggagacggtatgaccggctgaggtgtccaaattgaccagcacgtcagtgtgcattttgctctgctccaacgcctctttggcaagcagacgctcgtgatcgagctccttcttggcgtacttcacttccagttcgagttccttcttctgccgcttgacagactcgtccaccatttccttgcgatagtcaatgtcgcgcttgtactgcttctccagctccttgcggatc

SEQ ID NO: 149 - DNA, 1311 bp, Hypsibius dujardini
gagcaccacgacaatatcctcacaaatctacctcacgacattaaagtctccaaagtttttagctgagaacccgaccacattcccggaactttaccgacctttctgtgtaatacttccccacaaccgagaacccatcacatcaaaatgtcgagcattgagcaccccaatgtctatgtttccgaacgccaggacgtcttccgttccgccggagaggtccctccacccttgcccgcccgaccggtcggcggagagtttatccgtgaaacaatcactaccggacccgccggcacgatccacagcaccctcaccaccagcaccctaagccaaccgggaaccctaagcccacaaggaaccctaagcccgcagggaatcctaagccaaccgggaaccctaaaccaacagggaaccctaagccaacagggaaccctcaaccacagccatgtcgtcacgaccacgggcgacagcaccagctacacccacaccgaaatcaaagccccgctccacgtgacctcgcccatcctcatctcctcggcggagggtttggcgcaggagattgtcggtgaagggttcaccgcgtccgctgcacgagtggctggcacggcgacgcaggagtatgtccacgagacggtggagacctcacggcaggctgccttggatcgacagcgccgagaccgggagatggaagcggtggcgcggaggagtgaggaagaggtggcgaaaaagactgaggcctatcggaagaccgccgaggctgaggctgagaaaatccgccgggaactggagaaacaacatgcgcgagatgtggagttccgcaaagatctggtggagtctgccattgaccgccaaaagcgggaagttgacttggaagcgaaatacgccaaaacggagctggagcacgagcgaaaactggcactggaggccctggaacgatccaaacttgaaagcaacatcgaagtgaattttgacagcgctgccggtcggacagtgacggaaagccatgtcgtttcgcagcacaccgacatcagccatcccagaatgtaaaagtatattcgtgaaataaaatatttaatttaaaacgagagtactaaatatgcatcctgtagctagtgcatactaagttgtgatatggaatcttcattctaagtactctgcatagaaaagcaatttccggctcgcattgttattgatttttcctccggcatgtggacgtgagtatactatataatataagataaaaaacgttaaaaatatacaagctatcgtggcagtttactctaactgcagcataaaggtttcttgttttaaaaaaaa

SEQ ID NO: 150 - DNA, 669 bp, Hypsibius dujardini
atccgcaaggagctggagaagcagtacaagcgcgacattgactatcgcaaggaaatggtggacgagtctgtcaagcggcagaagaaggaactcgaactggaagtgaagtacgccaagaaggagctcgatcacgagcgtctgcttgccaaagaggcgttggagcagagcaaaatgcacactgacgtgctggtcaatttggacacctcagccggtcataccgtctccggcggcagtcacgtcaccgaggaggaatacagcgagcatcataccgagcataagaagaccatcgcggaaaagctcaaggaaacttttactggtcatcattaaaagtcggctccttactcggttgtctgcctctcttaaataggattcgttcagttgacatacgcatgtaggaaaaggaacattgttcacatcggtctcatccgctccgttacatacataacgcgtactgtttttttgttgaattatcctttgcgtgcagaagatactttccttgtttttttgtttcgttgatttatgaagaggctgtgtctttggcttatcatgttgttttgagttggtttagaagttattcgcacttgaacgaatctgcaccgcaccaaataacatgagtgtactaaacgtggtcgtgcgataatgttgctatcgatcgactgttggtgggag

SEQ ID NO: 151 - DNA, 1304 bp, Hypsibius dujardini
cagtcagtcagacagacagactgacattcttggcgaaaaaaggtttagccagtcgacatcaaatcaacttcaagtgatgtggtcgaagattcgacactggtatttcgtgcctattcgtttcactgaagtgttttacagaaattcgtttgagtgatgaaatcaattactgtacaacctggtactagaaaagattgaacggaacaaatatcctctataaaaacgtgcttgtaacataacactcttccactaatccgatggatgattatcggaattctgaggttgctgtaaagtgaccgttctgatggttgatagtttatttcttttccctggtgactttggtggactgcgacaccgtctggcctcccgacacagtgtgaccggctgcactgtcaaagttcacttcaacatttgtggccatcttgctctgttcaagtgcatcccgagcaagtttcgcttcacggtccaattcgcgtttagccatgatcgctccgagctcgacttgtttcttctggctgtcgatggttccctgaaccagatccttccggaactcgatgtctcgctcatgctgcttctcaagctccttgcggattcgctcagcctccgcctcagcggtctttcggtaggcctcggtcttgcgttccgtttcggactgcagacggttctgaattgcttcttgctcgcgagcatatcgctccttgtccatggcggcttcctgcaacagcttttcacttggctggatgttcatctcctgggagccaccggaaatgcgggcagcgctggcctggaatccttctccgaccagggcttgtccgagaccagcggacccggacatcatcggaggcgggatattgggcacgggagcgcggacttcagtgtggaggtagctggagttgtgctcctcctgacgggactcttcctcccggcacggctgctggcattcttggcgcacctcggaacgctcaacgactcgttcgaaattttcgttgttttgctgcattgttgctgcttaagctgactgtggaatcgatgtggtcgaacttgaaaaggagagaagttgtactgtgaaaaagtgagttggcttttcgttggtagcatagtgaggcttttatacgagctactacgaatgtccggcaacgtcgagatggatattgcccgggaaattacagtgtgggaatataccgttgtccgggtctcgtctggggaacaaaataactctacagctacgtacgtagctgtaacgcagattgacattttgcgtctttggaatgccgaacagaggtgaaataataaaccctctcagtggc

SEQ ID NO: 152 - DNA, 1067 bp, Hypsibius dujardini
cgcacacagtcactgtgctccccttcctccccgttctctaacatcaataaagacaaggcgcttccacatgcgaggaataatcaataccatcagcgcgaagcagcaaacaacacatcgttccacttttatgctgttgcttcgggagattgagagagctccgaaaactctgcaattatcgattttgccttccttctcccgctgtgccctataaacccagtcacacaccgaccgaccgccgtctcactttttccccccatacagacacgtacaaactcttactcccttatcccgtcattcaatcagtctcggttatttttcgatcagccttacaacttccagacacagacattcagacatagacactcagacgcagacacccagacacagataccagacacagacatctaggcataaagacatcgccattgacgcattctgccattctgagacagacattcaagacattcaagcactcaagcattcagacacttctgtttggacattcagacatgacgcactacaaggaagacgaggaactgcttgagcatctccgtgaagacagcggtttccaggccttcaagacgaaggccgttgacgacgtcgtggcaggcaacggaaatacccactcggaactgcacgaaacagtgaaggagaaggcatcggtgtcgtcagcgtcctcatcctcgtcgtcctcgccgccgtccaccggtcgcagcagcgtggaacgccatgtcacctacacgcacaccgaggcgaagagtgggcccttgattcacacgactcatccggtggtgttgagctcggcgtcgggcatgctggcgcatgagatcatggaggagcaatcggggttcatggcatcggcgacgcatgtctcgggcagcgaacacggcgtggcagcggcccacgagtcgccggagttgcgggaacaacggctgaaggatgaggccaagtatcgggagaaacaggacgagattgcgcgaaagcatgataaacacttggagaaggtgaccgaggagtaccgaaagaaaactgaagcggaagccgaaaagatccgcaaggagctggag

SEQ ID NO: 153 - DNA, 964 bp, Hypsibius dujardini
tccagctccttgcggatcttttcggcttccgcttcagttttctttcggtactcctcggtcaccttctccaagtgtttatcatgctttcgcgcaatctcgtcctgtttctcccgatacttggcctcatccttcagccgttgttcccgcaactccggcgactcgtgggccgctgccacgccgtgttcgctgcccgagacatgcgtcgccgatgccatgaaccccgattgctcctccatgatctcatgcgccagcatgcccgacgccgagctcaacaccaccggatgagtcgtgtgaatcaagggcccactcttcgcctcggtgtgcgtgtaggtgacatggcgttccacgctgctgcgaccggtggacggcggcgaggacgacgaggatgaggacgctgacgacaccgatgccttctccttcactgtttcgtgcagttccgagtgggtatttccgttgcctgccacgacgtcgtcaacggccttcgtcttgaaggcctggaaaccgctgtcttcacggagatgctcaagcagttcctcgtcttccttgtagtgcgtcatgtctgaatgtccaaacagaagtgtctgaatgcttgagtgcttgaatgtcttgaatgtctgtctcagaatggcagaatgcgtcaatggcggtgtctttatgcctagatgtctgtgtctggtatctgtgtctgggtgtctgcgtctgagtgtctatgtctgaatgtctgtgtctggaagttgtaaggctgatcgaaaaataaccgagactgattgaatgacgggataagggagtaagagtttgtacgtgtctgtatggggggaaaaagtgagacggcggtcggtcggtgtgtgactgggtttatagggcacagcgggagaaggaaggcaaaatcgataattgcagagttttcggagctctctcaatctcccgaagcaacagcataaaagtggaacgatgtgttgtttgc

SEQ ID NO: 154 - DNA, 1167 bp, Ramazzottius varieornatus
attttccgctcatgagttacgagaaaacggaacttgatatcacaagatagtcattacgttttctgcttattcccttttttcgttccagcagtaattactcggataatatttcacaatgccttacgaaaagcacgttgaacagacggtggtggaaaaaactgagcagcctggacactcgtcgacgcaccatgctcccgcccaaaggaccgtagctcgcgagcaggaggaagttgtccacaaagagtttacccataccgacattcgagttccccacatcgacgcacctcctccaatcatcgctgccagcgcagtgggcttggccgaggagatcgtcagtcatggtttccaagcctcggcggcccgcatcagcggtgcttccaccgaggtcgatatgcgcccaagtcccaagctagccgaggaagctcgtcgtgatgccgaacgatatcaaaaggagcacgagatgatcaacagacaagccgaagccacgctgcaaaagaaggcggaggagtaccgtcaccagactgaggcggaagccgagaagattcgccgcgaactggaaaagcagcatgaacgggacatccagttcaggaaggacctcatcgatcagaccatcgaaaagcagaagcgcgaagttgatctggaagccaagatggccaaacgcgagctggatcgcgaagcgcagttggctaaggaagccctggaacgttctcgaatggccaccaacgtggaagtcacgctggataccgcagcgggacatacagtaagtgggggaactaccgtctccagcgtcgacaaagttgaaaccgtccgcgagcgcaaacatcattaacgaccacaccacgattagaacttgcttgcctcattggatgaaccctttaattcacaaaatatctaagatactcaagatcgtaagatttagaatggttagcttcttcatttccgcgtcgacctgaatagtacgcgtacggacctcgtgaattagtcgcataacatcctaatattttcaaagtgataagcgagtctccaactataaacagggcgtcgtaccataatttccacttgacagtattgctcggcgcgacaactcgagccaactgaagccactgcgttgcagaactatgtgcgcgacctgtgtatagtggagttattcaagagttttatcgctaa

SEQ ID NO: 155 - DNA, 914 bp, Ramazzottius varieornatus
acctttcccagcacctcttgaaacgctctagccttagactttagctgtcgcaagtagccctcgcaagtctcaacatgtctcgagatcaaggaagcacggaatacgacgctaaccaacgccaggagcaacaccaggaacaacataatacctcttacacccacaccgatgtccgcaccaacatccctaatatccctgccccgttcatctctaccggtgtttcgggtctcggtcaacagctggtcggagaaggtttcaccgcctccgctgctcgcatctccggacaatcgtcggaaacccacgtacagatgacccccgagatggaagccgaagcgcgcaaggaccgcgagcgctacgaacgcgagctgcaagccatcaacgagcgacaccaacgagacatcgaaggcaagaccgaggcctaccgcaaacaggctgaacaagaagccgaacgtcttcgcaaagagctggagaagcaacaccaacgagatatcgagttccgcaaatcgctggtccagggtaccatcgagaaccagaaacgacaagttgagctcgaagcccagctcgccaagcgcgaactcgaccgagaagctcgccttgcaactcaggctctcgaccagtccaagatggccactgatgttcaagtcaattttgactcagcagtaggccacactgtttctggggctaccacagtctcccaatccgagaaagtcacccagtcgaagcactaaagaagaatttttataagacatgaataatcctaaattcagacctgtactggccatcttgaattccataccaaatggaattttcacaagccaaaatccacatactgaaatttagttacatttgactttctctcctgaaattcattcgcatcattccatgtttctataatacccataaaatgaagacctact

SEQ ID NO: 156 - DNA, 1154 bp, Ramazzottius varieornatus
cttgtttattctttcgcagcctttcgctaacgccctgtcgcaaacatgtcttcccgacagaaccagcaatcgtccagccaacactcgtcctccagccagcaaggtggtcaaggtggtcaaggtgttcaaggaagttccagctactcgcgcaccgaggtccacaccagcagtggaggacctaccatcggtggagcccagcgaactgtccccgtcccccctggatctcactccgaggtccatgaggagcgtgaggtcatcaagcatggtaccaaaaccgaaagcgagacccacgtcgtcaccgtcccagtgacaactttcggcagcaccaacatggaatctgtccgaaccggcttcaccgtcacccaagacaagaacttgaccgttgctgctcccaacatcgctgctcccatccacagcaacctcgaccttaacctcggcggtggagctcgcgctgaaattaccgcagggaccaccgttgacttgagcaagatccagcgcaaggatttgggacctgaagagtatgctcgctacaaggccaaggtcgagcaactggccaggcaagatgagcaagacgcgggtatgcgcgctgcccagtaccgagaggaagtggagcgtgatgccgaactcatccgacagatcttggagcgacaacacatccgtgatcttgaattccgcaaggaaatggttgagaaccaagtcaaccgacaagagagagaaatccagctggaggctgagtacgcaatgcgagccctcgagcttgagcgcaatgccgctaaagaggctttggaaagcgccaaggcccagactaacgtcaatgtcaaggtcgagtccgctattggtaccaccgtctccaagggtgcaatccagacttccgccgacaagagcagcaccaccaagactggacccaccaccgtcactcagattaaacataccgaacaacacactgaacgccgatagatttgctcataaccacaccatcatacaatttttcaccatttctaaaaagaaaatgttttcgtccagtcttttgtgcactctgaaatgtcatatcatgaataacttcagttttagcgccgcaaggaaaatgtgagacggttaatatcacgaacgccggtaaacaatattcgtggtcaataaaacgattaatttctaca

SEQ ID NO: 157 - DNA, 1541 bp, Milnesium tardigradum
tggccattacggccgggggccttccaaatcacagtcagttcaaatatctgttacatacgtgcttataccagtaaaccacttgacaaaagcagagaaatcaagatagaaaatgtctacccatcgtgaccgagactctgctaataacgaatatatcgctgagaccgtctcaagcgtgacgacctctacggccgccgatctgaccactggtcgtacgttatacgcaactcctgtgacctcaaccgcccgacaccacgatacgaccacatccagccataccagtcaacgtatggccactgactacaccaccggcgctggtacagtctacactgaaaagacagtgatgcgcgaaccggtcaatgtcgtccatactcaaattgaccgcgtcactgcagtgcccatcactgagacgcaagtgcacgctgaaacgcagcattatttgcatacgcagatgcgtacgcctgtagtagagtcgcatccgccgcaattgccggctcataccgatgtggccggttcgattctcaacgattcggcattttcttcaaccgcccatatctctacgaatgcgatgcatgcacaggccgtaccagtggatgcggcagaacgggcaagacaagaggaacacttccgtcgagaggccgaccggattgctttacagcatcagcgagcgatcgatgagaagtcggaagcgtatcgtagagatacagaggcacaggctgagagaattcgacaagaactcgaaaagcaacatcttcgagatgtgcagtttagacaagagttggtggacgatgcgatcaccagacagaaacgtgaagtgcagctggaagcacaggctgtgatggctgatctcgagttggaacgtcgaagagcacatgaggcgttggaacgcagcaaaatgtccacagacatcaacgtaaacatcgatactctggctggcagcactactgcaggtggcaccactgttatcgagaaaactgaagtgcaaaagggcgtcgcttaccacacgactccagtcggcgtcacgactcacgttgagtacgctgaccgtcccacaacaacccaccgaacggagaccaccactactgctcactcgactcacaccaccgaaggtcaccaccgcggatcagacgcatcgtacattggcggccgtgacgatgatcgtatgtcaattggtactcagggcagcgatgcagatgagcacaagaaacgtggacttttgggcaaaatcaaggattctttgacgaaataaactgttcgctctgtacttatgatcatgtcttgtttctgttgttctcttttgtattttcgattttaataatatgaagcttttcttcgagactctatcacgtctctgtgggatttctgaatgaaataacgtctcttcatgcttttgtgcgtacactgattctgtacgttcagtacgtttcgtgcagctgcttttccgtgtcctccaatgattgattgaatgttgctatttttctattgtttgacacaccctattcgtcccattaaattacgttgtttttgaaaaaaaaaaaaaaaaa

SEQ ID NO: 158 - DNA, 1283 bp, Milnesium tardigradum
agagtggccattacggccggggagtagtatcagtgtcagctcagagtcaagtggaccctatcttcgatcctcgaatcagccattttttcgtcttctttactcgactaaagccgtactcaaaatgtctcatcaacagacgcgcgaagtgactaaagagatccacgttgagtcgagtggtcaatcgggcgcatcgtcacatgcttccggtcatgtagtggccggacacgagacatcagcggtcgaacacaccaaatacctgcataccgagactaaggtgccaatggccacgcctgcgccgcccatcattcatgcgtcgtcgggtctgcaacatatggagggtatgaccgcctctgctgcgcgcatcactgcaggatccgctgagaccactaatgtccaggtttccgaggaagtcagacgacgtgatcaggctcagttcgaacgtgaggccgcagcaatcgctgctcgtcatgagaaggatgttcaggcgaagaccgaagcataccgtaaagagaccgaagaacaagccgagaaaattcgacgcgagttggaaaagcaacaccaaaaagatgtcgagtttcgaaaggatatggtcgacgacactatcaaccgtcagaagcgtgaagtcgagttagagtcagcgatggccaagcgtcagctcgagcgcgaagctgaagctgccaaggctgcacttgacaagagcaaactgtcgactgacattcatgttgaactgaacactgctgccggtaacaccgttgctggaggcactacgaccagtgtatcacagagtgagcgtcacgagtcagcgtcggtgcatgagtcgaagtcgttgggtgacaaggtcaaggacgcgcttggattcggttcaaagtagtcatgtgactcttattaacgattttctatgatcacaaatgctattggtaactgtgtattgatgtagtgtaactgtttctcgagtacgtttctctctgttcagaaatcaaaatgatcacaaatttatgtctgatttacgaaagatgcaaaaaagatatatttttattttgtgagaagtttgtgctgagaatcgatcgattgtatgtgtcatctcaacctttgcttatcgtacgtgtcatgtgattatgggccgtttcggtcgagggctaatctgcatatgagtaatggtggtttgtgctattttgttatggcaaactgtgaacattccataagttgtattgtgagaacgaacattgctgttctgagaatcaaatgaagttcaattaccaccaaaaaaaaaaaaaaaaaaaaaa

SEQ ID NO: 159 - DNA, 1378 bp, Milnesium tardigradum
agtggccattacggccggggagtcgtcagacgtcagtgttgcatctcagatcgtacgtacattttcactccagaatttcgaatcgatttttctctgtttttcacttaatttacataaaaaaaatctgacgaaaggatgaatcccacttctgagcatatctctgaaactacgacaactgtaaaaacgaccgataccggtgtcggactacagaatgtgtcggcgtcacaccacgcttccggtattcatcacgactcgagtgccgcttcgagcactgaatcgactaaattcgttcataccgaaacgaaggttcctatggccacaccagcgccacccattatttccgctgcaaccggtattgcggacagcattgtcagtgaaggaatgaccgcgtcggccgcgcgcatttctgccggagcgaatgaaagcatcgtacctgtggtggacacacaaaaggcggcggccgattatgacaaatatcagagagaagcggcggccatcgctgctgctcacgagcgtgacgttgcgaagaagactgaggcctatcgtaaagaaacggaggaacaggcagagaagatccgaaaagagctcgaaaagcaacacgcgaaagatatcgagtttcgaaaggatttggttgaggacgcgatcactcgtcagaaacgcgaaattgaattggaggcgaaaatggcaaagaaagaactggaacgcgaagctgaggctgcattggctgcgctcgacaagagtaaactgtccactgacatcgcagtcagcatcaacactgccgccggaagtacggtcgcaggaggcactgttacgacagtcactgagaagactgagagcactcactcacacgaacatgagcatgaacaccgaagcttgggtgagaaaatcaaagatacgcttttgggacgcaaatgagtggtggaaagtggaaggaaaatggccgccatttttctgcagtaattccacatgtttcagttgagttcgcgccattagtatcttttctacactaatgctatgttcctcttatcaacgtatactatcgcttattctgttgagttctgttgtgctgaagtctgtacttgagagttgtgcgtagagtggtgcagtcactgcactgcgctgtagtgatttaggttgtactgtattgcattgagtttctcggctgaccgcagctaatctgaccgcagctttgagagtcacttgtgcactctgaaatgtgctgtttttgtgagttctgacgagagtcgactgtcttaagtgtaacgaatcgcctctgtcattgcacccatttttcagttactttttttaccgtatttgcagttatcaaaatggcgatttgaataaagagaaagtatcgaaaaaaaaaaaaaaaaaaaaa

SEQ ID NO: 160 - DNA, 596 bp, Milnesium tardigradum
aaaagagctcgaaaagcaacacgcaaaagatattgagtttcggaaggagattcttgaagatacgattgctcgacagaagcgtgaagtggaattagaggcgaagatggcgaagcgagagctggacagagaggcggcggccgctcgtgaagcgctagatcgatcgaagctcgcgacggatatcagtgtttcgattgataccgcagctggtcacactgttgccactgaaactatgaagagtactgagcatactttcagtcatcaacgcatgtaactcactttattcgctttttgttgctgtacgtgattcttcattcgaagagtacagctgtcacctttaatagtagttgatgcattcaacagccttaactgcgaaacttttttatgaatattatactactgcgtactgcaaatatcattggcactaaagtgagaataagcatcgcaggaaagtggactacattgtttggtttaagtaatgcattgaatttgtagcagtaactctatcttctcgtaacccttgtgcttcttgtacgttgactgtatggccgtgacgacgttacaataaaacgcgggtaacaaaaaaaaaaaaaaaaaaa

SEQ ID NO: 161 - DNA, 1244 bp, Milnesium tardigradum
agttagaatcgatcataaaaaaattaatgaatgttatgcaatatagagagttactgaaaatgagttactggaaacgagtgtcctaaaatgtggacaaaaaagcatgagtaacaaagggcgtattatagtgcgattacgggcgaccgagaactatggttttcagcttatctcccaaagatctcacttcttcttgacttttcgcgtgagtttgtgtgccgttcatctcgagcatcgatgcttcgctcttcgagtctatagagatgccggtaatagtttcgccggcggacgtagagatggtgacctcgatttctgacgagagtcttgcctgatctgcggccgacttgagtgcctgttgagtgcgctctaactcagtgagtttagcgcgatacgcgagaagtgcctcctgcttttcacgcgcaatggtcgcttctgacaactcacgtcgatactgctgttcctggatgtgctgtttctgcatctctactcgtagcttctctgcctgcatttctgtctctttacgataggcttcagtgcgctgtttgacctttcgctcgtgttcctcagcgattcgactcattctttttcgtagaatgcccggtcaatgtcggtcaactcttggggcaccgtatcagcgattacatcctgagtttgggcactaaatgtcgacgtcgactgcaccacatcgatgtgctgttcttgaccattatgggcatattcaggcatatgagtggcacccgaaacctgtgcataaccctcagagcgtatctgcttctgcgacagagacgagctgtgctgttgctgtcctctgccctgagtcgtatcgtactcttgctgcctgtgatatgtacgttaatgccggccactgacgcagaggtcacctctggctgctgttcgcccgtctgatagacttgttcgcttgtgtgtccattctgtggtacgaactcgaccggctctttccattccgactgttgaatttgaacagattgctgctgatccattatagcgtcacttaagttaaagtagcaaagggtcaagtgaaactgttgcttgtgtcaagtaagactgttgcttctgtcaattgagactattgctgtgtaaagtgtgagaacagaaatgatgagtaaagtggtgtcagtgccatatatagacgacactagaccggatgcaaccggctgtaaatgcgatgattactctagtctattgttagtcagttctgtagtctatcgtgaaaccactggaaatta

SEQ ID NO: 162 - DNA, 665 bp, Milnesium tardigradum
ggccattacggccggggagattatcggaagggactcgcggccaaacaggtcgactgccagaaacaacaagtcgaactcgaggtacaccattatttgatcataacagactacgattctacggcaagaaatctaagtggtaagttgatatttgttgaacaggcaaaaatggcgaaaaaggaactggaacgcgagctgactgcggctaaggaggctctagacgccacaaagtctgcgacaaatattcatgtgaatatcgaaactcttgctggagtcacgatggccggcgctacgacacacagccaaattacggaagttttggatgaaagcgaaatggataacgatcgcaaactgactttgggtcagaagatcaaagagaaactatcaaagggaaaattataaatgcgcgattagggaagaatcaacggattcatataaaaagctacagccaatgattgtaaatcactttttctctagaaaaaatgccatttagttttgggttacgggttcttagtggaaatgcgctgtaatctataatttacgagttatcactctatctgaactgctaacttgacatcatccatattttgatctatcaaatttatgtactgtaaaacagaagccggcgatattgcattaaactgtataaatcggaaaaaaaaaaaaaa

SEQ ID NO: 163 - DNA, 592 bp, Milnesium tardigradum
ttgccattacggccggggacttctcacagtagtaatttttgccatacgaacttcaaatctcaccataacctaatcttaaaatgtctcataccgtagaaaagacagttgtccaagagacacgcacagtaggctcaaatgccgactactccagccctaatgttacctacatgaactgccccacgaccggtggtcagtgcataacgacctgctctaccgaacatacccatggcgctgaatgccacacacacgcccacagccacagcgacgtccaggcgacctccactctgcacaccgaaatcagaacaccaatcatcgcacctgcagcaccagtaatggttacctcaactggagtggcttcagaaattattggtgaatcgatgacggcctcagcgtcgagaatcagttctctgcgtcacaggctgtagttgcagaggtgccgcttgaagtgaggcgaagagatcaggaggctttcgaccgcgaagcagcggccattgctgcaagggccgagcatgacattgcgaaaaagaccgaacagtatcgtaaagaaacggaagcacaggccgaacgcatccgaaaagagctcgaaaagcaa

SEQ ID NO: 164 - DNA, 735 bp, Milnesium tardigradum
gattctgaaacagaaaaaatggcaaaagatatgtatacagatgcagaagaaaatgcgcacagaacagagtagatcaacgcagagatacaagacactcctaaacctttttaaagttctcattgcggtaacgttccctttcttgtaagtatgcacaaatccggtatcactaaagtgaaaattgctcgagaattccattgccggtcggccgccggccgcgtccgccttgaagtcagcatgcagcacaggtttatcatccttcgttttcagagtatacgtatagttgacggtagtattcttgatttggtgactaccggcctcaccgatcttaaagggtatatcttgatggtagtctgcttgcgggaagcagattacgtggtggtacttatcaccttctttttcagttcgaccgtcaactttcctgccgcgatttccgcagaatagggctttcgcacaccgagtgctcttaggtattcctcgaaattctcttcttgaccggtcgtctcatatttgcccaaatatttatcgtccagtttctggtcatcagcgatgaccagtacgaacgcaaacagagcagcgaagtagatcgaagcatacatgatttttcaagagtagtttatgatttgatgagaaatgttttggtgaaaagactgagttagagccaagtgaagccgtgctatatatgccgtattgggtgttatggtttttgccactccccggccgtaatgggatatgctg

SEQ ID NO: 165 - DNA, 660 bp, Milnesium tardigradum
gattctgaaacagaaaaaatggcaaaagatatgtatacagatgcagaagaaaatgcgcacagaacagagtagatcaacgcagagatacaagacactcctaaacctttttaaagttctcattgcggtaacgttccctttcttgtaagtatgcacaaatccggtatcactaaagtgaaaattgctcgagaattccattgccggtcggccgccggccgcgtccgccttgaagtcagcatgcagcacaggtttatcatccttcgttttcagagtatacgtatagttgacggtagtattcttgatttggtgactaccggcctcaccgatcttaaagggtatatcttgatggtagtctgcttgcgggaagcagattacgtggtggtacttatcaccttctttttcagttcgaccgtcaactttcctgccgcgatttccgcagaatagggctttcgcacaccgagtgctcttaggtattcctcgaaattctcttcttgaccggtcgtctcatatttgcccaaatatttatcgtccagtttctggtcatcagcgatgaccagtacgaacgcaaacagagcagcgaagtagatcgaagcatacatgatttttcaagagtagtttatgatttgatgagaaatgttttggtgaaaagactccccggccgta

SEQ ID NO: 166 - DNA, 752 bp, Ramazzottius varieornatus
tcattccaagttttcagtgttttctcagcgcattcctgccatacaaccaaaccagcatgtctcgcgcagctgtcgctatcgcccttctgggttgcgttgtggccgcttacggcgctccggctgaaggccacgacgatgccaaggcagaatggaccggaaagagctggatgggcaagtgggaatccactgatcgcatagagaactttgacgccttcatctccgcccttggtcttcctctcgaacagtacggtggaaaccacaagaccttccacaagatctggaaggagggtgaccactaccaccaccaaatcagcgtccccgacaagaactacaagaacgacgttaacttcaaactcaacgaggaaggaacaacccaacacaacaacacggagatcaagtacaagtacaccgaggatggcggaaacctgaaggctgaagtccacgttccatcccgaaacaaggttatccatgacgaatacaaagtcaatggagacgaactcgagaagacctacaaggttggagatgtcaccgccaagagatggtacaagaagagctcttcgtcgtagatgtttgacgaatgccatataactgttccatttttcgttctgatgtaaacactttttcgctgtcgcatttatccatctgtagctagaaatcttgacagctctttcctcattcagtttccctatgcaagttcgacctgcttttggcacgaactcttacattgaaaccgctcatcgaaacctccactc

SEQ ID NO: 167 - DNA, 918 bp, Ramazzottius varieornatus
atacgtacagtagacgcaaacagtacagactcctccagcaacggtacttcctggccgtatataaaatcaaggttcgggttccttccaacctgtacctctttacttcttgtaactttctcacggagaatactcgcagaaccatgcatcgatttgtccttgctctcgtcgtttttgcaggtgctgccatcgtctgggccgctgatgacgctgctcacgaagaaggcgtagaatggactgggaaaccgtggatgggcaaatgggaatccgacccatcgaaggacgagaacgttgaggaattcaaaaagaagctccagcttccgatgagccactcggaaatgaacaaaaactccaaagtttggatccatcactacaagaagggagacgagtaccatcacaaaatcatcatcaacgacgcccattacaaaaacgatatcgtcttcaagctgggtcaagagtccgccggttcgtataacggctcatctttcagcgtgaagtacgaggacaaagacggcgctctagtcggaagcgtccactacactggcaccaaagaacagtctcttgacaagaccatcaacaacgtcttcaagctcgaaggtgaccatctggttaagacttccaccatcgagggagtgaccatgaagcgccactacaacaaacgccagtgaagttgtcgttgcggctaaattttttcctttctgcaaattcatgcccgttttgtcgagtctctcctgcttcccatcgttctaaagatttttgcagtactgagttatcagggctttgtttctgttctcgttctatcctcgtattttcttttcgttcaccggatacagtaaagctgcgtttcaaagccaggttttttatctgcctgttggtcggacggattgtcggaccaactcagatatcgatcgggctgattgt

SEQ ID NO: 168 - DNA, 1167 bp, Paramacrobiotus richtersi
caaacacatccttccaaaccccggagataattccattactatgagatagcaaactgtctgtcatccttagcgcgaactgtgtactgatgccggaagcttgttgatctgtgtgacatgtacggaagtggattcctagcacagttcccatcaaagcgttgtaattttgcctaatgtagccagatattatcggtttttttatagtataaaaccaacagttgaactatcgtggtagacaggtggtgtgctactgactgaatgcgaggtgaaaatctcttacagctctctcatcatgacctttaaggtgtttattttgattgcacttgtggcggccgtcaaagcccgaccggccgagggcgaacacaaggatcagcaagacattgccgctgacgccgaccatccctggattggcaaatgggaatccattgacgggcgccaggaaaactttcagaacttcatcaatgccttaggcttcgcacactacacacacgagcacaaagtctggcacaaactgtggaaagagggcgatcactatcatcaccgcatcaaagtcccggagaagggttacaagctcgacgttgagttcaagctgggagaagaaggaaccggtagctacaataatacccagttcaagtacaaatacaccgaagagaataaagatttacatgtggagatcaacctggtcacgcacaacaaggtgatcaaggacgattaccacgtggaaggcgaggagctggtcaagacctacaaagtcggtgatgtcacggccaaacggtggtacaagcgcgcccagaagaagccgaaagcggaggcggcggccagtgcataactaatggatttttctaaggtgttcctgagtgttttaaatcagttaaatgtgcatgtttccgcttcgcaagatatggtgtaaaaacacgcggtttattagcccagtaccaaacaataacagatgcctgtatatttacgctttcgtatcgtatcgatatctgaataacaaagaacagaacgtgatgtttgttttcgtaaggaatcggcatcatagcgtgattattgataaacaaacaattgtcgagtgaaaccgattgatgatgcttggccttttcacatcagatcagtgatcacgtttaccgtatgaccgcgtcgttgattaaatataacggattaaaaaaa

SEQ ID NO: 169 - DNA, 1234 bp, Paramacrobiotus richtersi
cgggaaaagaacactccattgacttgaaaagtaatatcctctacagctttaaaatacgtagctaatccgttatatttaatcaacgacgcggtcatacggtaaacgtgatcactgatctgatgtgaaaaggccaagcatcatcaatcggtttcactcgacaattgtttgtttatcaataatcacgctatgatgccgattccttacgaaaacaaacatcacgttctgttctttgttattcagatatcgatacgatacgaaagcgtaaatatacaggcatctgttattgtttggtactgggctaataaaccgcgtgtttttacaccatatcttgcgaagcggaaacatgcacatttaactgatttaaaacactcaggaacaccttagaaaaatccattagttatgcactggccgccgcctccgctttcggcttcttctgggcgcgcttgtaccaccgtttggccgtgacatcaccgactttgtaggtcttgaccagctcctcgccttccacgtggtaatcgtccttgatcaccttgttgtgcgtgaccaggttgatctccacatgtaaatctttattctcttcggtgtatttgtacttgaattgagtattattgtagctcccggttccttcttctcccagcttgaactcaacgtcgagcttgtaacccttctccgggactttgatgcggtgatgatagtgatcgccctctttccacagtttgtgccagactttgtgctcatgtgtatagtgtgcgaagcctaaggcattgatgaagttctgaaagttttcctggcgcccgtcaatggattcccatttgccaatccagggatggtcggcgtcagcggcaatgtcttgctgatccttgtgttcgccctcggccggtcgggctttgacggccgccacaagtgcaatcaaaataaacaccttaaaggtcatgatgagagagctgtaagagattttcacctcgcattcagtcagtagcacaccacctgtctaccacgatagttcaactgttggttttatactataaaaaaaccgataatatctggctacattaggcaaaattacaacgctttgatgggaactgtgctaggaatccacttccgtacatgtcacacagatcaacaagcttccggcatcagtacacagttcgcgctaaggatgacagacagtttgctatctcatagtaatggaattatctgcagtcatatcaagaaaaacatggcaggaatgcaa

SEQ ID NO: 170 - DNA, 1182 bp, Paramacrobiotus richtersi
ccggtacacagtattggcgatcgaaagtttatgtcggtgacattaatgtagcaccttttactagacctactgaagatttccggacaatttgatctacacagattatctttcgagttaccagtcctgtgcaaccagaaccggttttcggtgtatggaccatgtttcactaacctttaagcccgacacaaatgtaacatttcgattcaaacggatacagaatttttgaacatagatctgtacaaggccgaaatcccagtttgttattatagcttacgaactgctgtgtttttaattacgcaaataacaaatgctgtaagacttttgtccagtactacaggaatttgtagtaacaaaaaatgacacagtagtggtcgattactgggtcccagattactgggtcccaggtcgagtcccagattactggtagtggtcgaggattataaaatgctctctgccggtgccttcttaaaccatcgtttggcttcgatagtgtccactttgtacgttttgaccatctcttccccctccacatggtaaacgtcatggatcaccttgttcttggatggaacgttcacttccacatgaagatccttgttttgttccgtgtatttatacttaaagtcggtaccattccacgtcagcgtgccttgttcgcctaatttgaattcaagaaactgcttgaaatttttatcgggaacaatgattccgtggtggaaatggtcttcttccttccagaatttgtggtagactttattctcagatttgtaatgtgccatgccgagatgttgaacaaaattgtcaaagttctcgtgacgatcagaaatggattcccatttgccgatccatggatgatctggatccgctgggatggttttgggatcagcttgtccatggcaggcaacgcacgtcaccacgccgaataataaagcagcaagcaaaaccatcctgcataaacggaattactgaactatttgcggatataaaaactgaaaagtttgtacacttttaaccttaaggtacgttcccaaatgtataagcggatgtcactaaattatgaggcagaaaagtaacagccgccagccgctgctttgactttggttcctattgaaaaaacgcggatggaaaatgaccgattaaaacgtccgtcggaaagatcaaaggctacactgcgcgttcgggtat

SEQ ID NO: 171 - DNA, 1200 bp, Paramacrobiotus richtersi
tggatatacagtgtatccatacccgaacgcgcagtgtagcctttgatctttccgacggacgttttaatcggtcattttccatccgcgttttttcaataggaaccaaagtcaaagcagcggctggcggctgttacttttctgcctcataatttagtgacatccgcttatacatttgggaacgtaccttaaggttaaaagtgtacaaacttttcagtttttatatccgcaaatagttcagtaattccgtttatgcaggatggttttgcttgctgctttattattcggcgtggtgacgtgcgttgcctgccatggacaagctgatcccaaaaccatcccagcggatccagatcatccatggatcggcaaatgggaatccatttctgatcgtcacgagaactttgacaattttgttcaacatctcggcatggcacattacaaatctgagaataaagtctaccacaaattctggaaggaagaagaccatttccaccacggaatcattgttcccgataaaaatttcaagcagtttcttgaattcaaattaggcgaacaaggcacgctgacgtggaatggtaccgactttaagtataaatacacggaacaaaacaaggatcttcatgtggaagtgaacgttccatccaagaacaaggtgatccatgacgtttaccatgtggagggggaagagatggtcaaaacgtacaaagtggacactatcgaagccaaacgatggtttaagaaggcaccggcagagagcattttataatcctcgaccactaccagtaatctgggactcgacctgggacccagtaatctgggacccagtaatcgaccactactgtgtcattttttgttactacaaattcctgtagtactggacaaaagtcttacagcatttgttatttgcgtaattaaaaacacagcagttcgtaagctataataacaaactgggatttcggccttgtacagatctatgttcaaaaattctgtatccgtttgaatcgaaatgttacatttgtgtcgggcttaaaggttagtgaaacatggtccatacaccgaaaaccggttctggttgcacaggactggtaactcgaaagataatctgtgtagatcaaattgtccggaaatcttcagtaggtctagtaaaaggtgctacattaatgtcaccgacataaacttttgatcgccaatactgtgtaccgg

SEQ ID NO: 172 - DNA, 944 bp, Paramacrobiotus richtersi
aaaaagaactcttgtctaatatttcataaactgctatttgagttttatttctgctcttcggaaacgatcgaagattttcttctctctgaatcatggttaacagttcacatacgctgctacggatagttaacagtctgctgattcatataggcagccgtgattaaaaaatgtacatcgtcgtatctcattcgttgttttagtcagtgcatatttggtggaggagcatgacaaaggtcacattaaatgcataaaaagcggcaatgccggtctaggcgttggcgggtttggacgcggccttcttgaaccatttcttggctttgacatcatcaacctggtaggacttgacgagctcctcaccctcgacatgatagacatcgtggacgaccttgttcttggatgggcatttcacttcagcatgcagatccttatctttctcggtataagtgtacttgaactcagtgttgttaaaagtgaagcttccttcttcgcccaacttgaactggaagaactttttgaactgcttgtcgggaatagcaattccgtggtggaaatggtctccctccttccacagtttgtggtagactttcatgtcatcggagtagttaatgggagcatctaacttcttgacaaagttggcaaaattctccgaacgtccctcgatggattcccatttaccgatccaagggtgtgccgggtcagcaggaatctccttgggatcgcttccgctttcgtcatgatggtcagcagcagcgaacaccgccagtcctaagaggacgaaagcaaggtacgacattcttgttgcacgtatgaggagcgtctgggaaaactctgacaactttccttctaatcgaaacctaaatgactatcgcataatgttttgcctatatatagggaaactgacgatagccgatgtcgcgcgttccatttttctttgcactttaaaa

SEQ ID NO: 173 - DNA, 1039 bp, Paramacrobiotus richtersi
ggcacacccttggatcatggaacgcgcgacatcggctatcgtcagtttccctatatataggcaaaacattctgcgatagtcgttcaggtttcgattagaaggaaagttgtcagagttttcccagacgctcctcatacgtgcaacaagaatgtcgtaccttgctttcgtcctcttaggactggcggtgttcgctgctgctgaccatcatgacgaaagcggaagcgatcccaaggagattcctgctgacccggcacacccttggatcggtaaatgggaatccatcgagggacgttcggagaattttgccaactttgtcaagaagttagatgctcccattaactactccgatgacatgaaagtctaccacaaactgtggaaggagggagaccatttccaccacggaattgctattcccgacaagcagttcaaaaagttcttccagttcaagttgggcgaagaaggaagcttcacttttaacaacactgagttcaagtacacttataccgagaaagataaggatctgcatgctgaagtgaaatgcccatccaagaacaaggtcgtccacgatgtctatcatgtcgagggtgaggagctcgtcaagtcctaccaggttgatgatgtcaaagccaagaaatggttcaagaaggccgcgtccaaacccgccaacgcctagaccggcattgccgctttttatgcatttaatgtgacctttgtcatgatcctccaccaaatatgcactgactaaaacaacgaatgagatacgacgatgtacattttttaatcacagctgcctatatgaatcagcagactgttaactatccgtagcagcgtatgtgaactgttaaccatgattcagagagaagaaaatcttcgatcgtttccgaagagcagaaataaaactcaaatagcagtttatgaaatattagacaagagttctttttcgtaataaatggtgatcgacttatggtgcgtgtaactcctgaaataaataagcatctagtgtaaccactgaattattgttaataaaaattaaagagca

SEQ ID NO: 174 - DNA, 1069 bp, Paramacrobiotus richtersi
ttttgatgcgccggcggaacactgcatcgggactaaatacgaagtgtactggacggtttttcagcattatttgcttgtggaatgtgaacgtctaattttcataaatagcgtaattgattacttttgttagctatctattccttgtgcagaattttatttacggccactatagtgcaagaatgaagtggttaatcgttgttgtattaggcatttctgcggccctggcggaggaccatccgacgccaaataacatcccactggacagtgcccaccaatggatcggtaaatggaaatcgactggacgccatgagcatttcgatgacttcatgaaggctttgggcctaccgaatcacgatgtggccgatccggaaaccacccatgtgttatggaaagaaggcgacaaatttcaccacaaaatctccgcaccgtctgtcaattacaagaagcatatctgttttacgttgggcgaggaaggaaacagctcctataatgggaccgcatttacgtacaagtataccgaactaccggacaaagatctggtgctggtagccacgcttccgtcgtacaacaagtcagtccatgccaccttccacgcgacggggaatgaactgatgaagaccttcaaagttgaccaggtggtcgccaaacgctggtatgctcgtgtggaccagactgccgctccaaagcccgccgcaaagtaacagctggagaacttcttcataagaatttgaagttgatggaagcgccacatatttacttaccttgtgcgcaactgaataacgaatggtccttgcctctttgcgtggttcttgcacaaaatctgtatcttgtatttctttgtcgtgtcgattagcacaaataacgcgttttattctgcttcgatgaatttggttcggtgaattgttcttcctaacggcagtcatcagattatgcgtcttttcaacagccagcatatgttccaacagaggcgtaaacaattgccataccgtccacaaaacaagcattgcaaaatgcagctcttttacattatgtatctgtagttcaaaacaagacatctacaagatttctg

SEQ ID NO: 175 - DNA, 1190 bp, Paramacrobiotus richtersi
cgtaaactgcaaactgtatctacgtggtaacaggcaggcaacatgtacatgctctggggaagaaaaacggcactgtgacttgactaacagaaattttgtagatgtcttgttttgaactacagatacataatgtaaaagagctgcattttgcaatgcttgttttgtggacggtatggcaattgtttacgcctctgttggaacatatgctggctgttgaaaagacgcataatctgatgactgccgttaggaagaacaattcaccgaaccaaattcatcgaagcagaataaaacgcgttatttgtgctaatcgacacgacaaagaaatacaagatacagattttgtgcaagaaccacgcaaagaggcaaggaccattcgttattcagttgcgcacaaggtaagtaaatatgtggcgcttccatcaacttcaaattcttatgaagaagttctccagctgttactttgcggcgggctttggagcggcagtctggtccacacgagcataccagcgtttggcgaccacctggtcaactttgaaggtcttcatcagttcattccccgtcgcgtggaaggtggcatggactgacttgttgtacgacggaagcgtggctaccagcaccagatctttgtccggtagttcggtatacttgtacgtaaatgcggtcccattataggagctgtttccttcctcgcccaacgtaaaacagatatgcttcttgtaattgacagacggtgcggagattttgtggtgaaatttgtcgccttctttccataacacatgggtggtttccggatcggccacatcgtgattcggtaggcccaaagccttcatgaagtcatcgaaatgctcatggcgtccagtcgatttccatttaccgatccattggtgggcactgtccagtgggatgttatttggcgtcggatggtcctccgccagggccgcagaaatgcctaatacaacaacgattaaccacttcattcttgcactatagtggccgtaaataaaattctgcacaaggaatagatagctaacaaaagtaatcaattacgctatttatgaaaattagacgttcacattccacaagcaaataatgctgaaaaaccgtccagtacacttcgtatttagtcccgatgcagtgttccgcaggcgcaacaaaaataacctttgatcgatgcagatcagtaattaaat

SEQ ID NO: 176 - DNA, 864 bp, Paramacrobiotus richtersi
cagacaggaatggatactcttttattttggtacgatatcctcgatgtactaagttcaagaaagcccaaagttcattgttgctctactgtctgcagtataaaaagttaatcaatgctcagttcagaacacgctagtaaacattctttcaaattccgaaccgtaggtttaacaacatgcaggtttccagtgttttattcgtccttggttgcgtgattgttaccatcgaaggcggtggcttacatcagttcttgggaaaatgggaatccacggagaagagggaaaatacccaggctttcgctgaagcgttaaatcaggtggatcaggtggatataaactcgaaaatcttcaatgagttctcgctggatcaggcgagtgctgatggataccaccacaaattttccgtccccgacaagaattacgtgcaagatgtaactttcaaactgggcgtcgaggggcaaaagactttcaatggaacaacctataagtacaagtatacactggatggtgataccctgaaatcgcactttgaacttcccgatcgacaagtcgatcaagagttcagtttggtcaacaacgaactggtcaagacgtacaaagtcaacaatgtcgtcgccaaggtctggtttaaaaaggtttaattaagctgtttcacaagtttgttggaaaagaaagctggcttgagtgtagttgttgtccttttagcgccgatatgtttgacaaaattaatgctgcttgtagtagggttgaaattcacgcggttttcgggtagagtgaagtgaagtgaaagtgaaagtgaaatgaagtgaagtgacgtgaaacgtgaaacagaaaaaaatcgttaattttacagttttacttctggac

SEQ ID NO: 177 - DNA, 807 bp, Paramacrobiotus richtersi
attcttttcgggtcccgaacaagaagtccatttgttgtaaccgtattacgattgtcttttctgccatgaatgtttgcattgcgtccttgtgccttggctgcctgattgttgctgtcgaaggagctggcttaggcatttttatggggaaatgggaatccactaacaagagggaaagcacccaggcattcgcggaagcggtcgaacatgtggatatcgactcaaagatcgtcaacgaattctcggtgaagaatggtggggaggaataccaccacaaattttccgtaccggataagaattacattcaagatctaccttttaaactgaacgaagaacgtcagacgacctttaatggaacaacctacaagtacaaatatacactggagggcgacactcttaaatcgcactttgaactgccggatcgtcaagtcgaccaggagttcaatttggtcagcaacgaactggtcaagacatacaaagtcaataacgagtccgctaaagtctggtttaagaaggtctagatgtgttcccagcttgaggtttcctaccacatcgatattttgtttctgtactctcgtaacatattacttcgtttcttgcttcagacacatttgcgagcgaaaaactcgccatttgttcggatatctgtacattgacttgctttggtcgtttagcattaccgaaacaaaatgcttgagttatacttcttaaatgcagtcttctgaaaaatgcaatgctgcagcggtaaatcttatgaaactgggaaaactgaaaactggaaatgagatacagagatagcgtggaatgtgg

SEQ ID NO: 178 - DNA, 852 bp, Paramacrobiotus richtersi
ccacattccacgctatctctgtatctcatttccagttttcagttttcccagtttcataagatttaccgctgcagcattgcatttttcagaagactgcatttaagaagtataactcaagcattttgtttcggtaatgctaaacgaccaaagcaagtcaatgtacagatatccgaacaaatggcgagtttttcgctcgcaaatgtgtctgaagcaagaaacgaagtaatatgttacgagagtacagaaacaaaatatcgatgtggtaggaaacctcaagctgggaacacatctagaccttcttaaaccagactttagcggactcgttattgactttgtatgtcttgaccagttcgttgctgaccaaattgaactcctggtcgacttgacgatccggcagttcaaagtgcgatttaagagtgtcgccctccagtgtatatttgtacttgtaggttgttccattaaaggtcgtctgacgttcttcgttcagtttaaaaggtagatcttgaatgtaattcttatccggtacggaaaatttgtggtggtattcctccccaccattcttcaccgagaattcgttgacgatctttgagtcgatatccacatgttcgaccgcttccgcgaatgcctgggtgctttccctcttgttagtggattcccatttccccataaaaatgcctaagccagctccttcgacagcaacaatcaggcagccaaggcacaaggacgcaatgcaaacattcatggcagaaaagacaatcgtaatacggttacaacaaatggacttcttgttcgggacccgaaaagaatgtttatcaacaagtagaagcaacattcacttgctatatggcggag

SEQ ID NO: 179 - DNA, 1285 bp, Paramacrobiotus richtersi
aagtagttctgtactagttgccaaaactgcatgtcacttaaccttgaactccttcaataacaacgacatcgtctgcatgattttcaatacaacccgttatcaaattcacatcgaacacccgttatacagtacttccggggcagtttaatcgcattggtgggaaactggctcgctggcggtcaaagtccatccagtagcctcgatcttgtccgttaacgataaacacatcacaaaatcaaagtaagcaatcaaatccgtaagccaaaaccgtaaatcaacaggtttcgcacaccaacaactcagtcaacgttataggtatcgacacaataccataaagtataagcaaatggagcaaccacagaggcaggcagttatggatttttcaacgcatgctctccgaacactaatcataacacataattacaagacataattactaattacaagagtcattcactgatgaatgaatgtacactttaaaataaaatttttatctgcaggctgcaggttaagatggctgggatttgattttcttgaaccaacgcttggcttgcacagctcctcgggtgaacgtcttgagcatatcttcgttttgaacgtgataaacatcagtgaaaacagtgctcgtggaagggatctttactattgcatgtaaatccttgcctttttcggaataaataaatttgtattcggtgccatttaggatgaccacactctcttcacccaatctaaaccggaattcggttcgaaaaccgcgctcaggtgctgcaacgacgtgatagaaatggtcgccttctctccagaactgacgatacactttggtattggcgtcgttcatataaggtgcaataccgatttcgttgcggtcagtatcaacactttgaaagcggccttcgatggattcccaactaccgatccagggatgttccggatctggatggatatcttttaaagacatgtggctgccttgttctggctccaaacgcgaacatgctgccagtcctaaaagcagcagtccaatatacctcatgttatcggaaaatttgatgtgtgcgctgaatttactggccggatttctgaacaaagcttcaaattttttcataagaaaaacagtgttagaagattgtttcctgttggttgaagttagacagcgggataccgtccttgcgctttgcagtaattccgcgcgtttcgaaagtttcgttcatcagatagtccgagctttgtttaccggtttacccatgttcagtgtctctttcgaatgcgattgccatacacactgttc

SEQ ID NO: 180 - DNA, 1010 bp, Paramacrobiotus richtersi
ccagttttgggcatcttgttcatgtatatgcgtacgtaaacgaatgttgtacatactgttttacttacactgaaccaaaatcttctagccattacagcaaaaacgcggcaccacactctgatgtcaaaaaatattgaagacatcgatgctgatcaacaaaacagtattactgttgctggtaccaacaatgtactacaagcagcattaattttgtcaaacatatcggcgctaaaaggacaacaactacactcaagccagctttcttttccaacaaacttgtgaaacagcttaattaaacctttttaaaccagaccttggcgacgacattgttgactttgtacgtcttgaccagttcgttgttgaccaaactgaactcttgatcgacctgccgatcggggagttcaaagtgcgatttcagggtgtcaccatccagtgtatatttgtacttataggttgttccattaaaagtcttttgaccttcttcgcccagcttaaaagttacgtcttgaacgtatttcttctcgggtacggaaaatttgtgatggtatccattaatattcgcctcatccaccgagaattcattgaagattttcgagttgatatccacctgatttaacgcttcagcgaaagcctgggtattttccctcttctccgtggattcccattttcccaagaactgatgtaagccaccgccttcgatggtaacaatcacgcaaccaaggacgaataaaacactggaaacctgcatgttgtcaaacctacggttcggaactcgaaagaatattaattaccggcatgttttgaactcagcattcaactttttatactgcatacagtagagttttaatgaactttgggcttccgtcttggacttggtacatcaaggatatcgtaccgaaataaaagagtgtccattcctgtctgctcgcaaactgtcagacaacttactgtgtactaacttactgtacgtgcacgcctagagtctgtaggctacagatattgagtgtgca

SEQ ID NO: 181 - DNA, 1242 bp, Paramacrobiotus richtersi
ccagcaaatattttctcggctcagaatttagaacagtgtattgcaatcgcattcgaaagagacagtgaacatgtgtaaaccggcaaacaaagctcggactatctgatgcacgaaactttcgaaacgcgctgaattgctgcaatgcgaggacaataataactcgcagtctataacttcgaccaacagaaaacaatattttgacagtgtttttcttagcaattaatttggaactctgttcagaaatccggccagaaaattcagcgtatacatcagatttcccgataaaatgaggtatattggactgctgcttttaggactggcagcatgttcgcgtttggagccagaacaaggcagccacatgtctttaaaagatatccatccagatccggaacatccctggatcggtagttgggaatccatcgaaggccgctttcaaagtgttaatactgaccgcaacaaaatcggtattgcaccttatatgaacgacgccaataccaaagtgtatcgtcagttctggagagaaggcgaccatttctatcacgtcgttgcagcacctgagcgcggttttcgaaccgaattccggtttagattgggtgaagagagtgtggtcatcctaaatggcaccgaatacaaatttatttattccgaaaaaggcaaggatttacatgcaatagtaaagatcccttccacgagcactgttttcactgatgtttatcacgttcaaaacgaagatatgctcaagacgttcacccgaggagctgtgcaagccaagcgttggttcaagaaaatcagatcctagccatcttgatctgcagcctgcacttaaaattcttcctgttcttgttctgcttcttgtaaagtgtacattcattcatcagtgaatgactcttgtaattagtaattatgtcttgtaattatgtgttatgattagtgttcggagagcatgcgttgaaaaatccataactgcctgcctctgtggttgctccatttgcttatactttatggtattgtgtcgatacctataacgttgactgagttgttggtgtgcgaaacctgttgatttacggttttggcttacggatttgattgcttactttgattttgtgatgtgtttatcgttaacggacaagatcgaggctactggatggactttgaccgccagcgagccagtttcccaccaatgtgattaaactgccccggaagtactgtgtaacgggtgtttgatgtgaatttgataacggg

SEQ ID NO: 182 - DNA, 881 bp, Paramacrobiotus richtersi
tttttttttagtattcatatgtcttgttgtagtattgatgtattttgtgacaatagtcagtcgtttatgttcgatctacgactcaacgtacaaacgctcgtttcagttaggctgcaggtcgtacacaattaataaactaaggttgcttcgcctttaattagaccaagcaaaaaaaaacagactgcgtcgcagcgcggttgatatgtgcacagaaatcgcttgtccaaatcgcggtaaaatctttagactgtatttggttaaatcagatactggtgtcagaaaaatggaatcatttctgcactttcttgtaaacacgtttagctttgacgcttccagattcgtactccttgttaattttgtctccttcaacgttatagcattccttcagctccttattctttgaagggatattcatatggactttgagtttatttggtccttcttccgtatatttatacttaatttccggttcctctccaaggtgccatgaatgttcctggttgagtttaaatttcacttcgtaattttgaccaccttcgatttggactttatgtacaaattcatcgccctgctcgatatactcgtgggtgattttcttggtggtataatgaggaatggcgtctttaatctggtcgacaagttgttgcatgttttcctcgccttcgggagtggattcccactttcccagccaggcgtgggaatggtcggagtggctgctctgattggcttgatcggccggtgcggcattcgcacaagccgctactaacccaaaaatcacgagtgaggcaaacatcgtattattaatactggcagtaaagaatcccgtaataccaaaagatttctgaaaacaatactgcactccagacgtacagaacggtccg

SEQ ID NO: 183 - DNA, 878 bp, Paramacrobiotus richtersi
accgttctgtacgtctggagtgcagtattgttttcagaaatcttttggtattacgggattctttactgccagtattaataatacgatgtttgcctcactcgtgatttttgggttagtagcggcttgtgcgaatgccgcaccggccgatcaagccaatcagagcagccactccgaccattcccacgcctggctgggaaagtgggaatccactcccgaaggcgaggaaaacatgcaacaacttgtcgaccagattaaagacgccattcctcattataccaccaagaaaatcacccacgagtatatcgagcagggcgatgaatttgtacataaagtccaaatcgaaggtggtcaaaattacgaagtgaaatttaaactcaaccaggaacattcatggcaccttggagaggaaccggaaattaagtataaatatacggaagaaggaccaaataaactcaaagtccatatgaatatcccttcaaagaataaggagctgaaggaatgctataacgttgaaggagacaaaattaacaaggagtacgaatctggaagcgtcaaagctaaacgtgtttacaagaaagtgcagaaatgattccatttttctgacaccagtatctgatttaaccaaatacagtctaaagattttaccgcgatttggacaagcgatttctgtgcacatatcaaccgcgctgcgacgcagtctgtttttttttgcttggtctaattaaaggcgaagcaaccttagtttattaattgtgtacgacctgcagcctaactgaaacgagcgtttgtacgttgagtcgtagatcgaacataaacgactgactattgtcacaaaatacatcaatactacaacaagacatatgaatactaaaaaaaaa

SEQ ID NO: 184 - DNA, 995 bp, Paramacrobiotus richtersi
ctttgctcggtgccaaataccatgaacgggtgccgtatgccgtgcttaattgagttgataaaaatataccaccagatgttatatttcgagctataattacttgttatttttgcgtgagtgcatttctgcgtgaccttttacctggcgctatttctgctcagccagctacgggagacatggcgcaaatgctgttcatcataggatttctgtgttgcgcgggcgtgtacggctcgcagtccgttgggcgtgttcatatgaaggatcattacgggaaccgcggggacagtttcgagaacgtcgcgcaccagtggctgggcaagtgggaatcagtggaaggaactgaagaaaacttcgaccagctcttggacgctatccgtgaagcgttcccgtattacagccaagcaacaatcatccatgatttcagcaaaaagagcgatgacgagtttatccacaagataaaaataggcagcgacgaagatcattatcagctgacgttcaaattggaccaggaaggtaccctacgcaagccaggcgcgccggaaatgaagtacacatacgaggaagtttcgggaaacaagctggtagtccaacaaagcgtcccatcaaagaatattatgctggaggaaagttacaaggttcagggtgatcagatccttaaggaatatgcaaccggaggtgtccgggccaagcggacgttccaaagaatgaaccatttgtgatttgctgctcccaccgtcccacgcaagctgccttcttactgcccgttgtagttgtatatggtcattttttcgcttcgatattcctgcgttgtggccttgttagtttttaattaagctgttctgtagcgagccccctaatcctgtgtgcgaattgcgtgatgtttcagaggttccgttatctttgtattttcgtaacaatatgttaaatgtacacctgcttcgttttttttaaaaactaagtacaccacgcaagtatgtgactaaattaaaccatat

SEQ ID NO: 185 - DNA, 1000 bp, Paramacrobiotus richtersi
atatggtttaatttagtcacatacttgcgtggtgtacttagtttttaaaaaaaacgaagcaggtgtacatttaacatattgttacgaaaatacaaagataacggaacctctgaaacatcacgcaattcgcacacaggattagggggctcgctacagaacagcttaattaaaaactaacaaggccacaacgcaggaatatcgaagcgaaaaaatgaccatatacaactacaacgggcagtaagaaggcagcttgcgtgggacggtgggagcagcaaatcacaaatggttcattctttggaacgtccgcttggcccggacacctccggttgcatattccttaaggatctgatcaccctgaaccttgtaactttcctccagcataatattctttgatgggacgctttgttggactaccagcttgtttcccgaaacttcctcgtatgtgtacttcatttccggcgcgcctggcttgcgtagggtaccttcctggtccaatttaaacgtcagctgataatgatcctcgtcgctgcctatttttatcttatggataaactcgtcatcgctctttttgctgaaatcatggatgattgtcgctgtgctgtaatacgggaacgcttcacggatagcgtccaagagctggtcgaagttttcttcagttccttccactgattcccacttgcccagccactggtgcgcgacgttctcgaaactgtccccgcggttcccgtaatgatccttcatatgaacacgcccaacggactgcgagccgtacacgcccgcgcaacacagaaatcctatgatgaacagcatttgcgccatgtctcccgtagctggctgagcagaaatagcgccaggtataaggtcacgcagcgacgaaatgcactcacgcaaaaataataagtaattacagctcgaaatataacgtctggtggtatatttttatcaactcaattaagcacggcatacggcacccgttcatggtatttggcaccgagcaaag

SEQ ID NO: 186 - DNA, 1484 bp, Paramacrobiotus richtersi
tccgcccgtttgtgttgtatgcgctttctttcgttgtgtaatataatgttgcgttcgtgttctatcagaaagagatttgtatatatttttcaggattttccgttagccgttgcacgatatacgtaatcttcgttactctcctgattattcttgactacagcttattcgttgattttctttgtcttgttcaaaagattgattaagcaagtacagctggtactttgtaaacggacagcaacgtgcaatgaactccttgtttaaatattgtagtatgcttgtccaaaacgtcataaaattacagtacactggtacagtatgtaatgcaccaatgattatgacaacttttgccgcagacgattgaagttttttgtaacgcaacctctcaccggccgctgatcagccagcgatcagcctgccaggaaccatccagaatttgtccattgtccagccgaaaaccagcctgcatccggccgttaaccagccagctatccatagccagccgtagccagtgatgagcagtacccacacataagatagcatttataaataatattatagtgtacgagcacagcagcaaaagtctaatccagtcaaaaggaaagaacaaagaggaggcgtcgcttctttcccgtctttgacctatttttcagttccttacggaatccttttgtacgcgcgcttcgctgttacgtctccaattttataggtcaaaacaaaaccgtccgggctggttgtgtacagatgttcgataggtaagggattatccgccggaacgtggaatacagcatgtagaccgagttgactgggttcttgataatatcggtacttgaagccagttccattatatgtttgctgatgttcttttccaagttcaaaactaagcttctgtttatatggggtgccagcaatccagaaagcatgggaatatttgttgtcgggtgcacgactaaattcatgttggacctttcctttcaaatttccggcggaaaaccctttgaattccacgcgtaaacttgacaaataactggcgaaattttccacttgatctgtggcttcaaattttccgaaaggtatcaacacaggtttagctgcttcgaccgcaatacttgtcccatttccggaaccccgcggcggagtagtttctgttgattccgctgtcacacagctaaagcaatagaacgcaataacaaaatacgcgctgctaggcctgggcacaccagtcatttctacgatgtcttgaaaagttcacaagatatctgctgtctaaaaccagtaaaccataaaataaaacgtgacacacaagtaaagacactataatataattatacgaaaaaatgatggaaatgtcaaaagacaagactaccgctaaaaataaaaaaagatggcgtcaagcagaatgataaataaaagcgcattggctgtttatttctgatgttttgtagctgaaaatattacactcatatataatataaccaagtcttgac

SEQ ID NO: 187 - DNA, 1296 bp, Hypsibius dujardini
cggaaaaaagatggaccctttttgccgaaaaactgtttatccgcgctccgctccgcttcacgcggccaccggacttcggacagtttttacggtatgtttttcgagatttctaggtcgtgatatcgtccgtgagaacagcaaatacgctgggctgtcgttccttgttttggcctcttagtaaattctataaaaccaaccgcggaattcggattgccaaagcagttttcttcaacctcgcgacctctcacgaaccctttgccaaattcacacaaaatgtctcgaacgatcgtcgccttgatcctcctcggccttgctgcgcttgccgcagcggaccaccatgaaggtcacggagcggaaaaagaatgggcaggcaaggcttggcttggaaaatgggtgtccaccgatcggtcagaaaattgggacgcctttgttgaggccctgggtcttcctctggcggcctatggcggaaatcacaagaccgtccacaagctctggaaggagggtgatcactatcaccatcaaatcatcattgcggacaagtcctacaagcaggacatccagttcaagctgggcgaggaaggccggaccgcgcacaacggcacggaagtcactttcaagtacaccgaggtcggtgacaacctccaaaacgaagtcaagatcccctccaagaacaagaccatctccgactcgtacgtcgtgaaaggagacgaactcgagaagacgtacaagatcaatgatgtcgtcgcgaagcgctggtacaaaaagcacgcccacgagcccagcacagcttgaatctctctcaagatgtcttcctgaataatcagtttcccggataaatttttcggtgcaattattatgtttttgtcgaatttgttttgtctgtgtttgatgcgacgaagtttttaaagcttggtttaacgagacgatgtgaatatttcgcgtattaccagtcggtattgtgattaaaaattgagagtatattcgtctggcaaagaagcgtgggagcaaactgcggaatcacacgccgcacgaccgacgagtttggctgtgtgtttttttagaaacgcaattttcaatcgttgagccagaaaaagtttaaaacaaatcctcctaagatttggaaggccgcgataaggggatgagtttgtccttataggatggttttcaagccatcactgactgactgatgaaacagaaatggccaacgaaaatgccacgaaacggagtgaaatgagtgaaatgaagggataaatgtgcgagtgctaagagaaataatagcggcaaagaagaagacat

SEQ ID NO: 188 - DNA, 1356 bp, Hypsibius dujardini
gttgttttctgccatgttatttcttcttcgttgccgctattatttctcttagcactcgcacatttatcccttcatttcactcatttcactccgtttcgtggcattttcgttggccatttctgtttcatcagtcagtcagtgatggcttgaaaaccatcctataaggacaaactcatccccttatcgcggccttccaaatcttaggaggatttgttttaaactttttctggctcaacgattgaaaattgcgtttctaaaaaaacacacagccaaactcgtcggtcgtgcggcgtgtgattccgcagtttgctcccacgcttctttgccagacgaatatactctcaatttttaatcacaataccgactggtaatacgcgaaatattcacatcgtctcgttaaaccaagctttaaaaacttcgtcgcatcaaacacagacaaaacaaattcgacaaaaacataataattgcaccgaaaaatttatccgggaaactgattattcaggaagacatcttgagagagattcaagctgtgctgggctcgtgggcgtgctttttgtaccagcgcttcgcgacgacatcattgatcttgtacgtcttctcgagttcgtctcctttcacgacgtacgagtcggagatggtcttgttcttggaggggatcttgacttcgttttggaggttgtcaccgacctcggtgtacttgaaagtgacttccgtgccgttgtgcgcggtccggccttcctcgcccagcttgaactggatgtcctgcttgtaggacttgtccgcaatgatgatttgatggtgatagtgatcaccctccttccagagcttgtggacggtcttgtgatttccgccataggccgccagaggaagacccagggcctcaacaaaggcgtcccaattttctgaccgatcggtggacacccattttccaagccaagccttgcctgcccattctttttccgctccgtgaccttcatggtggtccgctgcggcaagcgcagcaaggccgaggaggatcaaggcgacgatcgttcgagacattttgtgtgaatttggcaaagggttcgtgagaggtcgcgaggttgaagaaaactgctttggcaatccgaattccgcggttggttttatagaatttactaagaggccaaaacaaggaacgacagcccagcgtatttgctgttctcacggacgatatcacgacctagaaatctcgaaaaacataccgtaaaaactgtccgaagtccggtggccgcgtgaagcggaacggagcgcggataaacagtttttcggcaaaaagggtccatcttttttccgcagctccttacgcgaagaaacgtcaagtcatttcattcgttt

SEQ ID NO: 189 - DNA, 903 bp, Hypsibius dujardini
cgtgttttatttcccttttttgcttcagttggattattacgccgtgttttatttcccttttttgcgtaggcagaagatcgagccgaaatccgtttgagcaattgttcaggtcattccacaaaacatatttcttataaaattgcaaagattcaaagcttcaaagtacacaagcgaggtaaaaaggctactgctggtttaagcagtgggattggcctcccgcgtgaaccaccgcttggcgacgacatcaccgaccttgtacgtcttcgtgagggtgtctccctgaacttcatagatgtcgtggatttgcttgttcttggaggggatctgcacgtcagcgacgagcttctcaccgtcctcggtgtacttgagcttgatggtgtgcgggccgtgttggacgtcggtctcggtgccaagggtgtactcgatggccttcttgaagttcttcgaggggatgctcacctcgtggtggtacttgtcgccctgcttgtagatggtgtggatggtccgctggacctcacggggatactgatcggacggcagaccgagagcatcgacgaaggcttcccagttctccgcgcggtcggtggacgcccacgagccaagccaggtctttccagtccattctttttccgcagcagcgaaagccacgacaccgaaaagggcgacagcgacgaaaaggcgagccattttgagtttttagggaaggactggaaataagcttggacaaagttattgaggagaagagagaagtgaagtttgccgattgggtttctcgagttttatacacagggtcttctacacagggtctttagaacaaacagactattgtgcgctaatctcattgatccctatctctgattttcgtctaggctgagagacgcgtggagagcacaaaggttggtg

SEQ ID NO: 190 - DNA, 1006 bp, Hypsibius dujardini
cgattgtggtgagccgacacttttcgtcatgccattcgaacgctctcaccaacctttgtgctctccacgcgtctctcagcctagacgaaaatcagagatagggatcaatgagattagcgcacaatagtctgtttgttctaaagaccctgtgtagaagaccctgtgtataaaactcgagaaacccaatcggcaaacttcacttctctcttctcctcaataactttgtccaagcttatttccagtccttccctaaaaactcaaaatggctcgccttttcgtcgctgtcgcccttttcggtgtcgtggctttcgctgctgcggaaaaagaatggactggaaagacctggcttggctcgtgggcgtccaccgaccgcgcggagaactgggaagccttcgtcgatgctctcggtctgccgtccgatcagtatccccgtgaggtccagcggaccatccacaccatctacaagcagggcgacaagtaccaccacgaggtgagcatcccctcgaagaacttcaagaaggccatcgagtacacccttggcaccgagaccgacgtccaacacggcccgcacaccatcaagctcaagtacaccgaggacggtgagaagctcgtcgctgacgtgcagatcccctccaagaacaagcaaatccacgacatctatgaagttcagggagacaccctcacgaagacgtacaaggtcggtgatgtcgtcgccaagcggtggttcacgcgggaggccaatcccactgcttaaaccagcagtagcctttttacctcgcttgtgtactttgaagctttgaatctttgcaattttataagaaatatgttttgtggaatgacctgaacaattgctcaaacggatttcggctcgatcttctgcctacgcaaaaaagggaaataaaacacggcgtaataatccaactgaagcacatcgttgtgtgctctgtgttattatttatcttttctggttaacccgttaacaccccccaatgtcgtcaaacaacga

SEQ ID NO: 191 - DNA, 1104 bp, Hypsibius dujardini
ataaatccatattttgtattggtgcgttgcgttgtcgggatcataaatcggagtccgcccaactctagataattttccccgctccagcggtgttcagttgcactagttacggaggaacctttgctcatcaaccctggtaagagatagcaatcgaccggcgcgctaaggcgactgagtgaaacaacatcctttcatgggaactttcctgtataaaaatccgcgaccaattttacggcaaagccagtctaaagtttttcgcattgttcttttctgttctacaatcacgcccaaaaaaccgcgcaaccatgtctcgagtcctcgtcgccctcgctctctttggtgtggtggctctggccgcagccagtggcgatgcgcaaaaagaatggaccggcaagtcgtggcttggaaaatggcagagccttcccactgacaagtccgagaactgggaggcttttgttaatgccctcgctattcccgaacagtacacgcgtgatctccagaagaccgtccacactttctataaacagggtgaccactaccaccacatctttgccattcccgacaagaactttgagaagaacattgagttcaacctcggcgcggagtcgtcggccaagcacggcgaacacgaggtcaagatcaagtacgccgaggatggtgacaagctcgttgctgatgtgcgcattgccgcgaagaacaagcacattcatgacgtctacgaggttcaaggcgaagaactcgtcaagacatacaaggtcggcgacgttgtcgcgaagagatggttcaagaagatcgctcaataagcagtggattctaaaattctctgcaggagcttgtgctctattgctttattttgttttactgaaagaattgcgagaagtttgaaattacgaatgggtgccatcaagcttgattggtatgataataaactgtcttattagtgcttggtgtagagaactggcacttttaagcggaggaacgtactaactactgatacaataggacagcttacgcaggcgtggttaaaactgggaaataccggtgctcggcctgataaggcgctgacaaaaaaaaatgcttttggttaaaaaaaaagatcggaag

SEQ ID NO: 192 - DNA, 1262 bp, Hypsibius dujardini
gacacagacaaagacacagacactgacgaagacacaaacacatggcacacactctcacatagataccaacatagatatctctgcatgagttaggggcagacataacgacatacatgaacgtacatagtgacatatcaaaaacttggaaagaaccaaagatatatggacaaacttaaccaaaagcatttttttttgtcagcgccttatcaggccgagcaccggtatttcccagttttaaccacgcctgcgtaagctgtcctattgtatcagtagttagtacgttcctccgcttaaaagtgccagttctctacaccaagcactaataagacagtttattatcataccaatcaagcttgatggcacccattcgtaatttcaaacttctcgcaattctttcagtaaaacaaaataaagcaatagagcacaagctcctgcagagaattttagaatccactgcttattgagcgatcttcttgaaccatctcttcgcgacaacgtcgccgaccttgtatgtcttgacgagttcttcgccttgaacctcgtagacgtcatgaatgtgcttgttcttcgcggcaatgcgcacatcagcaacgagcttgtcaccatcctcggcgtacttgatcttgacctcgtgttcgccgtgcttggccgacgactccgcgccgaggttgaactcaatgttcttctcaaagttcttgtcgggaatggcaaagatgtggtggtagtggtcaccctgtttatagaaagtgtggacggtcttctggagatcacgcgtgtactgttcgggaatagcgagggcattaacaaaagcctcccagttctcggacttgtcagtgggaaggctctgccattttccaagccacgacttgccggtccattctttttgcgcatcgccactggctgcggccagagccaccacaccaaagagagcgagggcgacgaggactcgagacatggttgcgcggttttttgggcgtgattgtagaacagaaaagaacaatgcgaaaaactttagactggctttgccgtaaaattggtcgcggatttttatacaggaaagttcccatgaaaggatgttgtttcactcagtcgccttagcgcgccggtcgattgctatctcttaccagggttgatgagcaaaggttcctccgtaactagtgcaactgaacaccgctggagcggggaaaattatctagagttgggcggactccgatttatgatcccgacaacgcaacgcaccaatacaaaatatggatttatat

SEQ ID NO: 193 - DNA, 930 bp, Hypsibius dujardini
actctaatcgaagtctgtcaacctaaaaaactcgacctttaatctggcatggcggaagacacacgtgcacatttcctaccaattttattcggaaatcctgcttcgttttttgttttgagctcggtcattcggagcatcagatatttttcaaatatccgttttctatccagaaaaagctgttgaaaggcaattcatatttaattttaggtacaatttttaaatcacacgcagacaaatccagacacatgactgttaacgggcaaggacgctagggaaagactgcgtggtttaggcagcagggttggcgaccttcttgaaccacttcttggcgacgacatcgccgaccttatacgacttgatgagctgatctccctgaacatcatacacatcatggattgacttgcccttggcgggaattgaaacatcagcgacgagcttgttaccgtcctcgaagtacttgatcttcaggctgtgctcgccgtgtttgatctcaacctcctggccgagggtgaactcaatgtccttgttgatgttcttgtcgggcagggagagaatgtggtggtagtgctcaccctgcttgtagaacgagtggatggtcttcagctgggcacggggatactgatcgggcagaccaagagcatcaacgaaggcttcccagttttcagacttgtctgagacctcagcgacaaatttaccaagccaaggctttccagaccattcttttggcgcatcaccagaggcggcggtcatggcgaccacaccgaaaagagcgagagcgacgaggaagcgagccattgtgactgtgtttttttttgcggtctagaaaagcgaagactaagaaacagtatgaagaggcagacaaatctaaaaacaagtactgaattctaaagcgcccagaaaaggacgttttatagcatccctgtgctgttg

SEQ ID NO: 194 - DNA, 2006 bp, Hypsibius dujardini
cgtgtctctaagagtacgtgtctctaagggtacgtgtctttaagggtacgtgtctctatgctataacgaagggtggttcggcaaggatgacagtcagtaaaataataatacttttcctggctcgcctcttcggtctgttgatcgtaaacaaattcaacctttgcccatgcatggaaaaaggattgtgtcatggaatctgtcatgcagctttaccgtctacgaactcggaattattttgccgttttaatttgctgcggactccagagagccagtcttttgtttcctggtcattttcagcaaacaaaaaaatcacaacacgctcaaacgtcaagcaattaattcttaagtacaaatttaaaccctgcacagacaattccagacagatcgttgtttagacttcatggtacggagaagctggagaaatactgatctaggctgcttgggcaggggcctcggtggggttggccaccttcttgaaccatttcttggccacgatatcaccggtcttatatgtcttgatgagttgatctccctggacctcatattcactacggatggtcttgcccttggtggggattgagacgtcagcaacgagcttctcaccgtcctcggagtacttggtcttcgcgatgtgttcgccttgcttgatctccacctcctggccgagagtgaactcaatgtccttctcaaagttcttgtcgggaagggcgaagatgtgatggtagtggtcaccttgcttatagattttttggatggtcttcaccggggcacgaccaaattgttcgggcagaccaagcgcgtcgacgaaggcttcccagttctcggacttgtctgtgacctcagcgacaaatttaccgagccaaggttttccagaccactcttttggcgcatcaccagtggcggcggtcattgcgaccacaccgaaaagggcgagggcgacgagaaagcgagccattgcaatattgtttggaaggtctaggaagcgaaagctaaaacattgttggaagttcagcaaaccaaaacagaggcactgatttttaagtgtttcaatgagcgcttttcatacagtctcagtgttttctggcaatttttgccaggacttatctgcttcctctatcaaaaccatttatgagcaaaggttgcatttttcgttaccagacagtttagaacgttgtttgtgcctgttttatgtgagttgtgcgccttctcaatcagcctgatctgcaggtttaatttagttaaaattaaggaataattaagaataataataataagaaaataataataagaaaacacaatagtctcagtaatgtctaaacggaaagatttggctttctctaatttcttataaaatgatgaagttgctaaacagataaaagtgaaattccagttggcctaatttccgttaagttattaaagtgctttaattgtacgtgtaatcttttattttccctgtgtttttaatttgagggctgttaaatttccaagtgtgcacgctgtatgaacgtgaacgtgacaacttgttgacttgatgacaatcaaacggaggaatctgctggtcattttgagcggcctggagagcctcgctggtcatcacttacagtatatccgcggcaacaacaataacaacaacacaagtaacaaccctacttgccacatctcactctttactctttactgtaattaacatttcgcattttactgatataattttccgagatacctttttttccgtaaaagaaactcgagacaattgtctgattagaacatcggtatcaggccgctctgtatgtccatctcgaccgtctagtcagtgagtgagtcagttaatgcgtctgtctgtgattgaaatctgcttccggtgaggctctaccggtggaatgggaatgagccatgcgagcaaagagtgcttcgtccagttcgtccaacatgacaatcagaaaatgcttttccgcgagctcaactctaatcgaagtctgtcaaccaattttattcggaaatcctgc

SEQ ID NO: 195 - DNA, 2692 bp, Hypsibius dujardini
acgggtttaagttcccggaaaccgataaagatctgcgagtaaaccggtcaacagcacagggatgctataaaacgtccttttctgggcgctttagaattcagtacttgtttttagatttgtctgcctcttcatactgtttcttagtcttcgcttttctagaccgcaaaaaaaaacacagtcacaatggctcgcttcctcgtcgctctcgctcttttcggtgtggtcgccatgaccgccgcctctggtgatgcgccaaaagaatggtctggaaagccttggcttggtaaatttgtcgctgaggtctcagacaagtctgaaaactgggaagccttcgttgatgctcttggtctgcccgatcagtatccccgtgcccagctgaagaccatccactcgttctacaagcagggtgagcactaccaccacattctctccctgcccgacaagaacatcaacaaggacattgagttcaccctcggccaggaggttgagatcaaacacggcgagcacagcctgaagatcaagtacttcgaggacggtaacaagctcgtcgctgatgtttcaattcccgccaagggcaagtcaatccatgatgtgtatgatgttcagggagatcagctcatcaagtcgtataaggtcggcgatgtcgtcgccaagaagtggttcaagaaggtcgccaaccctgctgcctaaaccacgcagtctttccctagcgtccttgcccgttaacagtcatgtgtctggatttgtctgcgtgtgatttaaaaattgtacctaaaattaaatatgaattgcctttcaacagctttttctggatagaaaacggatatttgaaaaatatctgatgctccgaatgaccgagctcaaaacaaaaaacgaagcaggatttccgaataaaattggtaggaaatgtgcacgtgtgtcttccgccatgccagattaaaggtcgagttttttaggttgacagacttcgattagagttgagctcgcggaaaagcattttctgattgtcatgttggacgaactggacgaagcactctttgctcgcatggctcattcccattccaccggtagagcctcaccggaagcagatttcaatcacagacagacgcattaactgactcactcactgactagacggtcgagatggacatacagagcggcctgataccgatgttctaatcagacaattgtctcgagtttcttttacggaaaaaaaggtatctcggaaaattatatcagtaaaatgcgaaatgttaattacagtaaagagtaaagagtgagatgtggcaagtagggttgttacttgtgttgttgttattgttgttgccgcggatatactgtaagtgatgaccagcgaggctctccaggccgctcaaaatgaccagcagattcctccgtttgattgtcatcaagtcaacaagttgtcacgttcacgttcatacagcgtgcacacttggaaatttaacagccctcaaattaaaaacacagggaaaataaaagattacacgtacaattaaagcactttaataacttaacggaaattaggccaactggaatttcacttttatctgtttagcaacttcatcattttataagaaattagagaaagccaaatctttccgtttagacattactgagactattgtgttttcttattattattttcttattattattattcttaattattccttaattttaactaaattaaacctgcagatcaggctgattgagaaggcgcacaactcacataaaacaggcacaaacaacgttctaaactgtctggtaacgaaaaatgcaacctttgctcataaatggttttgatagaggaagcagataagtcctggcaaaaattgccagaaaacactgagactgtatgaaaagcgctcattgaaacacttaaaaatcagtgcctctgttttggtttgctgaacttccaacaatgttttagctttcgcttcctagaccttccaaacaatattgcaatggctcgctttctcgtcgccctcgcccttttcggtgtggtcgcaatgaccgccgccactggtgatgcgccaaaagagtggtctggaaaaccttggctcggtaaatttgtcgctgaggtcacagacaagtccgagaactgggaagccttcgtcgacgcgcttggtctgcccgaacaatttggtcgtgccccggtgaagaccatccaaaaaatctataagcaaggtgaccactaccatcacatcttcgcccttcccgacaagaactttgagaaggacattgagttcactctcggccaggaggtggagatcaagcaaggcgaacacatcgcgaagaccaagtactccgaggacggtgagaagctcgttgctgacgtctcaatccccaccaagggcaagaccatccgtagtgaatatgaggtccagggagatcaactcatcaagacatataagaccggtgatatcgtggccaagaaatggttcaagaaggtggccaaccccaccgaggcccctgcccaagcagcctagatcagtatttctccagcttctccgtaccatgaagtctaaacaacgatctgtctggaattgtctgtgcagggtttaaatttgtacttaagaattaattgcttgacgtttgagcgtgttgtgatttttttgtttgctgaaaatgaccaggaaacaaaagactggctctctggagtccgcagcaa

SEQ ID NO: 196 - DNA, 1388 bp, Hypsibius dujardini
tctgtatgtctgtctgtctgtctatctgtacttccaaacgattggctgcagcttcaaaaagttcattcacaagtgaataattagctgtcggcattcaatttggcgtcagcgtctaagaatagtgatattgatgggtttaaataccagaaacaaattaaggtccagatacaaagaaaaagaagtataaaatagcaaatgcggtagtcagcttaagcaagtactgcccaggcaatatttatgaaaatattgcaattccacgggcgtcctcagtggcagctgatcactttcttgtaccaacgcttggccactgtgtctcctgttttgtaggtcttttccaactcggttccattttggaaaacatggtaggtgtcactgacggtcttgttcttgctcggcacggtgatctcaacgtggaggtcgtttcccagctcggcgtagaggtacttgaattcgacattgttgaaagttgcctttcccggctggcccaatgtgaacttgtgttgcaaggtgtagttcttggtcggtacggtgatcttatgatggaaatgctcaccatcctgccagaacttttggaataccttctggtccgtcacgtgctccgcagacggataaccaagttggctgacgaaggtctcgaggttctcggtatggttggtggactcccattttcccagccaggatttcccatcgaacttgccgccgtgtttctctgccttatgatccgccacggtcgagacgaaaaaaccggtaacagacagggcaaggagaattgtgaggtgagccattatctagtgcttggaaaggctaatattaaatcaagcaaggtgggatggaaatgatgtgaggcaaaattctgaacgaaactgaaggtattatgatgaaagtatactatacctcgacatactttcgtaggttggtgaaagtatgtcgagggtgaaaagttgggggatgaaggggcagtgtactactgtgcatgcaggatcggattacagtcctttgtgtcgcgacggggtgaacaaaagagaggctcaagacacgataattgaaggcaacaaaggctcatttaatccccatgtcgtctggagaggaacaaaggaaacttttcccgataacttcttctcgaaaattttctacagagatctctcatgtagacgacaaaggaagcatactagtaataaggacagaaaacgaacgaataaacccaggacgaaaaaatatccaatcttttctgaacttcaagctaccatctgaaaattaattaactctggagcgtcaccatgggacacgtctgtttgattccttccttgtgggatttttacccactaacgaattaaggatttataatgttagttgtaagaagtctatgagttcaaatttcccgcttgaat

SEQ ID NO: 197 - DNA, 1613 bp, Hypsibius dujardini
ccatgacgaacgtcctactcccgggagttaattaattttcagatggtagcttgaagttcagaaaagattggatattttttcatcctgggtttattcgttcgttttctgtccttattactagtatgcttcctttgtcgtctacatgagagatctctgtagaaaattttcgagaagaagttatcgggaaaagtttcctttgttcctctccagacgacatggggattaaatgagcctttgttgccttcaattatcgtgtcttgagcctctcttttgttcaccccgtcgcgacacaaaggactgtaatccgatcctgcattaattatacatgcacagtagtacactgccccttcatcccccaacttttcaccctcgacatactttcaccaacctacgaaagtatgtcgaggtatagtatactttcatcataataccttcagtttcgttcagaattttgcctcacatcatttccatcccaccttgcttgatttaatattagcctttccaagcactagataatggctcacctcacaattctccttgccctgtctgttaccggttttttcgtctcgaccgtggcggatcataaggcagagaaacacggcggcaagttcgatgggaaatcctggctgggaaaatgggagtccaccaaccataccgagaacctcgagaccttcgtcagccaacttggttatccgtctgcggagcacgtgacggaccagaaggtattccaaaagttctggcaggatggtgagcatttccatcataagatcaccgtaccgaccaagaactacaccttgcaacacaagttcacattgggccagccgggaaaggcaactttcaacaatgtcgaattcaagtacctctacgccgagctgggaaacgacctccacgttgagatcaccgtgccgagcaagaacaagaccgtcagtgacacctaccatgttttccaaaatggaaccgagttggaaaagacctacaaaacaggagacacagtggccaagcgttggtacaagaaagtgatcagctgccactgaggacgcccgtggaattgcaatattttcataaatattgcctgggcagtacttgcttaagctgactaccgcatttgctattttatacttctttttctttgtatctggaccttaatttgtttctggtatttaaacccatcaatatcactattcttagacgctgacgccaaattgaatgccgacagctaattattcacttgtgaatgaactttttgaagctgcagccaatcgtttggaagtacagatagacagacagacagacattgactcagagaaagacagacagacattgacggactatttcgggagtcattcgggaaggctttaaaactgcttcgaatgaggggggaggtggcagggaagagggatctccgctgtaacgtggcgctgctgggtatcgtcgctcaattttccgcgacagtttcccacattttccgtccgatacaaatacacactaagacacatatatatatatccccatatacacgcacattaagggagatatgaatgagacgtgggggaggaagaagtggatgcggtgatgggaaatgagaaccacccggtagtcttcgcggttagttg

SEQ ID NO: 198 - DNA, 961 bp, Hypsibius dujardini
ctccggaattcaaggccatttgtttgattcttcgatgattaaacgcccacaaagatgtaacattgcacatttcaatgtaatcccactcccagtatagaaagaagctcaataggaaaaatgtaaccacttcattctaaatatgcatctatttggacacagcaagcgaacggctgtttaaacagcttgccttttcaagtatcgcttcgcgacgattccgtcgattttgtatgtcttaacgagctcatcgccggtcacttcgtaaacctcggtgacttccttgcccctagcggggatcttcaccgtcaccagcagtttctcgccctcctcggtgaaagtgtatttgacctcggtgttctcaaacttgggctccaccttggtgccttcctcgccgatcttgaaggtccacgtgctggagaagttgttggaggggacttccagtttgctggtgtaggtctctccctcgcgagtgatgctcagagtggatttggggttgccgccgtacgctgcgggcagaccaagagccgccacgacctgatcccagttctcgggcgccttatcggtggtggtccagcttccgagccacggctgcgaggccgatgcaacagcgacgacacccataagaacgatgagggacagacgagccatctttacagcgtggattgggggttttaggaaccgggaaaaaaaggaaggacgactaggtacaaggcaaaaaaactggtcttgacgactggacaacagtcgtttttatatgagaatttcgcgcgaaaagccatccaggggtggtgaaggccactttcaactcgcatgttcaaggtcgttccggaaggacaaagtttcttacttagcttttccttcatgagaccggaaatcagcgtagttcaatcagagtgaagtcgatcgcgactgtaccagacaaatgtaatgacataggcaatgctacagattcagacccaaatga

SEQ ID NO: 199 - DNA, 1077 bp, Hypsibius dujardini
tgtgcctgtgtctgggtctgggtcttcatttgggtctaaatctgtagtattgcctatgtcattatatttgtctggtactgtcgcgatcgacttcactctgaaccgcgctgatttccggtctcatggaggaaaagcttagtgagaaactttgtccttccggaacgaccttgaacatgcgagttgaaagtggccttcaccacccctggatggcttttcgctcgaaattctcatataaaaacgactgttgtccagtcgtcaagaccagtttttttgccttgtacctagtcgtccttcctttttttcccggttcctaaaacccccaatccacgctgtaaagatggctcgtctgtccctcatcgttcttatgggtgtcgtcgctgttgcatcggcctcgcagccgtggctcggaagctggaccaccaccgataaggcgcccgagaactgggatcaggtcgtggcggctcttggtctgcccgcagcgtacggcggcaaccccaaatccactctgagcatcactcgcgagggagagacctacaccagcaaactggaagtcccctccaacaacttctccagcacgtggaccttcaagatcggcgaggaaggcaccaaggtggagcccaagtttgagaacaccgaggtcaaatacactttcaccgaggagggcgagaaactgctggtgacggtgaagatccccgctaggggcaaggaagtcaccgaggtttacgaagtgaccggcgatgagctcgttaagacatacaaaatcgacggaatcgtcgcgaagcgatacttgaaaaggcaagctgtttaaacagccgttcgcttgctgtgtccaaatagatgcatatttagaatgaagtggttacatttttcctattgagcttctttctatactgggagtgggattacattgaaatgtgcaatgttacatctttgtgggcgtttaatcatcgaagaatcaaacaaatggccttgaattccggagttggctgccaagttttacactttctatttggtatcaaccgtcatcacagccataataaaacttcttgttttcaaaaaaaaaaaaaaaaaaaaaaa

SEQ ID NO: 200 - DNA, 1602 bp, Hypsibius dujardini
tttttctggtgtacccttgttattttatttgtctctattcctatgtctggccaaatacaaagaccccaacttaaaagagtacttggctcgagatggtttcctacgacatgaatcgtaatcaatgattgaacctaatcccgtttgagtaaacggcgttataacaaaacaacacttgacgaattatcacggaatatagtacagacaggaagggaagggtaaaaggccgatataaattgccgttaaggttggctcatgttcgatttggtttagacattacagacaaccaaaaggcttggctcagctgaataggaatagccctcccccgtttcaggaagattggtcccttttcaagaaacgcttagcaacgattccatccatcttgtatgttataatgagctcttgaccgaccacttcgtagttttcgttgtattgtttgccgatggcggatattttgacgtgggcttgcagtttgtctccgtcctcagtgaagttgtacctcatccccgaaccaaactcatccataaccatttcctcgcccagccttaagttccaggtgctagtgaaattgattgcgggcacatccagtaagaccttgtagttgtctccgtcgtcgcgggtgatggtgatggtggcttttgggttgccgctaaactgctccagggggaggccaagagctgccacaaactgatcccagttctcaggagccttctccgagttggtccagacacccaaccacggctgtgcagccgatgtaacagagaccataacgaagagggcgaggacaaggagaatccgagacatttttatgcgcctatagagaaatttatattggcaatcgtgaaaagagaaaaagtagggtaaaatggaaaagctttagtaagagtgggagaatgatttacggccacaagatttgagggcaagattggttggttgtctgtttgagcccgctgcggcgatcgattgcgatcagtggctgaggcaaagattcctccacaagatgtacgatgaatttggcataccttccaaccaagccctttttctattctaatagggaccgcggtctgtagattgaaataaactttaagtggagctgtttcatccacaattcagtttggaatatagtaagacatgggtagttgtggatggagctagtcatcgtttcaacaaaatgcacctacctctgcccctgcacctccgacaaaattatttggccagaatgccgacatacgaaagggaagaagccacaacagcagccccttgaatattgtacatgcgattgctttgttgaaagcggggacttgtttttgttgagttggaacggacggttgacagaccgcagtctatgttaaggtggccatggtttaataccaattccaccgtcaaagctcgagaacggtcccgacgatgatagtgtatcaacggctccggctccccgtgcgaaaaattgtccgtcttatgcggcgttgttaacgccgttctgggactgaagtgaccatcttctcgctctggtttaggccgtcgattcgaccgtaaataactgaacaaacacacagctgattttggctcccactgcggaagctgtcggcgttatgtagactttac

SEQ ID NO: 201 - DNA, 1022 bp, Hypsibius dujardini
aacccatcaaaacccatcaaaactatacatgaccctctgaactgtcaaagaagtctgtctaaacctccattgcctttacggattacgtaagaaagacattatggtatcattctgggaagccatgaactagattattactcctctttactaccgctgccgcacctgcttcggtacatgtatatgctgttggatttcctgtccgtcccaaattaccttttccactctgcgtcagcaacctcaaagatttagtttttctttcttatattcttgcactttcacacttcgagagtcctgaccgtgaacctcaaaaagttgcataagtcatggcagcaattgatcccacaccggcaacagtattgagtgtccagcaagagaattgcaggccatggctcgggatgtgggtttccgctggaaagaaagaaaactggcccgcagtcatggaagcgttaggtttgccggagatgtattctgagaaaaacactttcgtcctcaaattatggtgcgacggagaggactttcactatgatgccggtattctggaggcaaagtttaagcacagtgtcacgtttaagctggggactcctactgaacttaatcacgggaacaaaatcgtcattacctacaccgaagaggacggcaagctgatcgcggacggagtaattgcggctaagaatctgattttgcacaacgtattcgcggcccagggagacgtgctgatcaagacctatcgtgtggggaatgtcgtagccaagtcatggtaccgcagactttcctcaacggccgactcaaacattttatcctttttgtgaggagccaattacagcaaaagagtttcgtatgtacattttgtccgttgcgggatttcatgatgcagttgcccatgaaagtactgggcactggcccctggggcgtagtcgataaagttagaagttattgctccgttgcgcactacatttacaagggccggcgacgtaatgtattacgctatggcgatcctgtgttccgttaactccctttcagtg

SEQ ID NO: 202 - DNA, 993 bp, Hypsibius dujardini
cagtcgcacgcaacttcactgtccgtgcatgctggcacatcgatctaggtccatctagataaagtggaccgaccggaagagacctagacttgtccacgcgcgccatgggaaaaagacagtttccggcaataaagtgtctgtcgaattttttcctacgtcctagacgagcttttcagtactggggttcttcgacatttctgtaatttcagtttctttttctgtccactctcgcagtcaagcagttcagccatggagttcgcggcgtccatcttcgttctctgcttcggtctctcggctgtcacagcagccggtttgccgtttgtcggacactatgtgtcgacgggtcagcgctttaacactgctgcgtttgccgcggccaccggtttcgatgatccacccgtggaaaaccggctgcacaacgaattcctggaccaagggaacggcgagtacctctacaaatttcgcgtcgaaaatgccgcctataagcaggagctgccgttcaaactgggcgagacacgcaagtccacctacaacggaactgaattttcgtataaattcaccgtcgatggcgagctgctcaaatttgagtcgaagatccttcccgacggacgcgaagtcacccacacttactatcccaatgccgacgggttcgttaagcaattccaactgaaggacgtcatcgccaaggtgtggttcaaaaaggactctgcatagatgggatcaacagacgattgatgatccgacagcagactaccttttttagcttccttcgaagtatgagtataactaaagaaattggatgtactgagaataggagaagttattacagctcaagaggtcaggtatatttgtccgtttgtattggctacgagttttaatataaactgcaacaccagctggctaagtaaagaaatctataaatgatacagatcttatagtgaaatatgagtgtatagagcatagctatcgttgtgcgagaaggttacacacagg

SEQ ID NO: 203 - DNA, 2191 bp, Hypsibius dujardini
gttgttttgatgatgcagttgtatattcagcacaattacttttctatctttggcttatccaaatggttgttgtttgcctgtgtgtaaccttctcgcacaacgatagctatgctctatacactcatatttcactataagatctgtatcatttatagatttctttacttagccagctggtgttgcagtttatattaaaactcgtagccaatacaaacggacaaatatacctgacctcttgagctgtaataacttctcctattctcagtacatccaatttctttagttatactcatacttcgaaggaagctaaaaaaggtagtctgctgtcggatcatcaatcgtctgttgatcccatctatgcagagtcctttttgaaccacaccttggcgatgacgtccttcagttggaattgcttaacgaacccgtcggcattgggatagtaagtgtgggtgacttcgcgtccgtcgggaaggatcttcgactcaaatttgagcagctcgccatcgacggtgaatttatacgaaaattcagttccgttgtaggtggacttgcgtgtctcgcccagtttgaacggcagctcctgcttataggcggcattttcgacgcgaaatttgtagaggtactcgccgttcccttggtccaggaattcgttgtgcagccggttttccacgggtggatcatcgaaaccggtggccgcggcaaacgcagcagtgttaaagcgctgacccgtcgacacatagtgtccgacaaacggcaaaccggctgctgtgacggccgagagaccgaagcagagaacgaagatggacgccgcgaactccatggctgaactgcttgactgcgagagtggacagaaaaagaaactgaaattacagaaatgtcgaagaaccccagtactgaaaagctcgtctaggacgtaggaaaaaattcgacagacactttattgccggaaactgtctttttcccatggcgcgcgtggacaagtctaggtctcttccggtcggtccactttatctagatggacctagatcgatgtgccagcatgcacggacagtgaagttgcgtgcgactggccaacatgcgttgaatttttcttgactggaagacccagcaatgcttgtcactgcgttgatcaaacagtacacagtacattcagcatgcttcggggattggacgcagtgacctctttacagacgcgctcatcgcatctttcggacgtaaaagaagatcccggtcgttcgtgtggtgttctttattggtacttgcagctgtaaaggtgctatcccttattctactccggaagctcctataatgcgccggtgtttatgtatgcatcgtgaatctcatccatgccgtgtataagcgctagtatagtagtaccttgggaaaaaagtgaggcaagagaatcccacgtaaatactcctctcggcctttgctaaagaccgtcacttcacttatcattagtctctcgtttttacatctcaccaatctcttagaccattgatctttggtctccttattgtcaatcagtcacacagatctctacgtgtagcattaagatgagttcgagaatagtgttctccgtggcgatggtcatcatgaccttctgcgtgattctgggatcgtgctcgcgctacctccccactaaacgcagtcccagtccgccgacgttgcaggactacgaccaggacctctactcccacggcggtgctgactcgacgttcaatttacccggcgcggatttcgacccggtgtattcaatcagcggtcggcccaaattgtcggacgtggtgacggttccggtacttcgacgccctcgcaatcagccggaagcttactacagccgttggtaatgcaaggaagacaattgggatcgcacgtctgactggcgtgacctttagtgcaacaaccgcggtggcacccagcaaagagtatatccaatccgaaacttgattccagaatgttcaaaccttgtgtccttgcttgtgtgttcgttgcatgttcgttgcatgtccgttttgaagtcgttgtgaaatctgtgttgcgttgacattactgcattacctacaccggcttttcttcataccaaaatgaatcatacatattataatatactaggtagtaaaacgccatgtcataatgatcgcagtatgtgcgaataatgtcgaggttaaatctggggaacacaaaaaaa

SEQ ID NO: 204 - DNA, 992 bp, Hypsibius dujardini
tctgtccttactgtactgactgactgactagcaccctccgaagaacctcaaagacccaatatatcctcaagataacgattaccaaacatccgtcccattccctccacgttaaatttgtgcttgtagcaataaaagataacattataattattatttgtctgtcatccgtcaattgactgtcaaaggtcgaatctggattattgttagctgccttggactgcacgtgtatcaacccgcaaaatatttcgaggcatgatcagtttatttttgttattcgcagtaggtgggctcgcggttgacggggcgcttccacccggcgaggttgcagccgtgttactaccccccagcatggtgaatatcataccggtaccactgggagagtttgttcccactgggcagaaggaaaattacgccaactacgtgcacagtttggagtttgagttccgcgggctggctgcgcagggtattcttggagacaagggcaaggatgtgcggcataaattttcacggagtgccgatgggaaggagaactcgtacgtccacaagttcggcaatgacggtggtggcaaatacaaccacaccgtgccgttcgtgctggacgaggagaaactcgtccataccaatgcgacatccttgaagtacaagtattggttcgagcccggacaaggacttcatgccgactacaacatcccaccggagaatcccctgcagattcagcatctttatgccgtgacagacgagggtttcacactgatctacaagctgggaaatgtcattgcaaagaactattacaaacgtgcaccttcatcggatgctgcaccagaagtcacgtctaagacaaccgttgctccgatcaccacaaagaaaaaagcataattcctacgaggctatacacgtggaaaaaacccttgcgctgaccgaatcaggcactttaactcgggattgtgtacatcagccattgcgtcgatcgagtgtaatggcttaactcggtt

SEQ ID NO: 205 - DNA, 1041 bp, Hypsibius dujardini
aaccgagttaagccattacactcgatcgacgcaatggctgatgtacacaatcccgagttaaagtgtttaattcggtcagcgcaagggttttttccacgtgtatagcctcgtaggaattatgcttttttctttgtggtgatcggagcaacggttgtcttagacgtgacttctggtgcagcatccgatgaaggtgcacgtttgtaatagttctttgcaatgacatttcccagcttgtagatcagtgtgaaaccctcgtctgtcacggcataaagatgctgaatctgcaggggattctccggtgggatgttgtagtcggcatgaagtccttgtccgggctcgaaccaatacttgtacttcaaggatgtcgcattggtatggacgagtttctcctcgtccagcacgaacggcacggtgtggttgtatttgccaccaccgtcattgccgaacttgtggacgtacgagttctccttcccatcggcactccgtgaaaatttatgccgcacatccttgcccttgtctccaagaataccctgcgcagccagcccgcggaactcaaactccaaactgtgcacgtagttggcgtaattttccttctgcccagtgggaacaaactctcccagtggtaccggtatgatattcaccatgctggggggtagtaacacggctgcaacctcgccgggtggaagcgccccgtcaaccgcgagcccacctactgcgaataacaaaaataaactgatcatgcctcgaaatattttgcgggttgatacacgtgcagtccaaggcagctaacaataatccagattcgacctttgacagtcaattgacggatgacagacaaataataattataatgttatcttttattgctacaagcacaaatttaacgtggagggaatgggacggatgtttggtaatcgttatcttgaggatatattgggtctttgaggttcttcggagggtgctagtcagtcagtcagtacagtaaggacagatttaatctaatggagggtcccgaaaatgggcaaaaggggcggagcaggg

SEQ ID NO: 206 - DNA, 711 bp, Ramazzottius varieornatus
atgtccagatacctgctgcgcgatgtccaggctgtattacgcggagttcgcaaagtggccgagagtagcttaaagctggagacggagaaagtcagtctgcggcttggtgactttcggtcacagccttcccttcgcagtgtgcctgcttccctcacaagtcgatcacaggcatttagcctacaggagatagctgctcgtgccggagttgttctgcgaggagtgcaacaacagttccgaaacgtcactggagtgaatgccgctcctgttgtagcctttgataatggatcagttctatacagtgaaagaatccactcgcagagttcgcagaagcaggccccgactacagtaccaacaggatccgtcagcaattcccctcaaccggaaggaaaggcaaacgaagctgctgaacgcgcaaaacagtttatgaatcctccagttgcgccaatggatcctgtcgacaagaatgaatttgtcgccatgccggagatgggtcgtagtaatggaaatggagaaaacaaacaagctgctgatttcatgaaaaaccaaggtgacaccgatatggattcccagtacgcgcctgattcatcgaagaacacgaaatcggttcccacgaaggaaatcgttgctgaagatggttcgatgagcattgaggatatcaagaaagctacgcaggttactcctggagttgcagttaaaaacgagggtgtttag

SEQ ID NO: 207 - DNA, 1392 bp, Hypsibius dujardini
ttttttttttcgtatttgcattatattactacatctcgacccgactccaggggcactcataattatttttttataaacgtttattaacaaaaaaaattagtcgaaagcccagtcgaaagtggcgaaacctaactacagtaaaaaacggatcatacgacccgggatggaaaaaactggaaaatgtgctgcaccgcaggcgcaaaaaatgttcatgtacaagggtcccgacggcgacaaacggtgaaagcgggaccgaaaaaataaatataatgcacactatttaccgtctgtttctcaacagtcggcaagataaccggcttcgctgagctgggctttcgtccgtctccgccgccgcttccggcaagtgacgagtaaaatctctatatacacagcaacagagagcgcgctctaagaaaagctgtcagccttgactgggacgcccggtggaacctttgaaattttcttcaaattctccggtcggaataagttgtccccgccgctgtcgatgaaggtcttcttggagtccttactgaagcgatcctgcttgaggtgggacgtctcttccttgccttcagcctccgcagcgtccaaacttccgttattaaagttcttcaggttgtgagagtcatcaccctcgagcgagttattagcaagaactgagacgtcactttcgtccaatggtgcaaccggtgggttcataaatttctccgcgcgtttcgccgcctttccggcctcgccttccggcttgccatgtccattctcgtcgtcaatatccgccgtggtcggatggtcgcgtgtggccctattctgtgcgcttccaattttgtcgctgtagagggtaaagccgttatcgaaggccaaaatgggagccggggcctggatgcccgcgacgattttcacttgctcctccagcccacgcagcaccgcaccggcacgcgacgcaatttcctgtacgttgaagctggaccgcggggatgaagacgacgcaggagagctcgcggaagagctcgcaagattgctccatcgcggtcgcaggctggattgacacacacgctcgttgatttccgcggcttggattttcagactgatctgagcgacctgcttgatcccacgcagcatggcttgcatgtcgtgcagcaaatatttggccatcctcggagggaattaaacaaaacggtgcaaaataaaggatcctcctctttgcagtgaggtccacgagaaaaacctcccgcaaattcctaccctgtgattgttcagttcaacaactgcggattttgcgaaacagcaatgtgtgaagatgtcgaaagttttccactggtaaaatgttgattgacgatcggctgatctgttttcccggaaaacacgattaataccgaggcgaacaagtttgaa

SEQ ID NO: 208 - DNA, 1441 bp, Hypsibius dujardini
atcgtgttttccgggaaaacagatcagccgatcgtcaatcaacattttaccagtggaaaactttcgacatcttcacacattgctgtttctcaaaatccacagttggcaagtcaaaatcattctttttttgcagtgcaaataccagtagaggtgctaaattttttgtgttttcctttcagttgaactgaaaaatcacagggtaggaatttgcgggaggtttttctcgtggacctcactgcagagaggaggatcctttattttgcaccgttttgtttaattccctccgaggatggccaaatatttgctgcacgacatgcaagccatgctgcgtgggatcaagcaggtcgctcagatcagtctgaaaatccaagccgcggaaatcaacgagcgtgtgtgtcaatccagcctgcgaccgcgatggagcaatcttgcgagctcttccgcgagctctcctgcgtcgtcttcatccccgcggtccagcttcaacgtacaggaaattgcgtcgcgtgccggtgcggtgctgcgtgggctggaggagcaagtgaaaatcgtcgcgggcatccaggccccggctcccattttggccttcgataacggctttaccctctacagcgacaaaattggaagcgcacagaatagggccacacgcgaccatccgaccacggcggatattgacgacgagaatggacatggcaagccggaaggcgaggccggaaaggcggcgaaacgcgcggagaaatttatgaacccaccggttgcaccattggacgaaagtgacgtctcaattcttgctaacaactcgctcgagggtgatgactctcacaacctgaagaactttagtaacggaagtttggacgctgcggaggctgaaggcaaggaagagacgtcccacctcaagcaggatcgcttcagtaaggactccaagaagaccttcatcgacagcggcggggacaacttgttccgaccggagaatttgaagaaaatttcaaaggttccaccgggcgtcccagtcaaggctgacagcttttcttagagcgcgctctctgttgctgtgtatatagagattttactcgtcacttgccggaagcggcggcggagacggacgaaagcccagctcagcgaagccggttatcttgccgactgttgagaaacagacggtaaatagtgtgcattatatttattttttcggtcccgctttcaccgtttgtcgccgtcgggacccttgtacatgaacattttttgcgcctgcggtgcagcacattttccagttttttccatcccgggtcgtatgatccgttttttactgtagttaggtttcgccactttcgactgggctttcgactaattttttttgttaataaacgtttataaaaaaataattatgagtgcccctggagtcgggtcgagatgtagtaatataatgcaaatacgaaaaaaaaaa

SEQ ID NO: 209 - DNA, 1399 bp, Paramacrobiotus richtersi
cgttcgtacaatttgtttacaacttcgtgacttccgatgtttaaagttcgacgtaaattacaagtgtggtagttattgcagccagaatttttcgattaaaatagttgacgaataccgcagcaaaccagtttttcctcgatactgaagtagtggtctttaatttggcttgtgtttgccatagtgatctgttttcgaggttcctagagatggcacgattcatgataaaagatttgcaggcggtatttcgcggtttccagcaggtcgcgcaaagcagcgtggagcatcagctcaccgaaacagctctccgatggcatacgctgagtctgcgcccactggttcaaggatgcgtgaaccgaatgcaagaatctcagaggtcgaccgttccgctgcgagaattccctgcccgagtgggagctgtagtgcagggtattcaagagcagatgaagatcttggcgggtttcccctctcccgctctggtcacgccggagggattcgtcttctacaccgataaagtcaataaagatacccataaggaatatcccgctgtggctgatgaggtacactcggccaaactgcagggactgaaaccggaaagcggggaagcatgtgaagcggccaaacgtgccaaagagtttatgaatccaccagtgtcgccactggatccggaggataaaaacgaggttgtacgcacaccggaaatgtctggttcgaccgctgctgaggatcagaatgctgacgaatccggcaaagctgcgaagagactaggaaaattcatgaatgaggagatcgcacctgaaagtaagcccttcaaaccgtttgccaaagattcagccaagaccacagtgtctttcacggacgctacgggcgaaaacttccgcatacaggatttgaaaaaggttcaagtttttcccggcacacccgttgccttcgagagttgattatgcggaagaaatataaccgtatctgtaacccaaatgcagccatccgcgtcagcgccatcctcatcacgtcccgtagataattcatccgactcggccagttacaagcaacccctgcaggaacatactctgaaacagacggtaaactttaacatttgcacaagagctttccggtcgcgcttcaccgttttggatgcgggttaattcaattaaaaaatcgccattttgatgttctttttgtagtttaattataaaaaagcatacagtgcagtctttggataagatttcgagtgcctcagtttcctgcacgagaacatgtgtagttgtacatggtctgactcctgcgctgccaaaaatgattgttgctgttttgctttggctttctttattcatgtttatagtattttatattagagttattgtttattaataaatggaaggcttggaaaaaaaaaaaaaaa

SEQ ID NO: 210 - DNA, 1449 bp, Milnesium tardigradum
gtggccattacggccggggagtacaagacagatgtggctttagctgtgctgattctctgacgatttgttgttgttttctagagatcgaagagaagcctcagaagactcgctttcagtgcatcgtaaatagcactttttatgtgattcaaatcgttgacacgtgaattgcatcactattcactcaagtcgtgcagaaagcatatcgaaaaatgtcgcgatatttactcaacgatatggaaggcatcatcagaggtcttcgaagtgtcgctacaaatgcggcagcacttcacagaactgacctctcgacgcgtctgcagaactgcacatttgcagctcagtcgtccaacgcagtgccgtcactgttgcgtcaaatgcagaaagtacgaccaataaacaccacagacttcgtctcacgtacacgcacggttttacgaggtttgcgcgatcaatcgcagtccctcttcggctcttcgcagtctatccgtcatacgtcaggcacagcatcacccacaactaacgtcgtaacgaaaactgagaaagaaatgaaagaagcacagtcgaaagagcgaaattcgaaggatactcactcgaaagacagttacaacaaagatgcgcacatcacgccctccagtgactctaccgcttcaaacgctgcttccaacaaaagagacgcaaagaaaagtgatgacacaaatggtggagcgcgaatggtcgacgaaggcgcttttaacaacgaaaaaccaatcaaacagagctctgctaaggaccacagagaccagtcaccgcataaccgagaattggctgatgtcgaaaggaagtcagaagtggaaatgccagaggactctgaagatcagaagctaacagaggccgaacaggccgcaaagagaatcgaagaatttttgaacggaccgaaatcacctgaagatcccgcctctaaagataaaatcgtcgtaacaccagaaatgacgaaacatgaagagccgataccagaatcgaaggcagttcaggaaatatggatagaattttgaacacgagaaccacgtataaaactggaatacccacaggaaataaggacgtataaagtgctgtacactgcgagtccgtcgataccttgagctgcgctgcgctctgctgaaattgtgattttttcgacttttttgcttacgaaagacactgtattttgtatcgtatcttattgtgtaaatgagagttcagcagcagtgctcttaatcattagtgtaaattatggaataggattgtctagttcttgttcagacgtacaatggtccagtcgtcacttgatcaagacagttccagtctgtaattgcacagtaatcagcgcataagaaactgataagcttttcccgaaaatgctataaattttacttttgtacatgcgaaaatgggacataaaaactgaatgaatacaaaaaaaaaaaaaaaaaaaaa

SEQ ID NO: 211 - DNA, 801 bp, Paramacrobiotus richtersi
atgtccggacgtatcgagcaacacatggaagcggaggaatgccagggcggtgcctactgcccgcccaactgccgctaccacagtcgcgggatgaagcaggaacatgaggagaagcacgtgtaccgagagagtgtcacacctggtcatgcagaacggcgggaggaacgaagggatgagcagtatcagcggccgtctgagtcgtatcccgagtccaaccgacaggtcgagaaggaggcggttaacactgcccgtgttcacaccacggtgtcggcgccgattgtggcgccccctgccccggttatcagtgttgcgcccgttgcggaggagctggcttcgggttacacaggcagcgccgctcgctataccgccagcagcgaggtcaccatcctccccaaccccaaactgaccgaagaggcccgtcgcgatgagattgcccgccagaaagaggccgacgacattgcccgtcgacatgaacaggacctggccaaacgcagtgaacagtaccgtaagcaggccgaagccgaagcggagaagatccgcaaagaactggaaaagcaacacgacaaggatatcgccttccgcaagagtctcattgactcggccgtcacacggcaacaacgcgagattgacctcgaagccaagatggccaagaaggagctgctgcgcgaggcggaacaggctaaagagtcgctggagaagacgcgggcagccacgaccgtggaggtcgattttacgactgctgtgggccacacacattccgctggagtaaccgcatccgagacaatccgcaccaatcagtaa

SEQ ID NO: 212 - DNA, 804 bp, Paramacrobiotus richtersi
atgtccggacgtatcgagcaacacatggaagcggaggaatgccagggcggcgcctactgcccacccaactgtcgctaccacagtcggggaatgaagcaggaacacgaagagaagcaggtgtaccgggaaagtattacgcctggtcatgcagaacgccgggtggaggaacgcagggatgagcagtatcagcggccgtctgagtcgtatcccgagtccaatcgacaggtcgagaaggaggtggtcaacacagcccgtgttcacaccacggtgtcggcgccgattgtggcgccccctgccccggttatcactgttgcgcccgttgcggaggagctggcttcgggtttcacaggcagcgccgctcgctataccgccagcagcgaggtcaccatcctccccaaccccaaactgaccgaagaggcccgtcgcgatgagattgcccgccagaaagaggccgacgacattgcccgtcgacatgaacaggacctggccaagcgcagtgaacagtaccgtaaacaagccgaagccgaggcggagaagattcgtaaagaactggaaaagcagcacgacaaggatatcgccttccgcaagagtctcattgactcggccgtcacacggcaacaacgcgagattgacctcgaagccaagatggccaagaaggagctgctgcgcgaggcggaacaggctaaagagtcgctggagaagacgcgggcagccacgaccgtggaggtcgattttacgactgctgtgggccacacacattccgctggagtaaccgcatccgagacaatccgcaccaatcagtaa

SEQ ID NO: 213 - DNA, 837 bp, Paramacrobiotus richtersi
atggagaggaaagtcgtagagaaaatcgaagtccacacaaccggcaacgtgccacctgtgatccctgccgcctgttccaacgtaacctgtaccacggtctgcgatcccaagtgcacggaacgccatgaacaccaccatcatactggcgtcgcctgcaacaccgtctgcagtccggcatgtggtgagcgtcatgaacatcaccagcatcaccagcatcacgaacattccggcagctgcaccgagacgtcggagaaatcgacgcactacacccataccgaagtgaaggcacccgttctcaacccatcggctccgtttgtggtgacgtccgcctccggactagcgcaagagatcgtatccgaaggcttcagcgcatcggccgccagaataagtggtgagtctgtgggcactatagtccatgagtcggccgcttcgtcgaaacaagccgcagtcgatctggagaaatatgagcgagaaaaagccgccattgccaagcagcatgagaaggagctggaaaagaagaccgagagctacaggaagcaagcggaggccgaagcggaaaagatccgcaaggaactagagaagcaacacgcacgggatgtggaattccgcaaagatgtcctggagaccaccattgagcgccaaaagaaggaagttgaactggaggccaaaatggccaagaaggaactcgagcacgaaaagaaactggcaatggacgcactggagcattccaaaatgtcgacgaatatcgaagtcaagttcgattcggccgctggacacacaacgacggagggtgtggttgtctctgaaagcgtgaacgttgcacacccacggatgtga

SEQ ID NO: 214 - DNA, 261 bp, Paramacrobiotus richtersi
cgcaaggagctggagaagcagcaccagcgcgacatcgacttcaaggaggagctggtcggcgaggccattgcggcgcagaagaagcaggtggatctcgaggccaacctggccaagcgccagctggagcgggagggacaggcggcaagggatgctctcgagaagagcaagatgaccaccaatgtggaggtcaactttgatacggccatcgggcacgccgtctccggaagtagcaccgtcgagtccgcggtccacaagacccac

SEQ ID NO: 215 - DNA, 261 bp, Paramacrobiotus richtersi
cgcaaggagctggagaagcagcaccagcgcgacatcgacttcaaggaggagctggtcggcgaggccattgcggcgcagaagaagcaggtggatctcgaggccaacctggccaagcgccagctggagcgggagggacaggcggcaagggatgccctcgagaagagcaagatgaccaccaatgtggaggtcaactttgatacggccatcgggcacgccgtctccggaagtaccaccgtcgagtctgaggtccacaagacccac

SEQ ID NO: 216 - DNA, 1641 bp, Paramacrobiotus richtersi
atgccactattcgggtcttctaagaaggataagcataaggacgatatcgtcgtcaccaaccaggacatcgatgtcgagcgtgaccgggactcggtcgtctcgcgtgaccgcgactcggtcgtctcgacccatctggaccgtgaccttactacagtacccggtgacaagcattcccacgaattcaagtatgaacgcgtggaagagatccacgtggacgccgatggtaacgcggaactgcgtgatgtccgcgttgaccgcggtggagaggatcccggcatgaacttcaaggataaacgcccaccggccctggttcccggtgctcccgtgggatacgtcccggagatccacgaactggacagcgtcgctacacagcgccagggcatccagaactactttgccgatagtagctcggtcagtcatactcaacgaaagagtcgtgaaccgtcgttgctggaagaaagggaacaaactattattcgggaaggaagtatggcgagtggggcatctatcaacaataacgccggtattccgccaacggtcccactggagcgtttttcacagcgctcggtatcacaatcaggggatgacacgtcatccattgcctcgtctgtgtcgtctgtatcatccgtgtcctcggcgtccactgcaacggctgggtcccgtgtttcccagaacacaaccggcacacgggatcgtgtcaacattgcccgacaggaagcgccggctttgcaacgcgatgtggactacatccaacagggaattgaaaatctacaaaatttgcccataatgaacccggctcgcgatacatacgtcacggagcgtcgtacggacgcctcatacgtccagaatatccctcccgtagtggagatgggtcgcgctcccatttaccgtcaagaacaggatattatcattccaggacgacaccgtgaagtgatcgaacggacggaggtcatccagtcggccgctcctcgccaggggtcggtcgaggtcatccagatccccattcaccgtatggaggctgcgcagatggagcacatccggtcgggtgtgacgtacaccaacgacaaggaattgatcattcccggtgcgatgctggcgccacccattcccagtgtgacgcatgaccttttggcccagggaagcggcgggacccatgcggaaatctatgctgatacgaatatcgatttactggccaacacacagctgaagcagtcgcctgaagaatatgcccgctatcgggccagtgtcgaagcgttggcccgtgaacatgagatggacaccgcccagcgggcggccatgtaccgtaaccaagtggaagcggatgccgaactgattcgtcgcactctggagcgtcagcacattcgggatattgagttccgcaaggaaatggtcgagacagcggtggatcggcagcagcacgagatccagttggaagccgagtatgccatgcgcgcgctggagaaggaacgcgaagcggcgtcacgcgctctggaacaggccaaggcgcagactcatatcgacgttagagtggataccgcgattggaacgaccatcagtaagggtgatgtacaaacagcagctggacgggaaatccgggagaatgtgggccgggtggaaagctatccagctacacgcttctaa

SEQ ID NO: 217 - DNA, 1617 bp, Paramacrobiotus richtersi
atgccactattcgggtcttctaagaaggataagcataaggacgatatcgtcgtcaccaaccaggacatcgatgtcgagcgtgaccgggactcggtcgtctcgacccatctggaccgtgaccttactacagtacccggtgacaagcattcccacgaattcaagtatgaacgcgtggaagagatccacgtggacgccgatggtaacgcggaactgcgtgatgttcgcgtcgaccgcggcggagaggatcccggcatgaacttcaaggataaacgcccaccggccctggttcccggtgctcccgtgggatacgtcccggagatccacgaactggacagcgtcgctacacagcgccagggcatccagaactactttgccgatagtagctcggtcagtcatactcaacgaaagagtcgtgaaccgtcgttgctggaagaaagggaacaaactattattcgggaaggaagtatggcgagtggggcatctatcaacaataacgccggtattccgccaacggtcccactggagcgtttttcacagcgctcggtatcacaatcaggggatgacacgtcatccattgcctcgtctgtgtcgtctgtatcatccgtgtcctcggcgtccactgcaacggctgggtcccgtgtttctcagaacacaaccggcacacgggatcgtgtcaacattgcccgacaggaggcgccggctctacaacgtgatgtggattatatccaacaaggaattgagaatctacaaaatttgcccataatgaacccggctcgcgatacatacgtcacggagcgtcgtacggacgcctcatacgtccagaatatccctcccgtagtggagatgggtcgcgctcccatttaccgtcaagaacaggatattatcattccaggacgacaccgtgaagtgatcgaacggacggaggtcatccagtcggccgctcctcgccaggggtcggtcgaggtcatccagatccccattcaccggatggaggccgcgcagatggagcacatccgttcgggtgtaacgtacaccaacgataaggaattggtcattcccggcgcgatgctggcgccacccattcccagtgtgacgcatgaccttttggcccagggaagcggcgggacccatgcggaaatctatgctgatacgaatatcgatttactggccaacacacagctgaagcagtcgcctgaagaatatgcccgctatcgggccagtgtggaagccttggcccgtgaacatgagatggacaccgcccagcgggcggccatgtaccgtaaccaagtggaagcggatgccgaactgattcgtcgcactctggagcgtcagcacattcgggatattgagttccgtaaggaaatggtcgagaccgcggtggatcggcagcagcacgagatccagttggaagccgagtatgccatgcgcgcgctggagaaggaacgcgaagcggcgtcacgcgctctggaacaggccaaggcgcagactcatatcgacgttagagtggataccgcgattggaacgaccatcagtaagggtgatgtacaaacagcagctggacgggaaatccgggagaatgtgggccgggtggaaagctatccagctacacgcttctaa

SEQ ID NO: 218 - DNA, 897 bp, Paramacrobiotus richtersi
atgtcgcatcatcacgaagagaaatttgagcgtgtggaagagcgcaaagtggatccagcccgtggcgtgcaggaggtccgcgtcggtatggacaccggtcatggcgatccagccctcaacttccagcccaccgatgccactttggtcaagggcaggacggtcgttggcggagtggatgctgcgggcatgacgaccggagcgacccagtattccggtgcctctacggtgcaatcgggaaccaatacctttgaagccgagaagaatacttcctacactcataccgaagtgcgcgctccacttgtgacccccgccgctccgttcatttccacgggagtgactggcctggctcaggatgtcgttggtgaaggtttcacggcgtctgctgcccgcatcactgccggcagcgcgtccgccgttgtgaccgagaccgctgagatgcgtgacaagtcgatgaaggagcaggaacgttatttgcgcgagaaggaggccatcgctcgatctcatgagaaggatctggagaagaagactgaggcctaccgaaaggaagcggaggctgaagccgagaagatccgcaaggagctggagaagcagcacgcacgcgacgtggaattccgcaaggacatggtggatgagaccattgagcgccagaaacgcgaggtcgagctggaagccaagtacgccaagaaggagctggaacacgaacgccagatggctcagaatgccctggaccagagcaagatggccaccaacatcgaagtgtccatggacaccgctgccgggcgcaccgtgagcggcggtaccaccgtgtcagagtcctttgagacccaccacgaagagcacggtaaggagaagaaatccctgggcgagaagatcaaggacacctttttgggccgttaa

SEQ ID NO: 219 - DNA, 897 bp, Paramacrobiotus richtersi
atgtcgcatcatcacgaagagaaatttgagcgtgtggaagagcgcaaagtggatccagcccgtggcgtgcaggaggtccgcgtcggtatggacaccggtcatggcgatccagccctcaacttccagcccaccgatgccactttggtcaagggcaggacggtcgttggcggagtggatgctgcggggatgacgaccggagcaacccagtattccggtgcctccacggtgcagtcgggaaccaatacctatgaagccgagaagaatacttcctacactcataccgaagtgcgcgctccacttgtgacccccgccgctccgttcatttccacgggagtgactggcctggctcaggatgtcgttggtgaaggtttcacggcgtctgctgcccgcatcactgccggcagcgcgtccgccgttgtgaccgagaccgctgagatgcgtgacaagtcgatgaaggagcaggaacgttatttgcgcgagaaggaggccatcgctcgatctcatgagaaggatctggagaagaagactgaggcctaccgaaaggaagcggaggctgaagctgagaagatccgcaaggagctggagaagcagcacgcacgcgacgtggaattccgcaaggacatggtggatgagaccattgagcgccagaaacgcgaggtcgagctggaagccaagtacgccaagaaggagctggaacacgaacgccagatggctcagaatgccctggaccagagcaaaatggccaccaacatcgaagtgtccatggacaccgctgccgggcgcaccgtgagcggcggtaccaccgtgtcagagtccttcgagacccaccacgaagagcacggcaaggagaagaaatccctgggcgagaagatcaaggatacctttttgggccgttaa

SEQ ID NO: 220 - DNA, 669 bp, Paramacrobiotus richtersi
atgcctcatactcatgagcataaagaagttaaggaagtacgcacttccgacggtggccatttggtcgagtccattaaaaatgtatcctcaacaacgcacgtcgacacggatacactggacacagcgaccacacatacgaccattcacgcgccgttgatccatccgacgggcacggtctcggttcatgcggtcagtggactggcccaagagctgctgggtgaagggataaccgcatccgtggagcgggtgacggccggaactcgggatgaagtcatttacgagactcccgaacaactggaacggaaacgggaccgggatgagaaatactaccaggcaaaggaaaagattcgcgaaaagcatgagaaggaaattggaaagctgacggaagattaccgcgagaaaaccgaacgggaaacggctaagattcgcaaagagatggagaagcagcatgagcgcgatgtggagtttcgtagcaagctggtggaagatgcgatcaagaggcaaaaagaagaactggaactggaagcaaaatacgccaagaaagaactggagcggcaaagggaattggcactcgacgcactcgaaaacagccgcatgcacacggacatttccgttaacatggacaccacagtcgggcatactgttagcagcggccgaatcgatagttag

SEQ ID NO: 221 - DNA, 837 bp, Paramacrobiotus richtersi; identical to SEQ ID NO: 213

SEQ ID NO: 222 - DNA, 987 bp, Paramacrobiotus richtersi
atgcctctcttcggatctaacaaggacaaggacagtaaatcatcctacaaggaggagcatcacgagagccacacggagcgccgcatggaggacaactgcccaccgcccatgctgagcaaggacatgccgaccaatctcagcgggaaagtcgtcgtcgaacgccatgaaaccgtgtcgacgatccccgatgtcaaacccgtggtggagatgagtcgcacccccatgtaccgtcaggaggccgatatccatattcaggccggccaccgcgaggtggtggagcgcaccgatgtcatcaagtccaaggcgcaggcccagaaggaggtggagattgtcagcatcccgattcagaagatggccgcggcgcagatggagcatgtgcgtacgggtgtgacctttactcaggataaggagatgatcatccccggaccgatggttgctccgcccattcccagcgtgacccacgacctgctggtccagggctcgggcggtaccagcgccgagatccacgccagcaccaacgtggacctgttggccaacgcccaactcgccggacaatccccggaagaatacgcccgctaccgcgccggcgtcgaacaactggcccaccagcacgaagtggaaacgacccagaaagccgaggcctaccgccaccaggtcgaagccgacgccgagctgatccgccgcaccctggaacgccaacacgtccgcgacattgaattccgcaaggatatggtctccaccgccgtcgaccgccaacagcaggagatcaagatggaggccgagtatgccatgaaggcgctggagcaggagcgtatcgcggcggaacgggcgttggatcaggccaagatggagacgcatatcgatgtcaaggtggacacggccatcgggacgacgatcagtaagggggaggtgcggacggcggccgggcgggagattcgggaaagtgttggaccggtgacggttcaccatggggcgacgaggatctga

SEQ ID NO: 223 - DNA, 984 bp, Paramacrobiotus richtersi
atgcctctcttcggatctaacaaggacaaggacagtaaatcatcctacaaggaggagcatcacgagagccacacggagcgccgcatggaggacaactgcccaccgcccatgctgagcaaggacatgccgaccaatctcagcgagaaagtcatcgtcgaacgccacgagaccgtctcgacgatcccggatgttaagcccgtcgtggagatgagtcgcacccccatgtaccgtcaggaggccgatatccatattcaggccggccaccgcgaggtggtggagcgcaccgatgttatcaagtccaaggcgcaggcccagaaggaggtggagattgtcagcatcccgattcagaagatggccgccgcgcagatggagcatgtgcgcacgggtgtgacctttacccaggataaggagatgatcattcccggagccatggttgctccgcccattcccagcgtgacccacgacctgctggtccagggctcgggcggtaccagcgccgagatccacgccagcaccaacgtggacctgctagccaacgcccaactagccggacaatccccggaagaatacgcccgctaccgcgccggcgtcgaacaactggcccaccagcacgaagtggaaacgacccagaaagccgaggcctaccgccaccaggtcgaagccgacgccgagctgatccgccgcaccctggaacgccaacacgtccgcgacattgaattccgcaaggatatggtctccaccgccgtcgaccgccaacaacaggagatcaaaatggaggccgagtatgccatgaaggcgctggagcaggagcggatcgcggcggaacgggcgttggatcaggccaagatggagacgcatatcgatgtcaaggtggattccgccatcgggaccacggttagtaagggggatgtgttgacggccgccgggaaggagattcgggagaatgtcggaccggttacccgggatcatccggcgcgtcattaa

SEQ ID NO: 224 - DNA, 792 bp, Paramacrobiotus richtersi
atggagcacacggaagtgcataaaacgaccgagagcgccacgggccgctcgcacactatacagacggaaacgacggtcaaagaccagacatatgtcccgttacgcgagcaagccgaccattcgcccacatcctcgcacagatcgttccaggagagacaaacggtgcatacccacacggatgcgcggaaaccgagtctgggaacaatacatcctgtcagcatatcttccgcctcgggactggcccaagaaatcgtcgccgaaggatatcacgcatcggccgctagtgtccatagtacgacggccgctacgacaattgccgaatccccacaaacctacgaactgaaactgaaggacctggaacactatcgccgcgaacaggaagccattgcccgtaagtacgaaaaggaagtggagaaactgacggaaaagtatcgtcgaaagacggaagcggaggcggataagattcggaaagaactggagaagcagcatgcccgggatgtggagtttcgcgagaagctggtgcaggaggccattgcgcggcagaaagaggagattgtcctggaggccaagtatgcgacgaaggaactggacagacaacgaatgctggcattggaggcgctggagcggagtcgtcaccagtcgaatattcaggtaaatctggaaacggtggctggacacacggtcagtgagagccagaacgtcacgtcccattacgagtcacacgacagcattaacgaccacaagtcgatcggcgcgaaaatcaaagaagcgattatgggaaaacctgagtga

SEQ ID NO: 225 - DNA, 684 bp, Paramacrobiotus richtersi
atggaagcgatgaatatgaacattccccgggatgccatgtttgtgccgccgcccgagagcgagcaaaatgggtaccatgagaagagcgaggtccagcagaccagctacatgcagtcgcaggttaaggtgccccattataatttccccacgccctatttcaccacatctttctcggcacaagagctccttggcgagggatttcaggcatcgatttctcgcatcagcgccgtgacggaagatatgcaatcaatggagatccccgagttcgtcgaagaagcccgtcgcgattacgccgctaagaccagggagaatgagatgctggggcagcagtacgagaaggagttggagcggaaatcggaggcgtaccgaaagcatcaggaagtggaggccgacaagatccgtaaagaactggagaagcagcacatgcgcgacattgagttccgcaaggagatcgccgagctggccatcgagaaccagaagcgcatgatcgacctcgaatgccgctatgccaaaaaggatatggaccgtgaacgcaccaaggttcgaatgatgctcgaacaacaaaagttccacagcgatatccaggtcaatctggattcgtcggctgccggcacggaatctggtggtcatgtggtgtcacagtccgagaagtttaccgaacgaaaccgcgagatgaagcgataa

SEQ ID NO: 226 - DNA, 792 bp, Paramacrobiotus richtersi
atggagcacacggaagtgcataaaacaaccgagagcgccacgggccgctcgcacactatacagacggaaacgacggtcaaagaccagacatatgtcccgttacgcgagcaagccgaccattcgcccacatcctcgcacagatcgttccaggagagacaaacggtgcatacccacacggatgcgcggaaaccgagtctgggaacaatacatcctgtcagcatatcttccgcctcgggactggcccaagaaatcgtcgccgaaggatatcacgcatcggccgctagtgtccatagtacgacggccgctacgacaattgccgaatccccacaaacctacgaactgaaactgcgagacctggagcactaccgccgcgaacaggaagccattgcccgtaagtacgaaaaggaagtggagaaactgacggaaaagtatcgtcgaaagacggaagcggaggcggataagattcggaaagaactggagaagcagcatgcccgggatgtggagtttcgcgagaagctggtgcaggaggccattgcgcggcagaaagaggagattgtcctggaggccaagtatgcgacgaaggaactggacagacaacgaatgctggcattggaggcgctggagcggagtcgtcaccagtcgaatattcaggtaaatctggaaacggtggctggacacacggtcagtgagagccagaacgtcacgtcccattacgagtcacacgacagcattaacgaccacaagtcgatcggcgcgaaaatcaaagaagcgattatgggaaaacctgagtga

SEQ ID NO: 227 - DNA, 684 bp, Paramacrobiotus richtersi; identical to SEQ ID NO: 225

SEQ ID NO: 228 - DNA, 525 bp, Paramacrobiotus richtersi
attgttgctcatgctgccggaatcgccgaagaagttgtgggaaaaggattcactgcatccgccgcccggatcacgggaaccagtcagcaagtggacgttacgcccagtcctcagctgcaacaagaagtccgccgtgatgaagaacgttatatgcgcgaaaaagatgccatcgctgcgcagcatgagaaggaattagaaaggaaaacagaagcctaccgaaaaacggccgaagcggaggctgaaagaattcgcaaggaactagaaaagcaacatcaacgtgatgttgaattccgaaaagatcttgtggacagcgctataaataggcagaaacaagaagttgaactcgaggcaaaactggccaaaaaggagctggagcgtgaagctgctatggcaaaagaagcgctggaaaggtcaaaactatccaccaatatcgaggtcaacttcgacagcgctgtgggtcacacgcagtcggcaggcaccactgtgtccgaatcggaatcgatttccagaacggttaagaagtga

SEQ ID NO: 229 - DNA, 690 bp, Paramacrobiotus richtersi
atgtctgctgaagcgatgaacatgaacatgaaccaggacgccgtgtttattcccccgcccgagggtgagcagtacgagcgaaaggagaagcaggagatccagcagaccagctacctgcagtcccaggtcaaggtgccccttgtcaacctccccgctccgttcttcagcacttccttttctgcccaagaaattctcggcgaaggtttccaggcttcgatttcgcgcatcagcgccgtctcggaagagctgtcgtccatcgagattcccgaactggccgaagaggcccgtcgcgacttcgctgccaaaacccgtgagcaggagatgctgtcagccaattatcagaaggaagtggagcgcaagaccgaggcctaccgcaagcagcaggaagtcgaggccgacaagatccgcaaagaactggagaagcagcatctgcgtgacgttgagttccgcaaggacattgtcgagatggccatcgagaaccagaagaaaatgatcgacgtggagagccgctacgccaagaaggacatggaccgcgaacgcgtcaaggttcgaatgatgctcgagcagcaaaagttccacagcgacatccaggtcaatctggattcttcggctgctggcacggaaactggaggtcaggtggtgtcggaatctcaaaagttcaccgaacgaaaccgccagataaagcaataa

SEQ ID NO: 230 - DNA, 651 bp, Paramacrobiotus richtersi
atggaagccagacagcacgagcaacagttccagaagcaggaggtggagcagaccagctacatgcagacccaagtgaaagtgcccgttatgaaactctcggcgccctccatcatcacggtgcccctggcgcaggaactcgttggtgaaggatttcaggcttcgatttcgcgcatcagcggtgtctcccaggagatccagcagatcgactccgcacaattagacgaggaggtccgtcgggactatgaatctaaacagcgcgaagcggaactgctgcagcagcaattcgacaaggaggtggaaaagaagaccgaggcctaccgcaaacaacaggagattgaagccgagataatccgtaagatgttggaaaagcaacacatccgcgacgtggagttccgcaaggagctcgtcgagcatgccatcgagaaccagaaacgccagatcgacattgagagccgctatgccaagaaggagctggaacgggagcgcaccaaggccagaatgctgttggagcgacagaaattccacagcgacattcaggtcaatctggattccaccgccgcgaccacccatgcaggagagcaagtggtgtccgagtcggagaagttcacccagaactccaagatgtcgtgcggccaacagcgtgccggataa

SEQ ID NO: 231 - DNA, 690 bp, Paramacrobiotus richtersi
atgtctgctgaagcgatgaacatgaacatgaaccaggacgccgtgtttattcccccgcccgagggtgagcagtacgagcgaaaggagaagcaggagatccagcagaccagctacctgcagtcccaggtcaaggtgccccttgtcaacctccccgctccgttcttcagcacttccttttctgcccaagaaattctcggcgaaggtttccaggcttccatttcgcgcatcagcgccgtctcggaagagctgtcgtccatcgagattcccgaactggccgaagaggcccgtcgcgacttcgctgccaaaacccgtgagcaggagatgctgtcagccaattatcagaaggaagtggagcgcaagaccgaggcctaccgcaagcagcaggaagtcgaggccgacaagatccgcaaagaactggagaagcagcatctgcgtgacgttgagttccgcaaggacattgtcgagatggccatcgagaaccagaagaaaatgatcgacgtggagagccgctacgccaagaaggacatggaccgcgaacgcgtcaaggttcgaatgatgctcgagcagcaaaagttccacagcgacatccaggtcaatctggattcttcggctgctggcacggaaactggaggtcaggtggtgtcggaatctcaaaagttcaccgaacgaaaccgccagataaagcaataa

SEQ ID NO: 232 - DNA, 783 bp, Paramacrobiotus richtersi
atggcgcacgaactcaaccctcacgaaacccggaccgatttctcggatgcggagggaggcagctacgaaaaacaaattcattcggagttgcgagccccatcagcaacaccttccggtcattcccaaagtgataagagagaaactactgtgacttacacctacacagatgtccgaactccacagatgaatccacctgcacctgttttgatcattccatccgctgcgggactagctcaagaaatcgtcggcgagggattcaccgcatcggccgccagagtaacgggcgccagtccccaggtgaccgttactgaaacactcacctcacaagaaaaatatttacgtgagcaggagaactaccgtcgagagcaggaagccctggtccgcaagtacgaacggtctatcgagaagatgaacgaagagtaccgcaagaaaaccgaacaggaagcggacaagatccgcaaagaaatggagaagcaacacgagcgggacattgaattccgtaaggagctgatggacaaggccatcgaacggcagaaagaggagattgccctggaagccaagtatgcgcggaaggaactggaacgacaacgggagatggccatggaagcgttggataagacgaagaagcaggcggatgtgcaggttaatctggacacgttggccgggcatacggttagcgaaagccagagccagctgacgccggatgcggatatcccagccgatcacagagaaccgcataagtcgttaagctcgaaactgcgcgaaactttcacagggaaatag

SEQ ID NO: 233 - DNA, 669 bp, Paramacrobiotus richtersi
atggagcgaaaagtcgaagtgtgccaggagaagcatacccattccgaaaaatgccgcacggaaacgcatggacatcaagaaactgtgcatactggatatacacacacggaagtccgtgcgcctttggtggttcctccgccaccgattgttgcgcatgctgccggaatcgctgaagaagttgtgggaaaaggatttactgcatccgccgcccggatcacgggaaccagtcagcaagtggacgttacgcccagtcctcagctgcaacaagaagtccgccgtgatgaagaacgttatatgcgcgaaaaagatgccatcgctgcgcagcatgagaaggaattagaaaggaaaacagaagcctaccgaaaaacggccgaagcggaggctgaaagaattcgcaaggaactagaaaagcagcatcagcgtgacgttgagttccggaaagatcttgtggacagcaccataaacagacagaaacaagaagtcgaactcgaggcaaaattggccaaaaaagagttggagcgcgaagctgccatggcaaaggaagcgctggataaatcaaaacttgccaccagtatcgaggtcaacttcgacagtgctgttggccacacgcaatcagcaggcacaaccgtgtccgaatcggaatcggttaccagaacggtaaagaagtga

SEQ ID NO: 234 - DNA, 783 bp, Paramacrobiotus richtersi
atggcgcacgaactcaaccctcacgaaacccggaccgatttctcggatgcggagggaggcagctacgaaaaacaaattcattcggagttgcgagccccatcagcaacaccttccggtcattcccaaagtgataagagagaaactactgtgacttacacctacacagatgtccgaactccacagatgaatccacctgcacctgttttgatcatcccatccgctgcgggactagctcaagaaatcgtcggcgagggattcaccgcatcggccgccagagtaacgggcgccagtccccaggtgaccgttactgaaacactcacctcacaagaaaaatatctacgtgagcaggagaactaccgtcgagagcaggaagccctggtccgcaagtacgaacgctccatcgagaagatgaacgaagagtaccgcaagaaaaccgaacaggaagcggacaagatccgcaaagaaatggagaagcaacacgagcgggacattgaattccgtaaggagctgatggacaaggccatcgaacggcagaaagaggagattgccctggaagccaagtatgcgcggaaggaactggaacgacaacgggagatggccatggaagcgttggataagacgaagaagcaggcggatgtgcaggttaatctggacacgctggccgggcatacggttagcgagagccagagccagctgacgccggatgcggatatcccagccgatcacagagaaccgcataagtcgttaagctcgaaactgcgcgaaactttcacagggaaatag

SEQ ID NO: 235 - DNA, 777 bp, Paramacrobiotus richtersi
atgcctttattcggatcatccaagaaggacaagcaccatcacggcgctgaattccccatcaccgaccgggacaacgatattgaaaaccgcaacctggaacactttgaccgggaggtggaccaattccccgaccgctctccagggcgggaagggtcgcttatccaagagcgccacatttcccgtagtcccgttcacaaaacggtgacggagcgtcgttcggaagtgtcctatgttcagagtgtcccagccgcggtggagattagccgccagcccatgtatcatcaggaggcgaatatcatcatccctggggaacgccgggaggtggtggagaagacggaggtgatccggtcagccacgccccgacgagaatccgtcgaagtgatcagtataccaattcataaggtcagcggggcgcagatggaacatgtacgctcgggtgtgacgtatacgaatgataaggagttgattattcctggtgcgatgattgcgccaatgattcccagctgcacacaggatttactggctcgaggaagcggtggtacccacgccgaaatccacgccgacaccaacatcaacctcctagccaacgcccacctcgactcatcccccgaggagtacaaccgctaccgagccagcgtggaagaactcgcccaccagcacgaaatcgacacggcccagaaagccgccctctaccgcacccaagtcgaagccgacgcggaactgatccgtcgcaccctggaacgccaacacatccgcgacattgagttccgcaaggag

SEQ ID NO: 236 - DNA, 777 bp, Paramacrobiotus richtersi; identical to SEQ ID NO: 235

SEQ ID NO: 237 - DNA, 501 bp, Paramacrobiotus richtersi
atgcctcatactcatgagcataaagaagttaaggaagtacgcacttccgacggtggccatttggtcgaatccattaaaaatgtatcctcaacaacgcacgttgatacggacacactggacacagcgactacgcatacgaccattcacgcgccgttgatccatccgacgggcacggtctcggttcatgcggtcagtggactggcccaagagctgctgggtgaagggataaccgcatccgtggagcgggtgacggccggaactcgggatgaagtcatttacgagactcccgaacagctggaacgaaaacgggaccgggatgagaaatactaccaggcaaaggaaaagattcgcgaaaagcatgagaaggaaattggaaagctgacggaagattaccgcgagaaaaccgaacgggaaacggctaagattcgcaaggagatggagaagcagcatgagcgagatgtggagtttcgtagcaaactggtggaagatgcgatcaagagg

SEQ ID NO: 238 - DNA, 447 bp, Paramacrobiotus richtersi
atgtcgcacgcccaccacgcccacaactacgaatgctacacgcacacggcggacggcaaggcggcggccacggccaccgccacgtcgctggcgtcgggcgagaaggaggtgcacaccaccagctacacccacgtggacgccaaactgcccctgctgcaggacctcccgtcgcccctcaccaccacgggcatcgccggccttggccagaccctggtgggcgagggcttcacggcctcggtcgtccgtgcgtccggcgagtcggagcaggtgaccgtggcgcccagtgagcgcctgaccgaggaggcccgtcgcgaccaggagcgctaccagcgcgacaaggacgccatcaacgagcgccagaagcacagcgtggagaacaaggccgagaactaccgcaaggaggccgagcagcaggccgagcgcatccgcaaggagctggagaagcag

SEQ ID NO: 239 - DNA, 276 bp, Paramacrobiotus richtersi
aaggagctggagaagcagcacgaacgcgatgtcgagttccgcaagggattgattgatagtgcgattgaacgacaaaagcgggaggtcgagctggaggccaagatggccaaacgcgaactggaccgggaggcccagctggccaaggaggcgctcgagcggtccaagttggctacgaatgtcgaggtcaacttcgacagtgccgtcggtcacactgcttcggtgggaacaacggtctccgaatcggaatcgatcacgcgggatgtacgcaagaactga

SEQ ID NO: 240 - DNA, 446 bp, Paramacrobiotus richtersi
atgtcgcacgctcaccacgcccacaactacgagtgctacacgcacacggcggacggcaaggcggcggccacggccaccgccacgtcgctggcgtcgggcgagaaggaggtgcacaccaccagctacacccacgtggacgccaaactgcccctgctgcaggacctcccgtcgcccctcaccaccacgggcatcgccggccttggccagaccctggtgggcgagggcttcacggcctcggtcgtccgtgcgtccggcgagtcggagcaggtgaccgtggcgcccagtgagcgcctgaccgaggaggcccgtcgcgaccaggagcgctaccagcgcgacaaggacgccatcaacgagcgccagaagcacagcgtggagaacaaggccgagaactaccgcaaggaggccgagcagcaggccgagcgcatccgcaaggagctggagaagca

SEQ ID NO: 241 - DNA, 234 bp, Paramacrobiotus richtersi
atggagaagcagcatgagcgcgatgtggagtttcgtagcaagctggtggaagatgcgatcaagaggcaaaaagaagaactggaactggaagcaaaatacgccaagaaagaactggagcggcaaagggaattggcactcgacgcactcgaaaacagccgcatgcacacggacatttccgttaacatggacaccacagtcgggcatactgttagcagcggccgaatcgatagttag

SEQ ID NO: 242 - DNA, 675 bp, Hypsibius dujardini
atgtctaactaccagcaagaatccagctatcagtacagcgaccggagcaacaatggtcaacagcaagagcaacaggagaagaaggaggtcgaacactccagctatacccacaccgacgtcaaagtgaacatgcccaacttaatcgcacctttcatcagctcttccgcgggtttggctcaagaactggtcggggaaggtttccaggcgtccgtctctcgcatcaccggcgcatccggggagctcaccgtcatcgacaccgaggccgaaaccgaggaggcacgtcgggacatggaagccaaggcccgcgagcaggagctcctgtcgcgacagtttgagaaggagctggagcgaaagaccgaagcgtatcgcaagcagcaagaagttgagaccgagaagatccggaaggaacttgagaagcaacatctgcgggatgtcgagttccgcaaggagttgatggagcagaccatcgagaatcagaagcgtcagatcgacctggaggcacgctatgccaagaaggagcttgagcgggaacggaacaaggtcaagcgtgtgctggaacgctccaaattccacaccgatatccaggtcaacatggaagccgctgcgggttcaactcattccggatcatccagcgttgccgtgtcggagtcggaaaagttccagaccaacaactga

SEQ ID NO: 243 - DNA, 675 bp, Hypsibius dujardini
atgtctaactaccagcaagaatccagctatcagtacagcgaccggagcaacaatggtcaacagcaagagcaacaggagaagaaggaggtcgaacactccagctatacccacaccgacgtcaaagtgaacatgcccaacttaatcgcacctttcatcagctcttccgcgggtttggctcaagaactggtcggggaaggtttccaggcgtccgtctctcgcatcaccggcgcatccggggagctcaccgtcatcgacaccgaggccgaaaccgaggaggcacgtcgggacctggaagccaaggcccgcgagcaggagctcctgtcgcgacagtttgagaaggagctagagcgaaagaccgaagcgtatcgcaagcagcaagaagtcgagaccgagaagatccggaaggagcttgagaagcaacatctgcgggatgtcgagttccgcaaggagttgatggagcagaccatcgagaatcagaagcgtcagatcgacctggaggcacgctatgccaagaaggagcttgagcgggaacggaacaaggtcaagcgtgtgctggaacgctccaaattccacaccgatatccaggtcaacatggaagccgctgcgggttcaactcattccggatcatccagcgttgccgtgtcggagtcggaaaagttccagaccaacaactga

SEQ ID NO: 244 - DNA, 684 bp, Hypsibius dujardini
atgtctggacgtaacgtagaaagccacatggagcggaatgagaaggttgtggtcaacaattccggtcatgctgacgtgaagaagcaacagcaacaagtggagcacaccgaattcacccacaccgaggttaaagccccgttgatccatcccgcacctccgatcatctcaactggagctgccggactcgccgaggagattgtgggacaagggttcaccgcgagtgccgcgcgcatcagtggaggtaccgctgaagtacatctccagccttcggccgcaatgacggaagaggcccgtcgcgatcaagagcgctaccgccaggaacaggagtcgatcgccaagcagcaggaacgtgagatggaaaagaagactgaggcataccgcaagaccgccgaggcggaagctgagaagatccgaaaggagctggagaagcaacacgcgcgtgatgtcgagttccgtaaggatctcatcgagagcacgattgaccggcaaaaacgcgaggtcgatctggaagcgaaaatggccaagcgggaattggatcgtgaagggcagttggccaaggaggcgttggaacgatcacgtttggccaccaacgttgaggtcaatttcgacagtgcagctggtcataccgtgtccggtgggacgaccatctccagctctgacaagatggaaatcaagcgcaactag

SEQ ID NO: 245 - DNA, 684 bp, Hypsibius dujardini
atgtctggacgtaacgtagaaagccacatggagcggaatgagaaggttgtggtcaacaattccggtcatgctgacgtgaagaagcaacagcaacaagtggagcacaccgaattcacccacaccgaggttaaagccccgttgatccatcccgcacctccgatcatctcaactggagccgccggactcgccgaggagattgtgggacaagggttcaccgcgagtgccgcgcgcatcagtggaggtaccgctgaagtacatctccagccttcggccgcaatgacggaagaggcccgtcgcgatcaagagcgctaccgccaggaacaggagtcgatcgccaagcagcaggaacgtgagatggaaaagaagactgaggcataccgcaagaccgccgaggcggaagctgagaagatccgaaaggagctggagaagcaacacgcgcgtgatgtcgagttccgtaaggatctcatcgagagcacgattgaccggcaaaaacgcgaggtcgatctggaagcgaaaatggccaagcgggaattggatcgtgaagggcagttggccaaggaggcgttggaacgatcacgtttggccaccaacgttgaggtcaatttcgacagtgcagctggtcataccgtgtccggtgggacgaccgtatccacttcggacaagatggaaatcaagcgaaactag

SEQ ID NO: 246 - DNA, 714 bp, Hypsibius dujardini
atgtcgcagcaatatgagaagaaggttgagcggacggaagtcgtctacggaggagatcgtcgtgtcgagggatccgcgtccgcatccgccgagaagaccaccaactacacccacactgagatccgcgctccgatggtgaatcctctgccgccgatcatttcaacgggtgccgctggcctggcacaggagattgttggggagggctttacagcctctgccacacgtatatccggagctgccgccaccacccaagtcctcgaatctcaggcatcccgcgaacaggccttcaaggaccaggagaagtactcacgcgagcaggccgccatcgcccgggcgcacgacaaggaccttgagaagaagactgaggaatatcgcaagactgccgaggctgaagctgaaaagatccgcaaggagctggagaagcaacacgcccgcgatgtggaattccgcaaggatctcgtagaatccgccattgaccgccagaaacgggaggtggatttggaagccaagtacgccaagaaggagctggaacacgagcgtgaactggccatgaacgcgctagagcagtcgaagatggccaccaatgtgcaagtccaaatggacaccgctgctggtaccacggtcagcggaggaacgacagtctccgaacacactgaagtccatgatgggaaggagaaaaaaagcctcggcgagaagataaagtcccttttttga

SEQ ID NO: 247 - DNA, 1077 bp, Hypsibius dujardini
atgtccgagttaccgggcttttctatgcaccgcctttgccgccagtggaattttccgctggacaaatcccactgcgctgcaaatacattccatgctgggatttcccgttctctttcacgcactccgcagtttttaaattttcgctttctccaaatctttctcccattgtattttcgaagtttacttcacaggaaagaatctcccctcatcatcatgtcgcacacacacgagcaaaaattcgagcgagtcgaggagcgcactatcgacgagaagaagggcacggaggaggttcgcgtcggcatcgacaccggatacggcgatcccgcgctcaacttccagccgaccgatgcgactctggtgcggactccttgcgtgggcggagacgtcatgtcctccaacagatcgtccgcgtgctccagcggtgtcgccggggcctcgcagttcgcctcacactctatgcgtgacagctcttccgggaacgtagtgaaggaggcggagaagaccaccagctacacccacaccgaggctcatgcgccgctgattactccaagtcagccgttcatcgtgacgggagccgccggactggctcaggagattgtcggagagggtttcaccgcgtccgcttcccgcatcagcggtggcgccgtcaacaccaaagtcatcgagaccgctgagatgcgccagaaggaactccgcgagcaagaacaatttgcccgtgaacaggccgctatcatccagcatcatgataaggatctggctaggaagactgaaaagtaccagaaggaggccgaggcggaagcggagaagattcgtaaggagctggagaagcagcatgcgcgagatgtggaattccgaaaggacttagttgagactgcgatcgatcgccagaagcaggaaatcgacctggaagcgaagaaggccaaggccgacctggagcgagaacgacagatggccaaggaagcgctcgataacagcaagatgcagaccaacatcgaggttcagatgaactctgccgctggaatgaccaccagcggcggcacatccgtgtcagagtctcatgtgtcgaagaatttctag

SEQ ID NO: 248 - DNA, 1245 bp, Hypsibius dujardini
atggccaccaaggaatcaaagtatgaacgcgtggagaaggtcaacgtggacgcggatggcgccacactggtcaagaatatcggcgaggaccgcggcaaggaggatcccgggatgaatttccaggacaaacgcccggccaatttggtgcccggagcgccggccggagtcattcccaaccgtatcgagtcattgccgacggatcgtgccgggcaacgtctccgtgagcatctcagcgagagcgaacgccttcgcgtctcccgcagcagcaccagcagcaagtcttccagcttcgtggagcccagcctcaagtaccgcggtgagatcggaccgatcggaaaaaacggcgagttcgtcgcctcgtcgaaccgtcagaactccagcagcaacgtctcgtcctccgacaacagcgagcgtgcgtctccggcgtcccgcaactccaaccccgggatgaacaacggcatgacgacccaacgcaccacggtgatcacggagagctcggttcagggcctcggcgcgcaacgcaccgtcccgatccagccgcatcagcagcgcgaagaccacgaggtgatcacccacgaatcgcatgctcgagctccggaaacgaccgtggtgaccattcccacgacgcgcttcgagtccgcgcagctggagtcgcgccgcgacggcagaacctacaccgaggacaaggagctgaccattccggcgccggtggtcgcgccgcagatccacgcccaccagcaggttaacatgtccggaggaacttcggcgacgatccacgccacgaccgatttacatttggctagcgaggcacagatcaatgacatgggaccagaggagtatgagcgctaccgtgccaaggtggaagccttggctcgcatacatgaggacgaaacgtcgcgcaaagcggcggcctaccgtaatgccgttgaggccgatgcggaactcatccgccaaactctggagcgccaacacatgcgtgacattgaattccgcaaggatctagttgaaagctcggtggatcgtcaacagcaggagatccgcctggaggccgagtacgccatgcgagccctggaacaggaacgtgtcaatgctcgcgctgctcttgaccaagccatggcgtcgacgaacatcgacgtgaacattgactccgctatcggcacgacccattcccagggaagggttacgacgacctctgaaagccgaacaagtcaggcacgtggacccgccactgccgcagttatctga

SEQ ID NO: 249 - DNA, 1245 bp, Hypsibius dujardini
atggccaccaaggaatcaaagtatgaacgcgtggagaaggtcaacgtggacgcggatggcgccacactggtcaagaatatcggcgaggaccgcggcaaggaggatcccgggatgaatttccaggacaaacgcccggccaatttggtgcccggagcgccggccggagtcattcccaaccgtatcgagtcattgccgacggatcgtgccgggcaacgtctccgtgagcatctcagcgagagcgaacgccttcgcgtctcccgcagcagcaccagcagcaagtcttccagcttcgtggagcccagcctcaagtaccgcggtgagatcggaccgatcggaaaaaacggcgagttcgtcgcctcgtcgaaccgtcagaactccagcagcaacgtctcgtcctccgacaacagcgagcgtgcgtctccggcgtcccgcaactccaaccccgggatgaacaacggcatgacgacccaacgcaccacggtgatcacggagagctcggttcagggcctcggcgcgcaacgcaccgtcccgatccagccgcatcagcagcgcgaagaccacgaggtgatcacccacgaatcgcatgctcgagctccggaaacgaccgtggtgaccattcccacgacgcgcttcgagtccgcgcagctggagtcgcgccgcgacggcagaacctacaccgaggacaaggagctgaccattccggcgccggtggtcgcgccgcagatccacgcccaccagcaggttagcatgtccggaggaacttcggcgacgatccacgccacgaccgatttacatttggctagcgaggcacagatcaatgacatgggaccagaggagtatgagcgctaccgtgccaaggtggaagccttggctcgcatacatgaggacgaaacgtcgcgcaaagcggcggcctaccgtaatgccgtcgaggccgatgcggaactcatccgccaaactctggagcgccaacacatgcgtgacattgaattccgcaaggatctagttgaaagctcggtggatcgtcaacagcaggagatccgcctggaggccgagtacgccatgcgagccctggaacaggaacgtgtcaatgctcgcgctgctcttgaccaagccatggcgtcgacgaacatcgacgtgaacattgactccgctatcggcacgacccattcccagggaagggttacgacgacctctgaaagccgaacaagtcaggcacgtggacccgccactgccgcagttatctga

SEQ ID NO: 250 - DNA, 1077 bp, Hypsibius dujardini; identical to SEQ ID NO: 247

SEQ ID NO: 251 - DNA, 684 bp, Hypsibius dujardini
atgcagcaaaacaacgaaaatttcgaacgagtcgttgagcgttccgaggtgcgccaagaatgccagcagccgtgccgggaggaagagtcccgtcaggaggagcacaactccagctacctccacactgaagtccgcgctcccgtgcccaatatcccgcctccgatgatgtccgggtccgctggtctcggacaagccctggtcggagaaggattccaggccagcgctgcccgcatttccggtggctcccaggagatgaacatccagccaagtgaaaagctgttgcaggaagccgccatggacaaggagcgatatgctcgcgagcaagaagcaattcagaaccgtctgcagtccgaaacggaacgcaagaccgaggcctaccgaaagaccgctgaggcggaggctgagcgaatccgcaaggagcttgagaagcagcatgagcgagacatcgagttccggaaggatctggttcagggaaccatcgacagccagaagaaacaagtcgagctcggagcgatcatggctaaacgcgaattggaccgtgaagcgaaacttgctcgggatgcacttgaacagagcaagatggccacaaatgttgaagtgaactttgacagtgcagccggtcacactgtgtcgggaggccagacggtgtcgcagtccaccaaagtcaccagggaaaagaaataa

SEQ ID NO: 252 - DNA, 897 bp, Hypsibius dujardini
atgtcgagcattgagcaccccaatgtctatgtttccgaacgccaggacgtcttccgttccgccggagaggtccctccacccttgcccgcccgaccggtcggcggagagtttatccgtgaaacaatcactaccggacccgccggcacgatccacagcaccctcaccaccagcaccctaagccaaccgggaaccctaagcccacaaggaaccctaagcccgcagggaatcctaagccaaccgggaaccctaaaccaacagggaaccctaagccaacagggaaccctcaaccacagccatgtcgtcacgaccacgggcgacagcaccagctacacccacaccgaaatcaaagccccgctccacgtgacctcgcccatcctcatctcctcggcggagggtttggcgcaggagattgtcggtgaagggttcaccgcgtccgctgcacgagtggctggcacggcgacgcaggagtatgtccacgagacggtggagacctcacggcaggctgccttggatcgacagcgccgagaccgggagatggaagcggtggcgcggaggagtgaggaagaggtggcgaaaaagactgaggcctatcggaagaccgccgaggctgaggctgagaaaatccgccgggaactggagaaacaacatgcgcgagatgtggagttccgcaaagatctggtggagtctgccattgaccgccaaaagcgggaagttgacttggaagcgaaatacgccaaaacggagctggagcacgagcgaaaactggcactggaggccctggaacgatccaaacttgaaagcaacatcgaagtgaattttgacagcgctgccggtcggacagtgacggaaagccatgtcgtttcgcagcacaccgacatcagccatcccagaatgtaa

SEQ ID NO: 253 - DNA, 273 bp, Hypsibius dujardini
atggtggacgagtctgtcaagcggcagaagaaggaactcgaactggaagtgaagtacgccaagaaggagctcgatcacgagcgtctgcttgccaaagaggcgttggagcagagcaaaatgcacactgacgtgctggtcaatttggacacctcagccggtcataccgtctccggcggcagtcacgtcaccgaggaggaatacagcgagcatcataccgagcataagaagaccatcgcggaaaagctcaaggaaacttttactggtcatcattaa

SEQ ID NO: 254 - DNA, 897 bp, Hypsibius dujardini; identical to SEQ ID NO: 252

SEQ ID NO: 255 - DNA, 273 bp, Hypsibius dujardini; identical to SEQ ID NO: 253

SEQ ID NO: 256 - DNA, 684 bp, Hypsibius dujardini; identical to SEQ ID NO: 251

SEQ ID NO: 257 - DNA, 558 bp, Hypsibius dujardini
atgacgcactacaaggaagacgaggaactgcttgagcatctccgtgaagacagcggtttccaggccttcaagacgaaggccgttgacgacgtcgtggcaggcaacggaaatacccactcggaactgcacgaaacagtgaaggagaaggcatcggtgtcgtcagcgtcctcatcctcgtcgtcctcgccgccgtccaccggtcgcagcagcgtggaacgccatgtcacctacacgcacaccgaggcgaagagtgggcccttgattcacacgactcatccggtggtgttgagctcggcgtcgggcatgctggcgcatgagatcatggaggagcaatcggggttcatggcatcggcgacgcatgtctcgggcagcgaacacggcgtggcagcggcccacgagtcgccggagttgcgggaacaacggctgaaggatgaggccaagtatcgggagaaacaggacgagattgcgcgaaagcatgataaacacttggagaaggtgaccgaggagtaccgaaagaaaactgaagcggaagccgaaaagatccgcaaggagctggag

SEQ ID NO: 258 - DNA, 557 bp, Hypsibius dujardini
atgacgcactacaaggaagacgaggaactgcttgagcatctccgtgaagacagcggtttccaggccttcaagacgaaggccgttgacgacgtcgtggcaggcaacggaaatacccactcggaactgcacgaaacagtgaaggagaaggcatcggtgtcgtcagcgtcctcatcctcgtcgtcctcgccgccgtccaccggtcgcagcagcgtggaacgccatgtcacctacacgcacaccgaggcgaagagtgggcccttgattcacacgactcatccggtggtgttgagctcggcgtcgggcatgctggcgcatgagatcatggaggagcaatcggggttcatggcatcggcgacgcatgtctcgggcagcgaacacggcgtggcagcggcccacgagtcgccggagttgcgggaacaacggctgaaggatgaggccaagtatcgggagaaacaggacgagattgcgcgaaagcatgataaacacttggagaaggtgaccgaggagtaccgaaagaaaactgaagcggaagccgaaaagatccgcaaggagctgga

SEQ ID NO: 259 - DNA, 714 bp, Ramazzottius varieornatus
atgccttacgaaaagcacgttgaacagacggtggtggaaaaaactgagcagcctggacactcgtcgacgcaccatgctcccgcccaaaggaccgtagctcgcgagcaggaggaagttgtccacaaagagtttacccataccgacattcgagttccccacatcgacgcacctcctccaatcatcgctgccagcgcagtgggcttggccgaggagatcgtcagtcatggtttccaagcctcggcggcccgcatcagcggtgcttccaccgaggtcgatatgcgcccaagtcccaagctagccgaggaagctcgtcgtgatgccgaacgatatcaaaaggagcacgagatgatcaacagacaagccgaagccacgctgcaaaagaaggcggaggagtaccgtcaccagactgaggcggaagccgagaagattcgccgcgaactggaaaagcagcatgaacgggacatccagttcaggaaggacctcatcgatcagaccatcgaaaagcagaagcgcgaagttgatctggaagccaagatggccaaacgcgagctggatcgcgaagcgcagttggctaaggaagccctggaacgttctcgaatggccaccaacgtggaagtcacgctggataccgcagcgggacatacagtaagtgggggaactaccgtctccagcgtcgacaaagttgaaaccgtccgcgagcgcaaacatcattaa

SEQ ID NO: 260 - DNA, 651 bp, Ramazzottius varieornatus
atgtctcgagatcaaggaagcacggaatacgacgctaaccaacgccaggagcaacaccaggaacaacataatacctcttacacccacaccgatgtccgcaccaacatccctaatatccctgccccgttcatctctaccggtgtttcgggtctcggtcaacagctggtcggagaaggtttcaccgcctccgctgctcgcatctccggacaatcgtcggaaacccacgtacagatgacccccgagatggaagccgaagcgcgcaaggaccgcgagcgctacgaacgcgagctgcaagccatcaacgagcgacaccaacgagacatcgaaggcaagaccgaggcctaccgcaaacaggctgaacaagaagccgaacgtcttcgcaaagagctggagaagcaacaccaacgagatatcgagttccgcaaatcgctggtccagggtaccatcgagaaccagaaacgacaagttgagctcgaagcccagctcgccaagcgcgaactcgaccgagaagctcgccttgcaactcaggctctcgaccagtccaagatggccactgatgttcaagtcaattttgactcagcagtaggccacactgtttctggggctaccacagtctcccaatccgagaaagtcacccagtcgaagcactaa

SEQ ID NO: 261 - DNA, 912 bp, Ramazzottius varieornatus
atgtcttcccgacagaaccagcaatcgtccagccaacactcgtcctccagccagcaaggtggtcaaggtggtcaaggtgttcaaggaagttccagctactcgcgcaccgaggtccacaccagcagtggaggacctaccatcggtggagcccagcgaactgtccccgtcccccctggatctcactccgaggtccatgaggagcgtgaggtcatcaagcatggtaccaaaaccgaaagcgagacccacgtcgtcaccgtcccagtgacaactttcggcagcaccaacatggaatctgtccgaaccggcttcaccgtcacccaagacaagaacttgaccgttgctgctcccaacatcgctgctcccatccacagcaacctcgaccttaacctcggcggtggagctcgcgctgaaattaccgcagggaccaccgttgacttgagcaagatccagcgcaaggatttgggacctgaagagtatgctcgctacaaggccaaggtcgagcaactggccaggcaagatgagcaagacgcgggtatgcgcgctgcccagtaccgagaggaagtggagcgtgatgccgaactcatccgacagatcttggagcgacaacacatccgtgatcttgaattccgcaaggaaatggttgagaaccaagtcaaccgacaagagagagaaatccagctggaggctgagtacgcaatgcgagccctcgagcttgagcgcaatgccgctaaagaggctttggaaagcgccaaggcccagactaacgtcaatgtcaaggtcgagtccgctattggtaccaccgtctccaagggtgcaatccagacttccgccgacaagagcagcaccaccaagactggacccaccaccgtcactcagattaaacataccgaacaacacactgaacgccgatag

SEQ ID NO: 262 - DNA, 1137 bp, Milnesium tardigradum
atgtctacccatcgtgaccgagactctgctaataacgaatatatcgctgagaccgtctcaagcgtgacgacctctacggccgccgatctgaccactggtcgtacgttatacgcaactcctgtgacctcaaccgcccgacaccacgatacgaccacatccagccataccagtcaacgtatggccactgactacaccaccggcgctggtacagtctacactgaaaagacagtgatgcgcgaaccggtcaatgtcgtccatactcaaattgaccgcgtcactgcagtgcccatcactgagacgcaagtgcacgctgaaacgcagcattatttgcatacgcagatgcgtacgcctgtagtagagtcgcatccgccgcaattgccggctcataccgatgtggccggttcgattctcaacgattcggcattttcttcaaccgcccatatctctacgaatgcgatgcatgcacaggccgtaccagtggatgcggcagaacgggcaagacaagaggaacacttccgtcgagaggccgaccggattgctttacagcatcagcgagcgatcgatgagaagtcggaagcgtatcgtagagatacagaggcacaggctgagagaattcgacaagaactcgaaaagcaacatcttcgagatgtgcagtttagacaagagttggtggacgatgcgatcaccagacagaaacgtgaagtgcagctggaagcacaggctgtgatggctgatctcgagttggaacgtcgaagagcacatgaggcgttggaacgcagcaaaatgtccacagacatcaacgtaaacatcgatactctggctggcagcactactgcaggtggcaccactgttatcgagaaaactgaagtgcaaaagggcgtcgcttaccacacgactccagtcggcgtcacgactcacgttgagtacgctgaccgtcccacaacaacccaccgaacggagaccaccactactgctcactcgactcacaccaccgaaggtcaccaccgcggatcagacgcatcgtacattggcggccgtgacgatgatcgtatgtcaattggtactcagggcagcgatgcagatgagcacaagaaacgtggacttttgggcaaaatcaaggattctttgacgaaataa

SEQ ID NO: 263 - DNA, 750 bp, Milnesium tardigradum
atgtctcatcaacagacgcgcgaagtgactaaagagatccacgttgagtcgagtggtcaatcgggcgcatcgtcacatgcttccggtcatgtagtggccggacacgagacatcagcggtcgaacacaccaaatacctgcataccgagactaaggtgccaatggccacgcctgcgccgcccatcattcatgcgtcgtcgggtctgcaacatatggagggtatgaccgcctctgctgcgcgcatcactgcaggatccgctgagaccactaatgtccaggtttccgaggaagtcagacgacgtgatcaggctcagttcgaacgtgaggccgcagcaatcgctgctcgtcatgagaaggatgttcaggcgaagaccgaagcataccgtaaagagaccgaagaacaagccgagaaaattcgacgcgagttggaaaagcaacaccaaaaagatgtcgagtttcgaaaggatatggtcgacgacactatcaaccgtcagaagcgtgaagtcgagttagagtcagcgatggccaagcgtcagctcgagcgcgaagctgaagctgccaaggctgcacttgacaagagcaaactgtcgactgacattcatgttgaactgaacactgctgccggtaacaccgttgctggaggcactacgaccagtgtatcacagagtgagcgtcacgagtcagcgtcggtgcatgagtcgaagtcgttgggtgacaaggtcaaggacgcgcttggattcggttcaaagtag

SEQ ID NO: 264 - DNA, 780 bp, Milnesium tardigradum
atgaatcccacttctgagcatatctctgaaactacgacaactgtaaaaacgaccgataccggtgtcggactacagaatgtgtcggcgtcacaccacgcttccggtattcatcacgactcgagtgccgcttcgagcactgaatcgactaaattcgttcataccgaaacgaaggttcctatggccacaccagcgccacccattatttccgctgcaaccggtattgcggacagcattgtcagtgaaggaatgaccgcgtcggccgcgcgcatttctgccggagcgaatgaaagcatcgtacctgtggtggacacacaaaaggcggcggccgattatgacaaatatcagagagaagcggcggccatcgctgctgctcacgagcgtgacgttgcgaagaagactgaggcctatcgtaaagaaacggaggaacaggcagagaagatccgaaaagagctcgaaaagcaacacgcgaaagatatcgagtttcgaaaggatttggttgaggacgcgatcactcgtcagaaacgcgaaattgaattggaggcgaaaatggcaaagaaagaactggaacgcgaagctgaggctgcattggctgcgctcgacaagagtaaactgtccactgacatcgcagtcagcatcaacactgccgccggaagtacggtcgcaggaggcactgttacgacagtcactgagaagactgagagcactcactcacacgaacatgagcatgaacaccgaagcttgggtgagaaaatcaaagatacgcttttgggacgcaaatga

SEQ ID NO: 265 - DNA, 267 bp, Milnesium tardigradum
aaagagctcgaaaagcaacacgcaaaagatattgagtttcggaaggagattcttgaagatacgattgctcgacagaagcgtgaagtggaattagaggcgaagatggcgaagcgagagctggacagagaggcggcggccgctcgtgaagcgctagatcgatcgaagctcgcgacggatatcagtgtttcgattgataccgcagctggtcacactgttgccactgaaactatgaagagtactgagcatactttcagtcatcaacgcatg

SEQ ID NO: 266 - DNA, 444 bp, Milnesium tardigradum
atgagtcgaatcgctgaggaacacgagcgaaaggtcaaacagcgcactgaagcctatcgtaaagagacagaaatgcaggcagagaagctacgagtagagatgcagaaacagcacatccaggaacagcagtatcgacgtgagttgtcagaagcgaccattgcgcgtgaaaagcaggaggcacttctcgcgtatcgcgctaaactcactgagttagagcgcactcaacaggcactcaagtcggccgcagatcaggcaagactctcgtcagaaatcgaggtcaccatctctacgtccgccggcgaaactattaccggcatctctatagactcgaagagcgaagcatcgatgctcgagatgaacggcacacaaactcacgcgaaaagtcaagaagaagtgagatctttgggagataagctgaaaaccatagttctcggtcgcccgtaa

SEQ ID NO: 267 - DNA, 399 bp, Milnesium tardigradum
ggccattacggccggggagattatcggaagggactcgcggccaaacaggtcgactgccagaaacaacaagtcgaactcgaggtacaccattatttgatcataacagactacgattctacggcaagaaatctaagtggtaagttgatatttgttgaacaggcaaaaatggcgaaaaaggaactggaacgcgagctgactgcggctaaggaggctctagacgccacaaagtctgcgacaaatattcatgtgaatatcgaaactcttgctggagtcacgatggccggcgctacgacacacagccaaattacggaagttttggatgaaagcgaaatggataacgatcgcaaactgactttgggtcagaagatcaaagagaaactatcaaagggaaaattataa

SEQ ID NO: 268 - DNA, 138 bp, Milnesium tardigradum
atgccacacacacgcccacagccacagcgacgtccaggcgacctccactctgcacaccgaaatcagaacaccaatcatcgcacctgcagcaccagtaatggttacctcaactggagtggcttcagaaattattggtga

SEQ ID NO: 269 - DNA, 501 bp, Milnesium tardigradum
gacgaccggtcaagaagagaatttcgaggaatacctaagagcactcggtgtgcgaaagccctattctgcggaaatcgcggcaggaaagttgacggtcgaactgaaaaagaaggtgataagtaccaccacgtaatctgcttcccgcaagcagactaccatcaagatataccctttaagatcggtgaggccggtagtcaccaaatcaagaatactaccgtcaactatacgtatactctgaaaacgaaggatgataaacctgtgctgcatgctgacttcaaggcggacgcggccggcggccgaccggcaatggaattctcgagcaattttcactttagtgataccggatttgtgcatacttacaagaaagggaacgttaccgcaatgagaactttaaaaaggtttaggagtgtcttgtatctctgcgttgatctactctgttctgtgcgcattttcttctgcatctgtatacatatcttttgccattttttctgtttcagaatc

SEQ ID NO: 270 - DNA, 501 bp, Milnesium tardigradum; identical to SEQ ID NO: 269

SEQ ID NO: 271 - DNA, 510 bp, Ramazzottius varieornatus
atgtctcgcgcagctgtcgctatcgcccttctgggttgcgttgtggccgcttacggcgctccggctgaaggccacgacgatgccaaggcagaatggaccggaaagagctggatgggcaagtgggaatccactgatcgcatagagaactttgacgccttcatctccgcccttggtcttcctctcgaacagtacggtggaaaccacaagaccttccacaagatctggaaggagggtgaccactaccaccaccaaatcagcgtccccgacaagaactacaagaacgacgttaacttcaaactcaacgaggaaggaacaacccaacacaacaacacggagatcaagtacaagtacaccgaggatggcggaaacctgaaggctgaagtccacgttccatcccgaaacaaggttatccatgacgaatacaaagtcaatggagacgaactcgagaagacctacaaggttggagatgtcaccgccaagagatggtacaagaagagctcttcgtcgtag

SEQ ID NO: 272 - DNA, 525 bp, Ramazzottius varieornatus
atgcatcgatttgtccttgctctcgtcgtttttgcaggtgctgccatcgtctgggccgctgatgacgctgctcacgaagaaggcgtagaatggactgggaaaccgtggatgggcaaatgggaatccgacccatcgaaggacgagaacgttgaggaattcaaaaagaagctccagcttccgatgagccactcggaaatgaacaaaaactccaaagtttggatccatcactacaagaagggagacgagtaccatcacaaaatcatcatcaacgacgcccattacaaaaacgatatcgtcttcaagctgggtcaagagtccgccggttcgtataacggctcatctttcagcgtgaagtacgaggacaaagacggcgctctagtcggaagcgtccactacactggcaccaaagaacagtctcttgacaagaccatcaacaacgtcttcaagctcgaaggtgaccatctggttaagacttccaccatcgagggagtgaccatgaagcgccactacaacaaacgccagtga

SEQ ID NO: 273 - DNA, 537 bp, Paramacrobiotus richtersi
atgacctttaaggtgtttattttgattgcacttgtggcggccgtcaaagcccgaccggccgagggcgaacacaaggatcagcaagacattgccgctgacgccgaccatccctggattggcaaatgggaatccattgacgggcgccaggaaaactttcagaacttcatcaatgccttaggcttcgcacactacacacacgagcacaaagtctggcacaaactgtggaaagagggcgatcactatcatcaccgcatcaaagtcccggagaagggttacaagctcgacgttgagttcaagctgggagaagaaggaaccggtagctacaataatacccagttcaagtacaaatacaccgaagagaataaagatttacatgtggagatcaacctggtcacgcacaacaaggtgatcaaggacgattaccacgtggaaggcgaggagctggtcaagacctacaaagtcggtgatgtcacggccaaacggtggtacaagcgcgcccagaagaagccgaaagcggaggcggcggccagtgcataa

SEQ ID NO: 274 - DNA, 537 bp, Paramacrobiotus richtersi
atgacctttaaggtgtttattttgattgcacttgtggcggccgtcaaagcccgaccggccgagggcgaacacaaggatcagcaagacattgccgctgacgccgaccatccctggattggcaaatgggaatccattgacgggcgccaggaaaactttcagaacttcatcaatgccttaggcttcgcacactatacacatgagcacaaagtctggcacaaactgtggaaagagggcgatcactatcatcaccgcatcaaagtcccggagaagggttacaagctcgacgttgagttcaagctgggagaagaaggaaccgggagctacaataatactcaattcaagtacaaatacaccgaagagaataaagatttacatgtggagatcaacctggtcacgcacaacaaggtgatcaaggacgattaccacgtggaaggcgaggagctggtcaagacctacaaagtcggtgatgtcacggccaaacggtggtacaagcgcgcccagaagaagccgaaagcggaggcggcggccagtgcataa

SEQ ID NO: 275 - DNA, 507 bp, Paramacrobiotus richtersi
atggttttgcttgctgctttattattcggcgtggtgacgtgcgttgcctgccatggacaagctgatcccaaaaccatcccagcggatccagatcatccatggatcggcaaatgggaatccatttctgatcgtcacgagaactttgacaattttgttcaacatctcggcatggcacattacaaatctgagaataaagtctaccacaaattctggaaggaagaagaccatttccaccacggaatcattgttcccgataaaaatttcaagcagtttcttgaattcaaattaggcgaacaaggcacgctgacgtggaatggtaccgactttaagtataaatacacggaacaaaacaaggatcttcatgtggaagtgaacgttccatccaagaacaaggtgatccatgacgtttaccatgtggagggggaagagatggtcaaaacgtacaaagtggacactatcgaagccaaacgatggtttaagaaggcaccggcagagagcattttataa

SEQ ID NO: 276 - DNA, 507 bp, Paramacrobiotus richtersi; identical to SEQ ID NO: 275

SEQ ID NO: 277 - DNA, 525 bp, Paramacrobiotus richtersi
atgtcgtaccttgctttcgtcctcttaggactggcggtgttcgctgctgctgaccatcatgacgaaagcggaagcgatcccaaggagattcctgctgacccggcacacccttggatcggtaaatgggaatccatcgagggacgttcggagaattttgccaactttgtcaagaagttagatgctcccattaactactccgatgacatgaaagtctaccacaaactgtggaaggagggagaccatttccaccacggaattgctattcccgacaagcagttcaaaaagttcttccagttcaagttgggcgaagaaggaagcttcacttttaacaacactgagttcaagtacacttataccgagaaagataaggatctgcatgctgaagtgaaatgcccatccaagaacaaggtcgtccacgatgtctatcatgtcgagggtgaggagctcgtcaagtcctaccaggttgatgatgtcaaagccaagaaatggttcaagaaggccgcgtccaaacccgccaacgcctag

SEQ ID NO: 278 - DNA, 525 bp, Paramacrobiotus richtersi; identical to SEQ ID NO: 277

SEQ ID NO: 279 - DNA, 522 bp, Paramacrobiotus richtersi
atgaagtggttaatcgttgttgtattaggcatttctgcggccctggcggaggaccatccgacgccaaataacatcccactggacagtgcccaccaatggatcggtaaatggaaatcgactggacgccatgagcatttcgatgacttcatgaaggctttgggcctaccgaatcacgatgtggccgatccggaaaccacccatgtgttatggaaagaaggcgacaaatttcaccacaaaatctccgcaccgtctgtcaattacaagaagcatatctgttttacgttgggcgaggaaggaaacagctcctataatgggaccgcatttacgtacaagtataccgaactaccggacaaagatctggtgctggtagccacgcttccgtcgtacaacaagtcagtccatgccaccttccacgcgacggggaatgaactgatgaagaccttcaaagttgaccaggtggtcgccaaacgctggtatgctcgtgtggaccagactgccgctccaaagcccgccgcaaagtaa

SEQ ID NO: 280 - DNA, 522 bp, Paramacrobiotus richtersi; identical to SEQ ID NO: 279

SEQ ID NO: 281 - DNA, 465 bp, Paramacrobiotus richtersi
atgcaggtttccagtgttttattcgtccttggttgcgtgattgttaccatcgaaggcggtggcttacatcagttcttgggaaaatgggaatccacggagaagagggaaaatacccaggctttcgctgaagcgttaaatcaggtggatcaggtggatataaactcgaaaatcttcaatgagttctcgctggatcaggcgagtgctgatggataccaccacaaattttccgtccccgacaagaattacgtgcaagatgtaactttcaaactgggcgtcgaggggcaaaagactttcaatggaacaacctataagtacaagtatacactggatggtgataccctgaaatcgcactttgaacttcccgatcgacaagtcgatcaagagttcagtttggtcaacaacgaactggtcaagacgtacaaagtcaacaatgtcgtcgccaaggtctggtttaaaaaggtttaa

SEQ ID NO: 282 - DNA, 453 bp, Paramacrobiotus richtersi
atgaatgtttgcattgcgtccttgtgccttggctgcctgattgttgctgtcgaaggagctggcttaggcatttttatggggaaatgggaatccactaacaagagggaaagcacccaggcattcgcggaagcggtcgaacatgtggatatcgactcaaagatcgtcaacgaattctcggtgaagaatggtggggaggaataccaccacaaattttccgtaccggataagaattacattcaagatctaccttttaaactgaacgaagaacgtcagacgacctttaatggaacaacctacaagtacaaatatacactggagggcgacactcttaaatcgcactttgaactgccggatcgtcaagtcgaccaggagttcaatttggtcagcaacgaactggtcaagacatacaaagtcaataacgagtccgctaaagtctggtttaagaaggtctag

SEQ ID NO: 283 - DNA, 543 bp, Paramacrobiotus richtersi
atgttgcttctacttgttgataaacattcttttcgggtcccgaacaagaagtccatttgttgtaaccgtattacgattgtcttttctgccatgaatgtttgcattgcgtccttgtgccttggctgcctgattgttgctgtcgaaggagctggcttaggcatttttatggggaaatgggaatccactaacaagagggaaagcacccaggcattcgcggaagcggtcgaacatgtggatatcgactcaaagatcgtcaacgaattctcggtgaagaatggtggggaggaataccaccacaaattttccgtaccggataagaattacattcaagatctaccttttaaactgaacgaagaacgtcagacgacctttaatggaacaacctacaagtacaaatatacactggagggcgacactcttaaatcgcactttgaactgccggatcgtcaagtcgaccaggagttcaatttggtcagcaacgaactggtcaagacatacaaagtcaataacgagtccgctaaagtctggtttaagaaggtctag

SEQ ID NO: 284 - DNA, 594 bp, Paramacrobiotus richtersi
atgaaaaaatttgaagctttgttcagaaatccggccagtaaattcagcgcacacatcaaattttccgataacatgaggtatattggactgctgcttttaggactggcagcatgttcgcgtttggagccagaacaaggcagccacatgtctttaaaagatatccatccagatccggaacatccctggatcggtagttgggaatccatcgaaggccgctttcaaagtgttgatactgaccgcaacgaaatcggtattgcaccttatatgaacgacgccaataccaaagtgtatcgtcagttctggagagaaggcgaccatttctatcacgtcgttgcagcacctgagcgcggttttcgaaccgaattccggtttagattgggtgaagagagtgtggtcatcctaaatggcaccgaatacaaatttatttattccgaaaaaggcaaggatttacatgcaatagtaaagatcccttccacgagcactgttttcactgatgtttatcacgttcaaaacgaagatatgctcaagacgttcacccgaggagctgtgcaagccaagcgttggttcaagaaaatcaaatcccagccatcttaa

SEQ ID NO: 285 - DNA, 456 bp, Paramacrobiotus richtersi
atgcaggtttccagtgttttattcgtccttggttgcgtgattgttaccatcgaaggcggtggcttacatcagttcttgggaaaatgggaatccacggagaagagggaaaatacccaggctttcgctgaagcgttaaatcaggtggatatcaactcgaaaatcttcaatgaattctcggtggatgaggcgaatattaatggataccatcacaaattttccgtacccgagaagaaatacgttcaagacgtaacttttaagctgggcgaagaaggtcaaaagacttttaatggaacaacctataagtacaaatatacactggatggtgacaccctgaaatcgcactttgaactccccgatcggcaggtcgatcaagagttcagtttggtcaacaacgaactggtcaagacgtacaaagtcaacaatgtcgtcgccaaggtctggtttaaaaaggtttaa

SEQ ID NO: 286 - DNA, 513 bp, Paramacrobiotus richtersi
atgaggtatattggactgctgcttttaggactggcagcatgttcgcgtttggagccagaacaaggcagccacatgtctttaaaagatatccatccagatccggaacatccctggatcggtagttgggaatccatcgaaggccgctttcaaagtgttaatactgaccgcaacaaaatcggtattgcaccttatatgaacgacgccaataccaaagtgtatcgtcagttctggagagaaggcgaccatttctatcacgtcgttgcagcacctgagcgcggttttcgaaccgaattccggtttagattgggtgaagagagtgtggtcatcctaaatggcaccgaatacaaatttatttattccgaaaaaggcaaggatttacatgcaatagtaaagatcccttccacgagcactgttttcactgatgtttatcacgttcaaaacgaagatatgctcaagacgttcacccgaggagctgtgcaagccaagcgttggttcaagaaaatcagatcctag

SEQ ID NO: 287 - DNA, 504 bp, Paramacrobiotus richtersi
atgtttgcctcactcgtgatttttgggttagtagcggcttgtgcgaatgccgcaccggccgatcaagccaatcagagcagccactccgaccattcccacgcctggctgggaaagtgggaatccactcccgaaggcgaggaaaacatgcaacaacttgtcgaccagattaaagacgccattcctcattataccaccaagaaaatcacccacgagtatatcgagcagggcgatgaatttgtacataaagtccaaatcgaaggtggtcaaaattacgaagtgaaatttaaactcaaccaggaacattcatggcaccttggagaggaaccggaaattaagtataaatatacggaagaaggaccaaataaactcaaagtccatatgaatatcccttcaaagaataaggagctgaaggaatgctataacgttgaaggagacaaaattaacaaggagtacgaatctggaagcgtcaaagctaaacgtgtttacaagaaagtgcagaaatga

SEQ ID NO: 288 - DNA, 504 bp, Paramacrobiotus richtersi; identical to SEQ ID NO: 287

SEQ ID NO: 289 - DNA, 543 bp, Paramacrobiotus richtersi
atggcgcaaatgctgttcatcataggatttctgtgttgcgcgggcgtgtacggctcgcagtccgttgggcgtgttcatatgaaggatcattacgggaaccgcggggacagtttcgagaacgtcgcgcaccagtggctgggcaagtgggaatcagtggaaggaactgaagaaaacttcgaccagctcttggacgctatccgtgaagcgttcccgtattacagccaagcaacaatcatccatgatttcagcaaaaagagcgatgacgagtttatccacaagataaaaataggcagcgacgaagatcattatcagctgacgttcaaattggaccaggaaggtaccctacgcaagccaggcgcgccggaaatgaagtacacatacgaggaagtttcgggaaacaagctggtagtccaacaaagcgtcccatcaaagaatattatgctggaggaaagttacaaggttcagggtgatcagatccttaaggaatatgcaaccggaggtgtccgggccaagcggacgttccaaagaatgaaccatttgtga

SEQ ID NO: 290 - DNA, 543 bp, Paramacrobiotus richtersi
atggcgcaaatgctgttcatcataggatttctgtgttgcgcgggcgtgtacggctcgcagtccgttgggcgtgttcatatgaaggatcattacgggaaccgcggggacagtttcgagaacgtcgcgcaccagtggctgggcaagtgggaatcagtggaaggaactgaagaaaacttcgaccagctcttggacgctatccgtgaagcgttcccgtattacagcacagcgacaatcatccatgatttcagcaaaaagagcgatgacgagtttatccataagataaaaataggcagcgacgaggatcattatcagctgacgtttaaattggaccaggaaggtaccctacgcaagccaggcgcgccggaaatgaagtacacatacgaggaagtttcgggaaacaagctggtagtccaacaaagcgtcccatcaaagaatattatgctggaggaaagttacaaggttcagggtgatcagatccttaaggaatatgcaaccggaggtgtccgggccaagcggacgttccaaagaatgaaccatttgtga

SEQ ID NO: 291 - DNA, 570 bp, Paramacrobiotus richtersi
atgactggtgtgcccaggcctagcagcgcgtattttgttattgcgttctattgctttagctgtgtgacagcggaatcaacagaaactactccgccgcggggttccggaaatgggacaagtattgcggtcgaagcagctaaacctgtgttgatacctttcggaaaatttgaagccacagatcaagtggaaaatttcgccagttatttgtcaagtttacgcgtggaattcaaagggttttccgccggaaatttgaaaggaaaggtccaacatgaatttagtcgtgcacccgacaacaaatattcccatgctttctggattgctggcaccccatataaacagaagcttagttttgaacttggaaaagaacatcagcaaacatataatggaactggcttcaagtaccgatattatcaagaacccagtcaactcggtctacatgctgtattccacgttccggcggataatcccttacctatcgaacatctgtacacaaccagcccggacggttttgttttgacctataaaattggagacgtaacagcgaagcgcgcgtacaaaaggattccgtaa

SEQ ID NO: 292 - DNA, 519 bp, Hypsibius dujardini
atgtctcgaacgatcgtcgccttgatcctcctcggccttgctgcgcttgccgcagcggaccaccatgaaggtcacggagcggaaaaagaatgggcaggcaaggcttggcttggaaaatgggtgtccaccgatcggtcagaaaattgggacgcctttgttgaggccctgggtcttcctctggcggcctatggcggaaatcacaagaccgtccacaagctctggaaggagggtgatcactatcaccatcaaatcatcattgcggacaagtcctacaagcaggacatccagttcaagctgggcgaggaaggccggaccgcgcacaacggcacggaagtcactttcaagtacaccgaggtcggtgacaacctccaaaacgaagtcaagatcccctccaagaacaagaccatctccgactcgtacgtcgtgaaaggagacgaactcgagaagacgtacaagatcaatgatgtcgtcgcgaagcgctggtacaaaaagcacgcccacgagcccagcacagcttga

SEQ ID NO: 293 - DNA, 519 bp, Hypsibius dujardini; identical to SEQ ID NO: 292

SEQ ID NO: 294 - DNA, 492 bp, Hypsibius dujardini
atggctcgccttttcgtcgctgtcgcccttttcggtgtcgtggctttcgctgctgcggaaaaagaatggactggaaagacctggcttggctcgtgggcgtccaccgaccgcgcggagaactgggaagccttcgtcgatgctctcggtctgccgtccgatcagtatccccgtgaggtccagcggaccatccacaccatctacaagcagggcgacaagtaccaccacgaggtgagcatcccctcgaagaacttcaagaaggccatcgagtacacccttggcaccgagaccgacgtccaacacggcccgcacaccatcaagctcaagtacaccgaggacggtgagaagctcgtcgctgacgtgcagatcccctccaagaacaagcaaatccacgacatctatgaagttcagggagacaccctcacgaagacgtacaaggtcggtgatgtcgtcgccaagcggtggttcacgcgggaggccaatcccactgcttaa

SEQ ID NO: 295 - DNA, 492 bp, Hypsibius dujardini; identical to SEQ ID NO: 294

SEQ ID NO: 296 - DNA, 498 bp, Hypsibius dujardini
atgtctcgagtcctcgtcgccctcgctctctttggtgtggtggctctggccgcagccagtggcgatgcgcaaaaagaatggaccggcaagtcgtggcttggaaaatggcagagccttcccactgacaagtccgagaactgggaggcttttgttaatgccctcgctattcccgaacagtacacgcgtgatctccagaagaccgtccacactttctataaacagggtgaccactaccaccacatctttgccattcccgacaagaactttgagaagaacattgagttcaacctcggcgcggagtcgtcggccaagcacggcgaacacgaggtcaagatcaagtacgccgaggatggtgacaagctcgttgctgatgtgcgcattgccgcgaagaacaagcacattcatgacgtctacgaggttcaaggcgaagaactcgtcaagacatacaaggtcggcgacgttgtcgcgaagagatggttcaagaagatcgctcaataa

SEQ ID NO: 297 - DNA, 498 bp, Hypsibius dujardini; identical to SEQ ID NO: 296

SEQ ID NO: 298 - DNA, 507 bp, Hypsibius dujardini
atggctcgcttcctcgtcgctctcgctcttttcggtgtggtcgccatgaccgccgcctctggtgatgcgccaaaagaatggtctggaaagccttggcttggtaaatttgtcgctgaggtctcagacaagtctgaaaactgggaagccttcgttgatgctcttggtctgcccgatcagtatccccgtgcccagctgaagaccatccactcgttctacaagcagggtgagcactaccaccacattctctccctgcccgacaagaacatcaacaaggacattgagttcaccctcggccaggaggttgagatcaaacacggcgagcacagcctgaagatcaagtacttcgaggacggtaacaagctcgtcgctgatgtttcaattcccgccaagggcaagtcaatccatgatgtgtatgatgttcagggagatcagctcatcaagtcgtataaggtcggcgatgtcgtcgccaagaagtggttcaagaaggtcgccaaccctgctgcctaa

SEQ ID NO: 299 - DNA, 525 bp, Hypsibius dujardini
atggctcgctttctcgtcgccctcgcccttttcggtgtggtcgcaatgaccgccgccactggtgatgcgccaaaagagtggtctggaaaaccttggctcggtaaatttgtcgctgaggtcacagacaagtccgagaactgggaagccttcgtcgacgcgcttggtctgcccgaacaatttggtcgtgccccggtgaagaccatccaaaaaatctataagcaaggtgaccactaccatcacatcttcgcccttcccgacaagaactttgagaaggacattgagttcactctcggccaggaggtggagatcaagcaaggcgaacacatcgcgaagaccaagtactccgaggacggtgagaagctcgttgctgacgtctcaatccccaccaagggcaagaccatccgtagtgaatatgaggtccagggagatcaactcatcaagacatataagaccggtgatatcgtggccaagaaatggttcaagaaggtggccaaccccaccgaggcccctgcccaagcagcctag

SEQ ID NO: 300 - DNA, 507 bp, Hypsibius dujardini; identical to SEQ ID NO: 298

SEQ ID NO: 301 - DNA, 519 bp, Hypsibius dujardini
atggctcacctcacaattctccttgccctgtctgttaccggttttttcgtctcgaccgtggcggatcataaggcagagaaacacggcggcaagttcgatgggaaatcctggctgggaaaatgggagtccaccaaccataccgagaacctcgagaccttcgtcagccaacttggttatccgtctgcggagcacgtgacggaccagaaggtattccaaaagttctggcaggatggtgagcatttccatcataagatcaccgtaccgaccaagaactacaccttgcaacacaagttcacattgggccagccgggaaaggcaactttcaacaatgtcgaattcaagtacctctacgccgagctgggaaacgacctccacgttgagatcaccgtgccgagcaagaacaagaccgtcagtgacacctaccatgttttccaaaatggaaccgagttggaaaagacctacaaaacaggagacacagtggccaagcgttggtacaagaaagtgatcagctgccactga

SEQ ID NO: 302 - DNA, 519 bp, Hypsibius dujardini; identical to SEQ ID NO: 301

SEQ ID NO: 303 - DNA, 471 bp, Hypsibius dujardini
atggctcgtctgtccctcatcgttcttatgggtgtcgtcgctgttgcatcggcctcgcagccgtggctcggaagctggaccaccaccgataaggcgcccgagaactgggatcaggtcgtggcggctcttggtctgcccgcagcgtacggcggcaaccccaaatccactctgagcatcactcgcgagggagagacctacaccagcaaactggaagtcccctccaacaacttctccagcacgtggaccttcaagatcggcgaggaaggcaccaaggtggagcccaagtttgagaacaccgaggtcaaatacactttcaccgaggagggcgagaaactgctggtgacggtgaagatccccgctaggggcaaggaagtcaccgaggtttacgaagtgaccggcgatgagctcgttaagacatacaaaatcgacggaatcgtcgcgaagcgatacttgaaaaggcaagctgtttaa

SEQ ID NO: 304 - DNA, 471 bp, Hypsibius dujardini; identical to SEQ ID NO: 303

SEQ ID NO: 305 - DNA, 474 bp, Hypsibius dujardini
atgtctcggattctccttgtcctcgccctcttcgttatggtctctgttacatcggctgcacagccgtggttgggtgtctggaccaactcggagaaggctcctgagaactgggatcagtttgtggcagctcttggcctccccctggagcagtttagcggcaacccaaaagccaccatcaccatcacccgcgacgacggagacaactacaaggtcttactggatgtgcccgcaatcaatttcactagcacctggaacttaaggctgggcgaggaaatggttatggatgagtttggttcggggatgaggtacaacttcactgaggacggagacaaactgcaagcccacgtcaaaatatccgccatcggcaaacaatacaacgaaaactacgaagtggtcggtcaagagctcattataacatacaagatggatggaatcgttgctaagcgtttcttgaaaagggaccaatcttcctga

SEQ ID NO: 306 - DNA, 486 bp, Hypsibius dujardini
atggcagcaattgatcccacaccggcaacagtattgagtgtccagcaagagaattgcaggccatggctcgggatgtgggtttccgctggaaagaaagaaaactggcccgcagtcatggaagcgttaggtttgccggagatgtattctgagaaaaacactttcgtcctcaaattatggtgcgacggagaggactttcactatgatgccggtattctggaggcaaagtttaagcacagtgtcacgtttaagctggggactcctactgaacttaatcacgggaacaaaatcgtcattacctacaccgaagaggacggcaagctgatcgcggacggagtaattgcggctaagaatctgattttgcacaacgtattcgcggcccagggagacgtgctgatcaagacctatcgtgtggggaatgtcgtagccaagtcatggtaccgcagactttcctcaacggccgactcaaacattttatcctttttgtga

SEQ ID NO: 307 - DNA, 465 bp, Hypsibius dujardini
atggagttcgcggcgtccatcttcgttctctgcttcggtctctcggctgtcacagcagccggtttgccgtttgtcggacactatgtgtcgacgggtcagcgctttaacactgctgcgtttgccgcggccaccggtttcgatgatccacccgtggaaaaccggctgcacaacgaattcctggaccaagggaacggcgagtacctctacaaatttcgcgtcgaaaatgccgcctataagcaggagctgccgttcaaactgggcgagacacgcaagtccacctacaacggaactgaattttcgtataaattcaccgtcgatggcgagctgctcaaatttgagtcgaagatccttcccgacggacgcgaagtcacccacacttactatcccaatgccgacgggttcgttaagcaattccaactgaaggacgtcatcgccaaggtgtggttcaaaaaggactctgcatag

SEQ ID NO: 308 - DNA, 465 bp, Hypsibius dujardini
atggagttcgcggcgtccatcttcgttctctgcttcggtctctcggccgtcacagcagccggtttgccgtttgtcggacactatgtgtcgacgggtcagcgctttaacactgctgcgtttgccgcggccaccggtttcgatgatccacccgtggaaaaccggctgcacaacgaattcctggaccaagggaacggcgagtacctctacaaatttcgcgtcgaaaatgccgcctataagcaggagctgccgttcaaactgggcgagacacgcaagtccacctacaacggaactgaattttcgtataaattcaccgtcgatggcgagctgctcaaatttgagtcgaagatccttcccgacggacgcgaagtcacccacacttactatcccaatgccgacgggttcgttaagcaattccaactgaaggacgtcatcgccaaggtgtggttcaaaaaggactctgcatag

SEQ ID NO: 309 - DNA, 624 bp, Hypsibius dujardini
atgatcagtttatttttgttattcgcagtaggtgggctcgcggttgacggggcgcttccacccggcgaggttgcagccgtgttactaccccccagcatggtgaatatcataccggtaccactgggagagtttgttcccactgggcagaaggaaaattacgccaactacgtgcacagtttggagtttgagttccgcgggctggctgcgcagggtattcttggagacaagggcaaggatgtgcggcataaattttcacggagtgccgatgggaaggagaactcgtacgtccacaagttcggcaatgacggtggtggcaaatacaaccacaccgtgccgttcgtgctggacgaggagaaactcgtccataccaatgcgacatccttgaagtacaagtattggttcgagcccggacaaggacttcatgccgactacaacatcccaccggagaatcccctgcagattcagcatctttatgccgtgacagacgagggtttcacactgatctacaagctgggaaatgtcattgcaaagaactattacaaacgtgcaccttcatcggatgctgcaccagaagtcacgtctaagacaaccgttgctccgatcaccacaaagaaaaaagcataa

SEQ ID NO: 310 - DNA, 624 bp, Hypsibius dujardini; identical to SEQ ID NO: 309

SEQ ID NO: 311 - DNA, 711 bp, Ramazzottius varieornatus; identical to SEQ ID NO: 206

SEQ ID NO: 312 - DNA, 744 bp, Hypsibius dujardini
atggccaaatatttgctgcacgacatgcaagccatgctgcgtgggatcaagcaggtcgctcagatcagtctgaaaatccaagccgcggaaatcaacgagcgtgtgtgtcaatccagcctgcgaccgcgatggagcaatcttgcgagctcttccgcgagctctcctgcgtcgtcttcatccccgcggtccagcttcaacgtacaggaaattgcgtcgcgtgccggtgcggtgctgcgtgggctggaggagcaagtgaaaatcgtcgcgggcatccaggccccggctcccattttggccttcgataacggctttaccctctacagcgacaaaattggaagcgcacagaatagggccacacgcgaccatccgaccacggcggatattgacgacgagaatggacatggcaagccggaaggcgaggccggaaaggcggcgaaacgcgcggagaaatttatgaacccaccggttgcaccattggacgaaagtgacgtctcagttcttgctaataactcgctcgagggtgatgactctcacaacctgaagaactttaataacggaagtttggacgctgcggaggctgaaggcaaggaagagacgtcccacctcaagcaggatcgcttcagtaaggactccaagaagaccttcatcgacagcggcggggacaacttattccgaccggagaatttgaagaaaatttcaaaggttccaccgggcgtcccagtcaaggctgacagcttttcttag

SEQ ID NO: 313 - DNA, 744 bp, Hypsibius dujardini
atggccaaatatttgctgcacgacatgcaagccatgctgcgtgggatcaagcaggtcgctcagatcagtctgaaaatccaagccgcggaaatcaacgagcgtgtgtgtcaatccagcctgcgaccgcgatggagcaatcttgcgagctcttccgcgagctctcctgcgtcgtcttcatccccgcggtccagcttcaacgtacaggaaattgcgtcgcgtgccggtgcggtgctgcgtgggctggaggagcaagtgaaaatcgtcgcgggcatccaggccccggctcccattttggccttcgataacggctttaccctctacagcgacaaaattggaagcgcacagaatagggccacacgcgaccatccgaccacggcggatattgacgacgagaatggacatggcaagccggaaggcgaggccggaaaggcggcgaaacgcgcggagaaatttatgaacccaccggttgcaccattggacgaaagtgacgtctcaattcttgctaacaactcgctcgagggtgatgactctcacaacctgaagaactttagtaacggaagtttggacgctgcggaggctgaaggcaaggaagagacgtcccacctcaagcaggatcgcttcagtaaggactccaagaagaccttcatcgacagcggcggggacaacttgttccgaccggagaatttgaagaaaatttcaaaggttccaccgggcgtcccagtcaaggctgacagcttttcttag

SEQ ID NO: 314 - DNA, 732 bp, Paramacrobiotus richtersi
atggcacgattcatgataaaagatttgcaggcggtatttcgcggtttccagcaggtcgcgcaaagcagcgtggagcatcagctcaccgaaacagctctccgatggcatacgctgagtctgcgcccactggttcaaggatgcgtgaaccgaatgcaagaatctcagaggtcgaccgttccgctgcgagaattccctgcccgagtgggagctgtagtgcagggtattcaagagcagatgaagatcttggcgggtttcccctctcccgctctggtcacgccggagggattcgtcttctacaccgataaagtcaataaagatacccataaggaatatcccgctgtggctgatgaggtacactcggccaaactgcagggactgaaaccggaaagcggggaagcatgtgaagcggccaaacgtgccaaagagtttatgaatccaccagtgtcgccactggatccggaggataaaaacgaggttgtacgcacaccggaaatgtctggttcgaccgctgctgaggatcagaatgctgacgaatccggcaaagctgcgaagagactaggaaaattcatgaatgaggagatcgcacctgaaagtaagcccttcaaaccgtttgccaaagattcagccaagaccacagtgtctttcacggacgctacgggcgaaaacttccgcatacaggatttgaaaaaggttcaagtttttcccggcacacccgttgccttcgagagttga

SEQ ID NO: 315 - DNA, 819 bp, Milnesium tardigradum
atgtcgcgatatttactcaacgatatggaaggcatcatcagaggtcttcgaagtgtcgctacaaatgcggcagcacttcacagaactgacctctcgacgcgtctgcagaactgcacatttgcagctcagtcgtccaacgcagtgccgtcactgttgcgtcaaatgcagaaagtacgaccaataaacaccacagacttcgtctcacgtacacgcacggttttacgaggtttgcgcgatcaatcgcagtccctcttcggctcttcgcagtctatccgtcatacgtcaggcacagcatcacccacaactaacgtcgtaacgaaaactgagaaagaaatgaaagaagcacagtcgaaagagcgaaattcgaaggatactcactcgaaagacagttacaacaaagatgcgcacatcacgccctccagtgactctaccgcttcaaacgctgcttccaacaaaagagacgcaaagaaaagtgatgacacaaatggtggagcgcgaatggtcgacgaaggcgcttttaacaacgaaaaaccaatcaaacagagctctgctaaggaccacagagaccagtcaccgcataaccgagaattggctgatgtcgaaaggaagtcagaagtggaaatgccagaggactctgaagatcagaagctaacagaggccgaacaggccgcaaagagaatcgaagaatttttgaacggaccgaaatcacctgaagatcccgcctctaaagataaaatcgtcgtaacaccagaaatgacgaaacatgaagagccgataccagaatcgaaggcagttcaggaaatatggatagaattttga

SEQ ID NO: 316 - protein, 5 aa, Artificial
  Poly-Args tag sequence
RRRRR

SEQ ID NO: 317 - protein, 6 aa, Artificial
  Poly-Arg tag sequence
RRRRRR

SEQ ID NO: 318 - protein, 6 aa, Artificial
  Poly-His tag sequence
HHHHHH

SEQ ID NO: 319 - protein, 8 aa, Artificial
  FLAG tag sequence
DYKDDDDK

SEQ ID NO: 320 - protein, 8 aa, Artificial
  STREP-TAG II sequence
WSHPQFEK

SEQ ID NO: 321 - protein, 10 aa, Artificial
  C-myc tag sequence
EQKLISEEDL